SEQUENCE LISTING (132 sequences)

SEQ ID NO: 1 - DNA, 1710 bp, synthetic construct
  source 1..1710: mol_type = other DNA
atgaatcaatcggaattgattacactggcctccgcgaatgctctgaacaaagactttgaagtaaaatatcaaaacgtgatttccgacttcttttcccgtgaccctggaaaatggcccattttcaatcacccccaaatccaagcaattacacagttccgtcagaccacagatgcggatgtacagcagatcaatcgctaccctcagggtaaggacttgttcgcgcaactggcaggcgaatcgcacgtccgtcagaacgttatccgtccaggtgaaggtcaggacgatttacttgtgttcgcatcagccttatgcaaaaattgggagaaccccttagccgtagagaacgtcattgctatgccaagcgacccagcggtctatggctccatgttgggtttattaggaaatccgaacatggtatactgtgaatattctggcgtagcagacaatatggagaaaaccgtgattcgtaaggtggcaaatttaattggatacgatgctgacaaagctagcggcttgttcacccaggggggaactatgtgtaatttgtatggatacttatttggtattcgcaaatcgcttaaacaaagtaagcatttaggcatgtcagtcgaccaggattttcgtattatcaattcgcaggggggtcactacagcaacatgactaatctttctcttcttggggtagacatcacgaacaaaactatccgcatccgtgtggccagcgataacactattgatcttgcggaccttgagcaacaaattcgtgcctgttacactgttcattgcaaaattcctgtcatcttgcttaccgctgggacaaccgatacgtttggtgttgacgaaatcaaacaagtctacgatctgcgcaatcgtctttgcgaggagttcgagatcacggagaaaccgcatatccatgtagacgctgcggttgggtggccaatcattttctttatcgactatgatttcaatacgaatccccttgctattaatgatgcaaccttggcagggttgcgccataatgtagagaaattcaagcaattaaaatatgcggattccattacaatcgacttccacaaatggggctacgtcccatacacgagctccctggttatggtgcgcgacggagacgatttcaaggcgttagaaaatgatcctgaaaattttacatattttgaacatgcgcttgaagggcaaacacatttacaatcaacaattgaatgtagccgctcgggcgtaggggtgttcggagcgtacgcgggtcttcactaccttggtgtagagggctatcagactatcattgcccactgtctgcaaaacgcgaattatatgcgtaaccagctgttatcaatggggaacgcctgcgtcatggtccctgagaaccaaggtccctccgtcggtttccgtttatactcaccaaaactggtcaacgacccgcaagctatgtttgctcacgaattaacgtgtgctggggataagaccgcttacgacatgatggtgcgtaattcacgttggcaccgcgagttattcctgaaacgcggaaaggccggtttgttcactaactgggtggactctatcgcttgtagtgcctatgctgagcacaaccgctttgcctatattccgggagaaaaggcagtcttcatgaaccctgttacccaacgccatcacatcgacgcgttcgctaagatgctgaagacgatgtcagccgagtaa

SEQ ID NO: 2 - DNA, 1710 bp, synthetic construct
  source 1..1710: mol_type = other DNA
gtgaatcagtcagaacttatcacgctcgcctctgcgaatgcgttaaataaagatttcgaagttaaataccaaaacgtcatcagtgatttcttcagccgggatcccggcaaatggccgatttttaatcacccgcaaattcaggcaattactcagttccgccagaccaccgacgcggatgtgcagcagatcaaccgctatccgcagggtaaggatctttttgcacaactggccggtgagtcacatgtccgtcagaacgtgatccgtccgggagaagggcaggatgatttactggtgtttgcgtccgcgctgtgcaaaaactgggaaaacccgctggccgtggaaaacgttatcgccatgccgtcagacccggcagtttacggctctatgctgggcttgctgggtaacccgaacatggtgtactgcgaatactccggtgtggcggataacatggaaaaaaccgttatccgtaaagtggccaatctgattggctatgatgcggacaaagcatccggtctgttcacccagggcggaaccatgtgcaatctgtacggctatctcttcgggatccgtaaatcgctgaaacagtcaaaacatctcgggatgtccgttgatcaggatttccggatcatcaactcacagggcggtcactactccaatatgactaacctgtccctgttaggtgtggatatcacaaataaaacaatccgtatccgggtggcgtcagataacaccattgacctggcggatctggagcagcaaatccgtgcctgctataccgtccactgtaagatcccggtgatcctgctgaccgcgggaacaaccgatacctttggtgtcgatgaaatcaaacaggtttatgatctgcgcaaccgcctgtgtgaagagttcgaaattacagaaaaaccgcatattcacgtggatgcggcggtcggctggccgattattttctttattgattatgattttaacaccaacccgctggcgattaacgatgccaccctggctggtctgcgtcacaatgttgagaaatttaaacagctgaaatatgcagattccatcactatcgacttccacaaatggggttatgtgccgtatacctcgagtctggtgatggtgcgtgacggtgatgatttcaaagcactggaaaatgacccggaaaacttcacctattttgagcatgcgctggaaggacagactcacctgcaatccaccattgaatgcagccgcagcggtgtcggtgtgttcggggcttatgccggattgcattatctgggggttgagggttatcagaccatcattgcgcactgtctgcaaaatgcgaactacatgcgcaaccaactgctatccatgggcaatgcctgtgtgatggtgccggaaaaccaggggccgagtgtcggtttccgtctctattcaccgaaactggtgaatgatccgcaggcaatgtttgcacacgaactgacctgtgccggagataaaacggcctatgacatgatggtgcgcaacagccgctggcatcgtgagctgttcctgaaacgcggtaaagcaggactgtttaccaactgggtggattctatcgcctgctcggcctatgcggagcataaccggttcgcgtatattccgggggaaaaggcggtctttatgaacccggtgacgcagcgtcatcacatcgatgcgtttgcaaaaatgcttaaaaccatgagtgcagaataa

SEQ ID NO: 3 - protein, 569 aa, synthetic construct
MNQSELITLASANALNKDFEVKYQNVISDFFSRDPGKWPIFNHPQIQAITQFRQTTDADVQQINRYPQGKDLFAQLAGESHVRQNVIRPGEGQDDLLVFASALCKNWENPLAVENVIAMPSDPAVYGSMLGLLGNPNMVYCEYSGVADNMEKTVIRKVANLIGYDADKASGLFTQGGTMCNLYGYLFGIRKSLKQSKHLGMSVDQDFRIINSQGGHYSNMTNLSLLGVDITNKTIRIRVASDNTIDLADLEQQIRACYTVHCKIPVILLTAGTTDTFGVDEIKQVYDLRNRLCEEFEITEKPHIHVDAAVGWPIIFFIDYDFNTNPLAINDATLAGLRHNVEKFKQLKYADSITIDFHKWGYVPYTSSLVMVRDGDDFKALENDPENFTYFEHALEGQTHLQSTIECSRSGVGVFGAYAGLHYLGVEGYQTIIAHCLQNANYMRNQLLSMGNACVMVPENQGPSVGFRLYSPKLVNDPQAMFAHELTCAGDKTAYDMMVRNSRWHRELFLKRGKAGLFTNWVDSIACSAYAEHNRFAYIPGEKAVFMNPVTQRHHIDAFAKMLKTMSAE

SEQ ID NO: 4 - DNA, 87 bp, synthetic construct
  source 1..87: mol_type = other DNA
aatgatacggcgaccaccgagatctacactctttccctacacgacgctcttccgatctnnnngggttgagatgtgtataagagacag

SEQ ID NO: 5 - DNA, 88 bp, synthetic construct
  source 1..88: mol_type = other DNA
aatgatacggcgaccaccgagatctacactctttccctacacgacgctcttccgatctnnnnngggttgagatgtgtataagagacag

SEQ ID NO: 6 - DNA, 89 bp, synthetic construct
  source 1..89: mol_type = other DNA
aatgatacggcgaccaccgagatctacactctttccctacacgacgctcttccgatctnnnnnngggttgagatgtgtataagagacag

SEQ ID NO: 7 - DNA, 90 bp, synthetic construct
  source 1..90: mol_type = other DNA
aatgatacggcgaccaccgagatctacactctttccctacacgacgctcttccgatctnnnnnnngggttgagatgtgtataagagacag

SEQ ID NO: 8 - DNA, 24 bp, synthetic construct
  source 1..24: mol_type = other DNA
caagcagaagacggcatacgagat

SEQ ID NO: 9 - DNA, 27 bp, synthetic construct
  source 1..27: mol_type = other DNA
gtgactggagttcagacgtgtgctctt

SEQ ID NO: 10 - DNA, 59 bp, synthetic construct
  source 1..59: mol_type = other DNA
caagcagaagacggcatacgagataacgatgggtgactggagttcagacgtgtgctctt

SEQ ID NO: 11 - DNA, 59 bp, synthetic construct
  source 1..59: mol_type = other DNA
caagcagaagacggcatacgagataagcgcaagtgactggagttcagacgtgtgctctt

SEQ ID NO: 12 - DNA, 59 bp, synthetic construct
  source 1..59: mol_type = other DNA
caagcagaagacggcatacgagataattgccggtgactggagttcagacgtgtgctctt

SEQ ID NO: 13 - DNA, 59 bp, synthetic construct
  source 1..59: mol_type = other DNA
caagcagaagacggcatacgagatacagctcagtgactggagttcagacgtgtgctctt

SEQ ID NO: 14 - DNA, 59 bp, synthetic construct
  source 1..59: mol_type = other DNA
caagcagaagacggcatacgagataccttggagtgactggagttcagacgtgtgctctt

SEQ ID NO: 15 - DNA, 59 bp, synthetic construct
  source 1..59: mol_type = other DNA
caagcagaagacggcatacgagatacgaaggtgtgactggagttcagacgtgtgctctt

SEQ ID NO: 16 - DNA, 59 bp, synthetic construct
  source 1..59: mol_type = other DNA
caagcagaagacggcatacgagatacgagatggtgactggagttcagacgtgtgctctt

SEQ ID NO: 17 - DNA, 59 bp, synthetic construct
  source 1..59: mol_type = other DNA
caagcagaagacggcatacgagatacgtcaacgtgactggagttcagacgtgtgctctt

SEQ ID NO: 18 - DNA, 59 bp, synthetic construct
  source 1..59: mol_type = other DNA
caagcagaagacggcatacgagatacgttcctgtgactggagttcagacgtgtgctctt

SEQ ID NO: 19 - DNA, 59 bp, synthetic construct
  source 1..59: mol_type = other DNA
caagcagaagacggcatacgagatactcgagtgtgactggagttcagacgtgtgctctt

SEQ ID NO: 20 - DNA, 59 bp, synthetic construct
  source 1..59: mol_type = other DNA
caagcagaagacggcatacgagatactgactggtgactggagttcagacgtgtgctctt

SEQ ID NO: 21 - DNA, 59 bp, synthetic construct
  source 1..59: mol_type = other DNA
caagcagaagacggcatacgagatagaccactgtgactggagttcagacgtgtgctctt

SEQ ID NO: 22 - DNA, 59 bp, synthetic construct
  source 1..59: mol_type = other DNA
caagcagaagacggcatacgagatagactgtcgtgactggagttcagacgtgtgctctt

SEQ ID NO: 23 - DNA, 59 bp, synthetic construct
  source 1..59: mol_type = other DNA
caagcagaagacggcatacgagatagagacacgtgactggagttcagacgtgtgctctt

SEQ ID NO: 24 - DNA, 59 bp, synthetic construct
  source 1..59: mol_type = other DNA
caagcagaagacggcatacgagatagcagaacgtgactggagttcagacgtgtgctctt

SEQ ID NO: 25 - DNA, 59 bp, synthetic construct
  source 1..59: mol_type = other DNA
caagcagaagacggcatacgagatagcatcgtgtgactggagttcagacgtgtgctctt

SEQ ID NO: 26 - DNA, 59 bp, synthetic construct
  source 1..59: mol_type = other DNA
caagcagaagacggcatacgagataggtgttcgtgactggagttcagacgtgtgctctt

SEQ ID NO: 27 - DNA, 59 bp, synthetic construct
  source 1..59: mol_type = other DNA
caagcagaagacggcatacgagatagtctcaggtgactggagttcagacgtgtgctctt

SEQ ID NO: 28 - DNA, 59 bp, synthetic construct
  source 1..59: mol_type = other DNA
caagcagaagacggcatacgagatagtggacagtgactggagttcagacgtgtgctctt

SEQ ID NO: 29 - DNA, 59 bp, synthetic construct
  source 1..59: mol_type = other DNA
caagcagaagacggcatacgagatatatcggcgtgactggagttcagacgtgtgctctt

SEQ ID NO: 30 - DNA, 59 bp, synthetic construct
  source 1..59: mol_type = other DNA
caagcagaagacggcatacgagatatcgccttgtgactggagttcagacgtgtgctctt

SEQ ID NO: 31 - DNA, 59 bp, synthetic construct
  source 1..59: mol_type = other DNA
caagcagaagacggcatacgagatatgcggttgtgactggagttcagacgtgtgctctt

SEQ ID NO: 32 - DNA, 59 bp, synthetic construct
  source 1..59: mol_type = other DNA
caagcagaagacggcatacgagatatgcttccgtgactggagttcagacgtgtgctctt

SEQ ID NO: 33 - DNA, 59 bp, synthetic construct
  source 1..59: mol_type = other DNA
caagcagaagacggcatacgagatatggaagcgtgactggagttcagacgtgtgctctt

SEQ ID NO: 34 - DNA, 59 bp, synthetic construct
  source 1..59: mol_type = other DNA
caagcagaagacggcatacgagatcaaccttcgtgactggagttcagacgtgtgctctt

SEQ ID NO: 35 - DNA, 59 bp, synthetic construct
  source 1..59: mol_type = other DNA
caagcagaagacggcatacgagatcaactgcagtgactggagttcagacgtgtgctctt

SEQ ID NO: 36 - DNA, 59 bp, synthetic construct
  source 1..59: mol_type = other DNA
caagcagaagacggcatacgagatcacactctgtgactggagttcagacgtgtgctctt

SEQ ID NO: 37 - DNA, 59 bp, synthetic construct
  source 1..59: mol_type = other DNA
caagcagaagacggcatacgagatcacttcacgtgactggagttcagacgtgtgctctt

SEQ ID NO: 38 - DNA, 59 bp, synthetic construct
  source 1..59: mol_type = other DNA
caagcagaagacggcatacgagatcagagacagtgactggagttcagacgtgtgctctt

SEQ ID NO: 39 - DNA, 59 bp, synthetic construct
  source 1..59: mol_type = other DNA
caagcagaagacggcatacgagatcagtagaggtgactggagttcagacgtgtgctctt

SEQ ID NO: 40 - DNA, 59 bp, synthetic construct
  source 1..59: mol_type = other DNA
caagcagaagacggcatacgagatcagtctgagtgactggagttcagacgtgtgctctt

SEQ ID NO: 41 - DNA, 59 bp, synthetic construct
  source 1..59: mol_type = other DNA
caagcagaagacggcatacgagatcatcgtaggtgactggagttcagacgtgtgctctt

SEQ ID NO: 42 - DNA, 59 bp, synthetic construct
  source 1..59: mol_type = other DNA
caagcagaagacggcatacgagatcatgcatggtgactggagttcagacgtgtgctctt

SEQ ID NO: 43 - DNA, 59 bp, synthetic construct
  source 1..59: mol_type = other DNA
caagcagaagacggcatacgagatccggttaagtgactggagttcagacgtgtgctctt

SEQ ID NO: 44 - DNA, 59 bp, synthetic construct
  source 1..59: mol_type = other DNA
caagcagaagacggcatacgagatcctacgttgtgactggagttcagacgtgtgctctt

SEQ ID NO: 45 - DNA, 59 bp, synthetic construct
  source 1..59: mol_type = other DNA
caagcagaagacggcatacgagatccttaagggtgactggagttcagacgtgtgctctt

SEQ ID NO: 46 - DNA, 59 bp, synthetic construct
  source 1..59: mol_type = other DNA
caagcagaagacggcatacgagatcgaataccgtgactggagttcagacgtgtgctctt

SEQ ID NO: 47 - DNA, 59 bp, synthetic construct
  source 1..59: mol_type = other DNA
caagcagaagacggcatacgagatcgatccttgtgactggagttcagacgtgtgctctt

SEQ ID NO: 48 - DNA, 59 bp, synthetic construct
  source 1..59: mol_type = other DNA
caagcagaagacggcatacgagatcgccataagtgactggagttcagacgtgtgctctt

SEQ ID NO: 49 - DNA, 59 bp, synthetic construct
  source 1..59: mol_type = other DNA
caagcagaagacggcatacgagatcgtaccaagtgactggagttcagacgtgtgctctt

SEQ ID NO: 50 - DNA, 59 bp, synthetic construct
  source 1..59: mol_type = other DNA
caagcagaagacggcatacgagatctagaggagtgactggagttcagacgtgtgctctt

SEQ ID NO: 51 - DNA, 59 bp, synthetic construct
  source 1..59: mol_type = other DNA
caagcagaagacggcatacgagatctaggtacgtgactggagttcagacgtgtgctctt

SEQ ID NO: 52 - DNA, 59 bp, synthetic construct
  source 1..59: mol_type = other DNA
caagcagaagacggcatacgagatctctgactgtgactggagttcagacgtgtgctctt

SEQ ID NO: 53 - DNA, 59 bp, synthetic construct
  source 1..59: mol_type = other DNA
caagcagaagacggcatacgagatctgatgtggtgactggagttcagacgtgtgctctt

SEQ ID NO: 54 - DNA, 59 bp, synthetic construct
  source 1..59: mol_type = other DNA
caagcagaagacggcatacgagatcttcagctgtgactggagttcagacgtgtgctctt

SEQ ID NO: 55 - DNA, 59 bp, synthetic construct
  source 1..59: mol_type = other DNA
caagcagaagacggcatacgagatgaacacgagtgactggagttcagacgtgtgctctt

SEQ ID NO: 56 - DNA, 59 bp, synthetic construct
  source 1..59: mol_type = other DNA
caagcagaagacggcatacgagatgaagcaacgtgactggagttcagacgtgtgctctt

SEQ ID NO: 57 - DNA, 59 bp, synthetic construct
  source 1..59: mol_type = other DNA
caagcagaagacggcatacgagatgaaggttggtgactggagttcagacgtgtgctctt

SEQ ID NO: 58 - DNA, 59 bp, synthetic construct
  source 1..59: mol_type = other DNA
caagcagaagacggcatacgagatgactcagtgtgactggagttcagacgtgtgctctt

SEQ ID NO: 59 - DNA, 59 bp, synthetic construct
  source 1..59: mol_type = other DNA
caagcagaagacggcatacgagatgagaagtcgtgactggagttcagacgtgtgctctt

SEQ ID NO: 60 - DNA, 59 bp, synthetic construct
  source 1..59: mol_type = other DNA
caagcagaagacggcatacgagatgatgagcagtgactggagttcagacgtgtgctctt

SEQ ID NO: 61 - DNA, 59 bp, synthetic construct
  source 1..59: mol_type = other DNA
caagcagaagacggcatacgagatgatgtcgtgtgactggagttcagacgtgtgctctt

SEQ ID NO: 62 - DNA, 59 bp, synthetic construct
  source 1..59: mol_type = other DNA
caagcagaagacggcatacgagatgcaacctagtgactggagttcagacgtgtgctctt

SEQ ID NO: 63 - DNA, 59 bp, synthetic construct
  source 1..59: mol_type = other DNA
caagcagaagacggcatacgagatgcaattcggtgactggagttcagacgtgtgctctt

SEQ ID NO: 64 - DNA, 31 bp, synthetic construct
  source 1..31: mol_type = other DNA
tatggtaattgtgtgccagcmgccgcggtaa

SEQ ID NO: 65 - DNA, 20 bp, synthetic construct
  source 1..20: mol_type = other DNA
ggactachvgggtwtctaat

SEQ ID NO: 66 - DNA, 16 bp, synthetic construct
  source 1..16: mol_type = other DNA
aagtcgagaagcagca

SEQ ID NO: 67 - DNA, 16 bp, synthetic construct
  source 1..16: mol_type = other DNA
atacttcgaagcagca

SEQ ID NO: 68 - DNA, 16 bp, synthetic construct
  source 1..16: mol_type = other DNA
agctgctaaagcagca

SEQ ID NO: 69 - DNA, 16 bp, synthetic construct
  source 1..16: mol_type = other DNA
catagagaaagcagca

SEQ ID NO: 70 - DNA, 16 bp, synthetic construct
  source 1..16: mol_type = other DNA
cgtagatcaagcagca

SEQ ID NO: 71 - DNA, 16 bp, synthetic construct
  source 1..16: mol_type = other DNA
ctcgttacaagcagca

SEQ ID NO: 72 - DNA, 16 bp, synthetic construct
  source 1..16: mol_type = other DNA
gcgcacgtaagcagca

SEQ ID NO: 73 - DNA, 16 bp, synthetic construct
  source 1..16: mol_type = other DNA
ggtactataagcagca

SEQ ID NO: 74 - DNA, 16 bp, synthetic construct
  source 1..16: mol_type = other DNA
gtatacgcaagcagca

SEQ ID NO: 75 - DNA, 16 bp, synthetic construct
  source 1..16: mol_type = other DNA
tacgagcaaagcagca

SEQ ID NO: 76 - DNA, 16 bp, synthetic construct
  source 1..16: mol_type = other DNA
aagtcgagacgcgtga

SEQ ID NO: 77 - DNA, 16 bp, synthetic construct
  source 1..16: mol_type = other DNA
atacttcgacgcgtga

SEQ ID NO: 78 - DNA, 16 bp, synthetic construct
  source 1..16: mol_type = other DNA
agctgctaacgcgtga

SEQ ID NO: 79 - DNA, 16 bp, synthetic construct
  source 1..16: mol_type = other DNA
catagagaacgcgtga

SEQ ID NO: 80 - DNA, 16 bp, synthetic construct
  source 1..16: mol_type = other DNA
cgtagatcacgcgtga

SEQ ID NO: 81 - DNA, 16 bp, synthetic construct
  source 1..16: mol_type = other DNA
ctcgttacacgcgtga

SEQ ID NO: 82 - DNA, 16 bp, synthetic construct
  source 1..16: mol_type = other DNA
gcgcacgtacgcgtga

SEQ ID NO: 83 - DNA, 16 bp, synthetic construct
  source 1..16: mol_type = other DNA
ggtactatacgcgtga

SEQ ID NO: 84 - DNA, 16 bp, synthetic construct
  source 1..16: mol_type = other DNA
gtatacgcacgcgtga

SEQ ID NO: 85 - DNA, 16 bp, synthetic construct
  source 1..16: mol_type = other DNA
tacgagcaacgcgtga

SEQ ID NO: 86 - DNA, 16 bp, synthetic construct
  source 1..16: mol_type = other DNA
aagtcgagcgatctac

SEQ ID NO: 87 - DNA, 16 bp, synthetic construct
  source 1..16: mol_type = other DNA
atacttcgcgatctac

SEQ ID NO: 88 - DNA, 16 bp, synthetic construct
  source 1..16: mol_type = other DNA
agctgctacgatctac

SEQ ID NO: 89 - DNA, 16 bp, synthetic construct
  source 1..16: mol_type = other DNA
catagagacgatctac

SEQ ID NO: 90 - DNA, 16 bp, synthetic construct
  source 1..16: mol_type = other DNA
cgtagatccgatctac

SEQ ID NO: 91 - DNA, 16 bp, synthetic construct
  source 1..16: mol_type = other DNA
ctcgttaccgatctac

SEQ ID NO: 92 - DNA, 16 bp, synthetic construct
  source 1..16: mol_type = other DNA
gcgcacgtcgatctac

SEQ ID NO: 93 - DNA, 16 bp, synthetic construct
  source 1..16: mol_type = other DNA
ggtactatcgatctac

SEQ ID NO: 94 - DNA, 16 bp, synthetic construct
  source 1..16: mol_type = other DNA
gtatacgccgatctac

SEQ ID NO: 95 - DNA, 16 bp, synthetic construct
  source 1..16: mol_type = other DNA
tacgagcacgatctac

SEQ ID NO: 96 - DNA, 16 bp, synthetic construct
  source 1..16: mol_type = other DNA
aagtcgagtgcgtcac

SEQ ID NO: 97 - DNA, 16 bp, synthetic construct
  source 1..16: mol_type = other DNA
atacttcgtgcgtcac

SEQ ID NO: 98 - DNA, 16 bp, synthetic construct
  source 1..16: mol_type = other DNA
agctgctatgcgtcac

SEQ ID NO: 99 - DNA, 16 bp, synthetic construct
  source 1..16: mol_type = other DNA
catagagatgcgtcac

SEQ ID NO: 100 - DNA, 16 bp, synthetic construct
  source 1..16: mol_type = other DNA
cgtagatctgcgtcac

SEQ ID NO: 101 - DNA, 16 bp, synthetic construct
  source 1..16: mol_type = other DNA
ctcgttactgcgtcac

SEQ ID NO: 102 - DNA, 16 bp, synthetic construct
  source 1..16: mol_type = other DNA
gcgcacgttgcgtcac

SEQ ID NO: 103 - DNA, 16 bp, synthetic construct
  source 1..16: mol_type = other DNA
ggtactattgcgtcac

SEQ ID NO: 104 - DNA, 16 bp, synthetic construct
  source 1..16: mol_type = other DNA
gtatacgctgcgtcac

SEQ ID NO: 105 - DNA, 16 bp, synthetic construct
  source 1..16: mol_type = other DNA
tacgagcatgcgtcac

SEQ ID NO: 106 - DNA, 16 bp, synthetic construct
  source 1..16: mol_type = other DNA
aagtcgaggtctagtg

SEQ ID NO: 107 - DNA, 16 bp, synthetic construct
  source 1..16: mol_type = other DNA
atacttcggtctagtg

SEQ ID NO: 108 - DNA, 16 bp, synthetic construct
  source 1..16: mol_type = other DNA
agctgctagtctagtg

SEQ ID NO: 109 - DNA, 16 bp, synthetic construct
  source 1..16: mol_type = other DNA
catagagagtctagtg

SEQ ID NO: 110 - DNA, 16 bp, synthetic construct
  source 1..16: mol_type = other DNA
cgtagatcgtctagtg

SEQ ID NO: 111 - DNA, 16 bp, synthetic construct
  source 1..16: mol_type = other DNA
ctcgttacgtctagtg

SEQ ID NO: 112 - DNA, 16 bp, synthetic construct
  source 1..16: mol_type = other DNA
gcgcacgtgtctagtg

SEQ ID NO: 113 - DNA, 16 bp, synthetic construct
  source 1..16: mol_type = other DNA
ggtactatgtctagtg

SEQ ID NO: 114 - DNA, 16 bp, synthetic construct
  source 1..16: mol_type = other DNA
gtatacgcgtctagtg

SEQ ID NO: 115 - DNA, 16 bp, synthetic construct
  source 1..16: mol_type = other DNA
tacgagcagtctagtg

SEQ ID NO: 116 - DNA, 16 bp, synthetic construct
  source 1..16: mol_type = other DNA
aagtcgagctagtatg

SEQ ID NO: 117 - DNA, 16 bp, synthetic construct
  source 1..16: mol_type = other DNA
atacttcgctagtatg

SEQ ID NO: 118 - DNA, 16 bp, synthetic construct
  source 1..16: mol_type = other DNA
agctgctactagtatg

SEQ ID NO: 119 - DNA, 16 bp, synthetic construct
  source 1..16: mol_type = other DNA
catagagactagtatg

SEQ ID NO: 120 - DNA, 16 bp, synthetic construct
  source 1..16: mol_type = other DNA
cgtagatcctagtatg

SEQ ID NO: 121 - DNA, 16 bp, synthetic construct
  source 1..16: mol_type = other DNA
ctcgttacctagtatg

SEQ ID NO: 122 - DNA, 16 bp, synthetic construct
  source 1..16: mol_type = other DNA
gcgcacgtctagtatg

SEQ ID NO: 123 - DNA, 16 bp, synthetic construct
  source 1..16: mol_type = other DNA
ggtactatctagtatg

SEQ ID NO: 124 - DNA, 16 bp, synthetic construct
  source 1..16: mol_type = other DNA
gtatacgcctagtatg

SEQ ID NO: 125 - DNA, 16 bp, synthetic construct
  source 1..16: mol_type = other DNA
tacgagcactagtatg

SEQ ID NO: 126 - DNA, 25 bp, synthetic construct
  source 1..25: mol_type = other DNA
acctacaacaaagctctcatcaacc

SEQ ID NO: 127 - DNA, 42 bp, synthetic construct
  source 1..42: mol_type = other DNA
gagttcagacgtgtgctcttccgatctnnnnnnnnnngatat

SEQ ID NO: 128 - DNA, 41 bp, synthetic construct
  source 1..41: mol_type = other DNA
gagttcagacgtgtgctcttccgatctnnnnnnnnnnacgc

SEQ ID NO: 129 - DNA, 25 bp, synthetic construct
  source 1..25: mol_type = other DNA
gggttgagatgtgtataagagacag

SEQ ID NO: 130 - DNA, 27 bp, synthetic construct
  source 1..27: mol_type = other DNA
gtgactggagttcagacgtgtgctctt

SEQ ID NO: 131 - DNA, 24 bp, synthetic construct
  source 1..24: mol_type = other DNA
tgacattacctcagcgtaatttac

SEQ ID NO: 132 - DNA, 20 bp, synthetic construct
  source 1..20: mol_type = other DNA
cagcatagaccgtaaactgc

(19) United States
(12) Patent Application Publication
(10) Pub. No.: US 2026/0139328 A1
(43) Pub. Date: May 21, 2026

(54) NOVEL MICROBIAL GENOTOXINS

(71) Applicant: Yale University, New Haven, CT (US)

(72) Inventors: Yiyun Cao, New Haven, CT (US); Noah Palm, New Haven, CT (US); Jason Crawford, New Haven, CT (US); Joonseok Oh, New Haven, CT (US); Seth Herzon, New Haven, CT (US); Mengzhao Xue, New Haven, CT (US)

(21) Appl. No.: 19/118,615

(22) PCT Filed: Oct. 6, 2023
(86) PCT No.: PCT/US2023/076213

§ 371 Date: Apr. 4, 2025

Related U.S. Application Data
(60) Provisional application No. 63/378,744, filed on Oct. 7, 2022.

(51) Int. Cl. C12Q 1/689; A61K 45/06; C07D 209/40; C07K 16/40; C07K 16/44; C12N 15/113

(52) U.S. Cl. CPC C12Q 1/689; A61K 45/06; C07D 209/40; C07K 16/40; C07K 16/44; C12N 15/113; C12N 15/1137; C07K 2317/622; C12N 2310/11; C12Q 2600/142; C12Q 2600/156

(57) ABSTRACT

The present invention relates to compositions and methods for preventing or treating diseases or disorders involving nucleic acid damage, such as cancer, in a subject, as well as methods of identifying gut microbes, strains or metabolites thereof associated with nucleic acid damage and diseases or disorders involving nucleic acid damage, such as cancer.

### CROSS-REFERENCE TO RELATED APPLICATIONS

This application claims priority to U.S. Provisional Patent Application No. 63/378,744, filed Oct. 7, 2022, the contents of which are incorporated by reference herein in its entirety.

### REFERENCE TO A “SEQUENCE LISTING” SUBMITTED AS AN XML FILE

The Sequence Listing written in the XML text file: “047162-5335-00WO_SequenceListing.xml”; created on Sep. 21, 2023, and 123,193 bytes in size, is hereby incorporated by reference.

### STATEMENT REGARDING FEDERALLY SPONSORED RESEARCH OR DEVELOPMENT

This invention was made with government support under CA016359 awarded by National Institutes of Health. The government has certain rights in the invention.

### BACKGROUND OF THE INVENTION

Colorectal cancer (CRC) is the third most common malignancy and the second leading cause of cancer death worldwide (Keum and Giovannucci, 2019, Nat. Rev. Gastroenterol. Hepatol. 16, 713-732). Two-thirds of all CRC cases occur in individuals without a family history of CRC or inherited CRC-predisposing genetic mutations (Jasperson et al., 2010, Gastroenterology 138, 2044-2058). Thus, environmental risk factors that promote the acquisition and accumulation of somatic genetic and epigenetic aberrations are chief contributors to CRC development. The gut microbiome has been reported to modulate intestinal carcinogenesis through diverse mechanisms (Brennan and Garrett, 2016, Annu. Rev. Microbiol. 70, 395-411; Tilg et al., 2018, Cancer Cell 33, 954-964; Garrett, 2015, Science 348, 80-86). Examples include short-chain fatty acid-producing clostridia species (that induce regulatory T cells as well as temper inflammation-induced carcinogenesis (Koh, 2016, Cell 165, 1332-1345) and Fusobacterium nucleatum strains that enhance tumor growth by inducing epithelial proliferation through FadA-mediated engagement of E-cadherin and activation of Wnt/β-catenin signaling Rubinstein et al., 2013, Cell Host Microbe 14, 195-206). Microbial products may also trigger DNA modifications in intestinal epithelial cells (Allen and Sears, 2019, Genome Med. 11, 11). For example, the 20 kDa Bacteroides fragilis toxin induces DNA damage through induction of reactive oxygen species (ROS) (Goodwin et al., 2011, Proc. Natl. Acad. Sci. U.S.A. 108, 15354-15359), while cytolethal distending toxin (CDT) from pathogenic Gram-negative bacteria has direct DNase activity (Buc et al., 2013, PLoS One 8, e56964).

Small molecule metabolites from the microbiome may influence CRC risk by directly causing DNA damage. Select Escherichia coli strains produce the reactive small molecule genotoxin colibactin, which directly alkylates and crosslinks DNA, triggering double-strand DNA breaks (DSBs) that may facilitate intestinal carcinogenesis in mouse models (Nougayrede et al., 2006, Science 313, 848-851; Arthur et al., 2012, Science 338, 120-123; Cougnoux et al., 2014, Gut 63, 1932-1942; Cuevas-Ramos et al., 2010, Proc. Natl. Acad. Sci. U.S.A. 107, 11537-11542). The colibactin biosynthetic machinery is encoded by a 54 kb hybrid polyketide synthase-nonribosomal peptide synthetase (PKS-NRPS) gene cluster referred to as the pks or clb locus (Nougayrede et al., 2006, Science 313, 848-851), and the mature chemical structure of colibactin responsible for the pathway's DNA interstrand crosslinking activity was recently determined (Xue et al., 2019, Science 365, 6457; Wilson et al., 2019, Science 363, eaar7785). Human CRCs also contain mutational signatures consistent with colibactin-induced DNA damage, implicating colibactin in human CRC (Pleguezuelos-Manzano et al., 2020, Nature 580, 269-273; Dziubańska-Kusibab et al., Nat. Med. 26, 1063-1069). The colibactin paradigm illustrates the importance of microbiota metabolite-induced DNA damage in human CRC. However, aside from colibactin, the potential role of microbiota-derived small molecule genotoxins in CRC initiation or progression remains mostly unexplored.

Thus, there is a need in the art for systems and methods for identification of microbes and their metabolites that contribute to DNA damage and cancer. The present invention addresses this unmet need in the art.

### SUMMARY

The present invention is based, in part, on the discovery that the presence of particular pathogenic members of the gut microbiome, and their metabolites, can contribute to the development a disease or disorder involving DNA damage, such as cancer, in a subject.

Thus, in some embodiments, the invention is a method of identifying a gut microbe genotoxin able to damage nucleic acid, the method comprising one or more or all of the steps of a) culturing a population of gut microbes, b) contacting a test nucleic acid with the cultured population of gut microbes, or the culture supernatant thereof, c) assessing the test nucleic acid for damage, d) identifying damage to the test nucleic acid, e) isolating the gut microbe genotoxin from the gut microbe, or the culture supernatant thereof, when the test nucleic acid exhibits damage, f) identifying the gut microbe genotoxin. In some embodiments, the method also includes identifying the gene or genes responsible for genotoxin production. In some embodiments, the gut microbe genotoxin is an indolimine. In some embodiments, the indolimine is indolimine-214, indolimine-200, or indolimine-248. In some embodiments, the invention is gut microbe genotoxin identified by the methods described herein.

In one embodiment, the invention is an indolimine. In some embodiments, the indolimine is indolimine-214, indolimine-200, or indolimine-248. In some embodiments, synthesis of the indolimine is mediated by an enzyme encoded a codon-optimized nucleic acid sequence of Peg1085.

In one embodiment, the invention is a method of preventing or treating a disease or disorder in a subject in need thereof, comprising administering to the subject an effective amount of an inhibitor of indolimine, an inhibitor of an indolimine synthesis or an inhibitor of an indolimine-producing bacterium. In some embodiments, the inhibitor of indolimine is at least one selected from the groups consisting of an antibody, an antibody fragment, a single chain antibody (scFv), an anticalin, a nucleic acid, an antisense nucleic acid, an antibiotic, or probiotic. In some embodiments, the inhibitor of indolimine synthesis comprises an inhibitor of a decarboxylase. In some embodiments, the decarboxylase is encoded by Peg1085. In some embodiments, the inhibitor of indolimine synthesis is at least one selected from the groups consisting of an antibody, an antibody fragment, a single chain antibody (scFv), an anticalin, a nucleic acid, an antisense nucleic acid, an antibiotic, or probiotic.

In one embodiment, the invention is a method of identifying a subject at increased risk of developing cancer, comprising detecting in a biological sample of the subject, one or more species or strains of bacteria that produce a metabolite associated with DNA damage. In some embodiments, the metabolite associated with DNA damage is an indolimine. In some embodiments, the one or more species or strains of bacteria is selected from the group consisting of Bifidobacterium adolescentis, Bfidobacterium dentium, Bifidobacterium breve, Clostridium perfringens, Clostridium ramosum, Streptococcus mitis, Lactobacillus salivarius, Pediococcus acidilactici, Enterococcus asini, and Morganella morganii.

In one embodiment, the invention is a genetically modified bacteria, wherein the genetically modified bacteria is genetically modified so that it is unable to express one or more genes necessary to produce an indolimine. In some embodiments, the one of more genes necessary to produce an indolimine is a decarboxylase. In some embodiments, the decarboxylase is encoded by Peg1085.

### BRIEF DESCRIPTION OF THE DRAWINGS

The following detailed description of exemplary embodiments of the invention will be better understood when read in conjunction with the appended drawings. It should be understood, however, that the invention is not limited to the precise arrangements and instrumentalities of the illustrative exemplary embodiments shown in the drawings.

FIG. 1A through FIG. 1D depict experimental results depicting the establishment of a pipeline to identify genotoxic gut microbes from patients with inflammatory bowel disease. (FIG. 1A) Overview of functional screening of gut microbes for direct genotoxicity. 122 phylogenetically diverse bacterial isolates from 11 IBD patients (shaded based on phylum: Red, Actinobacteria; Blue, Bacteroidetes; Green, Firmicutes; Gray, Fusobacteria; Orange, Proteobacteria) were evaluated for genotoxicity via co-incubation with plasmid DNA followed by gel electrophoresis. Bacterial growth curves for all isolates were determined via OD₆₀₀ and individual isolates were co-cultured with linearized pUC19 DNA under indicated conditions (T_{E}, time point of exponential phase; Ts time point of stationary phase). DNA damage was assessed via gel electrophoresis after native or denaturing treatment of purified plasmid DNA. (FIG. 1B) Diverse human gut microbes exhibited direct DNA damaging activities (N=1). Bacterial genotoxicity was determined by calculating the relative intensity reduction (RIR, %) of linearized pUC19 DNA bands after co-incubation with 122 diverse human gut bacteria (as outlined in FIG. 1A) under indicated conditions as compared to medium only controls. Linearized pUC19 DNA was then purified via column purification and treated with or without gradient NaOH (0%, 0.2%, 0.4%, 1%) before evaluating DNA integrity via gel electrophoresis. 24 selected putative genotoxic isolates were labeled with *; colors were assigned based on phylum: Red, Actinobacteria; Blue, Bacteroidetes; Green, Firmicutes; Orange, Proteobacteria. (FIG. 1C) Selected isolates consistently exhibited direct DNA damaging activities under other culture conditions (N=1). Relative intensity reduction (RIR, %) of linearized pUC19 DNA bands after co-incubation with live bacteria or supernatants (SUP) of 42 isolates (24 genotoxic isolates selected from FIG. 1B and 18 phylogenetically-related non-genotoxic isolates) under indicated conditions as compared to medium only controls. Linearized pUC19 DNA was then purified via column purification and treated with or without gradient NaOH (0%, 0.2%, 0.4%, 1%) before evaluating DNA integrity via gel electrophoresis. 18 selected putative genotoxic isolates were labeled with *; colors were assigned based on phylum: Red, Actinobacteria; Green, Firmicutes; Orange, Proteobacteria. (FIG. 1D) Representative image of gel electrophoresis for 18 selected genotoxic isolates (N=2). Linearized pUC19 DNA damage was evaluated after co-incubation with 18 selected genotoxic isolates from FIG. 1C. Column-purified DNA was treated with or without 0.2% NaOH before gel electrophoresis. Ctrl, non-treated linearized pUC19 DNA; clb+ E. coli, colibactin-producing E. coli; clb− E. coli, colibactin-non-producing E. coli; Medium, medium-alone treated linearized pUC19 DNA.

FIG. 2A through FIG. 2F depict experimental results demonstrating that small molecule metabolites produced by gut microbes induce DNA damage. (FIG. 2A) MFI (geometric mean fluorescence intensity) and representative histograms of 7-H2AX staining of HeLa cells treated with 40% (v/v) PBS (Ctrl), <3 kDa SUP (small-molecule supernatants) of medium, C. perfringens, C. ramosum, M. morganii, clb+ E. coli, clb− E. coli isolates, or cisplatin for 5-6 h (n=3, N=3). * P<0.05; *** P<0.001; **** P<0.0001, one-way ANOVA. (FIG. 2B) Representative data of cell cycle arrest evaluated by propidium iodide (PI) staining (n=3, N=2). HeLa cells were treated with 40% (v/v) PBS or <3 kDa SUP of medium, C. perfringens, C. ramosum or M. morganii isolates. (FIG. 2C) Assessment of circular pUC19 DNA damage after co-incubation with ethyl-acetate extracts of C. perfringens, C. ramosum, M. morganii, clb+ E. coli, clb− E. coli supernatants or medium for 5-6 h (N=2). Ctrl, control pUC19 DNA in TE buffer. (FIG. 2D) MFI of γ-H2AX staining of HeLa cells treated with PBS (Ctrl), 5 mg/ml bacterial or medium extracts for 5-6 h (n=3, N=2). n.s., not significant; ** P<0.01; **** P<0.0001, one-way ANOVA. (FIG. 2E) Comet assay for genomic DNA damage evaluation. Single-cell gel electrophoresis was performed after treating HeLa cells with PBS (Ctrl), cisplatin, bacterial or medium extracts for 5-6 h (n=49, N=1). n.s., not significant; *** P<0.001; **** P<0.0001, one-way ANOVA. (FIG. 2F) Relative abundance of C. perfringens, C. ramosum and M. morganii in data from the Human Microbiome Project. noIBD, healthy controls (n=429); UC, Ulcerative Colitis patients (n=459); CD, Crohn's Disease patients (n=750). n.s., not significant; * P<0.05, one-way ANOVA. Data are means±SEM.

FIG. 3A through FIG. 3G depict experimental results demonstrating the Isolation and identification of a family of genotoxic metabolites derived from M. morganii. (FIG. 3A) Overview of isolation and identification of genotoxins derived from M. morganii. (FIG. 3B) Proposed 4 candidate ion features initially detected from M. morganii bacterial cultures. Rt, retention time. (FIG. 3C) Assessment of circular pUC19 DNA damage after co-incubation overnight with F1-F4 fractions enriched with ion features I-IV, respectively (N=1). Ctrl, control pUC19 DNA in TE buffer. (FIG. 3D) Chemical structures of compounds indolimine-214 (1) and 2. (FIG. 3E) MFI (geometric mean fluorescence intensity) of γ-H2AX staining of HeLa cells treated with synthetic compounds at indicated concentrations for 5 h (n=2, N=3). n.s., not significant; * P<0.05; ** P<0.01; **** P<0.0001, two-way ANOVA. (FIG. 3F) Single-cell genomic DNA comets in HeLa cells after treatment with 100 μg/ml synthetic compounds for 5-6 h (n=25, N=1). n.s., not significant; * P<0.05; ** P<0.01, one-way ANOVA. (FIG. 3G) Chemical structures of compounds indolimine-200 (3) and indolimine-248 (4). (FIG. 3H) MFI of γ-H2AX staining of HeLa cells treated with synthetic compounds at indicated concentrations for 5 h (n=2, N=2). n.s., not significant; * P<0.05; ** P<0.01; **** P<0.0001, two-way ANOVA. Data are means±SEM.

FIG. 4A through FIG. 4C depict experimental results demonstrating M. morganii decarboxylase encoded by aat gene enables indolimine synthesis. (FIG. 4A) Proposed biosynthesis of indolimines in M. morganii. (FIG. 4B) Candidate proteins in M. morganii NWP135 with significant orthology to valine decarboxylase. Peg, protein encoding gene. (FIG. 4C) QTOF-MS identification of indolimine-214 (1), indolimine-200 (3), and indolimine-248 (4) in E. coli BL21(DE3) (N=2). E. coli cells were separately transformed with the plasmid pET28-Peg harboring codon-optimized DNA sequences of Peg1085, Peg1320, or Peg3098. Indolimines were detected after IPTG induction and feeding with precursors (IAld and leucine, valine and phenylalanine, respectively). preIPTG, bacterial supernatants before IPTG induction; post-IPTG, bacterial supernatants after IPTG induction and precursor feeding.

FIG. 5A through FIG. 5F, depict experimental results demonstrating an isogenic M. morganii aat mutant fails to produce indolimines and lacks genotoxicity in vitro. (FIG. 5A) Schematic pipeline of random mutagenesis library construction and mutant identification. (FIG. 5B) Gel result of PCR products of aat gene in aat− or aat+ M. morganii (N=2). (FIG. 5C) QTOF-MS quantification of indolimine-214 (1), indolimine-200 (3), and indolimine-248 (4) in bacterial supernatants of aat+ M. morganii or aat− M. morganii (n=3, N=2). **** P<0.0001, Student's t-test. (FIG. 5D) Growth curves of aai+ M. morganii or aat− M. morganii (n=3, N=1). (FIG. 5E) Gel electrophoresis of cell-free DNA damage assay (N=2). Linearized pUC19 DNA was co-incubated with medium, aat+ M. morganii or aat− M. morganii for 7-8 h, isolated via column purification and treated with or without NaOH (0%, 0.2%, 0.4%, 1%) before evaluating DNA integrity via gel electrophoresis. (FIG. 5F) MFI (geometric mean fluorescence intensity) of γ-H2AX in HeLa cells treated with 40% (v/v)<3 kDa SUP of aat+ M. morganii, or aat− M. morganii for 5-6 h (n=3, N=2). n.s., not significant; * P<0.05; ** P<0.01, one-way ANOVA. Data are means±SEM.

FIG. 6A through FIG. 6E, depict experimental results demonstrating indolimine-producing M. morganii induces increased gut permeability and exacerbates colon tumor burden in gnotobiotic mice. (FIG. 6A)Evaluation of intestinal permeability of mice colonized with aat− or aat− M. morganii based on serum FITC-Dextran RFU (relative fluorescence units) (n=6). * P<0.05, Student's t-test. (FIG. 6B) GO-Slim biological process analysis based on gene ontology overrepresentation test for colonic epithelial cells from mice colonized with aat+ (n=5) or aat− M. morganii (n=6). Data are pooled from N=1 experiment. (FIG. 6C) Schematic of experimental design for AOM/DSS model in age-matched gnotobiotic mice colonized with clbP+ E. coli NC101 (n=4), clbP− E. coli NC101 (n=4), aat+ M. morganii (n=6) or aat− M. morganii (n=6) with non-genotoxic mock community (Geno-, constructed with 7 non-genotoxic isolates) (N=2). (D-E) Tumor number and tumor score (FIG. 6D), representative tumor histology images (scale bar=200 μm) and tumor grade evaluation (FIG. 6E), tumor number and tumor area per section of tissues), in gnotobiotic mice colonized with clbP+ E. coli NC101, clbP− E. coli NC101, aat+ M. morganii or aat− M. morganii with Geno- community. n.s., not significant; * P<0.05; ** P<0.01; *** P<0.001; **** P<0.0001, one-way ANOVA. Data are means±SEM.

FIG. 7 depicts experimental results of the assessment of plasmid DNA stability in diverse bacterial media. Linearized pUC19 DNA was incubated in 14 different media for 48 h, 24 h, or 12 h under anaerobic conditions. DNA integrity was evaluated by gel electrophoresis under native or gradient denaturing conditions (N=1).

FIG. 8A and FIG. 8B depict the clustering of human gut isolates into seven groups with similar growth dynamics. (FIG. 8A) Representative growth curves (top graph) and respective phylogenetic compositions (shaded phylogenetic trees) for seven groups of microbial isolates with similar growth dynamics (n=3, N=2). Bold dashed line, TE (time-point of exponential phase of bacterial growth); Light dashed line, TS (time-point of stationary phase of bacterial growth). Phylogenetic trees are shaded based on phylogeny (phyla). Red, Actinobacteria; Blue, Bacteroidetes; Green, Firmicutes; Gray, Fusobacteria; Orange, Proteobacteria. (FIG. 8B) Table summarizing average TE and TS for each group (N=2).

FIG. 9A through FIG. 9K depict experimental results demonstrating native and denaturing DNA gel electrophoresis images from primary and secondary screening of DNA damage induced by diverse human gut microbes. (FIG. 9A-FIG. 9G) Primary screening data (N=1). Linearized pUC19 DNA was co-incubated with 122 isolates under two culture conditions: co-incubation to Ts under anaerobic conditions or anaerobic co-incubation to T_{E} followed by aerobic co-incubation to Ts. pUC19 DNA was purified via column purification after co-incubation and treated with or without gradient NaOH (0%, 0.2%, 0.4%, 1%) before evaluating DNA integrity via gel electrophoresis. (FIG. 9H-FIG. 9J) Secondary screening data (N=1). Linearized pUC19 DNA was co-incubated with selected isolates (based on primary screening data) anaerobically to Ts or T_{E}, co-incubated anaerobically to T_{E} and then aerobically to Ts, or co-incubated with bacterial supernatants from isolates cultured anaerobically to Ts for 4 h. pUC19 DNA was purified via column purification after co-incubation and treated with or without gradient NaOH (0%, 0.2%, 0.4%, 1%) before evaluating DNA integrity via gel electrophoresis. (FIG. 9K) Representative image of gel electrophoresis for 18 selected genotoxic isolates (N=2). Linearized pUC19 DNA damage was evaluated after co-incubation with 18 selected genotoxic isolates. Column-purified DNA was treated with 0.4% or 1% NaOH before gel electrophoresis. Ctrl, non-treated linearized pUC19 DNA; clb+ E. coli, colibactin-producing E. coli; clb− E. coli, colibactin-non-producing E. coli; Medium, medium-alone treated linearized pUC19 DNA.

FIG. 10A through FIG. 10H depict experimental results demonstrating the selection of genotoxic isolates and evaluation of cell death. (FIG. 10A) 18 potential genotoxic isolates were selected based on two rounds of in vitro DNA gel electrophoresis screening. (FIG. 10B) MFI of γ-H2AX staining of HeLa cells treated with 40% (v/v) PBS (Ctrl) or <3 kDa SUP (small-molecule supernatants) of 18 selected genotoxic isolates, clb+ E. coli, clb− E. coli or medium for 5-6 h (n=3, N=2); MFI and representative histograms of 7-H2AX after infecting HeLa cells with colibactin-producing clb+ E. coli, or non-colibactin-producing clb− E. coli strains, for 4 h (n=3, N=3). * P<0.05; ** P<0.01; *** P<0.001; **** P<0.0001, one-way ANOVA. (FIG. 10C) MFI of γ-H2AX staining of HeLa cells treated with 40% (v/v) unfractionated bacterial supernatants (SUP), or >3 kDa SUP (large-molecules) for 5-6 h (n=3, N=2). n.c., no cell detected; * P<0.05; ** P<0.01; *** P<0.001; **** P<0.0001, one-way ANOVA. (FIG. 10D-FIG. 10E) Representative flow cytometry plots depicting cell size and granularity (FSC/SSC) (FIG. 10D, samples with obvious cell debris were labeled with red) or cell death, apoptosis and necrosis (FIG. 10E, samples with obvious Annexin V+ 7-AAD− apoptosis or Annexin V+ 7-AAD+ necrosis were labeled with red) of HeLa cells after treatment with 40% (v/v)<3 kDa SUP, unfractionated SUP or >3 kDa SUP for 5-6 h (n=3, N=1). (FIG. 10F-FIG. 10G) Percent of apoptotic cells (%) (FIG. 10F) or necrotic cells (%) (FIG. 10G) of HeLa cells treated with 40% (v/v)<3 kDa SUP, unfractionated SUP or >3 kDa SUP for 5-6 h (n=3, N=1). * P<0.05; ** P<0.01; * P<0.001; **** P<0.0001, one-way ANOVA. (FIG. 10H) MFI of γ-H2AX staining of HeLa, HCT116 or MC38 cells treated with ethyl-acetate extracts of C. perfringens, C. ramosum, M. morganii, clb+ E. coli, clb− E. coli supernatants or medium for 5-6 h (n=3, N=2). * P<0.05; ** P<0.01; *** P<0.001; **** P<0.0001, one-way ANOVA. Data are means±SEM.

FIG. 11A through FIG. 11C depict experimental results demonstrating genotoxic human gut microbes, including M. morganii, lack known genotoxin-encoding BGCs (e.g., the clb BGC encoding colibactin) and induce DNA damage via colibactin-independent mechanisms. (FIG. 11A) Biosynthetic gene clusters (BGCs) encoded by genotoxic isolates of C. perfringens, C. ramosum, and M. morganii. BGCs were identified using antiSMASH. (FIG. 11B) PCR-based detection of the colibactin synthesis-related genes, clbI and clbP, in M. morganii, clb+ E. coli, and clb− E. coli (N=1). (FIG. 11C) DNA damage induced by M. morganii, clb+E. coli and clb− E. coli based on linearized pUC19 DNA electrophoresis under native or denaturing conditions (N=2).

FIG. 12A through FIG. 12E depict the isolation and assessment of bioactive genotoxic metabolites derived from M. morganii. (FIG. 12A-FIG. 12B) Gel electrophoresis of two rounds of bioactive fraction screening. Evaluation of nicking of circular pUC19 DNA after overnight co-incubation. Active fractions were labeled with dash or solid red squares (N=1). (FIG. 12C) The integration ratio of H-3 of indolimine-214 (1, δ H 1.64; 4) and H-5′ of compound 2 (δ H 2.05; 6) in F2 (N=1). (FIG. 12D) Purity assessment of synthetic indolimine-214 (1) via integration values of H-8′ and corresponding DNA damage assessment. Circular DNA damage was induced by mixed (bottom, compound 1:2=4:6, consistent with the experimentally observed isolation ratio of F2) but not individual (top) synthetic compounds after overnight co-incubation with compounds (N=2). (FIG. 12E) MFI of γ-H2AX staining for HeLa cells treated with synthetic compounds (with indicated purity) at indicated concentrations for 5 h (n=3, N=2). Significant differences of pre-purified 1, purified 1-F5 (25% of compound 1), purified 1-F6 (70% of compound 1) and purified 1-F7 (70% of compound 1) compared to compound 2 were calculated. n.s., not significant; ** P<0.01; *** P<0.001; **** P<0.0001, two-way ANOVA. Data are means±SEM.

FIG. 13 depicts the 1H and 13C NMR spectroscopic data of natural and synthetic compounds, indolimine-214 (1) and compound 2 (600 MHz, DMSO-d6).

FIG. 14A through FIG. 14D depict experimental results of the Quantification of indolimines in vitro and in vivo. (FIG. 14A) UPLC-QTOF-MS quantification of indolimine-214 (1) in medium or bacterial supernatants of M. morganii, clbP+ or clbP− E. coli NC101 (n=3, N=2). **** P<0.0001, one-way ANOVA. (FIG. 14B) QTOF-MS quantification of indolimine-214 (1) in cecal contents of gnotobiotic mice colonized by M. morganii, or clbP− E. coli NC101 (n=5, N=1). **** P<0.0001, Student's t-test. (FIG. 14C) QTOF-MS quantification of indolimine-200 (3) and indolimine-248 (4) in cecal contents of gnotobiotic mice colonized by M. morganii, or clbP− E. coli NC101 (n=5, N=1). **** P<0.0001, Student's t-test. (FIG. 14D) UPLC-QTOF-MS quantification of indolimine-200 (3) and indolimine-248 (4) in bacterial supernatants of M. morganii or clbP− E. coli NC101 (n=3, N=1). **** P<0.0001, Student's t-test. Data are means±SEM.

FIG. 15 depicts experimental results of the ¹H and ¹³C NMR spectroscopic data of natural and synthetic compounds, indolimine-200 (3) and 248 (4) (600 MHz, methanol-d₄).

FIG. 16A through FIG. 16D depict the experimental identification of responsible gene for indolimine synthesis in M. morganii. (FIG. 16A) Schematic pipeline of transposon-based random mutagenesis and mutant identification. Briefly, 40 384-well plates were combinatorial pooled into 16 row libraries (R1-R16), 24 column libraries (R1-R24), 5 plate row libraries (PR1-PR5) and 8 plate column libraries (PC 1-PC8), 53 libraries in total. 1 H₂O library was used for sequencing control. Amplicon libraries were prepared through 2 rounds of nested-PCR. Single-end sequencing was performed for 100 cycles. UA, Illumina universal adaptor sequence; xN, a 4- to 7-bp random sequence added to avoid color saturation during sequencing; Index, Illumina index sequence; BC, unique 8-bp barcode sequences to label every library; BS, Illumina flow-cell-binding sequence. (FIG. 16B) Amplicon with aat gene. The 100 bp amplicon contained 32 bp EZ-Tn5 transposon primer sequence and 68 bp genomic sequence after the insertion site. The insertion site was 7 bp after the start codon of aat gene. (FIG. 16C) PCA plot of bulk RNA-seq data of colonic epithelial cells from mice colonized with aat+ (n=5) or aat− M. morganii (n=6). (FIG. 16D) Volcano plot of genes expressed by colonic epithelial cells from mice colonized with aat|(n=5) or aat− M. morganii (n=6). Representative genes were highlighted as cancer-associated genes with higher expression in aat+ M. morganii-colonized mice. Blue line, |log 2(Fold Change)|>0.4; Red line, P<0.05. Data are pooled from N=1 experiment.

FIG. 17A through FIG. 17L depict experimental results identifying indolimines produced by M. morganii do not alter overall inflammation or bacteria abundance. (FIG. 17A) Schematic of experimental design for DSS-induce acute colitis model in age-matched germ-free (GF) mice (n=5), mice colonized with clbP+ E. coli NC101 (n=4), clbP− E. coli NC101 (n=4), aat+ M. morganii (n=5) or aat− M. morganii (n=5) (N=1). (FIG. 17B) Relative weight (%) of GF or bacteria-colonizing mice in DSS-induce acute colitis model. n.s., not significant; * P<0.05, two-way ANOVA. (FIG. 17C-FIG. 17D) Colon lengths on day 7 of GF or bacteria-colonizing mice in DSS-induce acute colitis model. n.s., not significant; * P<0.05; ** P<0.01, one-way ANOVA. (FIG. 17E) CFU (colony-forming unit) on day 7 of GF or bacteria-colonizing mice in DSS-induce acute colitis model. n.d., no detection; n.s., not significant, two-way ANOVA. (FIG. 17F) Fecal lipocalin 2 levels on day 7 of GF or bacteria-colonizing mice in DSS-induce acute colitis model. n.s., not significant, two-way ANOVA. (FIG. 17G) Selected 7 non-genotoxic isolates for Geno- mock community construction (FIG. 1, FIG. 9). (FIG. 17H) Representative colon tissues from gnotobiotic mice colonized with clbP+ E. coli NC101 (n=4), clbP− E. coli NC101 (n=4), aat+ M. morganii (n=6) or aat− M. morganii (n=6) with Geno- community (N=2). (FIG. 17I) Relative abundance of each species in gnotobiotic mice colonized with clbP+ E. coli NC101, clbP− E. coli NC101, aat+ M. morganii or aat− M. morganii with Geno- community. n.s., not significant. *** P<0.001; **** P<0.0001, two-way ANOVA. (FIG. 17J) Colon lengths on day 78 of bacteria-colonizing mice under Geno-mock community background in AOM/DSS model. n.s., not significant, one-way ANOVA. (FIG. 17K) Fecal lipocalin 2 levels of bacteria-colonizing mice under Geno-mock community background in AOM/DSS model. n.s., not significant, one-way ANOVA. (FIG. 17L) Histopathologic colonic inflammation (colitis) scoring based on epithelial cell loss, crypt inflammation, lamina propria (L.P.) mononuclear cells, L.P. polymorphonuclear cells (PMNs), and epithelial hyperplasia (evaluated in 0-3 scale, total 0-15). Data are means±SEM.

FIG. 18A through FIG. 18H depict experimental results demonstrating that the Morganella genus is associated with human tumor in TCGA data and Clostridium species also promote colon tumor burden in gnotobiotic mice. (FIG. 18A) Normalized abundance of Morganella genus in primary tumors of TCGA data (adapted from cancermicrobiome.ucsd.edu/CancerMicrobiome_DataBrowser). (FIG. 18B) Normalized abundance of Morganella genus in primary tumors compared to solid normal tissues of TCGA-COAD (n=825 for primary tumor; n=70 for normal tissue), TCGA-READ (n=298 for primary tumor; n=15 for normal tissue), or TCGA-STAD (n=851 for primary tumor; n=113 for normal tissue). Student's t-test. (FIG. 18C) 51 of 52 M. morganii genomes in NCBI contain aat gene (with >90% identity and 100% query coverage). (FIG. 18D) Schematic of experimental design for AOM/DSS induction in age-matched gnotobiotic mice colonized with Geno- community (n=6), C. perfringens (n=4) or C. ramosum (n=5) (N=2). (FIG. 18E-FIG. 18G) Representative colon tissue and histology images (FIG. 18E), tumor number and tumor score (FIG. 18F), and fecal lipocalin 2 levels (FIG. 18G) in gnotobiotic mice colonized with Geno-community, C. perfringens or C. ramosum. n.s., not significant; * P<0.05; ** P<0.01, one-way ANOVA. (H) UPLC-QTOF-MS quantification of indolimines in medium or bacterial supernatants of C. perfringens NWP4, C. ramosum NWP50, M. morganii NWP69, and M. morganii NWP135 (n=3, N=2). ** P<0.01, one-way ANOVA. Data are means±SEM.

FIG. 19A and FIG. 19B depict (FIG. 19A) ¹H NMR spectrum of the mixture of natural indolimine-214 (1) and compound 2 in DMSO-d₆ and (FIG. 19B) COSY NMR spectrum of the mixture of natural indolimine-214 (1) and compound 2 in DMSO-d6

FIG. 20A and FIG. 20B depict (FIG. 20A) HSQC NMR spectrum of the mixture of natural indolimine-214 (1) and (FIG. 20B) HMBC NMR spectrum of the mixture of natural indolimine-214 (1) and compound 2 in DMSO-d₆

FIG. 21A and FIG. 21B depict (FIG. 21A) ¹H NMR spectrum of synthetic indolimine-214 (1) in DMSO-d₆ and (FIG. 21B) COSY NMR spectrum of synthetic indolimine-214 (1) in DMSO-d₆

FIG. 22A and FIG. 22B depict (FIG. 22A) HSQC NMR spectrum of synthetic indolimine-214 (1) in DMSO-d₆ and (FIG. 22B) HMBC NMR spectrum of synthetic indolimine-214 (1) in DMSO-d₆

FIG. 23A and FIG. 23B depict (FIG. 23A) ROESY NMR spectrum of synthetic indolimine-214 (1) in DMSO-d₆ (mixing time: 300 ms) and (FIG. 23B) ¹H NMR spectrum of synthetic compound 2 in DMSO-d₆

FIG. 24A and FIG. 24B depict (FIG. 24A) COSY NMR spectrum of synthetic compound 2 in DMSO-d₆ and (FIG. 24B) HSQC NMR spectrum of synthetic compound 2 in DMSO-d₆

FIG. 25A and FIG. 25B depict (FIG. 25A) HMBC NMR spectrum of synthetic compound 2 in DMSO-d₆ and (FIG. 25B) ¹H NMR spectrum of natural indolimine-214 (1) in DMSO-d₆

FIG. 26A and FIG. 26B depict (FIG. 26A) COSY NMR spectrum of natural indolimine-214 (1) in DMSO-d₆ and (FIG. 26B) HSQC NMR spectrum of natural indolimine-214 (1) in DMSO-d₆

FIG. 27A and FIG. 27B depict (FIG. 27A) HMBC NMR spectrum of natural indolimine-214 (1) in DMSO-d₆ (1) in DMSO-d₆ and FIG. 27B)¹H NMR spectrum of natural compound 2 in DMSO-d₆

FIG. 28A and FIG. 28B depict (FIG. 28A) COSY NMR spectrum of natural compound 2 in DMSO-d₆ and (FIG. 28B) HSQC NMR spectrum of natural compound 2 in DMSO-d₆

FIG. 29A and FIG. 29B depict (FIG. 29A) HMBC NMR spectrum of natural compound 2 in DMSO-d₆ and (FIG. 29B)¹H NMR spectrum of synthetic indolimine-200 (3) in methanol-d_{4 3334}

FIG. 30A and FIG. 30B depict (FIG. 30A) COSY NMR spectrum of synthetic indolimine-200 (3) in methanol-d₄ and (FIG. 30B) HSQC NMR spectrum of synthetic indolimine-200 (3) in methanol-d₄

FIG. 31A and FIG. 31B depict (FIG. 31A) HMBC NMR spectrum of synthetic indolimine-200 (3) in methanol-d₄ and (FIG. 31B) ROESY NMR spectrum of synthetic indolimine-200 (3) in methanol-d₄ (mixing time: 300 ms)

FIG. 32A and FIG. 32B depict (FIG. 32A)¹H NMR spectrum of synthetic indolimine-248 (4) in methanol-d₄ and (FIG. 32B) COSY spectrum of synthetic indolimine-248 (4) in methanol-d₄

FIG. 33A and FIG. 33B depict (FIG. 33A) HSQC NMR spectrum of synthetic indolimine-248 (4) in methanol-d₄ and (FIG. 33B) HMBC NMR spectrum of synthetic indolimine-248 (4) in methanol-d₄

FIG. 34A and FIG. 34B depict (FIG. 34A) ROESY NMR spectrum of synthetic indolimine-248 (4) in methanol-d₄ (mixing time: 300 ms) and (FIG. 34B) UV (left) and electronic circular dichroism (ECD, right) spectrum of natural compound 2 in methanol

FIG. 35 depicts the growths dynamics of all 7 groups of FIG. 1B.

FIG. 36 depicts the raw growth data of FIG. 122 of selected bacteria.

FIG. 37 depicts the quantification of relative intensity reduction (RIR, %) of linearized pUC19 plasmid DNA bands for in vitro gel electrophoresis-based screening.

FIG. 38 depicts Biosynthetic gene cluster (BGC) analyses of genotoxic Clostridium perfinges (NWP4)

FIG. 39 depicts Biosynthetic gene cluster (BGC) analyses of genotoxic Clostridium perfinges (NWP12)

FIG. 40 depicts Biosynthetic gene cluster (BGC) analyses of genotoxic Morganella morganii (NWP135 and NWP69)

FIG. 41 depicts Biosynthetic gene cluster (BGC) analyses of genotoxic Clostridium perfinges (NWP120 and NWP65)

FIG. 42 depicts Biosynthetic gene cluster (BGC) analyses of genotoxic Clostridium ramosum (NWP50)

FIG. 43 depicts the initial ion list for bacterial metabolites generated by comparative metabolomics with XCMS and MPP with the four finalized ion features (I-IV) are highlighted.

FIG. 44A and FIG. 44B depicts the M. morganii NWP135 encoded 18 predicted decarboxylases, including three decarboxylases (Peg1085, Peg1320, and Peg3098) that were partially homologous to a previously characterized valine decarboxylase from S. viridifaciens.

FIG. 45 depicts the 4 universal forward primers and diverse barcode sequence (unique barcode for every library). Reverse primers were constructed as adaptor_outer (SEQ ID NO:8), barcode, adaptor inner (SEQ ID NO:9), respectively.

FIG. 46 depicts the barcode assignment and pooling design of library construction.

FIG. 47 depicts the GO analysis of the upregulated genes in mice colonized with aat+ M. morganii were determined with a threshold of 0.4 on log 2 (fold change) and P<0.05.

FIG. 48 depicts data demonstrating that the 16S rRNA gene V4 region was amplified from each sample by PCR according to a dual index multiplexing strategy as previously described (Palm et al., 2014, Cell 158, 1000-1010). Amplicons were normalized and cleaned (Agencourt AMPure XP purification beads; Beckman Coulter, A63881) before pooling and library quantification (KAPA Biosystems KK4835; Applied Biosystems QuantStudio 6 Flex instrument). Sequencing was performed on an Illumina Miseq in 2×250 PE configuration, using a 500 cycle V2 reagent kit (MS-102-2003).

FIG. 49 depicts the Metadata of 16S rRNA sequencing and relative abundance results of AOM/DSS-induced tumor experiment by taxon.

FIG. 50A and FIG. 50B depicts Metadata of 16S rRNA sequencing and relative abundance results of AOMIDSS-induced tumor experiment by taxon.

### DETAILED DESCRIPTION

The present invention is based, in part, on the discovery that the presence of particular pathogenic members of the gut microbiome, and their metabolites, can contribute to the development a disease or disorder involving DNA damage, such as cancer, in a subject. The present invention relates to methods for identifying a microbe, strain, or metabolite thereof that is associated with a disease or disorder involving DNA damage, such as cancer, in a subject, as well as to inhibitors and methods of diagnosis, prevention and treatment of subjects having a disease or disorder associated with nucleic acid damage.

### Definitions

Unless defined otherwise, all technical and scientific terms used herein have the same meaning as commonly understood by one of ordinary skill in the art to which this invention belongs.

While the following terms are believed to be well understood by one of ordinary skill in the art, the following definitions are set forth to facilitate explanation of the presently disclosed subject matter.

As used herein, the term “a” or “an” can refer to one or more of that entity, i.e., can refer to a plural referents. As such, the terms “a” or “an”, “one or more” and “at least one” can be used interchangeably herein. In addition, reference to “an element” by the indefinite article “a” or “an” does not exclude the possibility that more than one of the elements is present, unless the context clearly requires that there is one and only one of the elements.

Unless the context requires otherwise, throughout the present specification and claims, the word “comprise” and variations thereof, such as, “comprises” and “comprising” are to be construed in an open, inclusive sense that is as “including, but not limited to”.

Reference throughout this specification to “one embodiment” or “an embodiment” means that a particular feature, structure or characteristic described in connection with the embodiment may be included in at least one embodiment of the present disclosure. Thus, the appearances of the phrases “in one embodiment” or “in an embodiment” in various places throughout this specification may not necessarily all referring to the same embodiment. It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

The terms “patient,” “subject,” “individual,” and the like are used interchangeably herein, and refer to any animal, or cells thereof whether in vitro or in vivo, amenable to the methods described herein. In certain non-limiting embodiments, the patient, subject or individual is, by way of non-limiting examples, a human, a dog, a cat, a horse, or other domestic mammal.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a sign or symptom of the disease or disorder, the frequency with which such a sign or symptom is experienced by a patient, or both, is reduced.

As used herein, “treating a disease or disorder” means reducing the severity and/or frequency with which at least one sign or symptom of the disease or disorder is experienced by a patient.

The term “dysbiosis,” as used herein, refers to imbalances in quality, absolute quantity, or relative quantity of members of the microbiota of a subject, which is sometimes, but not necessarily, associated with the development or progression of a disease or disorder.

As used herein, the term “gastrointestinal tract” (“GI”) or “gut” refers to the entire alimentary canal, from the oral cavity to the rectum. The term encompasses the tube that extends from the mouth to the anus, in which the movement of muscles and release of hormones and enzymes digest food. The gastrointestinal tract starts with the mouth and proceeds to the esophagus, stomach, small intestine, large intestine, rectum and, finally, the anus.

The term “microbiota,” as used herein, refers to the population of microorganisms present within or upon a subject. The microbiota of a subject includes commensal microorganisms found in the absence of disease and may also include pathobionts and disease-causing microorganisms found in subjects with or without a disease or disorder.

As used herein, the term “microbiome” refers to the totality of microbes (bacteria, fungae, protists), their genetic elements (genomes) in a defined environment. In one embodiment, the microbiome is a gut microbiome (e.g., intestinal microbiome). The term “gut microbiome” as used herein can refer to the totality of microorganisms, bacteria, viruses, protozoa and fungi and their collective genetic material present in the gastrointestinal tract (GIT).

The term “gut microbe” as used herein can refer to any commensal or pathogenic microorganisms, bacteria, viruses, protozoa and fungi that colonize the gastrointestinal tract (GIT) or gut. The term “gut microbiota” as used herein can refer to the collection or population of microorganisms, bacteria, viruses, protozoa and fungi, commensal and pathogenic, residing in the GIT.

Examples of gut microbes that make up the gut microbiota and gut microbiome can include, but not be limited to bacteria selected from Segmented Filamentous Bacteria (SFB), Helicobacter flexispira, Lactobacillus, Helicobacter, S24-7, Erysipelotrichaceae, Prevotellaceae, Paraprevotella, Prevotella, Acidaminococcus spp., Actinomyces spp., Akkermansia muciniphila, Allobaculum spp., Anaerococcus spp., Anaerostipes spp., Bacteroides spp., Bacteroides Other, Bacteroides acidifaciens, Bacteroides coprophilus, Bacteroides fragilis, Bacteroides ovatus, Bacteroides uniformis, Barnesiellaceae spp., Bifidobacterium adolescentis, Bifidobacterium Other, Bifidobacterium spp., Bilophila spp., Blautia obeum, Blautia producta, Blautia Other, Blautia spp., Bulleidia spp., Catenibacterium spp., Citrobacter spp., Clostridiaceae spp., Clostridiales Other, Clostridiales spp., Clostridium perfringens, Clostridium spp., Clostridium Other, Collinsella aerofaciens, Collinsella spp., Collinsella stercoris, Coprococcus catus, Coprococcus spp., Coriobacteriaceae spp., Desulfovibrionaceae spp., Dialister spp., Dorea formicigenerans, Dorea spp., Dorea Other, Eggerthella lenta, Enterobacteriaceae Other, Enterobacteriaceae spp., Enterococcus spp., Erysipelotrichaceae spp., Eubacterium biforme, Eubacterium biforme, Eubacterium dolichum, Eubacterium spp., Faecalibacterium prausnitzii, Fusobacterium spp., Gemellaceae spp., Haemophilus parainfluenzae, Haemophilus Other, Helicobacter spp., Helicobacter Lachnospiraceae Other, Lachnospiraceae spp., Lactobacillus reuteri, Lactobacillus mucosae, Lactobacillus zeae, Lactobacillus spp., Lactobacillaceae spp., Lactococcus spp., Leuconostocaceae spp., Megamonas spp., Megasphaera spp., Methanobrevibacter spp., Mitsuokella multacida, Mitsuokella spp., Mucispirillum schaedleri, Odoribacter spp., Oscillospira spp., Parabacteroides distasonis, Parabacteroides spp., Paraprevotella spp., Paraprevotellaceae spp., Parvimonas spp., Pediococcus spp., Pediococcus Other, Peptococcus spp., Peptoniphilus spp., Peptostreptococcus anaerobius, Peptostreptococcus Other, Phascolarctobacterium spp., Prevotella copri, Prevotella spp., Prevotella stercorea, Prevotellaceae, Proteus spp., Rikenellaceae spp., Roseburia faecis, Roseburia spp., Ruminococcaceae Other, Ruminococcaceae spp., Ruminococcus bromii, Ruminococcus gnavus, Ruminococcus spp., Ruminococcus Other, Ruminococcus torques, Slackia spp., S24-7 spp., SMB53 spp., Streptococcus anginosus, Streptococcus luteciae, Streptococcus spp., Streptococcus Other, Sutterella spp., Turicibacter spp., UC Bulleidia, UC Enterobacteriaceae, UC Faecalibacterium, UC Parabacteroides, UC Pediococcus, Varibaculum spp., Veillonella spp., Sutterella, Turicibacter, UC Clostridiales, UC Erysipelotrichaceae, UC Ruminococcaceae, Veillonella parvula, Veillonella spp., Veillonella dispar and Weissella.

The terms “pathobiont” or “pathogenic microbe” are used interchangeably and refer to potentially disease-or disorder-causing members of the microbiota that are present in the microbiota of a non-diseased or a diseased subject, and which has the potential to contribute to the development or progression of a disease or disorder.

The term “beneficial microbe,” as used herein, refers to members of the microbiota that are present in the microbiota of a non-diseased or a diseased subject, and which has the potential to contribute to the reduction of the severity and/or frequency with which at least one sign or symptom of the disease or disorder is experienced by a subject having a disease or disorder.

“Isolated” means altered or removed from the natural state. For example, a microbe naturally present in its normal context in a living animal is not “isolated,” but the same microbe partially or completely separated from the coexisting materials of its natural context is “isolated.” An isolated microbe can exist in substantially purified form, or can exist in a non-native environment such as, for example, a gastrointestinal tract.

An “effective amount” or “therapeutically effective amount” of a compound is that amount of a compound which is sufficient to provide a beneficial effect to the subject to which the compound is administered.

A “therapeutic” treatment is a treatment administered to a subject who exhibits at least one sign or symptom of a disease or disorder, or is at risk of developing at least one sign or symptom of a disease or disorder, for the purpose of diminishing or eliminating those signs or symptoms, or reducing the likelihood of developing at least one sign or symptom of a disease or disorder.

As used herein, the term “pharmaceutical composition” refers to a mixture of at least one compound useful within the invention with a pharmaceutically acceptable carrier. The pharmaceutical composition facilitates administration of the compound to a patient or subject. Multiple techniques of administering a compound exist in the art including, but not limited to, intravenous, oral, rectal, aerosol, parenteral, ophthalmic, pulmonary and topical administration.

As used herein, the term “pharmaceutically acceptable” refers to a material, such as a carrier or diluent, which does not abrogate the biological activity or properties of the compound, and is relatively non-toxic, i.e., the material may be administered to an individual without causing an undesirable biological effect or interacting in a deleterious manner with any of the components of the composition in which it is contained.

The term “regulating” or “modulating” as used herein can mean any method of altering the level or activity of a substrate (e.g., microbiome). Non-limiting examples of regulating with regard to a microbiome or microbiota further include affecting the microbiome or microbiota activity.

The term “regulator” or “modulator” refers to a molecule whose activity includes affecting the level or activity of a substrate (e.g., microbiome). A regulator can be direct or indirect. A regulator can function to activate or inhibit or otherwise modulate its substrate (e.g., microbiome).

The terms “silence”, “silencing”, “inhibit”, and “inhibition,” as used herein, means to reduce, suppress, diminish, or block an activity or function relative to a control value. For example, in one embodiment, the activity is suppressed or blocked by at least about 10% relative to a control value. In some embodiments, the activity is suppressed or blocked by at least about 50% compared to a control value. In some embodiments, the activity is suppressed or blocked by at least about 75%. In some embodiments, the activity is suppressed or blocked by at least about 95%.

The term “homology” refers to a degree of complementarity. There may be partial homology or complete homology (i.e., identity). Homology is often measured using sequence analysis software (e.g., Sequence Analysis Software Package of the Genetics Computer Group. University of Wisconsin Biotechnology Center. 1710 University Avenue. Madison, Wis. 53705). Such software matches similar sequences by assigning degrees of homology to various substitutions, deletions, insertions, and other modifications.

A “fragment” of a peptide sequence or a nucleic acid sequence that encodes an antigen may be 100% identical to the full length except missing at least one amino acid or at least one nucleotide from the 5′ and/or 3′ end, in each case with or without sequences encoding signal peptides and/or a methionine at position 1. Fragments may comprise 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 91% or more, 92% or more, 93% or more, 94% or more, 95% or more, 96% or more, 97% or more, 98% or more, 99% or more percent of the length of the particular full length coding sequence, excluding any heterologous signal peptide added. The fragment may comprise a fragment that encode a polypeptide that is 95% or more, 96% or more, 97% or more, 98% or more or 99% or more identical to the antigen and additionally optionally comprise sequence encoding an N terminal methionine or heterologous signal peptide which is not included when calculating percent identity.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting there from. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some versions contain an intron(s).

As used herein, a “probiotic” refers live, non-pathogenic microorganisms, e.g., bacteria, which can confer health benefits to a host organism that contains an appropriate amount of the microorganism. In some embodiments, the host organism is a mammal. In some embodiments, the host organism is a human. Non-pathogenic bacteria may be genetically engineered to enhance or improve desired biological properties, e.g., survivability. Non-pathogenic bacteria may be genetically engineered to provide probiotic properties. Probiotic bacteria may be genetically engineered to enhance or improve probiotic properties. Some species, strains, and/or subtypes of non-pathogenic bacteria are currently recognized as probiotic bacteria. Examples of probiotic bacteria include, but are not limited to, Bifidobacteria, Escherichia coli, Lactobacillus, and Saccharomyces, e.g., Bifidobacterium bifidum, Enterococcus faecium, Escherichia coli strain Nissle, Lactobacillus acidophilus, Lactobacillus bulgaricus, Lactobacillus paracasei, Lactobacillus plantarum, and Saccharomyces boulardii (Dinleyici et al., 2014; U.S. Pat. Nos. 5,589,168; 6,203,797; 6,835,376). The probiotic may be a variant or a mutant strain of bacterium (Arthur et al., 2012, Science 338, 120-123; Cuevas-Ramos et al., 2010, Proc. Natl. Acad. Sci. U.S.A. 107, 11537-11542; Nougayr6de et al., 2006, Science 313, 848-851). Non-pathogenic bacteria may be genetically engineered to enhance or improve desired biological properties, e.g., survivability. Non-pathogenic bacteria may be genetically engineered to provide probiotic properties. Probiotic bacteria may be genetically engineered to enhance or improve probiotic properties.

As used herein, a “prebiotic” refers to an ingredient that allows specific changes both in the composition and/or activity in the gastrointestinal microbiota that may (or may not) confer benefits upon the host. In some embodiments, a prebiotic can be a comestible food or beverage or ingredient thereof. Prebiotics may include complex carbohydrates, amino acids, peptides, minerals, or other essential nutritional components for the survival of the bacterial composition. Prebiotics include, but are not limited to, amino acids, biotin, fructooligosaccharide, galactooligosaccharides, hemicelluloses (e.g., arabinoxylan, xylan, xyloglucan, and glucomannan), inulin, chitin, lactulose, mannan oligosaccharides, oligofructose-enriched inulin, gums (e.g., guar gum, gum arabic and carregenaan), oligofructose, oligodextrose, tagatose, resistant maltodextrins (e.g., resistant starch), trans-galactooligosaccharide, pectins (e.g., xylogalactouronan, citrus pectin, apple pectin, and rhamnogalacturonan-I), dietary fibers (e.g., soy fiber, sugarbeet fiber, pea fiber, corn bran, and oat fiber) and xylooligosaccharides.

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. The antibodies in the present invention may exist in a variety of forms including, for example, polyclonal antibodies, monoclonal antibodies, intracellular antibodies (“intrabodies”), Fv, Fab and F(ab)2, as well as single chain antibodies (scFv), heavy chain antibodies, such as camelid antibodies, and humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, New York; Houston et al., 1988, Proc. Natl. Acad. Sci. USA 85:5879-5883; Bird et al., 1988, Science 242:423-426).

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

As used herein, the term “nanobody”, “heavy chain antibody” or “heavy chain antibodies” comprises immunoglobulin molecules derived from camelid species, either by immunization with a peptide and subsequent isolation of sera, or by the cloning and expression of nucleic acid sequences encoding such antibodies. The term “heavy chain antibody” or “heavy chain antibodies” further encompasses immunoglobulin molecules isolated from an animal with heavy chain disease, or prepared by the cloning and expression of VH (variable heavy chain immunoglobulin) genes from an animal.

The term “antigen” or “Ag” as used herein is defined as a molecule that provokes an adaptive immune response. This immune response may involve either antibody production, or the activation of specific immunogenically-competent cells, or both. The skilled artisan will understand that any macromolecule, including virtually all proteins or peptides, can serve as an antigen. Furthermore, antigens can be derived from recombinant or genomic DNA or RNA. A skilled artisan will understand that any DNA or RNA, which comprises a nucleotide sequences or a partial nucleotide sequence encoding a protein that elicits an adaptive immune response therefore encodes an “antigen” as that term is used herein. Furthermore, one skilled in the art will understand that an antigen need not be encoded solely by a full-length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a microbiota sample, tissue sample, a tumor sample, a cell or a biological fluid.

The term “adjuvant” as used herein is defined as any molecule to enhance an antigen-specific adaptive immune response.

As used herein, an “immunoassay” refers to any binding assay that uses an antibody capable of binding specifically to a target molecule to detect and quantify the target molecule.

By the term “specifically binds,” as used herein with respect to an antibody, is meant an antibody which recognizes and binds to a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific.

In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The phrase “biological sample” as used herein, is intended to include any sample comprising a cell, a tissue, feces, or a bodily fluid in which the presence of a microbe, nucleic acid or polypeptide is present or can be detected. Samples that are liquid in nature are referred to herein as “bodily fluids.” Biological samples may be obtained from a patient by a variety of techniques including, for example, by scraping or swabbing an area of the subject or by using a needle to obtain bodily fluids. Methods for collecting various body samples are well known in the art.

As used herein, the terms “nutritional supplement” and “dietary supplement” refer to any product that is added to the diet. In some particularly preferred embodiments, nutritional supplements are taken by mouth and often contain one or more dietary ingredients, including but not limited to vitamins, minerals, herbs, amino acids, enzymes, and cultures of organisms.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention is based, in part, on the discovery that the presence and level (e.g., activity, expression, concentration, amount, etc.) of a pathogenic member of the gut microbiome can contribute to the development a disease or disorder involving DNA damage, such as cancer, in a subject. The present invention relates to methods for identifying a microbe, strain, or metabolite thereof that is associated with a disease or disorder involving DNA damage, such as cancer, in a subject. In one embodiment, the present invention provides methods of identifying a metabolite that is associated with DNA damage that is directly or indirectly produced by a microbe.

The present invention also relates to compositions and methods for preventing or treating diseases or disorders involving nucleic acid damage, such as cancer, in a subject, as well as methods of identifying gut microbes, strains or metabolites thereof associated with nucleic acid damage and diseases or disorders involving nucleic acid damage, such as cancer.

In some embodiments, the microbe metabolite is a genotoxin. In some embodiments, the microbe metabolite is a small molecule. In some embodiments, the small molecule metabolite is an indolimine. In some embodiments, the indolimine is indolimine-214. In some embodiments, the indolimine is indolimine-200. In some embodiments, the indolimine is indolimine-248.

In some embodiments, the invention is an inhibitor, wherein the inhibitor diminishes the level (e.g., activity, expression, concentration, amount, etc.) of the pathogenic microbe, strain or metabolite thereof.

In certain aspects, the present invention also provides a method for diagnosing or assessing the risk of developing a disease or disorder involving DNA damage, such as cancer, that is induced by at least one microbe, strain or metabolite thereof in a subject. In one embodiment, the method comprises detecting the presence or amount of at least one microbe, strain or metabolite, or gene(s) responsible for the production of a metabolite thereof that associated with the disease or disorder, such as cancer, in a biological sample of the subject.

In various embodiments, the understanding of the specific microbes that contribute to disease can guide the selection of particular therapeutic treatments (e.g., inhibitors, probiotics, etc.), or predict disease trajectory that can be useful for the development of precision medicine-based therapeutic approaches to treat or prevent microbe-modulated diseases, or as companion diagnostics to determine treatment selection. In other embodiments, the present invention relates to methods of predicting or monitoring the effectiveness of a treatment of a disease or disorder (e.g., DNA damage, cancer, etc.) in a subject.

### Methods of Screening and Identifying Pathogenic Microbes and Their Genotoxic Metabolites

In some embodiments, the present invention relates, in part, to methods of screening and/or identifying a microbe, strain, or metabolite thereof that is associated with a disease or disorder associated with nucleic acid damage (i.e., DNA damage) such as cancer.

In one embodiment, the invention is a method of identifying microbes by screening microbes, such as human gut microbes, for their ability to damage nucleic acid. In some embodiments the nucleic acid is DNA. In some embodiments, the nucleic acid is plasmid DNA.

In one aspect, the present invention provides methods of identifying a nucleic acid-damaging metabolite produced, directly or indirectly, by a microbe. In one embodiment, the present invention relates, in part, to methods of assessing and/or identifying a microbe, strain, or metabolite, directly or indirectly produced by the microbe, for its ability to cause nucleic acid damage. In some embodiments the nucleic acid is DNA. In some embodiments, the nucleic acid is plasmid DNA.

In one aspect, the present invention provides methods of identifying microbes, strains, or metabolites thereof, by detecting the gene(s) responsible for genotoxic metabolite production. In some embodiments, the gene(s) responsible for genotoxic metabolite production is a gene encoding a decarboxylase. In some embodiments, the gene(s) responsible for genotoxic metabolite production is aat.

In some embodiments the microbes to be screened are isolated from subjects that have, or at risk of developing, a disease or disorder, such as inflammatory bowel disease (IBD) or cancer, such as colorectal cancer (CRC). In some embodiments, the microbe is a gut microbe. In some embodiments, the gut microbe is isolated from a stool sample. In some embodiments, the microbe is a gram-negative bacterium. In some embodiments, the microbe is a gram-positive bacterium.

In one embodiment, the microbe, strain, or a metabolite thereof is identified as a microbe, strain, or a metabolite thereof that induces a disease or disorder associated with nucleic acid damage, such as cancer, when the presence or level (e.g., activity, expression, concentration, amount, etc.) of the microbe, strain, or metabolite thereof is assessed and found to contribute to nucleic acid damage when compared to a comparator. In various embodiments, the comparator us a positive control, a negative control, a historical control, a historical norm, or the level of another reference molecule in the biological sample.

In some embodiments, the nucleic acid to be assessed for damage is co-incubated with pooled microbes or strains thereof. In some embodiments, the nucleic acid to be assessed for damage is co-incubated with individual isolates of the microbe or strain thereof. In some embodiments, prior to co-incubating nucleic acid with the microbe(s), or strain(s) thereof, to be assessed, the growth curve of the microbe, or strain thereof, to be assessed is measured to establish the T_{E} (time point of exponential phase) and/or T_{S} (time point of stationary phase) for each microbe or strain thereof.

In some embodiments, the microbe or strain thereof is co-incubated with nucleic acid under anaerobic conditions to T_{S}. In some embodiments, the microbe or strain thereof is co-incubated with nucleic acid under anaerobic conditions to T_{E}. In some embodiments, the microbe or strain thereof is co-incubated with nucleic acid under anaerobic conditions to T_{E}, and then the microbe or strain thereof is co-incubated with nucleic acid under aerobic conditions to Ts. In some embodiments, the nucleic acid is co-incubated with bacterial supernatants from the microbe or strain thereof cultured anaerobically to T_{S}, for 1, 2, 3, 4, 5, 6, 7, 8, 9, 10, 11, 12, 16, 20, 24 or more hours.

In some embodiments, the nucleic acid to be assessed for damage is isolated after co-incubation with the microbe or strain thereof. In some embodiments, the nucleic acid to be assessed for damage is isolated after co-incubation with the microbe or strain thereof using column purification.

In some embodiments, the genotoxic activity of each microbe or strain thereof is assessed in an assay to assess nucleic acid damage. In some embodiments, the nucleic acid assessed for damage is DNA. In some embodiments, the nucleic acid assessed for damage is plasmid DNA. In some embodiments, the nucleic acid assessed for damage is pUC19 plasmid DNA. In some embodiments, the nucleic acid assessed for damage is circular pUC19 plasmid DNA. In some embodiments, the nucleic acid assessed for damage is linearized pUC19 plasmid DNA.

In some embodiments, the type and/or extent nucleic acid damage is assessed using electrophoresis. In some embodiments, the type and/or extent nucleic acid damage is assessed using electrophoresis under native conditions. In some embodiments, the type and/or extent of nucleic acid damage is assessed with electrophoresis under denaturing conditions. By way of one non-limiting example, double-stranded linearized pUC19 DNA migrates as a slow-moving band under native electrophoresis, while denaturing treatment separates the DNA into single-stranded DNA, leading to a higher-mobility band.

In some embodiments, the nucleic acid to be assessed for damage, isolated after co-incubation with the microbe, is subjected to electrophoresis, such as gel electrophoresis, under native or denaturing conditions. In some embodiments, denaturing conditions are produced using selected concentrations of NaOH, such as about 0%, 0.1%, 0.2%, 0.3%, 0.4%, 0.5%, 0.6%, 0.7%, 0.8%, 0.9% 1.0%, 1.1%, 1.2%, 1.3%, 1.4%, 1.5% or more NaOH.

In various embodiments, electrophoresis under native and/or denaturing conditions allows for the assessment of the type and/or extent of nucleic acid damage after exposure to a microbe, strain or metabolite having nucleic acid damaging activity. By way of one non-limiting example, the formation of nucleic acid interstrand cross-links (ICLs), prevents unwinding under denaturing conditions, thereby resulting in a band that has the same mobility as duplexed nucleic acid. By way of another non-limiting example, alkylation at many of the residues of nucleic acid is known to decrease the stability of glycoside bonds, resulting in deglycosylation and fragmentation. Such damaged nucleic acid products can be detected as smaller fragments of higher mobility following electrophoresis. By way of another non-limiting example, extensive nucleic acid damage, for example, such as by DNase-mediated degradation, results in a loss of nucleic even under native. By way of another non-limiting example, nucleic acid damage induced by restriction enzyme-like molecules produces multiple bands under native conditions, and even smaller fragments under denaturing conditions when combined with damage induced by alkylation or DNase-like molecules.

In some embodiments, the relative intensity reduction (RIR, %) of nucleic after co-incubation with the microbe, strain, or metabolite is used as a measure of microbe-induced nucleic acid damage.

In various embodiments, electrophoresis of nucleic acid (e.g., DNA, circular plasmid, linearized plasmid, etc.) allows for the assessment of the type and/or extent of nucleic acid damage after exposure to a microbe, strain or metabolite having nucleic acid damaging activity. By way of non-limiting examples, undamaged circular plasmid is supercoiled and has the highest mobility, open nicked pUC19 plasmid has lower motility, and linear pUC19 plasmid exhibits motility between supercoiled and nicked circular pUC19.

In some embodiments, a microbe or strain thereof identified as exhibiting nucleic acid damaging activity is assessed for the presence of a metabolite able to induce nucleic acid damage. In some embodiments, supernatant (i.e., SUP) from a microbe or strain thereof identified as exhibiting nucleic acid damaging activity is assessed for the presence of a metabolite able to induce nucleic acid damage. In some embodiments, a metabolite present in a supernatant can be enriched. In some embodiments, enrichment is accomplished using ethyl-acetate extraction, or the like. In some embodiments, the metabolite could be de novo synthesized.

In some embodiments, the supernatant of a microbe, or strain thereof, identified as exhibiting nucleic acid damaging activity is assessed for the presence of a metabolite able to induce nucleic acid damage. In various embodiments, the supernatant is collected from a microbe, or strain thereof, cultured under anaerobic conditions, cultured under anaerobic conditions to T_{E}, cultured under anaerobic conditions to T_{S}, or cultured under anaerobic conditions to T_{E} and then under aerobic conditions to Ts.

In some embodiments, the supernatant is separated into fractions according to molecular weight, for assessing the fractions for genotoxicity and/or nucleic acid damage. In some embodiments, the supernatant is separated into small- (e.g., <3 kDa) and large- (e.g., >3 kD) molecular weight fractions for assessment of genotoxicity and/or nucleic acid damage. In some embodiments, genotoxicity is assessed using cells, such as, by way of a non-limiting example, HeLa cells.

In some embodiments, genotoxicity and/or nucleic acid damage is assessed by measuring the level of γ-H2AX, a known marker of DNA double-strand breaks (DSBs), in cells contacted with the microbes supernatant. In some embodiments, genotoxicity and/or nucleic acid damage is assessed by measuring the level of apoptosis in cells contacted with the supernatant. In some embodiments, genotoxicity and/or nucleic acid damage is assessed by measuring the level of necrosis in cells contacted with the supernatant. In various embodiments, the level of apoptosis and/or necrosis is measured by measuring cell size, cell granularity, Annexin V staining, 7-AAD staining, or any combination thereof.

In various embodiments of the methods of the invention, the level (e.g., activity, expression, concentration, amount, etc.) of the microbe, strain, or metabolite thereof is determined to be increased when the level of microbe, strain, or metabolite thereof (e.g., activity, expression, concentration, amount, etc.) in the biological sample is increased by at least 0.1%, by at least 1%, by at least 10%, by at least 20%, by at least 30%, by at least 40%, by at least 50%, by at least 60%, by at least 70%, by at least 80%, by at least 90%, by at least 100%, by at least 125%, by at least 150%, by at least 175%, by at least 200%, by at least 250%, by at least 300%, by at least 400%, by at least 500%, by at least 600%, by at least 700%, by at least 800%, by at least 900%, by at least 1000%, by at least 1500%, by at least 2000%, by at least 2500%, by at least 3000%, by at least 4000%, or by at least 5000%, when compared with a comparator. In various embodiments, the comparator us a positive control, a negative control, a historical control, a historical norm, a predetermined threshold, or the level of another reference molecule in the biological sample.

In various embodiments of the methods of the invention, the level (e.g., activity, expression, concentration, amount, etc.) of microbe, strain, or metabolite thereof is determined to be increased when the level (e.g., activity, expression, concentration, amount, etc.) of microbe or strain thereof in the biological sample is determined to be increased by at least 1 fold, at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 11 fold, at least 12 fold, at least 13 fold, at least 14 fold, at least 15 fold, at least 20 fold, at least 25 fold, at least 30 fold, at least 40 fold, at least 50 fold, at least 75 fold, at least 100 fold, at least 200 fold, at least 250 fold, at least 500 fold, or at least 1000 fold, or at least 10000 fold, when compared with a comparator. In various embodiments, the comparator us a positive control, a negative control, a historical control, a historical norm, a predetermined threshold, or the level of another reference molecule in the biological sample.

In other embodiments, the method comprises identifying microbes in subjects having a disease or disorder associated with nucleic acid damage (e.g., cancer, etc.) and from subjects from publicly available large-scale human microbiome datasets (e.g., American Gut Project data, Cancer Microbiome database, etc.) through the QIITA repository and analysis suite.

In some embodiments, the metabolite associated with nucleic acid damage is identified. In some embodiments, the metabolite is identified using ultra-performance liquid chromatography quadrupole time-of-flight mass spectrometry (UPLC-QTOF-MS)-based untargeted metabolomics, bioactivity-guided fractionation using small-scale cultures, large-scale cultivation and isolation, structure elucidation, genotoxicity analyses, magnetic resonance (NMR) spectroscopy, or any combination thereof.

In some embodiments, comparative metabolomics is used to generate a candidate ion list of abundant microbe-derived metabolites relative to a comparator, such as, by way of a non-limiting example, a culture medium negative control. In some embodiments, at least one round of activity-guided fractionation using preparative high-performance liquid chromatography (HPLC), and a nucleic acid-based genotoxicity assay is used, before profiling the resulting fractions and subfractions using UPLC-QTOF-MS-based metabolomics. In some embodiments, a metabolite is identified, in part, by excluding ions present in inactive fractions.

In various embodiments, the present invention relates to the isolation and identification of a microbe, strain, or a metabolite thereof that influence the development and progression of a disease or disorder associated with nucleic acid damage, such as cancer. In one embodiment, the invention relates to compositions and methods for detecting and determining the identity of the pathogenic microbe, strain or metabolite thereof. In some embodiments, the detection and identification of the pathogenic microbe, strain, or metabolite thereof is used to diagnose the subject as having, or as at risk of developing, a disease or disorder associated with nucleic acid damage, such as cancer. In other embodiments, the detection and identification of the pathogenic microbe, strain, or metabolite thereof is used to diagnose the subject as having, or as at risk of developing, a recurrence or flare of a disease or disorder associated with nucleic acid damage, such as cancer. In other embodiments, the detection and identification of the pathogenic microbe, strain, or metabolite thereof is used to diagnose the subject as having, or as likely to have, remission or a disease or disorder associated with nucleic acid damage, such as cancer. In various embodiments, the diseases and disorders associated with nucleic acid damage include, but are not limited to, at least one of: anal cancer, appendix cancer, bile duct cancer, biliary cancer, carcinoid tumors, cholangiocarcinoma, colon adenocarcinoma, colon cancer, colorectal cancer, diffuse gastric cancer (HDGC), duodenal cancer, esophageal cancer, fibrolamellar hepatocellular carcinoma, gallbladder cancer, gastric adenocarcinoma and proximal polyposis of the stomach (GAPPS), gastric cancer, gastroesophageal (GE) junction cancer, gastrointestinal carcinoid tumor, gastrointestinal neuroendocrine tumor, gastrointestinal stromal tumor, hepatocellular carcinoma (HCC), intrahepatic cholangiocarcinoma (ICC), liver cancer, mucinous adenocarcinoma, neuroendocrine carcinoid tumor, pancreatic cancer, pancreatic islet cell tumor, pancreatic neuroendocrine tumor, primary gastrointestinal tumor, pseudomyxoma peritonei, rectal cancer, rectum adenocarcinoma, SDH-deficient gastrointestinal stromal tumor (GIST), signet ring cell adenocarcinoma, small bowel cancer, small intestine cancer, stomach (gastric) cancer, stomach adenocarcinoma, and stomach carcinoid tumor.

In some embodiments, the microbe is a gut microbe. In some embodiments, the gut microbe is isolated from a stool sample. In some embodiments, the microbe is a gram-negative bacterium. In some embodiments, the microbe is a gram-positive bacterium.

In various embodiments, the microbe associated with the development or progression of a disease or disorder associated with nucleic acid damage, such as cancer, in the subject is at least one selected from the group consisting of Bifidobacterium adolescentis, Bifidobacterium dentium, Bifidobacterium breve, Clostridium perfringens, Clostridium ramosum, Streptococcus mitis, Lactobacillus salivarius, Pediococcus acidilactici, Enterococcus asini, and Morganella morganii, or any combination thereof.

In various embodiments, the metabolite associated with the development or progression of a disease or disorder associated with nucleic acid damage, such as cancer, is produced directly or indirectly by at least one selected from the group consisting of Bifidobacterium adolescentis, Bifidobacterium dentium, Bifidobacterium breve, Clostridium perfringens, Clostridium ramosum, Streptococcus mitis, Lactobacillus salivarius, Pediococcus acidilactici, Enterococcus asini, and Morganella morganii, or any combination thereof.

Specific alterations in a subject's microbiota, including the presence of the pathogenic microbes or strains thereof, can be detected using various methods, including without limitation quantitative PCR or high-throughput sequencing methods which detect relative proportions of microbial genetic markers in a total heterogeneous microbial population. In some embodiments, the microbial genetic marker is a bacterial genetic marker. In particular embodiments, the bacterial genetic marker is at least some portion of the16S rRNA. In some embodiments, the relative proportion of particular constituent bacterial phyla, classes, orders, families, genera, and species present in the microbiota of a subject is determined. In other embodiments, the relative proportion of pathogenic and/or beneficial bacterial phyla, classes, orders, families, genera, and species present in the microbiota of a subject is determined. In some embodiments, the relative proportion of particular pathogenic and/or beneficial bacterial phyla, classes, orders, families, genera, and species present in the microbiota of a subject is determined and compared with that of a comparator normal microbiota. In various embodiments, the comparator normal microbiota is, by way of non-limiting examples, a microbiota of a subject known to be free of a disease or disorder associated with nucleic acid damage induced by the pathogenic microbe or strain thereof, free of a pathogenic microbe or strain thereof inducing a disease or disorder associated with nucleic acid damage, or a historical norm, or a typical microbiota of the population of which the subject is a member.

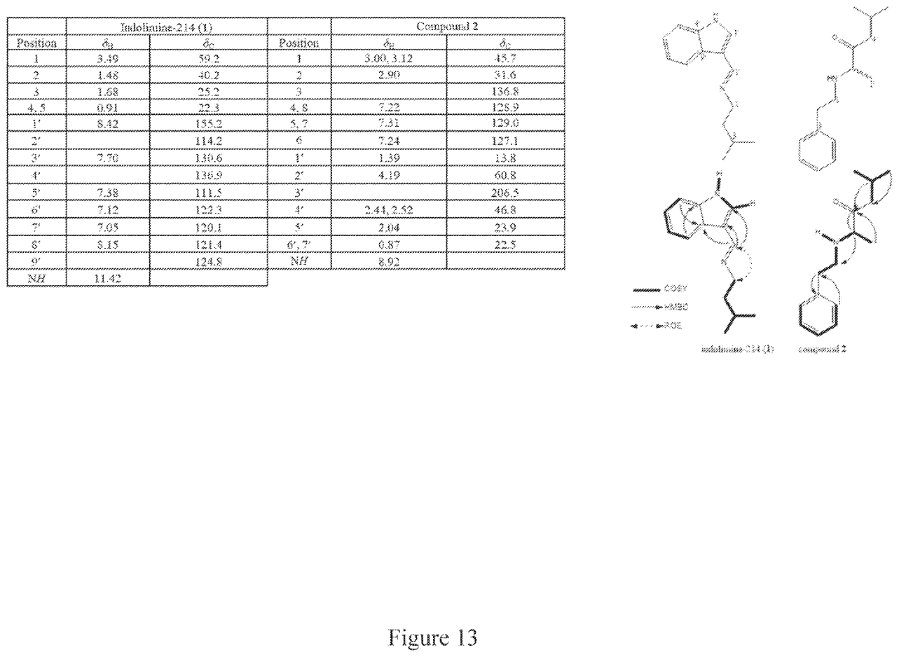

In one embodiment, the invention is a microbial metabolite associated with nucleic acid damage (i.e., genotoxin). In one embodiment, the invention is a microbial metabolite associated with nucleic acid damage identified using the methods described herein. In one embodiment, the microbial metabolite associated with nucleic acid damage is an indolimine. In one embodiment, the microbial metabolite associated with nucleic acid damage is indolmine-214. In one embodiment, the microbial metabolite associated with nucleic acid damage is indolimine-200. In one embodiment, the microbial metabolite associated with nucleic acid damage is indolimine-248. In one embodiment, the microbial metabolite associated with nucleic acid damage is indolimine-214 in combination with compound 2 (see FIG. 13).

In one embodiment, the invention is a codon-optimized nucleic acid sequence of the decarboxylase aat (i.e., Peg1085; pyridoxal-dependent decarboxylase or aspartate aminotransferase (aat)). In one embodiment, the invention is an indolimine produced by a codon-optimized nucleic acid sequence of the decarboxylase aat (i.e., pyridoxal-dependent decarboxylase or aspartate aminotransferase (aat); Peg1085).

In one embodiment, the invention is a bacterium that is genetically modified so that they cannot express one or more genes required for the production of a bacterial genotoxin. In one embodiment, the invention is a bacterium that is genetically modified so that they cannot express aat. In one embodiment, the bacterium that is genetically modified so that they cannot express one or more genes required for the production of a bacterial genotoxin is Morganella morganii. In one embodiment, the bacterium that is genetically modified so that they cannot express one or more genes required for the production of a bacterial genotoxin is Morganella morganii, which has been genetically modified so that it is unable to express aat. In one embodiment, the invention is a bacterium having a disabled or knocked-out aat gene (i.e., aat−). In one embodiment, the invention is a bacterium having a disabled or knocked-out aat gene, wherein the aat gene is disabled or knocked-out by a transposon insertion 7 bp after the aat gene start codon.

In one embodiment, the invention is an inhibitor of a gene responsible for the production of the genotoxin. In various embodiments, the gene responsible for the production of the genotoxin is a decarboxylase. In various embodiments, the gene responsible for the production of the genotoxin is a decarboxylase encoded by Peg1085 (i.e., pyridoxal-dependent decarboxylase or aspartate aminotransferase (aat)).

In one embodiment, the invention is a method of decreasing the level of one or more genotoxins in a subject in need thereof. In some embodiments, the subject in need has been diagnosed with cancer or at risk of developing cancer. In various embodiments, the cancer is any type of cancer formed of cells exposed to a genotoxic microbe, strain or metabolite thereof. In some embodiments, the cancer is at least one selected from the group consisting of anal cancer, appendix cancer, bile duct cancer, biliary cancer, carcinoid tumors, cholangiocarcinoma, colon adenocarcinoma, colon cancer, colorectal cancer, diffuse gastric cancer (HDGC), duodenal cancer, esophageal cancer, fibrolamellar hepatocellular carcinoma, gallbladder cancer, gastric adenocarcinoma and proximal polyposis of the stomach (GAPPS), gastric cancer, gastroesophageal (GE) junction cancer, gastrointestinal carcinoid tumor, gastrointestinal neuroendocrine tumor, gastrointestinal stromal tumor, hepatocellular carcinoma (HCC), intrahepatic cholangiocarcinoma (ICC), liver cancer, mucinous adenocarcinoma, neuroendocrine carcinoid tumor, pancreatic cancer, pancreatic islet cell tumor, pancreatic neuroendocrine tumor, primary gastrointestinal tumor, pseudomyxoma peritonei, rectal cancer, rectum adenocarcinoma, SDH-deficient gastrointestinal stromal tumor (GIST), signet ring cell adenocarcinoma, small bowel cancer, small intestine cancer, stomach (gastric) cancer, stomach adenocarcinoma, and stomach carcinoid tumor.

### Methods of Diagnosis

In various embodiments, the present invention relates to methods of diagnosing a subject as having, or assessing the risk of a subject for developing, a disease or disorder associated with nucleic acid damage.

In one embodiment, the present invention provides a method of diagnosing a disease or disorder associated with nucleic acid damage (e.g., cancer), in a subject by identifying a type or types of microbes, strains, or metabolites thereof (e.g., a pathogenic microbe or strain), or the gene(s) responsible for genotoxin production, in sample of the subject that contribute to the development or progression of the disease or disorder associated with nucleic acid damage. In some embodiments, after the subject is diagnosed, the subject is then treated, as described elsewhere herein.

In one embodiment, the present invention provides a method of diagnosing a disease or disorder associated with nucleic acid damage (e.g., cancer), in a subject by the level of microbes, strains, or metabolites thereof (e.g., a pathogenic microbe or strain) in sample of the subject that contribute to the development or progression of the disease or disorder associated with nucleic acid damage. In some embodiments, after the subject is diagnosed, the subject is then treated, as described elsewhere herein.

In one embodiment, the method of the invention is a diagnostic assay for diagnosing a disease or disorder associated with nucleic acid damage (e.g., cancer) in a subject in need thereof, by determining the types and level of particular types of pathogenic microbes or strains thereof present in a biological sample derived from the subject. For example, in some embodiments, the subject is diagnosed as having a disease or disorder associated with nucleic acid damage associated with a specific pathogenic microbe or strain thereof when the specific pathogenic microbe or strains thereof are determined to be present in the biological sample derived from the subject with increased relative abundance as compared with a comparator. In various embodiments, the comparator us a positive control, a negative control, a historical control, a historical norm, a predetermined threshold, or the level of another reference molecule in the biological sample.

For example, in various embodiments, the presence or amount of a microbe, strain, or metabolite thereof in a sample of a subject is indicative of a disease or disorder associated with nucleic acid damage. In some embodiments, the detection of the presence or an increased amount of a microbe or strain thereof, as compared to a control or comparator as provided herein, is used to diagnose the subject as having, or as at risk of developing, a disease or disorder associated with nucleic acid damage, such as cancer.

In various embodiments, the detection of the presence or amount of a microbe or strain thereof is used to assess the progression of a disease or disorder associated with nucleic acid damage (such as cancer), or to assess the efficacy of a treatment method.

In various embodiments, the type or level of microbe that is measured or assessed is at least one of Bifidobacterium adolescentis, Bifidobacterium dentium, Bifidobacterium breve, Clostridium perfringens, Clostridium ramosum, Streptococcus mitis, Lactobacillus salivarius, Pediococcus acidilactici, Enterococcus asini, and Morganella morganii, or any combination thereof.

In various embodiments of the method of the invention, a subject is diagnosed as having, or at risk for developing, a disease or disorder associated with nucleic acid damage induced by a microbe, strain, or metabolite thereof when the level detected is increased by at least 10%, by at least 20%, by at least 30%, by at least 40%, by at least 50%, by at least 60%, by at least 70%, by at least 80%, by at least 90%, by at least 100%, by at least 125%, by at least 150%, by at least 175%, by at least 200%, by at least 250%, by at least 300%, by at least 400%, by at least 500%, by at least 600%, by at least 700%, by at least 800%, by at least 900%, by at least 1000%, by at least 1500%, by at least 2000%, by at least 2500%, by at least 3000%, by at least 4000%, or by at least 5000%, in the biological sample when compared with a comparator. In various embodiments, the comparator us a positive control, a negative control, a historical control, a historical norm, a predetermined threshold, or the level of another reference molecule in the biological sample.

In various embodiments of the method of the invention, a subject is diagnosed as having, or at risk for developing, a disease or disorder associated with nucleic acid damage induced by a microbe, strain, or metabolite thereof when level of the microbe, strain, or metabolite thereof is detected at a level that is increased by at least 1 fold, at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 11 fold, at least 12 fold, at least 13 fold, at least 14 fold, at least 15 fold, at least 20 fold, at least 25 fold, at least 30 fold, at least 40 fold, at least 50 fold, at least 75 fold, at least 100 fold, at least 200 fold, at least 250 fold, at least 500 fold, or at least 1000 fold, in the biological sample when compared with a comparator. In various embodiments, the comparator us a positive control, a negative control, a historical control, a historical norm, a predetermined threshold, or the level of another reference molecule in the biological sample.

In one embodiment, the method of the invention is a diagnostic assay for diagnosing a disease or disorder associated with nucleic acid damage, or predicting the likelihood of developing a disease or disorder associated with nucleic acid damage, by determining the absolute amount or relative abundance of the microbe, strain, or metabolite thereof in a biological sample derived from the subject. In some embodiments, the subject is diagnosed as having a disease or disorder associated with nucleic acid damage induced by a microbe, strain, or metabolite thereof when the microbe, strain, or metabolite thereof are determined to be presented at an increased abundance, relative to a comparator. In various embodiments, the comparator us a positive control, a negative control, a historical control, a historical norm, a predetermined threshold, or the level of another reference molecule in the biological sample.

In various embodiments, the cancer is any type of cancer formed of cells exposed to a genotoxic microbe, strain or metabolite thereof. In some embodiments, the cancer is at least one selected from the group consisting of anal cancer, appendix cancer, bile duct cancer, biliary cancer, carcinoid tumors, cholangiocarcinoma, colon adenocarcinoma, colon cancer, colorectal cancer, diffuse gastric cancer (HDGC), duodenal cancer, esophageal cancer, fibrolamellar hepatocellular carcinoma, gallbladder cancer, gastric adenocarcinoma and proximal polyposis of the stomach (GAPPS), gastric cancer, gastroesophageal (GE) junction cancer, gastrointestinal carcinoid tumor, gastrointestinal neuroendocrine tumor, gastrointestinal stromal tumor, hepatocellular carcinoma (HCC), intrahepatic cholangiocarcinoma (ICC), liver cancer, mucinous adenocarcinoma, neuroendocrine carcinoid tumor, pancreatic cancer, pancreatic islet cell tumor, pancreatic neuroendocrine tumor, primary gastrointestinal tumor, pseudomyxoma peritonei, rectal cancer, rectum adenocarcinoma, SDH-deficient gastrointestinal stromal tumor (GIST), signet ring cell adenocarcinoma, small bowel cancer, small intestine cancer, stomach (gastric) cancer, stomach adenocarcinoma, and stomach carcinoid tumor.

In one embodiment, the method comprises detecting the level of a microbe, strain, or metabolite thereof in a test sample of a subject. In various embodiments, the test sample is a biological sample (e.g., fluid, tissue, fecal, cell, cellular component, etc.) of the subject. In some embodiments, the biological sample is blood, serum, plasma, saliva, sweat, stool, vaginal fluid, or urine. A biological sample can be obtained by appropriate methods, such as, by way of examples, blood draw, fluid draw, or biopsy. A biological sample can be used as the test sample; alternatively, a biological sample can be processed to enhance access to the target of detection and the processed biological sample can then be used as the test sample.

The methods of detecting a microbe, strain, or metabolite thereof may be carried out using any assay or methodology known in the art. In various embodiments of the invention, methods of measuring a microbe, strain, or metabolite thereof in a biological sample include, but are not limited to, PCR, sequencing, an immunochromatography assay, an immunodot assay, a Luminex assay, an ELISA assay, an ELISPOT assay, a protein microarray assay, a ligand-receptor binding assay, an immunostaining assay, a Western blot assay, a mass spectrophotometry assay, a radioimmunoassay (RIA), a radioimmunodiffusion assay, a liquid chromatography-tandem mass spectrometry assay, an ouchterlony immunodiffusion assay, reverse phase protein microarray, a rocket immunoelectrophoresis assay, an immunohistostaining assay, an immunoprecipitation assay, a complement fixation assay, FACS, an enzyme-substrate binding assay, an enzymatic assay, an enzymatic assay employing a detectable molecule, such as a chromophore, fluorophore, or radioactive substrate, a substrate binding assay employing such a substrate, a substrate displacement assay employing such a substrate, and a protein chip assay (see also, 2007, Van Emon, Immunoassay and Other Bioanalytical Techniques, CRC Press; 2005, Wild, Immunoassay Handbook, Gulf Professional Publishing; 1996, Diamandis and Christopoulos, Immunoassay, Academic Press; 2005, Joos, Microarrays in Clinical Diagnosis, Humana Press; 2005, Hamdan and Righetti, Proteomics Today, John Wiley and Sons; 2007).

The results of the diagnostic assay can be used alone, or in combination with other information from the subject, or from the biological sample derived from the subject. The test biological sample can be an in vitro sample or an in vivo sample.

In various embodiments, the subject is a human subject, and may be of any race, sex and age. Representative subjects include those who are suspected of having microbe, strain, or metabolite thereof, associated with a disease or disorder associated with nucleic acid damage (e.g., cancer), those who have been diagnosed with having microbe, strain, or metabolite thereof, associated with a disease or disorder associated with nucleic acid damage (e.g., cancer), those whose have a microbe, strain, or metabolite thereof, associated with a disease or disorder associated with nucleic acid damage (e.g., cancer), those who are at risk of a recurrence of having microbe, strain, or metabolite thereof, associated with a disease or disorder associated with nucleic acid damage (e.g., cancer), those who at risk of a flare form having microbe, strain, or metabolite thereof, associated with a disease or disorder associated with nucleic acid damage (e.g., cancer), and those who are at risk of developing having a microbe, strain, or metabolite thereof, associated with a disease or disorder associated with nucleic acid damage (e.g., cancer).

In some embodiments, the test biological sample is prepared from a biological sample obtained from the subject. In some instances, a heterogeneous population of microbes will be present in the biological samples. Enrichment of a population for microbes can be accomplished using separation technique. For example, microbes of interest may be enriched by separation the microbes of interest from the initial population using affinity separation techniques. Techniques for affinity separation may include magnetic separation using magnetic beads conjugated with an affinity reagent, affinity chromatography, “panning” with an affinity reagent attached to a solid matrix, e.g., plate, or other convenient technique. Other techniques providing separation include fluorescence activated cell sorting, which can have varying degrees of sophistication, such as multiple color channels, low angle and obtuse light scattering detecting channels, impedance channels, etc. One example of an affinity reagent useful in the methods of the invention is an antibody, such as anti-species antibody or anti-isotype (e.g., anti-IgA, anti-IgM) antibody. The details of the preparation of such antibodies and their suitability for use as affinity reagents are well-known to those skilled in the art. In some embodiments, labeled antibodies are used as affinity reagents. Conveniently, these antibodies are conjugated with a label for use in separation. Labels include magnetic beads, which allow for direct separation; biotin, which can be removed with avidin or streptavidin bound to a support; fluorochromes, which can be used with a fluorescence activated cell sorter; or the like, to allow for ease of separation of the particular cell type.

In various embodiments, the initial population of microbes is contacted with one or more affinity reagent(s) and incubated for a period of time sufficient to permit the affinity reagent to specifically bind to its target. The microbes in the contacted population that become labeled by the affinity reagent are selected for by any convenient affinity separation technique, e.g., as described elsewhere herein or as known in the art. Compositions highly enriched for a microbe of interest (e.g., secretory antibody-bound bacteria) are achieved in this manner. The affinity enriched microbes will be about 70%, about 75%, about 80%, about 85% about 90%, about 95% or more of the composition. In other words, the enriched composition can be a substantially pure composition of the microbes of interest.

In one embodiment, the test biological sample is a sample containing at least a fragment of a microbial nucleic acid. The term, “fragment,” as used herein, indicates that the portion of a nucleic acid (e.g., DNA, RNA) that is sufficient to identify it as comprising a microbial nucleic acid.

In some embodiments, the biological sample can be a sample from any source which contains a microbe, or strain thereof, nucleic acid (e.g., DNA, RNA), such as a bodily fluid or fecal sample, or a combination thereof. A biological sample can be obtained by any suitable method. In some embodiments, a biological sample containing bacterial DNA is used. In other embodiments, a biological sample containing bacterial RNA is used. The biological sample can be used as the test sample; alternatively, the biological sample can be processed to enhance access to nucleic acids, or copies of nucleic acids, and the processed biological sample can then be used as the test sample. For example, in various embodiments, a nucleic acid is prepared from a biological sample, for use in the methods. Alternatively, or in addition, if desired, an amplification method can be used to amplify nucleic acids comprising all or a fragment of an RNA or DNA in a biological sample, for use as the test biological sample in the assessment of the presence, absence and proportion of particular types of microbes present in the sample.

In some embodiments, hybridization methods, such as Southern analysis, Northern analysis, or in situ hybridizations, can be used (see Current Protocols in Molecular Biology, Ausubel, F. et al., eds., John Wiley & Sons, including all supplements). For example, the presence of nucleic acid from a particular type of microbe can be determined by hybridization of nucleic acid to a nucleic acid probe. A “nucleic acid probe,” as used herein, can be a DNA probe or an RNA probe.

The nucleic acid probe can be, for example, a full-length nucleic acid molecule, or a portion thereof, such as an oligonucleotide of at least 15, 30, 50, 100, 250 or 500 nucleotides in length and sufficient to specifically hybridize under stringent conditions to appropriate target RNA or DNA. The hybridization sample is maintained under conditions which are sufficient to allow specific hybridization of the nucleic acid probe to RNA or DNA. Specific hybridization can be performed under high stringency conditions or moderate stringency conditions, as appropriate. In a preferred embodiment, the hybridization conditions for specific hybridization are high stringency. More than one nucleic acid probe can also be used concurrently in this method. Specific hybridization of any one of the nucleic acid probes is indicative of the presence of the particular type of bacteria of interest, as described herein.

In Northern analysis (see Current Protocols in Molecular Biology, Ausubel, F. et al., eds., John Wiley & Sons, supra), the hybridization methods described above are used to identify the presence of a sequence of interest in an RNA, such as unprocessed, partially processed or fully processed rRNA. For Northern analysis, a test sample comprising RNA is prepared from a biological sample from the subject by appropriate means. Specific hybridization of a nucleic acid probe, as described above, to RNA from the biological sample is indicative of the presence of the particular type of bacteria of interest, as described herein.

Alternatively, a peptide nucleic acid (PNA) probe can be used instead of a nucleic acid probe in the hybridization methods described herein. PNA is a DNA mimic having a peptide-like, inorganic backbone, such as N-(2-aminoethyl)glycine units, with an organic base (A, G, C, T or U) attached to the glycine nitrogen via a methylene carbonyl linker (see, for example, 1994, Nielsen et al., Bioconjugate Chemistry 5.1). The PNA probe can be designed to specifically hybridize to a particular microbial nucleic acid sequence. Hybridization of the PNA probe to a nucleic acid sequence is indicative of the presence of the particular type of bacteria of interest.

Direct sequence analysis can also be used to detect a microbial nucleic acid of interest. A sample comprising DNA or RNA can be used, and PCR or other appropriate methods can be used to amplify all or a fragment of the nucleic acid, and/or its flanking sequences, if desired. The microbial nucleic acid, or a fragment thereof, is determined, using standard methods.

In another embodiment, arrays of oligonucleotide probes that are complementary to target microbial nucleic acid sequences can be used to detect and identify microbial nucleic acids. For example, in one embodiment, an oligonucleotide array can be used. Oligonucleotide arrays typically comprise a plurality of different oligonucleotide probes that are coupled to a surface of a substrate in different known locations. These oligonucleotide arrays, also known as “Genechips,” have been generally described in the art, for example, U.S. Pat. No. 5,143,854 and PCT patent publication Nos. WO 90/15070 and 92/10092. These arrays can generally be produced using mechanical synthesis methods or light directed synthesis methods which incorporate a combination of photolithographic methods and solid phase oligonucleotide synthesis methods. See Fodor et al., 1991, Science 251, 767-777, Pirrung et al., U.S. Pat. No. 5,143,854 (see also PCT Application No. WO 90/15070) and Fodor et al., PCT Publication No. WO 92/10092 and U.S. Pat. No. 5,424,186. Techniques for the synthesis of these arrays using mechanical synthesis methods are described in, e.g., U.S. Pat. No. 5,384,261.

After an oligonucleotide array is prepared, a nucleic acid of interest is hybridized with the array and scanned for particular microbial nucleic acids. Hybridization and scanning are generally carried out by methods described herein and also in, e.g., Published PCT Application Nos. WO 92/10092 and WO 95/11995, and U.S. Pat. No. 5,424,186, the entire teachings of which are incorporated by reference herein. In brief, a target microbial nucleic acid sequence is amplified by well-known amplification techniques, e.g., PCR. Typically, this involves the use of primer sequences that are complementary to the target sequence. Amplified target, generally incorporating a label, is then hybridized with the array under appropriate conditions. Upon completion of hybridization and washing of the array, the array is scanned to determine the position on the array to which the target sequence hybridizes. The hybridization data obtained from the scan is typically in the form of fluorescence intensities as a function of location on the array.

Other methods of nucleic acid analysis can be used to detect microbial nucleic acids of interest. Representative methods include direct manual sequencing (1988, Church and Gilbert, Proc. Natl. Acad. Sci. USA 81:1991-1995; 1977, Sanger et al., Proc. Natl. Acad. Sci. 74:5463-5467; Beavis et al. U.S. Pat. No. 5,288,644); automated fluorescent sequencing; single-stranded conformation polymorphism assays (SSCP); clamped denaturing gel electrophoresis (CDGE); denaturing gradient gel electrophoresis (DGGE) (1981, Sheffield et al., Proc. Natl. Acad. Sci. USA 86, 232-236), mobility shift analysis (Orita et al., 1981, Proc. Natl. Acad. Sci. USA 86, 2766-2770; 1987, Rosenbaum and Reissner, 1991, Biophys. Chem. 265, 1275; Keen et al., 1991, Trends Genet. 7, 5); restriction enzyme analysis (Flavell et al., 1978, Cell 15, 25; Geever et al., 1981, Proc. Natl. Acad. Sci. USA 78:5081); heteroduplex analysis; chemical mismatch cleavage (CMC) (Cotton et al., 1985, Proc. Natl. Acad. Sci. USA 85:4397-4401); RNase protection assays (Myers, et al., 1985, Science 230,1242); use of polypeptides which recognize nucleotide mismatches, such as E. coli mutS protein (see, for example, U.S. Pat. No. 5,459,039); Luminex xMAP™ technology; high-throughput sequencing (HTS) (Gundry and Vijg, 2011, Mutat Res doi:10.1016/j.mrfmmm.2011.10.001); next-generation sequencing (NGS) (Voelkerding et al., 2009, Clinical Chemistry 55:641-658; Su et al., 2011, Expert Rev Mol Diagn. 11:333-343; Ji and Myllykangas, 2011, Biotechnol Genet Eng Rev 27, 135-158); ion semiconductor sequencing (Rusk, 2011, Nature Methods doi:10.1038/nmeth.f.330; Rothberg et al., 2011, Nature 475:348-352) and/or allele-specific PCR, for example. These and other methods can be used to identify the presence of one or more microbial nucleic acids of interest, in a biological sample derived from a subject. In various embodiments of the invention, the methods of assessing a biological sample for the presence or absence of a particular nucleic acid sequence, as described herein, are used to detect, identify or quantify particular constituents (e.g., a pathogenic microbe or strain thereof) of a subject's sample, and to aid in the diagnosis of a disease or disorder associated with nucleic acid damage, such as cancer, in a subject in need thereof.

The probes and primers according to the invention can be labeled directly or indirectly with a radioactive or nonradioactive compound, by methods well known to those skilled in the art, in order to obtain a detectable and/or quantifiable signal; the labeling of the primers or of the probes according to the invention is carried out with radioactive elements or with nonradioactive molecules. Among the radioactive isotopes used, mention may be made of ³²P, ³³P, ³⁵S or ³H. The nonradioactive entities are selected from ligands such as biotin, avidin, streptavidin or digoxigenin, haptens, dyes, and luminescent agents such as radioluminescent, chemoluminescent, bioluminescent, fluorescent or phosphorescent agents.

Nucleic acids can be obtained from the biological sample using known techniques. Nucleic acid herein refers to RNA, including mRNA, and DNA, including genomic DNA. The nucleic acid can be double-stranded or single-stranded (i.e., a sense or an antisense single strand) and can be complementary to a nucleic acid encoding a polypeptide. The nucleic acid content may also be an RNA or DNA extraction performed on a fresh or fixed biological sample.

Routine methods also can be used to extract DNA from a biological sample, including, for example, phenol extraction. Alternatively, genomic DNA can be extracted with kits such as the QIAamp™. Tissue Kit (Qiagen, Chatsworth, Calif.), the Wizard™ Genomic DNA purification kit (Promega, Madison, Wis.), the Puregene DNA Isolation System (Gentra Systems, Inc., Minneapolis, Minn.), and the A.S.A.P.™ Genomic DNA isolation kit (Boehringer Mannheim, Indianapolis, Ind.).

There are many methods known in the art for the detection of specific nucleic acid sequences and new methods are continually reported. A great majority of the known specific nucleic acid detection methods utilize nucleic acid probes in specific hybridization reactions. Preferably, the detection of hybridization to the duplex form is a Southern blot technique. In the Southern blot technique, a nucleic acid sample is separated in an agarose gel based on size (molecular weight) and affixed to a membrane, denatured, and exposed to (admixed with) the labeled nucleic acid probe under hybridizing conditions. If the labeled nucleic acid probe forms a hybrid with the nucleic acid on the blot, the label is bound to the membrane.

In the Southern blot, the nucleic acid probe is preferably labeled with a tag. That tag can be a radioactive isotope, a fluorescent dye or the other well-known materials. Another type of process for the specific detection of nucleic acids of exogenous organisms in a body sample known in the art are the hybridization methods as exemplified by U.S. Pat. Nos. 6,159,693 and 6,270,974, and related patents. To briefly summarize one of those methods, a nucleic acid probe of at least 10 nucleotides, preferably at least 15 nucleotides, more preferably at least 25 nucleotides, having a sequence complementary to a desired region of the target nucleic acid of interest is hybridized in a sample, subjected to depolymerizing conditions, and the sample is treated with an ATP/luciferase system, which will luminesce if the nucleic sequence is present. In quantitative Southern blotting, levels of the target nucleic acid can be determined.

A further process for the detection of hybridized nucleic acid takes advantage of the polymerase chain reaction (PCR). The PCR process is well known in the art (U.S. Pat. Nos. 4,683,195, 4,683,202, and 4,800,159). To briefly summarize PCR, nucleic acid primers, complementary to opposite strands of a nucleic acid amplification target nucleic acid sequence, are permitted to anneal to the denatured sample. A DNA polymerase (typically heat stable) extends the DNA duplex from the hybridized primer. The process is repeated to amplify the nucleic acid target. If the nucleic acid primers do not hybridize to the sample, then there is no corresponding amplified PCR product. In this case, the PCR primer acts as a hybridization probe.

In PCR, the nucleic acid probe can be labeled with a tag as discussed before. Most preferably the detection of the duplex is done using at least one primer directed to the target nucleic acid. In yet another embodiment of PCR, the detection of the hybridized duplex comprises electrophoretic gel separation followed by dye-based visualization.

DNA amplification procedures by PCR are well known and are described in U.S. Pat. No. 4,683,202. Briefly, the primers anneal to the target nucleic acid at sites distinct from one another and in an opposite orientation. A primer annealed to the target sequence is extended by the enzymatic action of a heat stable DNA polymerase. The extension product is then denatured from the target sequence by heating, and the process is repeated. Successive cycling of this procedure on both DNA strands provides exponential amplification of the region flanked by the primers.

Amplification is then performed using a PCR-type technique, that is to say the PCR technique or any other related technique. Two primers, complementary to the target nucleic acid sequence are then added to the nucleic acid content along with a polymerase, and the polymerase amplifies the DNA region between the primers.

The expression “specifically hybridizing in stringent conditions” refers to a hybridizing step in the process of the invention where the oligonucleotide sequences selected as probes or primers are of adequate length and sufficiently unambiguous so as to minimize the amount of non-specific binding that may occur during the amplification. The oligonucleotide probes or primers herein described may be prepared by any suitable methods such as chemical synthesis methods.

Hybridization is typically accomplished by annealing the oligonucleotide probe or primer to the DNA under conditions of stringency that prevent non-specific binding but permit binding of this DNA which has a significant level of homology with the probe or primer.

Among the conditions of stringency is the melting temperature (Tm) for the amplification step using the set of primers, which is in the range of about 55° C. to about 70° C. Preferably, the Tm for the amplification step is in the range of about 59° C. to about 72° C. Most preferably, the Tm for the amplification step is about 60° C.

Typical hybridization and washing stringency conditions depend in part on the size (i.e., number of nucleotides in length) of the DNA or the oligonucleotide probe, the base composition and monovalent and divalent cation concentrations (Ausubel et al., 1997, eds Current Protocols in Molecular Biology).

In one embodiment, the process for determining the quantitative and qualitative profile according to the present invention is characterized in that the amplifications are real-time amplifications performed using a labeled probe, preferably a labeled hydrolysis-probe, capable of specifically hybridizing in stringent conditions with a segment of a nucleic acid sequence, or polymorphic nucleic acid sequence. The labeled probe is capable of emitting a detectable signal every time each amplification cycle occurs.

The real-time amplification, such as real-time PCR, is well known in the art, and the various known techniques will be employed in the best way for the implementation of the present process. These techniques are performed using various categories of probes, such as hydrolysis probes, hybridization adjacent probes, or molecular beacons. The techniques employing hydrolysis probes or molecular beacons are based on the use of a fluorescence quencher/reporter system, and the hybridization adjacent probes are based on the use of fluorescence acceptor/donor molecules.

Hydrolysis probes with a fluorescence quencher/reporter system are available in the market and are for example commercialized by the Applied Biosystems group (USA). Many fluorescent dyes may be employed, such as FAM dyes (6-carboxy-fluorescein), or any other dye phosphoramidite reagents.

Among the stringent conditions applied for any one of the hydrolysis-probes of the present invention is the Tm, which is in the range of about 65° C. to 75° C. Preferably, the Tm for any one of the hydrolysis-probes of the present invention is in the range of about 67° C. to about 70° C. Most preferably, the Tm applied for any one of the hydrolysis-probes of the present invention is about 67° C.

In one embodiment, the process for determining the quantitative and qualitative profile according to the present invention is characterized in that the amplification products can be elongated, wherein the elongation products are separated relative to their length. The signal obtained for the elongation products is measured, and the quantitative and qualitative profile of the labeling intensity relative to the elongation product length is established.

The elongation step, also called a run-off reaction, allows one to determine the length of the amplification product. The length can be determined using conventional techniques, for example, using gels such as polyacrylamide gels for the separation, DNA sequencers, and adapted software. Because some mutations display length heterogeneity, some mutations can be determined by a change in length of elongation products.

In one aspect, the invention includes a primer that is complementary to a target microbial nucleic acid, and more particularly the primer includes 12 or more contiguous nucleotides substantially complementary to the sequence flanking the nucleic acid sequence of interest. Preferably, a primer featured in the invention includes a nucleotide sequence sufficiently complementary to hybridize to a nucleic acid sequence of about 12 to 25 nucleotides. More preferably, the primer differs by no more than 1, 2, or 3 nucleotides from the target flanking nucleotide sequence. In another aspect, the length of the primer can vary in length, preferably about 15 to 28 nucleotides in length (e.g., 16, 17, 18, 19, 20, 21, 22, 23, 24, 25, 26, or 27 nucleotides in length).

### Inhibitors and Methods of Treating and Preventing

The present invention further relates to a method of decreasing or inhibiting a level of microbe, strain, or metabolite thereof. In one aspect, the invention relates, in part, to a method of decreasing or inhibiting the level of a pathogenic microbe, strain, or metabolite thereof in a subject in need thereof by administering to the subject an effective amount of an inhibitor, or a composition comprising an inhibitor. In one embodiment, the method comprises decreasing the level of a pathogenic microbe, strain, or metabolite thereof in a subject in need thereof. In one embodiment, the method comprises decreasing the level (e.g., activity, expression, concentration, amount, etc.) of a pathogenic microbe, strain, or metabolite thereof in a subject in need thereof by administering to the subject an effective amount of an inhibitor, or a composition comprising an inhibitor.

In various embodiments, the method comprises administering a therapeutically effective amount of at least one inhibitor, or composition comprising an inhibitor, described herein. In one embodiment, the method comprises administering a therapeutically effective amount of an inhibitor composition of the pathogenic microbe, strain, or metabolite thereof to the subject.

In some various embodiments, the microbe is Bifidobacterium adolescentis, Bifidobacterium dentium, Bifidobacteriun breve, Clostridium perfringens, Clostridium ramosum, Streptococcus mitis, Lactobacillus salivarius, Pediococcus acidilactici, Enterococcus asini, and Morganella morganii, or any combination thereof.

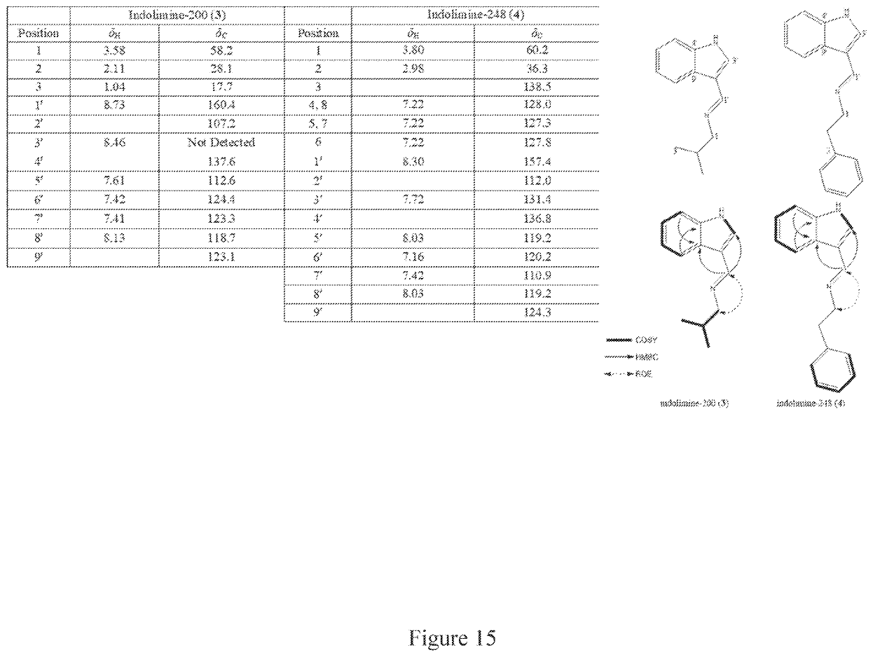

In various embodiments, the microbe metabolite is a genotoxin. In some embodiments, the microbe metabolite is a small molecule. In some embodiments, the small molecule metabolite is an indolimine. In some embodiments, the indolimine is indolimine-214. In some embodiments, the indolimine is indolimine-200. In some embodiments, the indolimine is indolimine-248. (See FIGS. 13 and 15)

In some embodiments, the method comprises administering at least one inhibitor of a gene involved in the synthesis of a genotoxin. In some embodiments, the gene involved in the synthesis of a genotoxin is a decarboxylase. In some embodiments, the gene involved in the synthesis of a genotoxin is Peg1085. Therefore, in some embodiments the invention relates to a method of administering an inhibitor of Peg1085 to a subject in need thereof.

The present invention also relates, in part, to a method of preventing or treating a disease or disorder associated with nucleic acid damage (such as cancer) associated with a pathogenic microbe, strain, or metabolite thereof in a subject in need thereof by administering to the subject an effective amount of an inhibitor, or a composition comprising an inhibitor. In various embodiments, the disease or disorder associated with nucleic acid damage induced the pathogenic microbe, strain, or metabolite thereof is cancer. In various embodiments, the cancer is at least one selected from anal cancer, appendix cancer, bile duct cancer, biliary cancer, carcinoid tumors, cholangiocarcinoma, colon adenocarcinoma, colon cancer, colorectal cancer, diffuse gastric cancer (HDGC), duodenal cancer, esophageal cancer, fibrolamellar hepatocellular carcinoma, gallbladder cancer, gastric adenocarcinoma and proximal polyposis of the stomach (GAPPS), gastric cancer, gastroesophageal (GE) junction cancer, gastrointestinal carcinoid tumor, gastrointestinal neuroendocrine tumor, gastrointestinal stromal tumor, hepatocellular carcinoma (HCC), intrahepatic cholangiocarcinoma (ICC), liver cancer, mucinous adenocarcinoma, neuroendocrine carcinoid tumor, pancreatic cancer, pancreatic islet cell tumor, pancreatic neuroendocrine tumor, primary gastrointestinal tumor, pseudomyxoma peritonei, rectal cancer, rectum adenocarcinoma, SDH-deficient gastrointestinal stromal tumor (GIST), signet ring cell adenocarcinoma, small bowel cancer, small intestine cancer, stomach (gastric) cancer, stomach adenocarcinoma, and stomach carcinoid tumor.

In some embodiments, the method comprises the steps of detecting the presence of the pathogenic microbe, strain, or metabolite thereof in the subject; identifying a beneficial microbe, strain, or metabolite thereof; and administering to the subject an effective amount of an inhibitor, or a composition comprising an inhibitor, to the subject.

In some embodiments, the method comprises the step of administering to the subject an inhibitor composition comprising at least one compound that reduces the level of the pathogenic microbe, strain, or metabolite thereof prior to the step of administering to the subject the composition comprising a probiotic microbe or strain thereof.

In some embodiments, the method comprises reducing the level (e.g., activity, expression, concentration, amount, etc.) of the pathogenic microbe, strain, or metabolite thereof. In some embodiments, the method comprises administering to the subject at least one compound that reduces the level (e.g., activity, expression, concentration, amount, etc.) of the pathogenic microbe, strain, or metabolite thereof. Examples of such compounds include, but are not limited to, a polyclonal antibody, a monoclonal antibody, an intracellular antibody, an antibody fragment, a single chain antibody (scFv), a heavy chain antibody, a synthetic antibody, a chimeric antibody, a humanized antibody, an anticalin, a nucleic acid, an antisense nucleic acid, an siRNA, an miRNA, a chemical compound, an antibiotic, a protein, a peptide, a peptidomemetic, a ribozyme, a small molecule chemical compound, prebiotic, or probiotic, or any combination thereof.

The method comprises administering a combination of compositions in any suitable ratio. For example, in one embodiment, the method comprises administering two individual compositions at a 1:1 ratio. However, the method is not limited to any particular ratio. Rather any ratio that is shown to be effective is encompassed.

The regimen of administration may affect what constitutes an effective amount. The therapeutic formulations may be administered to the subject either prior to or after a diagnosis of disease. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

In some embodiments, the method of treatment or prevention comprises monitoring the microbe level (e.g., the level of a pathogenic microbe, strain, or metabolite thereof) during the course of treatment or prevention of a disease or disorder associated with nucleic acid damage. In some embodiments, the method of treatment or prevention comprises an assessment of the effectiveness of the treatment regimen or prevention regimen for a disease or disorder associated with nucleic acid damage, such as cancer, by detecting the microbe level (e.g., the level of a pathogenic microbe, strain, or metabolite thereof) from samples obtained from a subject over time and comparing the microbe level detected. In some embodiments, a first sample is obtained prior to the subject receiving treatment/prevention and one or more subsequent samples are taken after or during treatment/prevention of the subject. In some embodiments, changes in microbe level over time provide an indication of effectiveness of the therapy.

In another aspect, the present invention relates to a method of predicting the effectiveness of a treatment or prevention of a disease or disorder associated with nucleic acid damage (e.g., cancer) in a subject, the treatment or prevention comprising administering to the subject having the disease or disorder associated with nucleic acid damage, a composition comprising an inhibitor composition. In various embodiments, the method comprises the steps of detecting the level (e.g., activity, expression, concentration, amount, etc.) of the microbe, strain, or metabolite thereof in the subject. In various embodiments, the method comprises the steps of comparing the level (e.g., activity, expression, concentration, amount, etc.) of the microbe, strain, or metabolite thereof to a comparator. In one embodiment, the method comprises the step of determining that the composition is effective when the level (e.g., activity, expression, concentration, amount, etc.) of the microbe, strain, or metabolite thereof is higher when compared to a comparator.

In some embodiments, the invention is an inhibitor, or a composition comprising an inhibitor, wherein the inhibitor diminishes the level (e.g., activity, expression, concentration, amount, etc.) of the pathogenic microbe, strain or metabolite thereof. In various embodiments, the inhibitor is at least one of a polyclonal antibody, a monoclonal antibody, an intracellular antibody, an antibody fragment, a single chain antibody (scFv), a heavy chain antibody, a synthetic antibody, a chimeric antibody, a humanized antibody, an anticalin, a nucleic acid, an antisense nucleic acid, an siRNA, an miRNA, a chemical compound, an antibiotic, a protein, a peptide, a peptidomemetic, a ribozyme, a small molecule chemical compound, prebiotic, or probiotic. In various embodiments, the composition comprises two or more inhibitors.

In other embodiments, the inhibitor composition comprises a therapeutically effective amount of antibiotic composition comprising an effective amount of at least one antibiotic, or a combinations of several types of antibiotics, wherein the administered antibiotic diminishes the number or pathogenic effects of at least one type (e.g., genus, species, strain, sub-strain, etc.) of pathogenic microbe or strain.

The type and dosage of the administered antibiotic will vary widely, depending upon the nature of the inflammatory disease or disorder, the character of subject's altered microbiota, the subject's medical history, the frequency of administration, the manner of administration, and the like. The initial dose may be larger, followed by smaller maintenance doses. The dose may be administered as infrequently as weekly or biweekly, or fractionated into smaller doses and administered daily, semi-weekly, etc., to maintain an effective dosage level. In various embodiments, the administered antibiotic is at least one of lipopeptide, fluoroquinolone, ketolide, cephalosporin, amikacin, gentamicin, kanamycin, neomycin, netilmicin, paromomycin, streptomycin, tobramycin, cefacetrile, cefadroxil, cefalexin, cefaloglycin, cefalonium, cefaloridine, cefalotin, cefapirin, cefatrizine, cefazaflur, cefazedone, cefazolin, cefradine, cefroxadine, ceftezole, cefaclor, cefamandole, cefmetazole, cefonicid, cefotetan, cefoxitin, cefprozil, cefuroxime, cefuzonam, cefcapene, cefdaloxime, cefdinir, cefditoren, cefetamet, cefixime, cefmenoxime, cefodizime, cefotaxime, cefpimizole, cefpodoxime, cefteram, ceftibuten, ceftiofur, ceftiolene, ceftizoxime, ceftriaxone, cefoperazone, ceftazidime, cefclidine, cefepime cefluprenam, cefoselis, cefozopran, cefpirome, cefquinome, cefaclomezine, cefaloram, cefaparole, cefcanel, cefedrolor, cefempidone, cefetrizole, cefivitril, cefmatilen, cefmepidium, cefovecin, cefoxazole, cefrotil, cefsumide, ceftaroline, ceftioxide, cefuracetime, imipenem, primaxin, doripenem, meropenem, ertapenem, flumequine, nalidixic acid, oxolinic acid, piromidic acid pipemidic acid, rosoxacin, ciprofloxacin, enoxacin, lomefloxacin, nadifloxacin, norfloxacin, ofloxacin, pefloxacin, rufloxacin, balofloxacin, gatifloxacin, grepafloxacin, levofloxacin, moxifloxacin, pazufloxacin, sparfloxacin, temafloxacin, tosufloxacin, clinafloxacin, gemifloxacin, sitafloxacin, trovafloxacin, prulifloxacin, azithromycin, erythromycin, clarithromycin, dirithromycin, roxithromycin, telithromycin, amoxicillin, ampicillin, bacampicillin, carbenicillin, cloxacillin, dicloxacillin, flucloxacillin, mezlocillin, nafcillin, oxacillin, penicillin g, penicillin v, piperacillin, pivampicillin, pivmecillinam, ticarcillin, sulfamethizole, sulfamethoxazole, sulfisoxazole, trimethoprim-sulfamethoxazole, demeclocycline, doxycycline, minocycline, oxytetracycline, tetracycline, linezolid, clindamycin, metronidazole, vancomycin, vancocin, mycobutin, rifampin, nitrofurantoin, chloramphenicol, or derivatives thereof.

In one embodiment, the invention is a method of treating disease or disorder associated with nucleic acid damage, such as cancer, in a subject in need thereof, including the step of administering to the subject at least one type (e.g., genus, species, strain, sub-strain, etc.) of probiotic bacteria, or a combinations of several types of bacteria, that is desired, preferred, neutral, beneficial, and/or under-represented in the subject.

Probiotic bacteria administered according to the methods of the present invention can comprise live bacteria. One or several different types of bacteria can be administered concurrently or sequentially. Such bacteria can be obtained from any source, including being isolated from a microbiota and grown in culture using known techniques.

In certain embodiments, the administered bacteria used in the methods of the invention further comprise a buffering agent. Examples of useful buffering agents include sodium bicarbonate, milk, yogurt, infant formula, and other dairy products.

Administration of a bacterium can be accomplished by any method suitable for introducing the organisms into the desired location. The bacteria can be mixed with a carrier and (for easier delivery to the digestive tract) applied to a liquid or to food. The carrier material should be non-toxic to the bacteria as wells as the subject. Preferably, the carrier contains an ingredient that promotes viability of the bacteria during storage. The formulation can include added ingredients to improve palatability, improve shelf-life, impart nutritional benefits, and the like.

The dosage of the administered bacteria (e.g., probiotic, surgical probiotic) will vary widely, depending upon the nature of the inflammatory disease or disorder associated with nucleic acid damage, the character of subject's altered microbiota, the subject's medical history, the frequency of administration, the manner of administration, the clearance of the agent from the host, and the like. The initial dose may be larger, followed by smaller maintenance doses. The dose may be administered as infrequently as weekly or biweekly, or fractionated into smaller doses and administered daily, semi-weekly, etc., to maintain an effective dosage level. It is contemplated that a variety of doses will be effective to achieve colonization of the gastrointestinal tract with the desired bacteria. In some embodiments, the dose ranges from about 10⁶ to about 10¹⁰ CFU per administration. In other embodiments, the dose ranges from about 10⁴ to about 10⁶ CFU per administration.

In certain embodiments, the probiotic bacteria administered in the therapeutic methods of the invention comprise administration of a combination of organisms.

While it is possible to administer a bacteria for therapy as is, it may be preferable to administer it in a pharmaceutical formulation, e.g., in admixture with a suitable pharmaceutical excipient, diluent or carrier selected with regard to the intended route of administration and standard pharmaceutical practice. The excipient, diluent and/or carrier must be “acceptable” in the sense of being compatible with the other ingredients of the formulation and not deleterious to the recipient thereof. Acceptable excipients, diluents, and carriers for therapeutic use are well known in the pharmaceutical art, and are described, for example, in Remington: The Science and Practice of Pharmacy. Lippincott Williams & Wilkins (A. R. Gennaro edit. 2005). The choice of pharmaceutical excipient, diluent, and carrier can be selected with regard to the intended route of administration and standard pharmaceutical practice.

Although there are no physical limitations to delivery of the formulations of the present invention, oral delivery is preferred for delivery to the digestive tract because of its ease and convenience, and because oral formulations readily accommodate additional mixtures, such as milk, yogurt, and infant formula. For delivery to colon, bacteria can be also administered rectally or by enema.

In one embodiment, the inhibitor composition comprises a probiotic. For example, in certain embodiments, the inhibitor composition comprises a probiotic composition that comprises one or more bacterium. In certain embodiments the one or more bacterium are indigenous members of the human gut microbiome. In some embodiments, the inhibitor composition comprises one or more bacterium from one or more bacterial species of Akkermansia sp., Bifidobacterium longum, Bifidobacterium breve, Bifidobacterium sp., Bifidobacterium infantis, Bidobacterium animalis, Bifidobacterium bifidum, Bifidobacterium adelocentis, Bifidobacterium lactis, Bifidobacterium pseudocatenulatum, Eggerthella lenta, Bacteroides sarotrii, Bacteroides fragilis, Bacteroides uniformis, Lactobacillus sp., Bifidobacterium sp., Lactococcus sp., Lactobacillus acidophilus, Lactobacillus rhamnosus, Lactobacillus fermentum, Lactobacillus casei, Lactobacillus bulgaricus, Lactobacillus gasseri, Lactobacillus helveticus, Lactobacillus johnsonii, Lactobacillus lactis, Lactobacillus plantarum, Lactobacillus reuteri, Lactobacillus salivarius, Lactobacillus paracasei, Parabacteroides distasonis, Dorea longicatena, Ruminococcus faecis, Blautia producta, Clostridium citroniae, Anaerostipes hadrus, Coprococcus comes, Roseburia faecis, Oscillospira plautii, and Clostridium spiroforme.

In some embodiments, the inhibitor composition comprises one or more bacterium, wherein the bacterium is genetically modified so that they cannot express one or more genes required for the production of a bacterial genotoxin. In some embodiments, the bacterium is genetically modified so that they cannot express one or more genes required for the production of a bacterial genotoxin because it is engineered to be unable to express a decarboxylase such as Peg1085; pyridoxal-dependent decarboxylase or aspartate aminotransferase (aat)). In some embodiments, the genetically modified bacteria is selected from one or more bacterial species of Bifidobacterium adolescentis, Bifidobacterium dentium, Bifidobacterium breve, Clostridium peifringens, Clostridium ramosum, Streptococcus mitis, Lactobacillus salivarius, Pediococcus acidilactici, Enterococcus asini, and Morganella morganii, or any combination thereof. In one embodiment, the bacterium that is genetically modified so that they cannot express one or more genes required for the production of a bacterial genotoxin is Morganella morganii. In one embodiment, the bacterium that is genetically modified so that they cannot express one or more genes required for the production of a bacterial genotoxin is Morganella morganii. In one embodiment, the bacterium that is genetically modified so that they cannot express one or more genes required for the production of a bacterial genotoxin is Morganella morganii, which has been genetically modified so that it is unable to express aat.

It should be appreciated that the inhibitor compositions may include bacterium from multiple species or from multiple strains of a particular species.

In various embodiments, the inhibitor composition is a probiotic composition for use as a food or drink additive. In some embodiments, the inhibitor composition is a probiotic beverage or drink. In some embodiments, the composition is soluble or suspendable in a liquid medium.

In various embodiments, the inhibitor composition comprises probiotic microorganisms in about 1×10⁹ cfu/g, about 2×10⁹ cfu/g, about 3×10⁹ cfu/g, about 4×10⁹ cfu/g, about 5×10⁹ cfu/g, about 6×10⁹ cfu/g, about 7×10⁹ cfu/g, about 8×10⁹ cfu/g, about 9×10⁹ cfu/g, about 1×10¹⁰ cfu/g, about 2×10¹⁰ cfu/g, about 3×10¹⁰ cfu/g, about 4×10¹⁰ cfu/g, about 5×10¹⁰ cfu/g, about 6×10¹⁰ cfu/g, about 7×10¹⁰ cfu/g, about 8×10¹⁰ cfu/g, about 9×10¹⁰ cfu/g, or about 1×10¹¹ cfu/g. In some embodiments, the probiotic composition comprises about 1×10¹⁰ cfu of probiotic microorganisms in each gram of bulk, dried raw powder where each gram contains about 60% or less of bacterial mass and about 40% carrier system. In some embodiments, each gram contains about 70% or less of bacterial mass and about 30% carrier system, about 80% or less of bacterial mass and about 20% carrier system, about 90% or less of bacterial mass and about 10% carrier system, about 50% or less of bacterial mass and about 50% carrier system, about 40% or less of bacterial mass and about 60% carrier system, about 30% or less of bacterial mass and about 70% carrier system, about 20% or less of bacterial mass and about 80% carrier system, or about 10% or less of bacterial mass and about 90% carrier system.

In some embodiments of the inhibitor compositions provided herein, the inhibitor compositions do not include probiotic bacterial species or strains that are resistant to one or more antibiotics. It should be appreciated that in certain instances, it may be desirable to have a mechanism to remove the bacterial compositions provided herein from the body of the subject after administration. One such mechanism is to remove the bacterial compositions by antibiotic treatment. Thus, in some embodiments, the compositions do not include bacterial species or strains that are resistant to one or more antibiotics. In some embodiments, the compositions do not include bacterial species or strains that are resistant to one or more antibiotics selected from the group consisting of penicillin, benzylpenicillin, ampicillin, sulbactam, amoxicillin, clavulanate, tazobactam, piperacillin, cefmetazole, vancomycin, imipenem, meropenem, metronidazole and clindamycin.

In some embodiments, the probiotic bacterial species or strain is known to be, or engineered to be, susceptible to one or more antibiotics. In some embodiments, the inhibitor compositions include bacterial species or strains that are susceptible to one, or two, or three, or four, or more antibiotics that are efficacious in humans. In some embodiments, the inhibitor compositions include bacterial species or strains that are susceptible to at least four antibiotics that are efficacious in humans. In some embodiments, the inhibitor compositions include bacterial species or strains that are susceptible to at least three antibiotics that are efficacious in humans. In some embodiments, the inhibitor compositions include bacterial species or strains that are susceptible to at least two antibiotics that are efficacious in humans. In some embodiments, the inhibitor compositions include bacterial species or strains that are susceptible to at least one antibiotic that is efficacious in humans. As used herein, an “antibiotic that is efficacious in a human” refers to an antibiotic that has been used to successfully treat bacterial infections in a human.

In some embodiments, the inhibitor compositions described herein comprise spore forming and non-spore forming bacterial species or strains. In some embodiments, the inhibitor compositions described herein comprise spore forming bacterial species or strains. In some embodiments, the inhibitor compositions described herein comprise only spore forming bacterial species or strains. In some embodiments, the inhibitor compositions described herein comprise only non-spore forming bacterial species or strains. The spore-forming bacteria can be in spore form (i.e., as spores) or in vegetative form (i.e., as vegetative cells). In spore form, bacteria are generally more resistant to environmental conditions, such as heat, acid, radiation, oxygen, chemicals, and antibiotics. In contrast, in the vegetative state or actively growing state, bacteria are more susceptible to such environmental conditions, compared to in the spore form. In general, bacterial spores are able to germinate from the spore form into a vegetative/actively growing state, under appropriate conditions. For instance, bacteria in spore format may germinate when they are introduced in the intestine.

In any of the inhibitor compositions provided herein, in some embodiments, the bacterial species or strains are purified. In any of the inhibitor compositions provided herein, in some embodiments, the bacterial species or strains are isolated. Any of the bacterial species or strains described herein may be isolated and/or purified, for example, from a source such as a culture or a microbiota sample (e.g., fecal matter). The bacterial strains used in the inhibitor compositions provided herein generally are isolated from the microbiome of healthy individuals. However, bacterial strains can also be isolated from individuals that are considered not to be healthy. In some embodiments, the inhibitor compositions include strains originating from multiple individuals.

As used herein, the term “isolated” bacteria that have been separated from one or more undesired component, such as another bacterium or bacterial species or strain, one or more component of a growth medium, and/or one or more component of a sample, such as a fecal sample. In some embodiments, the bacteria are substantially isolated from a source such that other components of the source are not detected.

As also used herein, the term “purified” refers to a bacterial species or strain or inhibitor composition comprising such that has been separated from one or more components, such as contaminants. In some embodiments, the bacterial species or strain is substantially free of contaminants. In some embodiments, one or more bacterial species or strains of a inhibitor composition may be independently purified from one or more other bacteria produced and/or present in a culture or a sample containing the bacterial species or strain. In some embodiments, a bacterial species or strain is isolated or purified from a sample and then cultured under the appropriate conditions for bacterial replication, e.g., under anaerobic culture conditions. The bacteria that is grown under appropriate conditions for bacterial replication can subsequently be isolated/purified from the culture in which it is grown.

In some embodiments, the one or more of the bacteria of the inhibitor compositions provided herein colonize or recolonize the intestinal tract or parts of the intestinal tract (e.g., the colon or the cecum) of a subject. Such colonization or recolonization may also be referred to as grafting. In some embodiments, the one or more of the bacterium of the inhibitor compositions recolonize the intestinal tract (e.g., the colon or the cecum) of a subject after the naturally present microbiome has been partially or completely removed, e.g., because of administration of antibiotics. In some embodiments, the one or more of the bacterium of the inhibitor compositions colonize a dysbiotic gastrointestinal tract.

The bacterial species or strains used in the inhibitor compositions provided herein generally are isolated from the microbiome of healthy individuals. In some embodiments, the inhibitor compositions include bacteria from species or strains originating from a single individual. In some embodiments, the inhibitor compositions include bacteria from species or strains originating from multiple individuals. In some embodiments, the bacterial strains are obtained from multiple individuals, isolated and grown up individually. The bacterial compositions that are grown up individually may subsequently be combined to provide the inhibitor compositions of the disclosure. It should be appreciated that the origin of the bacterial species or strains of the inhibitor compositions provided herein is not limited to the human microbiome from a healthy individual. In some embodiments, the bacterial species or strains originate from a human with a microbiome in dysbiosis. In some embodiments, the bacterial species or strains originate from non-human animals or the environment (e.g., soil or surface water). In some embodiments, the combinations of bacterial species or strains provided herein originate from multiple sources (e.g., human and non-human animals).

Any of the inhibitor compositions described herein, including the pharmaceutical compositions and food products comprising the compositions, may contain one or more bacterium in any form, for example in an aqueous form, such as a solution or a suspension, embedded in a semi-solid form, in a powdered form or freeze dried form. In some embodiments, the inhibitor composition or the one or more bacterium of the inhibitor composition are lyophilized. In some embodiments, a subset of the bacteria in a inhibitor composition is lyophilized. Methods of lyophilizing compositions, specifically compositions comprising bacteria, are well known in the art. See, e.g., U.S. Pat. Nos. 3,261,761; 4,205,132; PCT Publications WO 2014/029578 and WO 2012/098358, herein incorporated by reference in their entirety. The bacteria may be lyophilized as a combination and/or the bacteria may be lyophilized separately and combined prior to administration. One or more bacterium may be combined with a pharmaceutical excipient prior to combining it with the other bacterial or multiple lyophilized bacteria may be combined while in lyophilized form and the mixture of bacteria, once combined may be subsequently be combined with a pharmaceutical excipient. In some embodiments, the bacteria is a lyophilized cake. In some embodiments, the inhibitor compositions comprising the one or more bacterium are a lyophilized cake.

The bacterial species or strains of the inhibitor composition can be manufactured using fermentation techniques well known in the art. In some embodiments, the active ingredients are manufactured using anaerobic fermenters, which can support the rapid growth of anaerobic bacterial species. The anaerobic fermenters may be, for example, stirred tank reactors or disposable wave bioreactors. Culture media such as BL media and EG media, or similar versions of these media devoid of animal components, can be used to support the growth of the bacterial species. The bacterial product can be purified and concentrated from the fermentation broth by traditional techniques, such as centrifugation and filtration, and can optionally be dried and lyophilized by techniques well known in the art.

In some embodiments, the inhibitor composition may further comprise one or more additional therapeutic compositions. For example, in some embodiments, the composition further comprises a corticosteroids, mesalazine, mesalamine, sulfasalazine, sulfasalazine derivatives, immunosuppressive drugs, cyclosporin A, mercaptopurine, azathiopurine, prednisone, methotrexate, antihistamines, glucocorticoids, epinephrine, theophylline, cromolyn sodium, anti-leukotrienes, anti-cholinergic drugs for rhinitis, anti-cholinergic decongestants, mast-cell stabilizers, monoclonal anti-IgE antibodies, vaccines (preferably vaccines used for vaccination where the amount of an allergen is gradually increased), anti-TNF inhibitors such as infliximab, adalimumab, certolizumab pegol, golimumab, etanercept, or combinations thereof.

One of skill in the art will appreciate that the compositions of the invention can be administered singly or in any combination. Further, the compositions of the invention can be administered singly or in any combination in a temporal sense, in that they may be administered concurrently, or before, and/or after each other. One of ordinary skill in the art will appreciate, based on the disclosure provided herein, that the compositions of the invention can be used to prevent or to treat a disease or disorder associated with nucleic acid damage, and that the composition can be used alone or in any combination with another modulator to affect a therapeutic result. In various embodiments, any of the compositions of the invention described herein can be administered alone or in combination with other modulators of other molecules associated with the diseases and disorders described herein.

In one embodiment, the invention includes a method comprising administering a combination of compositions described herein. In some embodiments, the method has an additive effect, wherein the overall effect of the administering a combination of compositions is approximately equal to the sum of the effects of administering each individual composition. In other embodiments, the method has a synergistic effect, wherein the overall effect of administering a combination of compositions is greater than the sum of the effects of administering each individual composition.

### Pharmaceutical Compositions

In some embodiments, the inhibitor composition may be formulated for administration as a pharmaceutical inhibitor composition. The formulations of the pharmaceutical inhibitor compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

The term “pharmaceutical composition” as used herein means a product that results from the mixing or combining of at least one active ingredient, such as any two or more purified bacterial strains described herein, and one or more inactive ingredients, which may include one or more pharmaceutically acceptable excipient.

An “acceptable” excipient refers to an excipient that must be compatible with the active ingredient and not deleterious to the subject to which it is administered. In some embodiments, the pharmaceutically acceptable excipient is selected based on the intended route of administration of the composition, for example a composition for oral or nasal administration may comprise a different pharmaceutically acceptable excipient than a composition for rectal administration. Examples of excipients include sterile water, physiological saline, solvent, a base material, an emulsifier, a suspending agent, a surfactant, a stabilizer, a flavoring agent, an aromatic, an excipient, a vehicle, a preservative, a binder, a diluent, a tonicity adjusting agent, a soothing agent, a bulking agent, a disintegrating agent, a buffer agent, a coating agent, a lubricant, a colorant, a sweetener, a thickening agent, and a solubilizer.

Pharmaceutical compositions of the invention can be prepared in accordance with methods well known and routinely practiced in the art (see e.g., Remington: The Science and Practice of Pharmacy, Mack Publishing Co. 20th ed. 2000). The pharmaceutical compositions described herein may further comprise any carriers or stabilizers in the form of a lyophilized formulation or an aqueous solution. Acceptable excipients, carriers, or stabilizers may include, for example, buffers, antioxidants, preservatives, polymers, chelating reagents, and/or surfactants. In certain instances, pharmaceutical compositions are manufactured under GMP conditions. The pharmaceutical compositions can be used orally, nasally or parenterally, for instance, in the form of capsules, tablets, pills, sachets, liquids, powders, granules, fine granules, film-coated preparations, pellets, troches, sublingual preparations, chewables, buccal preparations, pastes, syrups, suspensions, elixirs, emulsions, liniments, ointments, plasters, cataplasms, transdermal absorption systems, lotions, inhalations, aerosols, injections, suppositories, and the like.

In some embodiments, the inhibitor composition is formulated with an enteric coating that increases the survival of the bacteria through the harsh environment in the stomach. The enteric coating is one which resists the action of gastric juices in the stomach so that the bacteria which are incorporated therein will pass through the stomach and into the intestines. The enteric coating may readily dissolve when in contact with intestinal fluids, so that the bacteria enclosed in the coating will be released in the intestinal tract. Enteric coatings may consist of polymer and copolymers well known in the art, such as commercially available EUDRAGIT (Evonik Industries). (See e.g., Zhang, 2016, AAPS PharmSciTech 17, 56-67). The inhibitor composition can be selected from the group consisting of a protein, an amino acid, a metabolite, a nucleic acid and any combination thereof.

In some embodiments, the inhibitor composition comprising a beneficial microbe or strain thereof is formulated for rectal delivery to the intestine (e.g., the colon). Thus, in some embodiments, the compositions may be formulated for delivery by suppository, colonoscopy, endoscopy, sigmoidoscopy or enema. A pharmaceutical preparation or formulation and particularly a pharmaceutical preparation for oral administration, may include an additional component that enables efficient delivery of the compositions of the disclosure to the intestine (e.g., the colon). A variety of pharmaceutical preparations that allow for the delivery of the compositions to the intestine (e.g., the colon) can be used. Examples thereof include pH sensitive compositions, more specifically, buffered sachet formulations or enteric polymers that release their contents when the pH becomes alkaline after the enteric polymers pass through the stomach. In certain embodiments, when a pH sensitive composition is used for formulating the pharmaceutical preparation, the pH sensitive composition is a polymer whose pH threshold of the decomposition of the composition is between about 6.8 and about 7.5. Such a numeric value range is a range in which the pH shifts toward the alkaline side at a distal portion of the stomach, and hence is a suitable range for use in the delivery to the colon. It should further be appreciated that each part of the intestine (e.g., the duodenum, jejunum, ileum, cecum, colon and rectum), has different biochemical and chemical environment. For instance, parts of the intestines have different pHs, allowing for targeted delivery by compositions that have a specific pH sensitivity. Thus, the compositions provided herein may be formulated for delivery to the intestine or specific parts of the intestine (e.g., the duodenum, jejunum, ileum, cecum, colon and rectum) by providing formulations with the appropriate pH sensitivity. (See e.g., Villena et al., 2015, Int J Pharm 487 (1-2): 314-9). The inhibitor composition can be selected from the group consisting of a protein, an amino acid, a metabolite, a nucleic acid and any combination thereof.

Another embodiment of a pharmaceutical preparation useful for delivery of the compositions to the intestine (e.g., the colon) is one that ensures the delivery to the colon by delaying the release of the contents (e.g., the beneficial microbe or strain thereof) by approximately 3 to 5 hours, which corresponds to the small intestinal transit time. In one embodiment of a pharmaceutical preparation for delayed release, a hydrogel is used as a shell. The hydrogel is hydrated and swells upon contact with gastrointestinal fluid, with the result that the contents are effectively released (released predominantly in the colon). Delayed release dosage units include drug-containing compositions having a material which coats or selectively coats a drug or active ingredient to be administered. Examples of such a selective coating material include in vivo degradable polymers, gradually hydrolyzable polymers, gradually water-soluble polymers, and/or enzyme degradable polymers. A wide variety of coating materials for efficiently delaying the release is available and includes, for example, cellulose-based polymers such as hydroxypropyl cellulose, acrylic acid polymers and copolymers such as methacrylic acid polymers and copolymers, and vinyl polymers and copolymers such as polyvinylpyrrolidone.

Additional examples of pharmaceutical compositions that allow for the delivery to the intestine (e.g., the colon) include bioadhesive compositions which specifically adhere to the colonic mucosal membrane (for example, a polymer described in the specification of U.S. Pat. No. 6,368,586) and compositions into which a protease inhibitor composition is incorporated for protecting particularly a biopharmaceutical preparation in the gastrointestinal tracts from decomposition due to an activity of a protease.

Another example of a system enabling the delivery to the intestine (e.g., the colon) is a system of delivering a composition to the colon by pressure change in such a way that the contents are released by utilizing pressure change caused by generation of gas in bacterial fermentation at a distal portion of the stomach. Such a system is not particularly limited, and a more specific example thereof is a capsule which has contents dispersed in a suppository base and which is coated with a hydrophobic polymer (for example, ethyl cellulose).

A further example of a system enabling the delivery of a composition to the intestine (e.g., the colon), is a composition that includes a coating that can be removed by an enzyme present in the gut (e.g., the colon), such as, for example, a carbohydrate hydrolase or a carbohydrate reductase. Such a system is not particularly limited, and more specific examples thereof include systems which use food components such as non-starch polysaccharides, amylose, xanthan gum, and azopolymers.

The compositions provided herein can also be delivered to specific target areas, such as the intestine, by delivery through an orifice (e.g., a nasal tube) or through surgery. In addition, the compositions provided herein that are formulated for delivery to a specific area (e.g., the cecum or the colon), may be administered by a tube (e.g., directly into the small intestine). Combining mechanical delivery methods such as tubes with chemical delivery methods such as pH specific coatings, allow for the delivery of the compositions provided herein to a desired target area (e.g., the cecum or the colon).

The compositions are formulated into pharmaceutically acceptable dosage forms by conventional methods known to those of skill in the art. Dosage regimens are adjusted to provide the optimum desired response (e.g., the prophylactic or therapeutic effect). In some embodiments, the dosage form of the composition is a tablet, pill, capsule, powder, granules, solution, or suppository. In some embodiments, the pharmaceutical composition is formulated for oral administration. In some embodiments, the pharmaceutical composition is formulated such that the bacteria of the composition, or a portion thereof, remain viable after passage through the stomach of the subject. In some embodiments, the pharmaceutical composition is formulated for rectal administration, e.g. as a suppository. In some embodiments, the pharmaceutical composition is formulated for delivery to the intestine or a specific area of the intestine (e.g., the colon) by providing an appropriate coating (e.g., a pH specific coating, a coating that can be degraded by target area specific enzymes, or a coating that can bind to receptors that are present in a target area).

Dosages of the active ingredients in the pharmaceutical compositions of the present invention can be varied so as to obtain an amount of the active ingredient which is effective to achieve the desired pharmaceutical response for a particular subject, composition, and mode of administration, without being toxic or having an adverse effect on the subject. The selected dosage level depends upon a variety of factors including the activity of the particular compositions of the present invention employed, the route of administration, the time of administration, the duration of the treatment, other drugs, compounds and/or materials used in combination with the particular compositions employed, the age, sex, weight, condition, general health and prior medical history of the subject being treated, and like factors.

A physician, veterinarian or other trained practitioner, can start doses of the pharmaceutical composition at levels lower than that required to achieve the desired therapeutic effect and gradually increase the dosage until the desired effect (e.g., treatment of a disease or disorder associated with nucleic acid damage, weight loss, decreased blood glucose, etc.) is achieved. In general, effective doses of the compositions of the present invention, for the prophylactic treatment of groups of people as described herein vary depending upon many different factors, including routes of administration, physiological state of the subject, whether the subject is human or an animal, other medications administered, and the therapeutic effect desired. Dosages need to be titrated to optimize safety and efficacy. In some embodiments, the dosing regimen entails oral administration of a dose of any of the compositions described herein. In some embodiments, the dosing regimen entails oral administration of multiple doses of any of the compositions described herein. In some embodiments, the composition is administered orally the subject once, twice, 3 times, 4 times, 5 times, 6 times, 7 times, 8 times, 9 times, or at least 10 times.

The inhibitor compositions, including the pharmaceutical inhibitor compositions disclosed herein, include compositions with a range of active ingredients. The amount of bacteria in the compositions may be expressed in weight, number of bacteria and/or CFUs (colony forming units). In some embodiments, the pharmaceutical compositions disclosed herein contain about 10, about 10², about 10³, about 10⁴, about 10⁵, about 10⁶, about 10⁷, about 10⁸, about 10⁹, about 10¹⁰, about 10¹¹, about 10¹², about 10¹³ or more of each of the bacteria of the composition per dosage amount. In some embodiments, the pharmaceutical compositions disclosed herein contain about 10, about 10², about 10³, about 10⁴, about 10⁵, about 10⁶, about 10⁷, about 10⁸, about 10⁹, about 10¹⁰, about 10¹¹, about 10¹², about 10¹³ or more total bacteria per dosage amount. It should further be appreciated that the bacteria of the compositions may be present in different amounts. In some embodiments, the pharmaceutical compositions disclosed herein contain about 10, about 10², about 10³, about 10⁴, about 10⁵, about 10⁶, about 10⁷, about 10⁸, about 10⁹, about 10¹⁰, about 10¹¹, about 10¹², about 10¹³ or more CFUs of each of the bacteria in the composition per dosage amount. In some embodiments, the pharmaceutical compositions disclosed herein contain about 10¹, about 10², about 10³, about 10⁴, about 10⁵, about 10⁶, about 10⁷, about 10⁸, about 10⁹, about 10¹⁰, about 10¹¹, about 10¹², about 10¹³ or more CFUs in total for all of the bacteria combined per dosage amount. As discussed above, bacteria of the compositions may be present in different amounts. In some embodiments, the pharmaceutical compositions disclosed herein contain about 10⁻⁷, about 10⁻⁶, about 10⁻⁵, about 10⁻⁴, about 10⁻³, about 10⁻², about 10⁻¹ or more grams of each of the bacteria in the composition per dosage amount. In some embodiments, the pharmaceutical compositions disclosed herein contain about 10⁻⁷, about 10⁻⁶, about 10⁻⁵, about 10⁻⁴, about 10⁻³, about 10⁻², about 10⁻¹ or more grams in total for all of the bacteria combined per dosage amount. In some embodiment, the dosage amount is one administration device (e.g., one table, pill or capsule). In some embodiment, the dosage amount is the amount that is administered in a particular period (e.g., one day or one week).

In some embodiments, the pharmaceutical compositions disclosed herein contain between 10 and 10¹³, between 10² and 10¹³, between 10³ and 10¹³, between 10⁴ and 10¹³, between 10⁵ and 10¹³, between 10⁶ and 10¹³, between 10⁷ and 10¹³, between 10⁸ and 10¹³, between 10⁹ and 10¹³, between 10¹⁰ and 10¹³, between 10¹¹ and 10¹³, between 10¹² and 10³, between 10 and 10¹², between 10² and 10¹², between 10³ and 10¹², between 10⁴ and 10¹² between 10⁵ and 10¹², between 10⁶ and 10¹², between 10⁷ and 10¹², between 10⁸ and 10¹² between 10⁹ and 10¹², between 10¹⁰ and 10¹², between 10¹¹ and 10¹², between 10 and 10¹¹, between 10² and 10¹¹, between 10³ and 10¹³, between 10⁴ and 10¹³, between 10⁵ and 10¹³, between 10⁶ and 10¹³, between 10⁷ and 10¹¹, between 10⁸ and 10¹¹, between 10⁹ and 10¹¹, between 10¹⁰ and 10¹¹, between 10 and 10¹⁰, between 10² and 10¹⁰, between 10³ and 10¹⁰, between 10⁴ and 10¹⁰, between 10⁵ and 10¹⁰, between 10⁶ and 10¹⁰, between 10⁷ and 10¹⁰, between 10⁸ and 10¹⁰, between 10⁹ and 10¹⁰, between 10 and 10⁹, between 10² and 10⁹, between 10³ and 10⁹, between 10⁴ and 10⁹, between 10⁵ and 10⁹, between 10⁶ and 10⁹, between 10⁷ and 10⁹, between 10⁸ and 10⁹, between 10 and 10⁸, between 10² and 10⁸, between 10³ and 10⁸, between 10⁴ and 10⁸, between 10⁵ and 10⁸, between 10⁶ and 10⁸, between 10⁷ and 10, between 10 and 10⁷, between 10² and 10⁷, between 10³ and 10⁷, between 10⁴ and 10⁷, between 10⁵ and 10⁷, between 10⁶ and 10⁷, between 10 and 10⁶, between 10² and 10⁶, between 10³ and 10⁶, between 10⁴ and 10⁶, between 10⁵ and 10⁶, between 10 and 10⁵, between 10² and 10⁵, between 10³ and 10⁵, between 10⁴ and 10⁵, between 10 and 10⁴, between 10² and 10⁴, between 10³ and 10⁴, between 10 and 10³, between 10² and 10³, or between 10 and 10² of each of the bacteria of the composition per dosage amount. In some embodiments, the pharmaceutical compositions disclosed herein contain between 10 and 10¹³, between 10² and 10¹³, between 10³ and 10¹³, between 10⁴ and 10¹³, between 10⁵ and 10¹³, between 10⁶ and 10¹³, between 10⁷ and 10¹³, between 10⁸ and 10¹³, between 10⁹ and 10¹³, between 10¹⁰ and 10¹³, between 10¹¹ and 10¹³, between 10¹² and 10¹³, between 10 and 10¹², between 10² and 10¹², between 10³ and 10¹², between 10⁴ and 10¹² between 10⁵ and 10¹², between 10⁶ and 10¹², between 10⁷ and 10¹², between 10⁸ and 10¹² between 10⁹ and 10¹², between 10¹⁰ and 10¹², between 10¹ and 10², between 10 and 10¹¹, between 10² and 10¹¹, between 10³ and 10¹³, between 10⁴ and 10¹³, between 10⁵ and 10¹³, between 10⁶ and 10¹³, between 10⁷ and 10¹, between 10⁸ and 10¹², between 10⁹ and 10¹¹, between 10¹⁰ and 10¹¹, between 10 and 10¹⁰, between 10² and 10¹⁰, between 10³ and 10¹⁰, between 10⁴ and 10¹⁰, between 10⁵ and 10¹⁰, between 10⁶ and 10¹⁰, between 10⁷ and 10¹⁰, between 10⁸ and 10¹⁰, between 10⁹ and 10¹⁰, between 10 and 10⁹, between 10² and 10⁹, between 10³ and 10⁹, between 10⁴ and 10⁹, between 10⁵ and 10⁹, between 10⁶ and 10⁹, between 10⁷ and 10⁹, between 10⁸ and 10⁹, between 10 and 10⁸, between 10² and 10⁸, between 10³ and 10⁸, between 10⁴ and 10⁸, between 10⁵ and 10⁸, between 10⁶ and 10⁸, between 10⁷ and 10⁸, between 10 and 10⁷, between 10² and 10⁷, between 10³ and 10⁷, between 10⁴ and 10⁷, between 10⁵ and 10⁷, between 10⁶ and 10⁷, between 10 and 10⁶, between 10² and 10⁶, between 10³ and 10⁶, between 10⁴ and 10⁶, between 10⁵ and 10⁶, between 10 and 10⁵, between 10² and 10⁵, between 10³ and 10⁵, between 10⁴ and 10⁵, between 10 and 10⁴, between 10² and 10⁴, between 10³ and 10⁴, between 10 and 10³, between 10² and 10³, or between 10 and 10² total bacteria per dosage amount.

In some embodiments, the pharmaceutical compositions disclosed herein contain between 10 and 10¹³, between 10² and 10¹³, between 10³ and 10¹³, between 10⁴ and 10¹³, between 10⁵ and 10¹³, between 10⁶ and 10¹³, between 10⁷ and 10¹³, between 10⁸ and 10¹, between 10⁹ and 10¹³, between 101° and 10³, between 10¹² and 10¹³, between 10¹² and 10¹³, between 10 and 10¹², between 10² and 10¹², between 10³ and 10¹², between 10⁴ and 10¹² between 10⁵ and 10¹², between 10⁶ and 10¹², between 10⁷ and 10¹², between 10⁸ and 10¹² between 10⁹ and 10¹², between 10¹⁰ and 10¹², between 10¹ and 10², between 10 and 10¹¹, between 10² and 10¹¹, between 10³ and 10¹³, between 10⁴ and 10¹³, between 10⁵ and 10¹³, between 10⁶ and 10¹³, between 10⁷ and 10¹³, between 10⁸ and 10¹³, between 10⁹ and 10¹¹, between 10¹⁰ and 10¹¹, between 10 and 10¹⁰, between 10² and 10¹⁰, between 10³ and 10¹⁰, between 10⁴ and 10¹⁰, between 10⁵ and 10¹⁰, between 10⁶ and 10¹⁰, between 10⁷ and 10¹⁰, between 10 and 10¹⁰, between 10⁹ and 10¹⁰, between 10 and 10⁹, between 10² and 10⁹, between 10³ and 10⁹, between 10⁴ and 10⁹, between 10⁵ and 10⁹, between 10⁶ and 10⁹, between 10⁷ and 10⁹, between 10⁸ and 10⁹, between 10 and 10⁸, between 10² and 10⁸, between 10³ and 10⁸, between 10⁴ and 10⁸, between 10⁵ and 10⁸, between 10⁶ and 10⁸, between 10⁷ and 10⁸, between 10 and 10⁷, between 10² and 10⁷, between 10³ and 10⁷, between 10⁴ and 10⁷, between 10⁵ and 10⁷, between 10⁶ and 10⁷, between 10 and 10⁶, between 10² and 10⁶, between 10³ and 10⁶, between 10⁴ and 10⁶, between 10⁵ and 10⁶, between 10 and 10⁵, between 10² and 10, between 10³ and 10⁵, between 10⁴ and 10⁵, between 10 and 10⁴ between 10² and 10⁴, between 10³ and 10⁴, between 10 and 10³, between 10² and 10³, or between 10 and 10² CFUs of each of the bacteria of the composition per dosage amount. In some embodiments, the pharmaceutical compositions disclosed herein contain between 10 and 10¹³, between 10² and 10¹³, between 10³ and 10¹³, between 10⁴ and 10¹³, between 10⁵ and 10¹³, between 10⁶ and 10¹³, between 10⁷ and 10¹³, between 10⁸ and 10¹³, between 10⁹ and 10¹³, between 10¹⁰ and 10¹³, between 10¹ and 10¹³, between 10¹² and 10¹³, between 10 and 10¹⁹, between 10 and 10¹², between 10³ and 10¹², between 10⁴ and 10¹², between 10⁵ and 10¹², between 10⁶ and 10¹², between 10⁷ and 10¹², between 10⁸ and 10¹², between 10⁹ and 10¹², between 10¹⁰ and 10¹², between 1⁰ and 10², between 10 and 10¹¹, between 10² and 10¹¹, between 10³ and 10¹³, between 10⁴ and 10¹³, between 10⁵ and 10¹³, between 10⁶ and 10¹³, between 10⁷ and 10¹³, between 10⁸ and 10¹¹, between 10⁹ and 10¹¹, between 10¹⁰ and 10¹¹, between 10 and 10¹⁰, between 10² and 10¹⁰, between 10³ and 10¹⁰, between 10⁴ and 10¹⁰, between 10⁵ and 10¹⁰, between 10⁶ and 10¹⁰, between 10⁷ and 10¹⁰, between 10⁸ and 10¹⁰, between 10⁹ and 10¹⁰, between 10 and 10⁹, between 10² and 10⁹, between 10³ and 10⁹, between 10⁴ and 10⁹, between 10⁵ and 10⁹, between 10⁶ and 10⁹, between 10⁷ and 10⁹, between 10⁸ and 10⁹, between 10 and 10⁸, between 10² and 10⁸, between 10³ and 10⁸, between 10⁴ and 10⁸, between 10⁵ and 10⁸, between 10⁶ and 10⁸, between 10⁷ and 10⁸, between 10 and 10⁷, between 10² and 10⁷, between 10³ and 10⁷, between 10⁴ and 10⁷, between 10⁵ and 10⁷, between 10⁶ and 10⁷, between 10 and 10⁶, between 10² and 10⁶, between 10³ and 10⁶, between 10⁴ and 10⁶, between 10⁵ and 10⁶, between 10 and 10⁵, between 10² and 10⁵, between 10³ and 10⁵, between 10⁴ and 10′, between 10 and 10⁴, between 10² and 10⁴, between 10³ and 10⁴, between 10 and 10³, between 10² and 10³, or between 10 and 10² total CFUs per dosage amount.

In some embodiments, the pharmaceutical compositions disclosed herein contain between 10⁻⁷ and 10⁻¹, between 10⁻⁶ and 10⁻¹, between 10⁻⁵ and 10⁻¹, between 10⁻⁴ and 10⁻¹, between 10⁻³ and 10⁻¹, between 10⁻² and 10⁻¹, between 10⁻⁷ and 10⁻², between 10⁻⁶ and 10⁻², between 10⁻⁵ and 10⁻², between 10⁻⁴ and 10⁻², between 10⁻³ and 10⁻², between 10⁻⁷ and 10⁻³ between 10⁻⁶ and 10⁻³, between 10⁻⁵ and 10⁻³, between 10⁻⁴ and 10⁻³, between 10⁻⁷ and 10⁻⁴ between 10⁻⁶ and 10⁻⁴, between 10⁻⁵ and 10⁻⁴, between 10⁻⁷ and 10⁻⁵, between 10⁻⁶ and 10⁻⁵, or between 10⁻⁷ and 10⁻⁶ grams of each of the bacteria in the composition per dosage amount. In some embodiments, the pharmaceutical compositions disclosed herein contain between 10⁻⁷ and 10⁻¹, between 10⁻⁶ and 10⁻¹, between 10⁻⁵ and 10⁻¹, between 10⁻⁴ and 10⁻¹, between 10⁻³ and 10⁻¹, between 10⁻² and 10⁻¹, between 10⁻⁷ and 10⁻², between 10⁻⁶ and 10⁻², between 10⁻⁵ and 10⁻², between 10⁻⁴ and 10⁻², between 10⁻³ and 10⁻², between 10⁻⁷ and 10⁻³, between 10⁻⁶ and 10⁻³, between 10⁻⁵ and 10⁻³, between 10⁻⁴ and 10⁻³, between 10⁻⁷ and 10⁻⁴, between 10⁻⁶ and 10⁻⁴, between 10⁻⁵ and 10⁻⁴, between 10⁻⁷ and 10⁻⁵, between 10⁻⁶ and 10⁻⁵, or between 10⁻⁷ and 10⁻⁶ grams of all of the bacteria combined per dosage amount.

Also with the scope of the present disclosure are food products comprising any of the prebiotics and/or bacterial species or strains described herein and a nutrient. Food products are, in general, intended for the consumption of a human or an animal. Any of the prebiotics and/or bacterial species or strains described herein may be formulated as a food product. In some embodiments, the one or more bacterium are formulated as a food product in spore form. In some embodiments, the one or more bacterium are formulated as a food product in vegetative form. In some embodiments, the food product comprises both vegetative bacteria and bacteria in spore form. The compositions disclosed herein can be used in a food or beverage, such as a health food or beverage, a food or beverage for infants, a food or beverage for pregnant women, athletes, senior citizens or other specified group, a functional food, a beverage, a food or beverage for specified health use, a dietary supplement, a food or beverage for patients, or an animal feed. Non-limiting examples of the foods and beverages include various beverages such as juices, refreshing beverages, tea beverages, drink preparations, jelly beverages, and functional beverages; alcoholic beverages such as beers; carbohydrate-containing foods such as rice food products, noodles, breads, and pastas; paste products such as fish hams, sausages, paste products of seafood; retort pouch products such as curries, food dressed with a thick starchy sauces, soups; dairy products such as milk, dairy beverages, ice creams, cheeses, and yogurts; fermented products such as fermented soybean pastes, yogurts, fermented beverages, and pickles; bean products; various confectionery products such as Western confectionery products including biscuits, cookies, and the like, Japanese confectionery products including steamed bean-jam buns, soft adzuki-bean jellies, and the like, candies, chewing gums, gummies, cold desserts including jellies, cream caramels, and frozen desserts; instant foods such as instant soups and instant soy-bean soups; microwavable foods; and the like. Further, the examples also include health foods and beverages prepared in the forms of powders, granules, tablets, capsules, liquids, pastes, and jellies.

Food products containing the prebiotics and/or bacterial species or strains described herein may be produced using methods known in the art and may contain the same amount of prebiotic or bacteria (e.g., by weight, amount or CFU) as the pharmaceutical compositions provided herein. Selection of an appropriate amount of prebiotic or bacteria in the food product may depend on various factors, including for example, the serving size of the food product, the frequency of consumption of the food product, the specific prebiotic or bacteria contained in the food product, the amount of water in the food product, and/or additional conditions for survival of the bacteria in the food product.

Examples of food products which may be formulated to contain any of the prebiotic and/or bacterial species or strains described herein include, without limitation, a beverage, a drink, a bar, a snack, a dairy product, a confectionery product, a cereal product, a ready-to-eat product, a nutritional formula, such as a nutritional supplementary formulation, a food or beverage additive.

Suitable routes of administration may, for example, include topical, oral, rectal, transmucosal, or intestinal administration; parenteral delivery, including intramuscular, subcutaneous, intravenous, intramedullary injections, as well as intrathecal, direct intraventricular, intraperitoneal, intranasal, or intraocular injections.

Alternatively, one may administer the compound in a local rather than systemic manner, for example, via injection of the compound directly into the area of pain, often in a depot or sustained release formulation. Furthermore, one may administer the drug in a targeted drug delivery system, for example, in a liposome coated with a tissue-specific antibody. The liposomes will be targeted to and taken up selectively by the organ.

The compositions disclosed herein may be manufactured in a manner that is itself known, e.g., by means of conventional mixing, dissolving, granulating, dragee-making, levigating, emulsifying, encapsulating, entrapping or tableting processes.

Inhibitor compositions for use in accordance with the present disclosure thus may be formulated in a conventional manner using one or more physiologically acceptable carriers comprising excipients and auxiliaries, which facilitate processing of the active compounds into preparations, which can be used pharmaceutically. Proper formulation is dependent upon the route of administration chosen. Any of the well-known techniques, carriers, and excipients may be used as suitable and as understood in the art, e.g., in Remington's Pharmaceutical Sciences, above.

For injection, the agents disclosed herein may be formulated in aqueous solutions, preferably in physiologically compatible buffers, such as Hank's solution, Ringer's solution, or physiological saline buffer. For transmucosal administration, penetrants appropriate to the barrier to be permeated are used in the formulation. Such penetrants are generally known in the art.

For oral administration, either solid or fluid unit dosage forms can be prepared. For preparing solid compositions, such as tablets, the compound of Formula (I) or derivatives thereof, disclosed above herein, is mixed into formulations with conventional ingredients, such as talc, magnesium stearate, dicalcium phosphate, magnesium aluminum silicate, calcium sulfate, starch, lactose, acacia, methylcellulose, and functionally similar materials as pharmaceutical diluents or carriers. For oral administration, the compounds can be also formulated readily by combining the active compounds with pharmaceutically acceptable carriers well known in the art. Such carriers enable the compounds disclosed herein to be formulated as tablets, pills, dragees, capsules, liquids, gels, syrups, slurries, suspensions and the like, for oral ingestion by a subject to be treated. Pharmaceutical preparations for oral use can be obtained by mixing one or more solid excipient with pharmaceutical combination disclosed herein, optionally grinding the resulting mixture, and processing the mixture of granules, after adding suitable auxiliaries, if desired, to obtain tablets or dragee cores. Suitable excipients are, in particular, fillers, such as sugars, including lactose, sucrose, mannitol, or sorbitol; cellulose preparations, such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof, such as sodium alginate.

Capsules are prepared by mixing the compound with an inert pharmaceutical diluent and filling the mixture into a hard gelatin capsule of appropriate size. Soft gelatin capsules are prepared by machine encapsulation of slurry of the compound with an acceptable vegetable oil, light liquid petrolatum or other inert oil. Fluid unit dosage forms for oral administration, such as syrups, elixirs, and suspensions, can be prepared. The water-soluble forms can be dissolved in an aqueous vehicle together with sugar, aromatic flavoring agents and preservatives to form syrup. An elixir is prepared by using a hydro alcoholic (e. g., ethanol) vehicle with suitable sweeteners, such as sugar and saccharin, together with an aromatic flavoring agent. Suspensions can be prepared with an aqueous vehicle with the aid of a suspending agent, such as acacia, tragacanth, methylcellulose, and the like.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Starch microspheres can be prepared by adding a warm aqueous starch solution, e. g., of potato starch, to a heated solution of polyethylene glycol in water with stirring to form an emulsion. When the two-phase system has formed (with the starch solution as the inner phase) the mixture is then cooled to room temperature under continued stirring whereupon the inner phase is converted into gel particles. These particles are then filtered off at room temperature and slurred in a solvent, such as ethanol, after which the particles are again filtered off and laid to dry in air. The micro spheres can be hardened by well-known cross-linking procedures, such as heat treatment or by using chemical cross-linking agents. Suitable agents include dialdehydes, including glyoxal, malondialdehyde, succinic aldehyde, adipaldehyde, glutaraldehyde and phthalaldehyde, diketones, such as butadione, epichlorohydrin, polyphosphate, and borate. Dialdehydes are used to crosslink proteins, such as albumin, by interaction with amino groups, and diketones form schiff bases with amino groups. Epichlorohydrin activates compounds with nucleophiles, such as amino or hydroxyl, to an epoxide derivative.

Pharmaceutical preparations, which can be used orally, include push-fit capsules made of gelatin, as well as soft, sealed capsules made of gelatin and a plasticizer, such as glycerol or sorbitol. The push-fit capsules can contain the active ingredients in admixture with filler, such as lactose, binders, such as starches, and/or lubricants, such as talc or magnesium stearate, and, optionally, stabilizers. In soft capsules, the active compounds may be dissolved or suspended in suitable liquids, such as fatty oils, liquid paraffin, or liquid polyethylene glycols. In addition, stabilizers and/or antioxidants may be added. All formulations for oral administration should be in dosages suitable for such administration.

Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents, such as suspending, stabilizing, and/or dispersing agents.

Slow or extended-release delivery systems, including any of a number of biopolymers (biological-based systems), systems employing liposomes, colloids, resins, and other polymeric delivery systems or compartmentalized reservoirs, can be utilized with the compositions described herein to provide a continuous or long-term source of therapeutic compound. Such slow-release systems are applicable to formulations for delivery via topical, intraocular, oral, and parenteral routes.

Compositions of the present invention also include aqueous solutions of the active compounds in water-soluble form. Additionally, suspensions of the active compounds may be prepared as appropriate oily suspensions. Suitable lipophilic solvents or vehicles include fatty oils, such as sesame oil, or synthetic fatty acid esters, such as ethyl oleate or triglycerides, or liposomes. Aqueous suspensions may contain substances, which increase the viscosity of the suspension, such as sodium carboxymethyl cellulose, sorbitol, or dextran. Optionally, the suspension may also contain suitable stabilizers or agents, which increase the solubility of the compounds to allow for the preparation of highly, concentrated solutions.

Alternatively, the active ingredient may be in powder form for constitution with a suitable vehicle, e.g., sterile pyrogen-free water, before use.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out exemplary embodiments of the present invention, and are not to be construed as limiting in any way the remainder of the disclosure.

### Example 1

Microbiota-derived metabolites that elicit DNA damage can contribute to colorectal cancer (CRC). However, the full spectrum of genotoxic chemicals produced by indigenous gut microbes remains to be defined. These experiments established a pipeline to systematically evaluate the genotoxicity of an extensive collection of gut commensals from inflammatory bowel disease patients. The examples presented here identified isolates from divergent phylogenies whose metabolites caused DNA damage and discovered a unique family of genotoxins—termed the indolimines—produced by the CRC-associated species Morganella morganii. A non-indolimine-producing M. morganii mutant lacked genotoxicity and failed to exacerbate colon tumorigenesis in mice. These studies reveal the existence of a previously unexplored universe of genotoxic small molecules from the microbiome that may impact host biology in homeostasis and disease.

Aside from a small number of case studies (Unterhauser et al., 2019, Proc. Natl. Acad. Sci. U.S.A. 116, 3774-3783; Dougherty et al., 2021, Toxins (Basel) 13, 346), the taxonomic distribution and repertoire of small molecule genotoxins produced by the microbiota remain mostly unexplored. The experiments undertook a systematic evaluation of the genotoxicity of a diverse selection of human gut microbes based on prior evidence that colibactin-producing E. coli induces DNA damage and facilitates intestinal tumorigenesis (Cuevas-Ramos et al., 2010, Proc. Natl. Acad. Sci. U.S.A. 107, 11537-11542). The experiments demonstrate that diverse taxa from the human gut microbiota exhibited genotoxicity. Further a previously undescribed family of genotoxic M. morganii-derived small molecules termed the indolimines was identified and characterized. The indolimine production pathway in M. morganii was decoded and an isogenic non-indolimine-producing M. morganii mutant was generated. Finally it was found that indolimine-producing M. morganii exacerbated colon tumorigenesis in gnotobiotic mice.

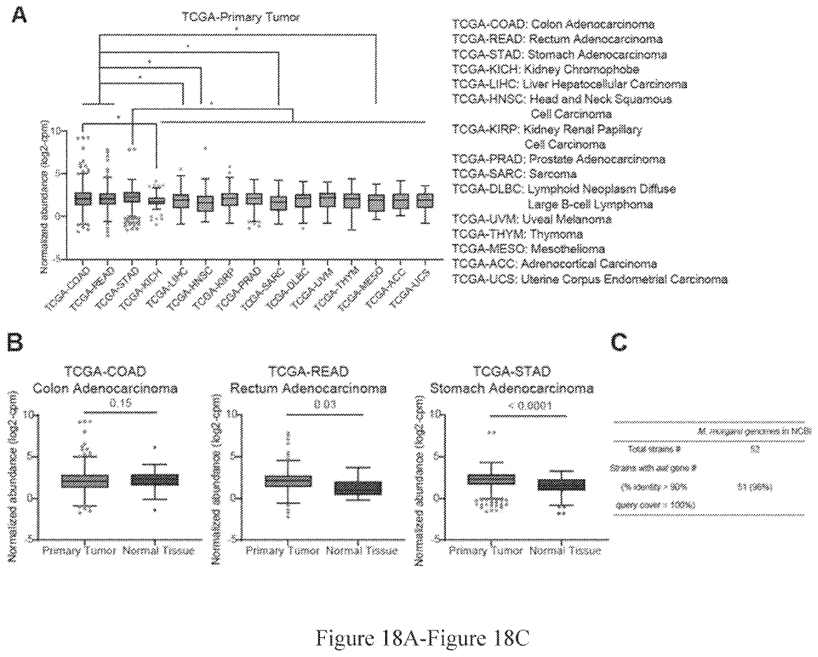

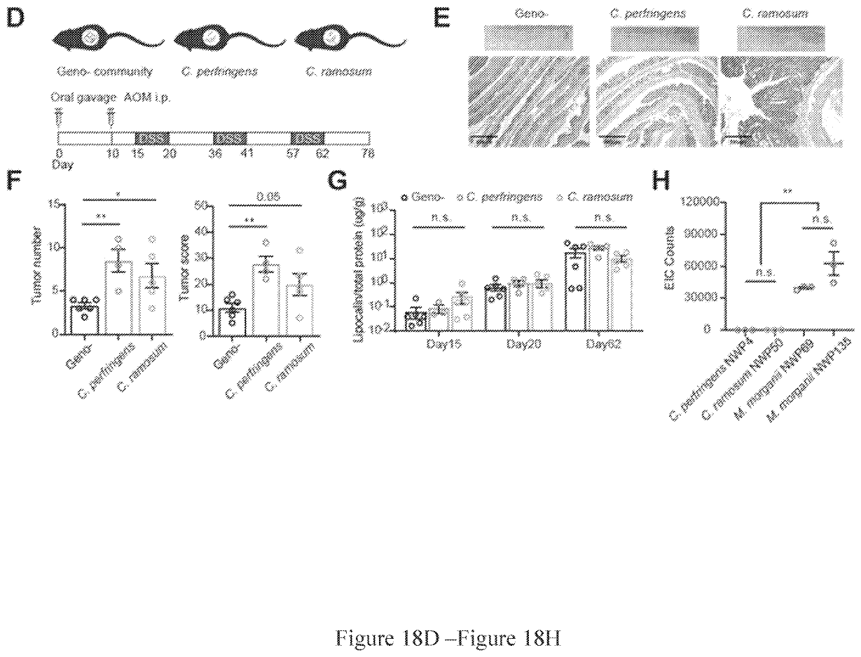

By revealing the existence of a previously uncharted universe of microbiota-derived genotoxins and defining the indolimines as a previously undescribed family of bioactive microbiota-derived small molecules, these studies imply an expanded role for genotoxic metabolites in CRC. Most of the studies on M. morganii because it is enriched in both CRC patients (Thomas et al., 2019, Nature Medicine 25, 667-678; Wirbel et al., 2019, Nature Medicine 25, 679-689; Poore et al., 2020, Nature 579, 567-574; Chen et al., 2012, PLoS One 7, e39743) and in patients with IBD, who are at increased risk of CRC diagnosis (Oldn et al., 2020, Lancet 395, 123-131; Olen et al., 2020, Lancet Gastroenterol. Hepatol. 5, 475-484). However, additional genotoxic species identified in the initial screens may also contribute to CRC. Indeed, genotoxic C. perfringens and C. ramosum strains also promoted colorectal tumor burden in gnotobiotic mice as compared to a non-genotoxic mock community (FIG. 18D-G), but did not produce indolimines (FIG. 18H), suggesting that additional microbiota-derived genotoxins remain to be characterized.

Notably, somatic mutations can be detected in colonic epithelial cells even in early life, which suggests persistent mutagenesis throughout the lifespan of an individual (Martincorena and Campbell, 2015, Science 349, 1483-1489Lee-Six et al., 2019, Nature 574, 532-537), and colitis-related expansions of mutated clones may influence both IBD pathogenesis and CRC susceptibility (Olafsson et al., 2020, Cell 182, 672-684). Furthermore, while CRC patients display increased carriage of clb+ E. coli, clb+ taxa (including E. coli relatives, such as Klebsiella species) are also found in healthy individuals (Putze et al., 2009, Infect. Immun. 77, 4696-4703). Recent studies also revealed that increased epithelial oxygenation during colitis could drive clb+E. coli expansion through aerobic respiration, increasing colibactin-mediated CRC-inducing activity (Cevallos et al., 2019, MBio. 10, e02244-19). These observations support a model whereby genotoxic gut microbes contribute to CRC development by persistently inducing DNA damage in host epithelial cells, which synergizes with chronic inflammation in the gut microenvironment, along with additional environmental factors, and eventually facilitates the initiation and progression of CRC.

Microbiota-derived genotoxins may also impact diverse aspects of host biology beyond tumor initiation. Recent studies revealed that colibactin also influences gut microbiome composition (Tronnet et al., 2020, mSphere 5, e00589-20), exacerbates lymphopenia and septicemia (Marcq et al., 2014, J. Infect. Dis. 210, 285-294), triggers prophage induction through the bacterial SOS response (Silpe et al., 2022, Nature 603, 315-320), and restricts Vibrio cholerae colonization (Chen et al., 2022, Proc. Natl. Acad. Sci. U.S.A. 119, e2121180119). Thus, commensal-derived genotoxins, including the indolimines, may also mediate diverse biological functions. Overall, the studies underscore the power of function-based assessments of the microbiome to provide new insights into the diverse impacts of indigenous microbes on host biology and disease susceptibility.

Given the enormous complexity and diversity of metabolites produced by bacteria (da Silva et al., 2015, Proc. Natl. Acad. Sci. U.S.A. 112, 12549-12550), it was hypothesized that diverse taxa from the human gut microbiome may produce previously undiscovered small molecules that cause DNA damage in intestinal epithelial cells and contribute to the development of CRC. Described herein are methods to evaluate the genotoxicity of small molecule metabolites derived from phylogenetically diverse human gut microbes. A diverse set of microbes that produced genotoxic small molecule metabolites are described, including the Gram-positive bacteria Clostridium perfringens and Clostridium ramosum, and the Gram-negative bacteria Morganella morganii. However, none of these isolates produced already known genotoxins, such as colibactin, or encoded known genotoxin-producing biosynthetic gene clusters. Combined untargeted metabolomics and bioactivity-guided natural product discovery techniques were combined to isolate and characterize a family of previously undescribed genotoxic metabolites—termed the indolimines—produced by CRC-associated M. morganii. In addition, the pathway for indolimine synthesis is described and an isogenic non-indolimine-producing mutant of M. morganii that lacked genotoxicity in vitro and in vivo was constructed and was shown to fail to exacerbate colon tumorigenesis in a mouse model of CRC.

The results of the experiments are now described.

Establishing a Pipeline to Identify Genotoxic Gut Microbes from Patients with Inflammatory Bowel Disease

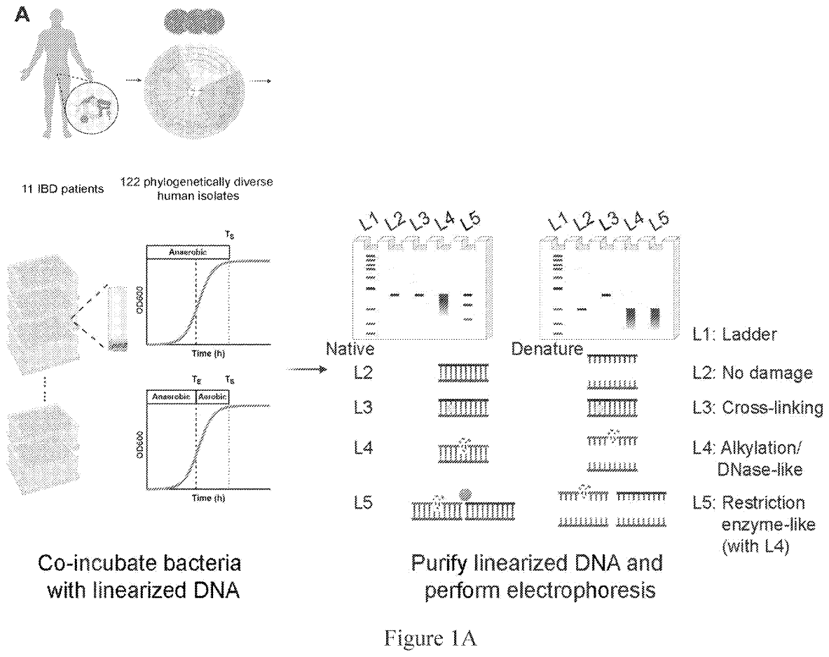

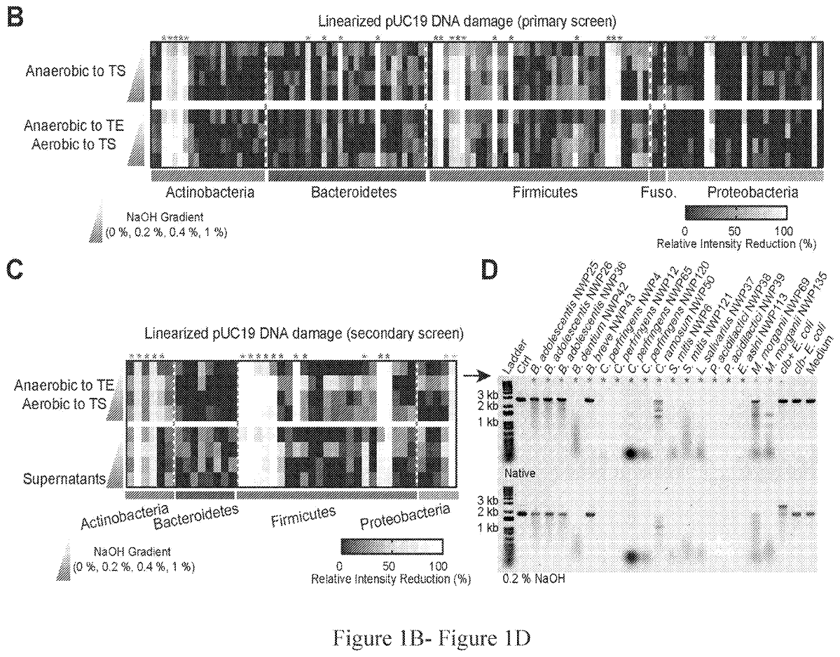

The established pipeline screened diverse human gut microbes based on their ability to directly damage DNA. The pipeline was applied to a gut microbiota culture collection assembled by anaerobic culturomics of stool samples from 11 inflammatory bowel disease (IBD) patients (Palm et al., 2014, Cell 158, 1000-1010), as IBD patients are at a significantly increased risk of developing CRC (Olen et al., 2020, Lancet 395, 123-131; Olen et al., 2020, Lancet Gastroenterol. Hepatol. 5, 475-484). This collection consists of 122 unique bacterial isolates that span 5 phyla, 9 classes, 10 orders, and 17 families, as well as multiple strains that were assigned to the same species (FIG. 1A).

To evaluate genotoxicity, the activity of each isolate in a plasmid DNA damage assay. As genotoxic metabolites such as colibactin can be recalcitrant to isolation (Xue et al., 2019, Science 365, 6457), the primary studies on co-incubation of individual bacterial isolates with linearized pUC19 plasmid DNA (FIG. 1A). This assay is based on the principle that the extent and modes of DNA damage can be assessed by electrophoresis under native and denaturing conditions (Bossuet-Greif et al., 2018, mBio 9, e02393-17): Double-stranded linearized pUC19 DNA migrates as a slow-moving band under native electrophoresis, while denaturing treatment separates the DNA into single-stranded DNA, leading to a higher-mobility band (FIG. 1A, L2). The formation of DNA interstrand cross-links (ICLs), for example, by colibactin, prevents unwinding under denaturing conditions, thereby resulting in a band that has the same mobility as duplexed DNA (FIG. 1A, L3). Alkylation at many of the sites in DNA is known to decrease the stability of the glycoside bonds, resulting in deglycosylation and fragmentation. These damaged DNA products are detected as smaller fragments of higher mobility following electrophoresis (Gates et al., 2009, Chemical Research in Toxicology 22, 1747-1760) (FIG. 1A, L4). Extensive DNA damage, for example, by DNase-mediated degradation, results in a loss of DNA even under native conditions (FIG. 1A, L4). Finally, DNA damage induced by restriction enzyme-like molecules produces multiple bands under native conditions and even smaller fragments under denaturing conditions when combined with damage induced by alkylation or DNase-like molecules (FIG. 1A, L5).

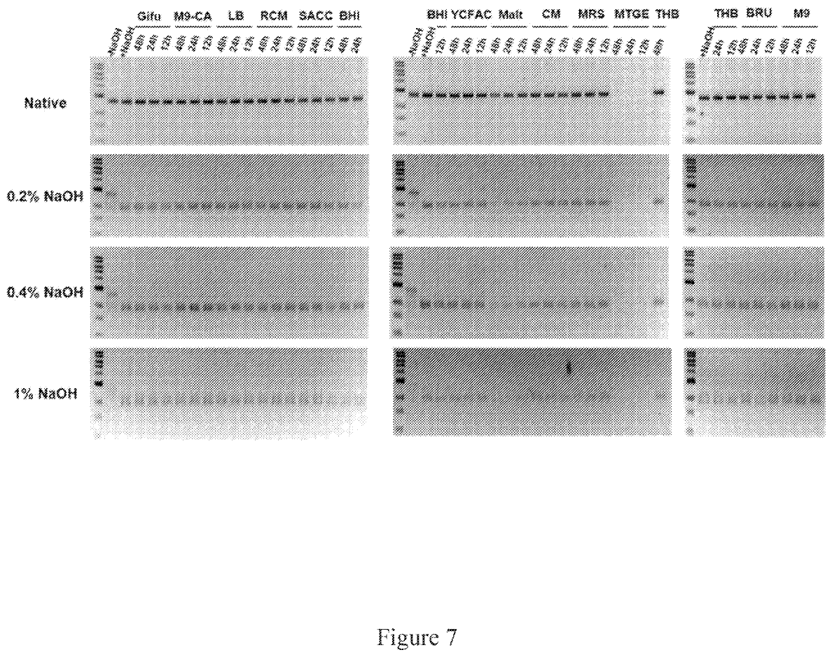

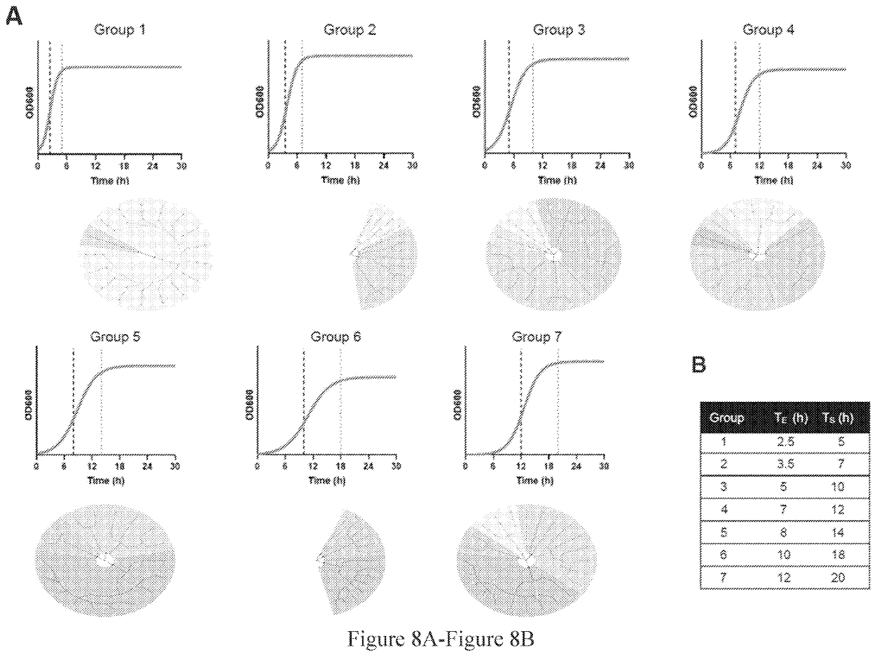

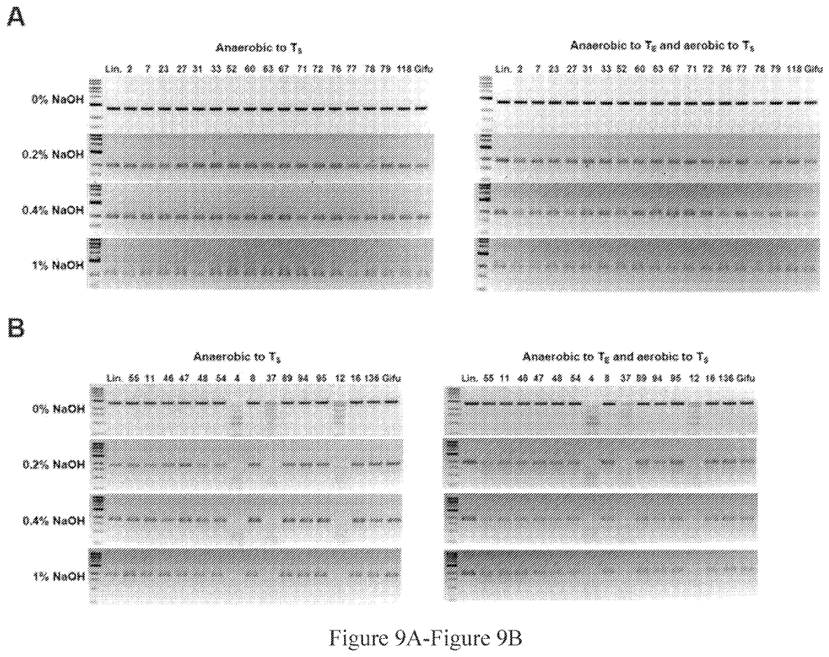

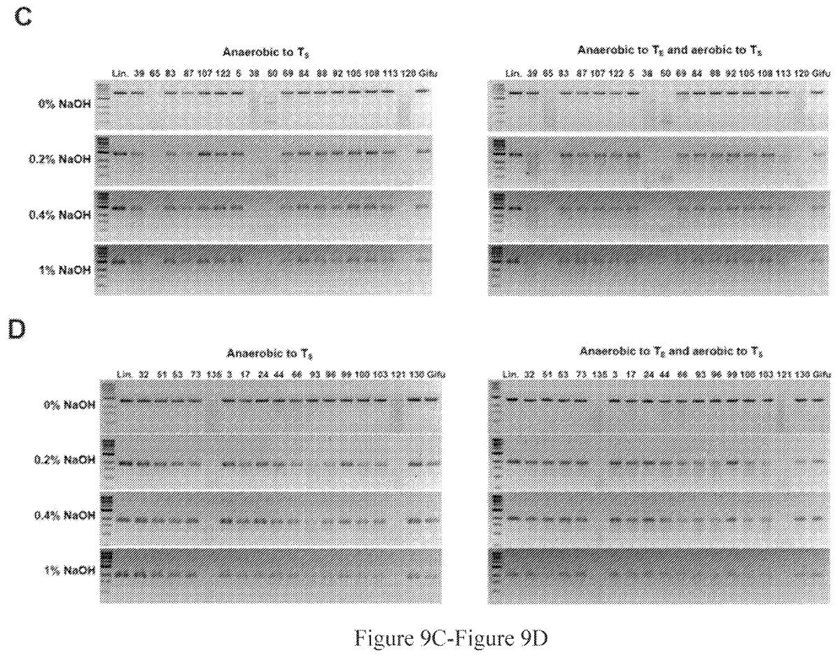

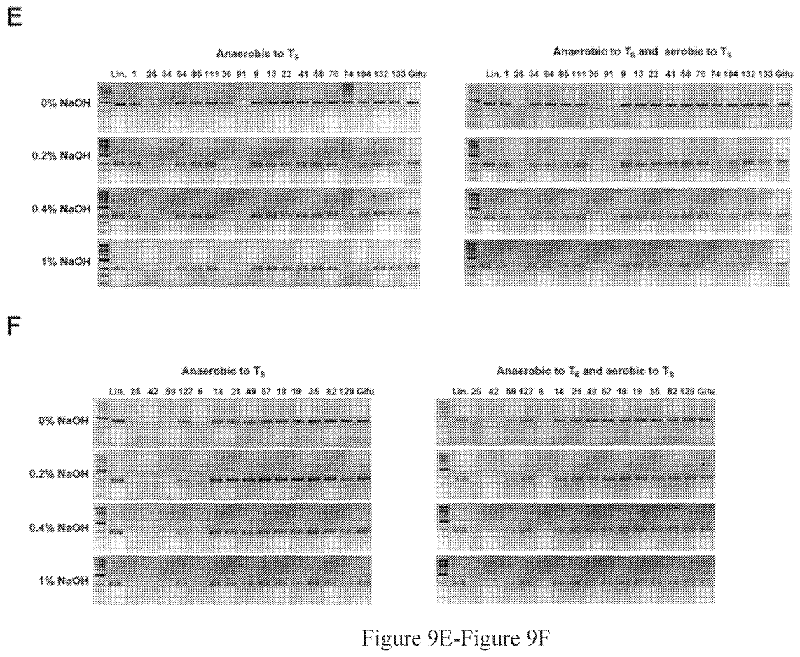

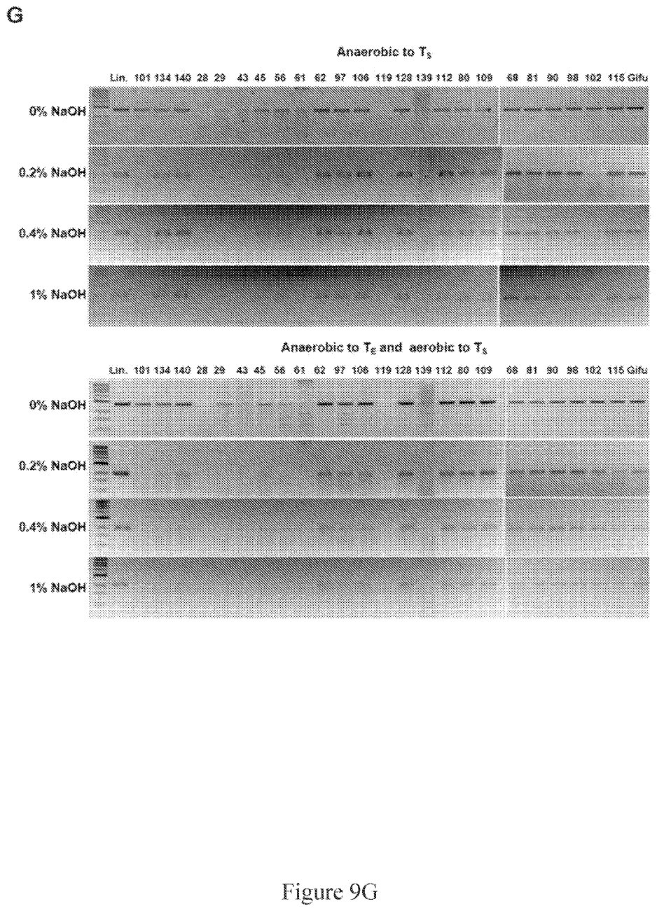

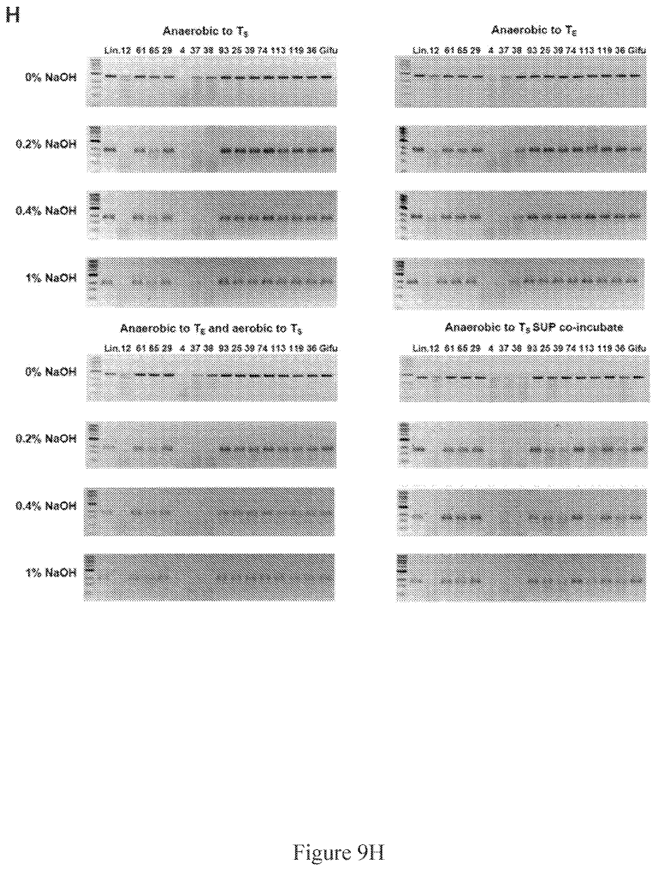

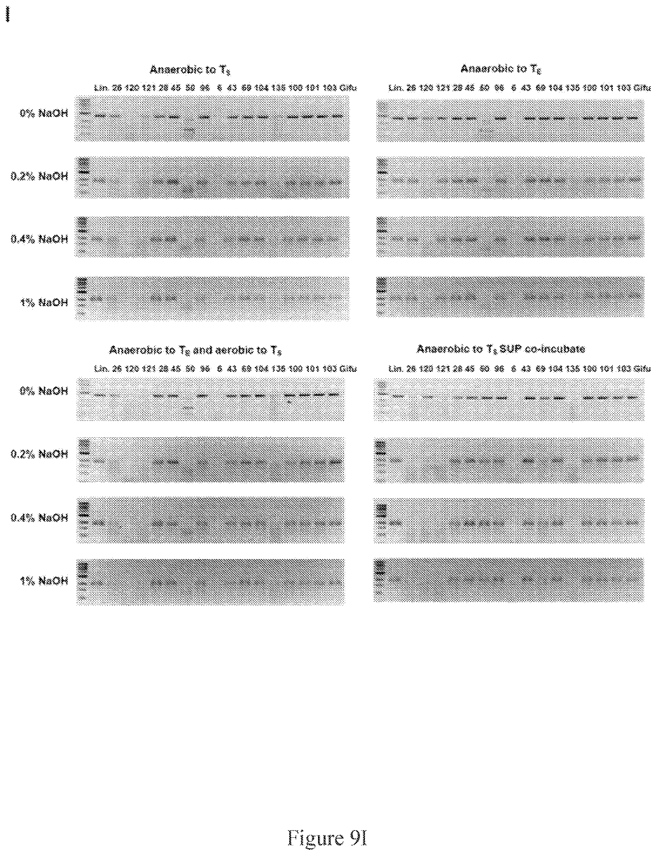

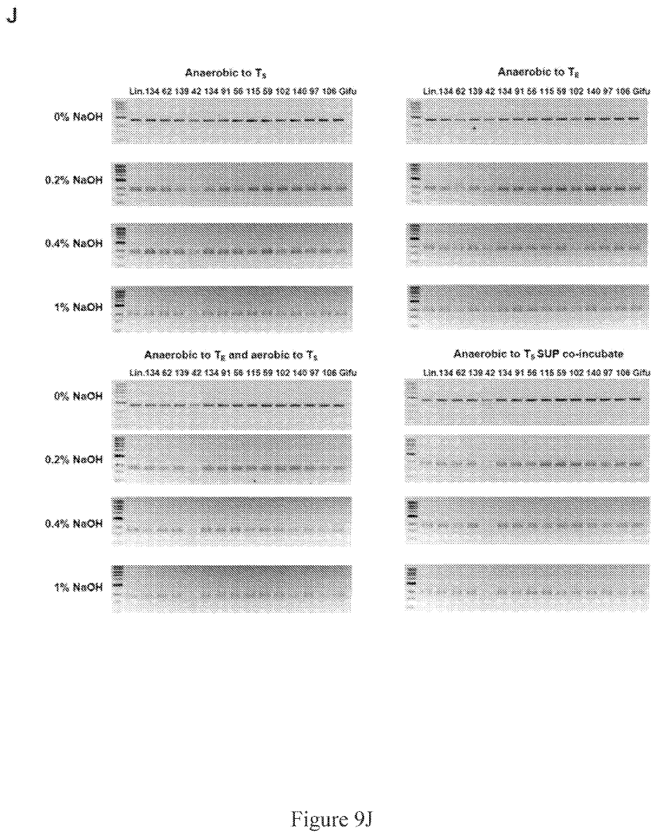

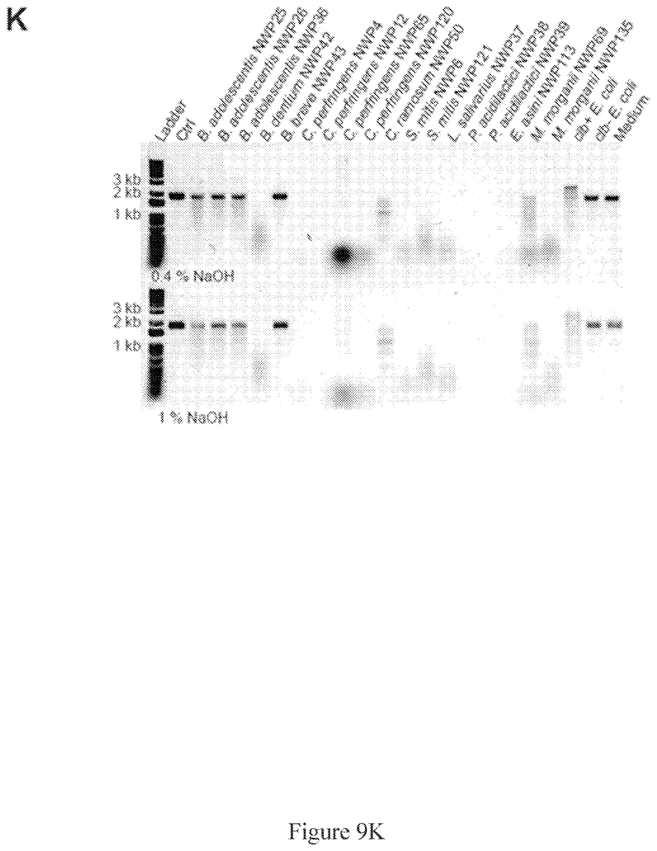

The plasmid DNA was confirmed to be stable under diverse anaerobic cultivation conditions including incubation in Gifu medium, which supports the growth of all isolates in the collections (FIG. 7). To minimize the damage caused by bacterial DNases that are often produced in the stationary phase of bacterial growth, growth curves were measured for all 122 isolates in the collection and established a T_{E} (time point of exponential phase) and Ts (time point of stationary phase) for each isolate (Table 1). The isolates were then clustered into 7 groups that exhibited similar growth dynamics (FIG. 8). Two culture conditions were selected for the initial screening: anaerobic co-incubation with DNA to Ts; or anaerobic co-incubation with DNA to T_{E} which was followed by aerobic co-incubation to Ts to approximate the oxygen stress encountered in an inflammatory gut environment. Finally, linearized pUC19 DNA was purified from the bacterial cultures via column purification and performed gel electrophoresis under native and gradient denaturing conditions (0%, 0.2%, 0.4% and 1% NaOH) (FIG. 1A, FIG. 9A-G).

TABLE 1
Growth curves of 122 collected isolates.
Based on Qiime 1.6, GreenGenes database:
Grow in Gifu
medium

| | | | | | 48 h | | |
|---|---|---|---|---|---|---|---|
| Nr | Phylum | Family | Genus | Species | OD600 | TE(h) | TS(h) |
| 1 | Bacteroidetes | Bacteroidaceae | Bacteroides | fragilis | 5.4 | 11 | 14 |
| 2 | Proteobacteria | Enterobacteriaceae | Other | Other | 2.33 | 2.5 | 4 |
| 3 | Firmicutes | Streptococcaceae | Streptococcus | luteciae | 1.98 | 4.5 | 10 |
| 4 | Firmicutes | Clostridiaceae | Clostridium | perfringens | 3.91 | 4 | 6 |
| 5 | Fusobacteria | Fusobacteriaceae | Fusobacterium | spp. | 2.33 | 5.5 | 8 |
| 6 | Firmicutes | Streptococcaceae | Streptococcus | spp. | 1.28 | 10 | 16 |
| 7 | Proteobacteria | Enterobacteriaceae | UC | UC | 2.27 | 2.5 | 4 |
| 8 | Proteobacteria | Enterobacteriaceae | UC | UC | 2.56 | 3 | 6 |
| 9 | Bacteroidetes | Bacteroidaceae | Bacteroides | fragilis | 4.94 | 9 | 12 |
| 11 | Firmicutes | Veillonellaceae | Megasphaera | spp. | 0.21 | 5 | 7 |
| 12 | Firmicutes | Clostridiaceae | Clostridium | perfringens | 4.15 | 3 | 5 |
| 13 | Bacteroidetes | Bacteroidaceae | Bacteroides | fragilis | 4.71 | 8.5 | 12 |
| 14 | Actinobacteria | Bifidobacteriaceae | Bifidobacterium | UC | 1.41 | 10.5 | 16 |
| 16 | Proteobacteria | Enterobacteriaceae | | | 2.78 | 3.5 | 5 |
| 17 | Proteobacteria | Enterobacteriaceae | Morganella | spp. | 2.15 | 6 | 10 |
| 18 | Bacteroidetes | Bacteroidaceae | Bacteroides | spp. | 3.81 | 9.5 | 15 |
| 19 | Bacteroidetes | Bacteroidaceae | Bacteroides | ovatus | 4.31 | 12.5 | 15 |
| 21 | Actinobacteria | Bifidobacteriaceae | Bifidobacterium | UC | 2.96 | 12 | 16 |
| 22 | Bacteroidetes | Bacteroidaceae | Bacteroides | fragilis | 4.88 | 9 | 12 |
| 23 | Proteobacteria | Enterobacteriaceae | UC | UC | 2.28 | 2.5 | 4 |
| 24 | Fusobacteria | Fusobacteriaceae | Fusobacterium | spp. | 2.63 | 7.5 | 10 |
| 25 | Actinobacteria | Actinobacteria | Bifidobacterium | adolescentis | 3.41 | 6 | 18 |
| 26 | Actinobacteria | Actinobacteria | Bifidobacterium | adolescentis | 3.29 | 8 | 14 |
| 27 | Proteobacteria | Enterobacteriaceae | UC | UC | 2.32 | 2.5 | 4 |
| 28 | Proteobacteria | Enterobacteriaceae | Morganella | spp. | 2.04 | 13 | 24 |
| 29 | Proteobacteria | Enterobacteriaceae | UC | UC | 6.51 | 5 | 24 |
| 31 | Proteobacteria | Enterobacteriaceae | UC | UC | 2.37 | 2.5 | 4 |
| 32 | Bacteroidetes | Bacteroidaceae | Bacteroides | spp. | 4.38 | 7.5 | 11 |
| 33 | Proteobacteria | Enterobacteriaceae | UC | UC | 2.29 | 2.5 | 4 |
| 34 | Bacteroidetes | Bacteroidaceae | Bacteroides | spp. | 4.16 | 10 | 14 |
| 35 | Bacteroidetes | Bacteroidaceae | Bacteroides | fragilis | 5.39 | 11.5 | 15 |
| 36 | Actinobacteria | Actinobacteria | Bifidobacterium | adolescentis | 3.81 | 8.5 | 13 |
| 37 | Firmicutes | Lactobacilluscac | Lactobacillus | UC | 1.5 | 4.5 | 6 |
| 38 | Firmicutes | Lactobacilloae | Pediococcus | UC | 0.88 | 5 | 8 |
| 39 | Firmicutes | Lactobacilloae | Pediococcus | UC | 0.94 | 6 | 9 |
| 41 | Bacteroidetes | Bacteroidaceae | Bacteroides | fragilis | 4.75 | 8.5 | 12 |
| 42 | Actinobacteria | Bifidobacteriaceae | Bifidobacterium | spp. | 4.69 | 13 | 18 |
| 43 | Actinobacteria | Bifidobacteriaceae | Bifidobacterium | UC | 3.62 | 8 | 24 |
| 44 | Proteobacteria | Enterobacteriaceae | Morganella | spp. | 1.62 | 6 | 10 |
| 45 | Firmicutes | Streptococcaceae | Streptococcus | spp. | 2.33 | 6 | 24 |
| 46 | Firmicutes | Erysipelotrichales | Erysipelotrichaceae | spp. | 1.14 | 5.5 | 7 |
| 47 | Firmicutes | Erysipelotrichales | Erysipelotrichaceae | spp. | 1.34 | 5.5 | 7 |
| 48 | Firmicutes | Clostridiaceae | Clostridium | perfringens | 8.44 | 4 | 7 |
| 49 | Actinobacteria | Bifidobacteriaceae | Bifidobacterium | UC | 0.79 | 12 | 16 |
| 50 | Firmicutes | Erysipelotrichales | Erysipelotrichaceae | spp. | 0.9 | 6 | 8 |
| 51 | Actinobacteria | Coriobacteriaceae | Collinsella | aerofaciens | 1.33 | 8 | 11 |
| 52 | Proteobacteria | Enterobacteriaceae | UC | UC | 2.06 | 2.5 | 4 |
| 53 | Actinobacteria | Coriobacteriaceae | Collinsella | aerofaciens | 1.63 | 8.5 | 11 |
| 54 | Firmicutes | Clostridiaceae | Clostridium | perfringens | 7.54 | 4 | 7 |
| 55 | Firmicutes | Streptococcaceae | Streptococcus | spp. | 0.87 | 5 | 7.5 |
| 56 | Bacteroidetes | Bacteroidaceae | Bacteroides | ovatus | 4.14 | 12.5 | 24 |
| 57 | Bacteroidetes | Bacteroidaceae | Bacteroides | spp. | 4.59 | 12 | 16 |
| 58 | Bacteroidetes | Bacteroidaceae | Bacteroides | fragilis | 5.12 | 8 | 12 |
| 59 | Bacteroidetes | Bacteroidaceae | Bacteroides | UC | 4.12 | 13.5 | 17 |
| 60 | Proteobacteria | Enterobacteriaceae | UC | UC | 2.14 | 2.5 | 4 |
| 61 | Proteobacteria | Enterobacteriaceae | UC | UC | 2.31 | 5 | 24 |
| 62 | Bacteroidetes | Bacteroidaceae | Bacteroides | fragilis | 4.13 | 7 | 24 |
| 63 | Proteobacteria | Enterobacteriaceae | UC | UC | 2.1 | 2.5 | 4 |
| 64 | Bacteroidetes | Porphyromonadaceae | Parabacteroides | distasonis | 3.34 | 8 | 14 |
| 65 | Firmicutes | Clostridiaceae | Clostridium | perfringens | 9.21 | 6 | 9 |
| 66 | Proteobacteria | Enterobacteriaceae | Morganella | spp. | 1.58 | 6.5 | 10 |
| 67 | Proteobacteria | Enterobacteriaceae | UC | UC | 2.08 | 2.5 | 4 |
| 68 | Firmicutes | [Tissierellaceae] | Peptoniphilus | spp. | 0.98 | 10 | 20 |
| 69 | Proteobacteria | Enterobacteriaceae | Morganella | spp. | 1.59 | 5 | 8 |
| 70 | Firmicutes | [Tissierellaceae] | Peptoniphilus | spp. | 0.96 | 8 | 12 |
| 71 | Proteobacteria | Enterobacteriaceae | UC | UC | 1.96 | 2.5 | 4 |
| 72 | Proteobacteria | Enterobacteriaceae | UC | UC | 2.01 | 2.5 | 4 |
| 73 | Actinobacteria | Coriobacteriaceae | Collinsella | aerofaciens | 2.07 | 8 | 11 |
| 74 | Firmicutes | Lachnospiraceae | [Ruminococcus] | gnavus | 2.53 | 5 | 12 |
| 76 | Firmicutes | Lachnospiraceae | [Ruminococcus] | gnavus | 2.1 | 2.5 | 4 |
| 77 | Proteobacteria | Enterobacteriaceae | UC | UC | 1.93 | 2.5 | 4 |
| 78 | Proteobacteria | Enterobacteriaceae | UC | UC | 2.07 | 2.5 | 4 |
| 79 | Proteobacteria | Enterobacteriaceae | UC | UC | 1.93 | 2.5 | 4 |
| 80 | Bacteroidetes | Bacteroidaceae | Bacteroides | spp. | 3.69 | 15.5 | 21 |
| 81 | Bacteroidetes | Bacteroidaceae | Bacteroides | uniformis | 2.07 | 11.5 | 20 |
| 82 | Bacteroidetes | Bacteroidaceae | Bacteroides | ovatus | 4.3 | 11.5 | 15 |
| 83 | Actinobacteria | Coriobacteriaceae | Collinsella | stercoris | 1.58 | 6.5 | 9 |
| 84 | Actinobacteria | Coriobacteriaceae | Collinsella | stercoris | 1.71 | 6 | 8 |
| 85 | Firmicutes | Clostridiales (order) | UC | UC | 0.99 | 9 | 14 |
| 87 | Actinobacteria | Coriobacteriaceae | Collinsella | stercoris | 1.68 | 6.5 | 9 |
| 88 | Firmicutes | Lactobacilluseae | Lactobacillus | reuteri | 1.34 | 5 | 8 |
| 89 | Firmicutes | Lactobacilluseae | Lactobacillus | reuteri | 1.42 | 3.5 | 6 |
| 90 | Bacteroidetes | Bacteroidaceae | Bacteroides | uniformis | 1.55 | 11.5 | 20 |
| 91 | Bacteroidetes | Bacteroidaceae | Bacteroides | UC | 3.7 | 9.5 | 13 |
| 92 | Proteobacteria | Sinobacteriaceae | Nevskia | spp. | 0.15 | 5 | 8 |
| 93 | Firmicutes | Lactobacilloae | Lactobacillus | reuteri | 1.2 | 4 | 10 |
| 94 | Firmicutes | Lactobacilluseae | Lactobacillus | reuteri | 1.27 | 4 | 6 |
| 95 | Proteobacteria | Sinobacteriaceae | Nevskia | spp. | 0.08 | 4 | 6 |
| 96 | Firmicutes | Veillonellaceae | Acidaminococcus | spp. | 0.53 | 7 | 10 |
| 97 | Bacteroidetes | Bacteroidaceae | Bacteroides | uniformis | 1.6 | 14 | 24 |
| 98 | Bacteroidetes | Bacteroidaceae | Bacteroides | uniformis | 1.8 | 11.5 | 20 |
| 99 | Actinobacteria | Bifidobacteriaceae | Bifidobacterium | UC | 0.81 | 7 | 10 |
| 100 | Firmicutes | Veillonellaceae | Acidaminococcus | spp. | 0.52 | 6.5 | 10 |
| 101 | Firmicutes | Veillonellaceae | Acidaminococcus | spp. | 0.45 | 16.5 | 30 |
| 102 | Bacteroidetes | Bacteroidaceae | Bacteroides | UC | 1.66 | 12 | 20 |
| 103 | Firmicutes | Veillonellaceae | Acidaminococcus | spp. | 0.4 | 6 | 10 |
| 104 | Firmicutes | Veillonellaceae | Acidaminococcus | spp. | 0.45 | 7 | 12 |
| 105 | Actinobacteria | Bifidobacteriaceae | Bifidobacterium | UC | 0.98 | 5 | 8 |
| 106 | Actinobacteria | Bifidobacteriaceae | Bifidobacterium | UC | 0.84 | 14.5 | 24 |
| 107 | Actinobacteria | Bifidobacteriaceae | Bifidobacterium | UC | 0.77 | 6 | 9 |
| 108 | Actinobacteria | Bifidobacteriaceae | Bifidobacterium | UC | 1 | 5 | 8 |
| 109 | Bacteroidetes | Bacteroidaceae | Bacteroides | uniformis | 2 | 13 | 21 |
| 111 | Firmicutes | Veillonellaceae | Megasphaera | spp. | 2.22 | 9 | 14 |
| 112 | Firmicutes | Lachnospiraceae | [Ruminococcus] | gnavus | 1.45 | 12 | 22 |
| 113 | Firmicutes | Lactobacilluseae | Pediococcus | UC | 0.75 | 5 | 8 |
| 115 | Bacteroidetes | Porphyromonadaceae | Parabacteroides | distasonis | 2.09 | 12.5 | 20 |
| 118 | Proteobacteria | Enterobacteriaceae | UC | UC | 1.84 | 2.5 | 4 |
| 119 | Firmicutes | Ruminococcaceae | Faecalibacterium | prausnitzii | 0.64 | 7 | 24 |
| 120 | Firmicutes | Clostridiaceae | Clostridium | perfringens | 7.08 | 5.5 | 8 |
| 121 | Firmicutes | Streptococcaceae | Streptococcus | spp. | 1.53 | 6 | 10 |
| 122 | Actinobacteria | Coriobacteriaceae | Eggerthela | lenta | 0.13 | 6.5 | 9 |
| 127 | Bacteroidetes | Bacteroidaceae | Bacteroides | uniformis | 1.77 | 10 | 17 |
| 128 | Firmicutes | Erysipelotrichaceae | Allobaculum | spp. | 0.6 | 15 | 24 |
| 129 | Bacteroidetes | Porphyromonadaceae | Parabacteroides | spp. | 2.38 | 10.5 | 15 |
| 130 | Actinobacteria | Coriobacteriaceae | Collinsella | stercoris | 1.73 | 7.5 | 10 |
| 132 | Firmicutes | [Tissierellaceae] | Peptoniphilus | spp. | 0.79 | 8 | 12 |
| 133 | Bacteroidetes | Bacteroidaceae | Bacteroides | fragilis | 4.29 | 8 | 12 |
| 134 | Firmicutes | Ruminococcaceae | Oscillospira | spp. | 0.73 | 9 | 30 |
| 135 | Proteobacteria | Enterobacteriaceae | Morganella | spp. | 1.21 | 6 | 11 |
| 136 | Proteobacteria | Enterobacteriaceae | UC | UC | 2.88 | 3 | 5 |
| 139 | Firmicutes | Ruminococcaceae | Oscillospira | spp. | 4.2 | 8 | 24 |
| 140 | Firmicutes | Ruminococcaceae | Oscillospira | spp. | 0.85 | 17.5 | 30 |

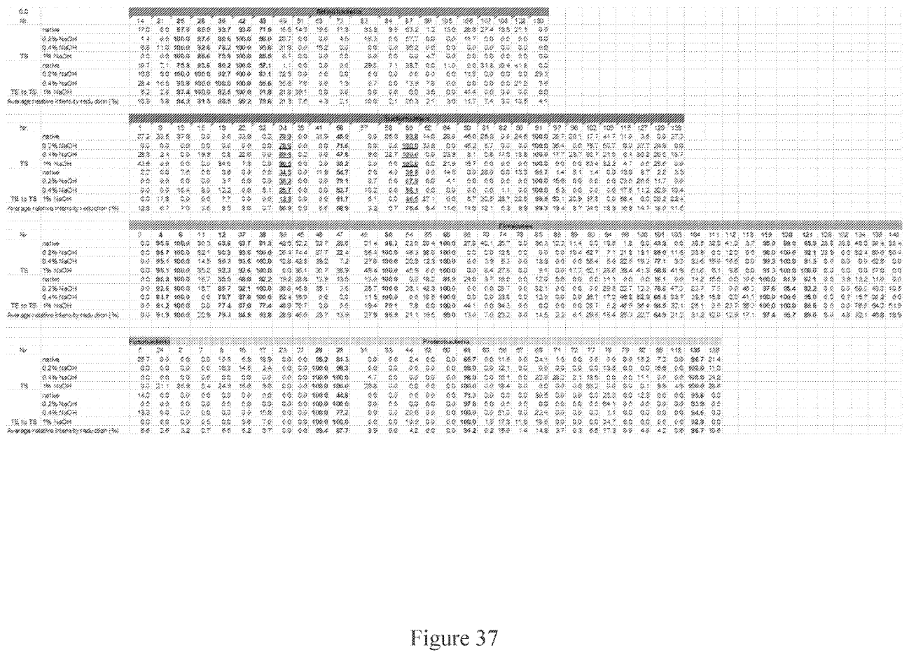

The relative intensity reduction (RIR, %) of DNA was used after co-incubation with bacteria as a general measure of bacterially induced DNA damage (FIG. 1B, FIG. 37). Diverse gut microbes were found that exhibited DNA damaging activities, which suggests that microbiota-mediated genotoxicity may be more widespread than previously appreciated. While previously described microbiota-derived genotoxins discovered in a case-by-case manner are primarily produced by Gram-negative bacteria (e.g., E. coli, B. fragilis and K. oxytoca) (Dejea et al., 2018, Science 359, 592-597; Unterhauser et al., 2019, Proc. Natl. Acad. Sci. U.S.A. 116, 3774-3783), multiple Gram-positive microbes were observed from the phyla Actinobacteria and Firmicutes also caused significant DNA damage. DNA damaging activity was largely independent of culture conditions, although select microbes displayed varied genotoxicity in the presence or absence of oxygen stress (FIG. 1B, FIG. 37).

TABLE 2
Twenty-four isolates that exhibited strong DNA damaging activities
in the primary screen and 18 phylogenetically related non-genotoxic
isolates for evaluation in a secondary screening.

| Based on [symbol] Database | | | | | | | No. of |
|---|---|---|---|---|---|---|---|
| [symbol] | Phylum | Class | Order | Family | Genus | Species | Strains |
| [symbol] | Actinobacteria | [symbol] | [symbol] | [symbol] | Biffdobacterium | [symbol] | [symbol] |
| [symbol] | [symbol] | [symbol] | [symbol] | [symbol] | [symbol] | [symbol] | 1 |
| [symbol] | [symbol] | [symbol] | [symbol] | [symbol] | [symbol] | [symbol] | 1 |
| [symbol] , 12, [symbol] , [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | 1 |
| [symbol] | [symbol] | [symbol] | [symbol] | [symbol] | [symbol] | [symbol] | 2 |
| [symbol] | [symbol] | [symbol] | [symbol] | [symbol] | [symbol] | [symbol] | 1 |
| [symbol] , [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | 1 |
| [symbol] | [symbol] | [symbol] | [symbol] | [symbol] | [symbol] | [symbol] | [symbol] |

[symbol]

indicates data missing or illegible when filed

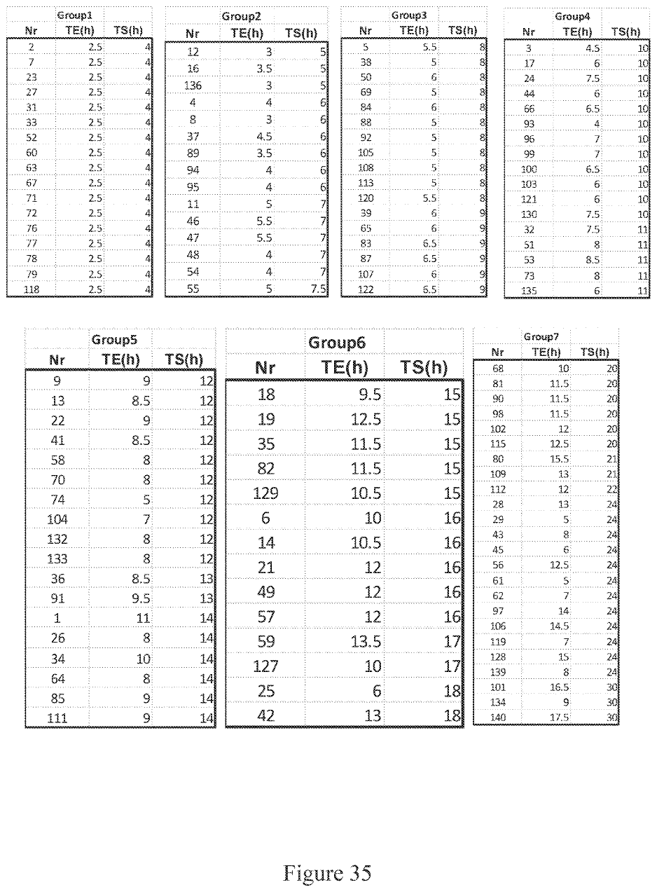

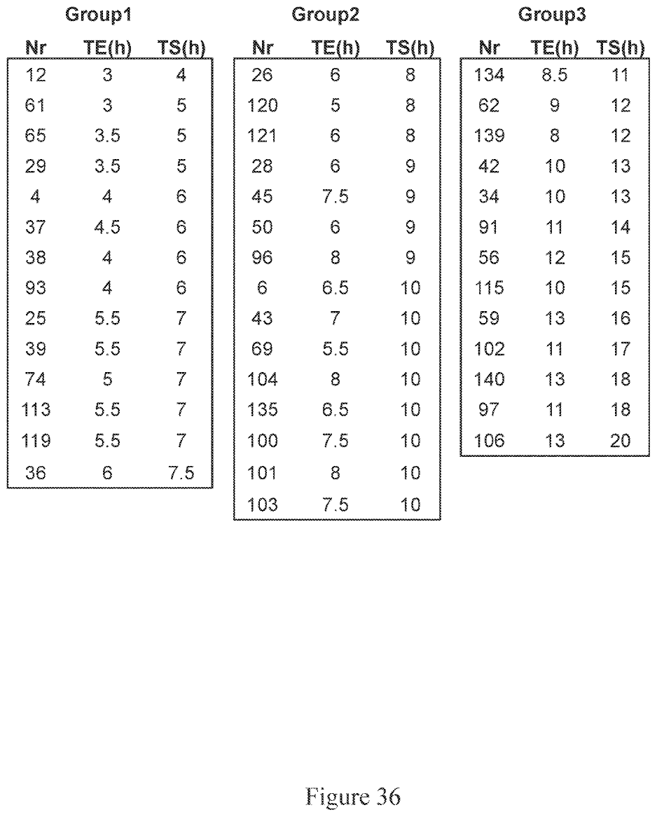

Twenty-four isolates were selected that exhibited strong DNA damaging activities in the primary screen and 18 phylogenetically related non-genotoxic isolates for evaluation in a secondary screening (FIG. 1C). the precise growth curve was re-established for each isolate (FIG. 35-36) and re-screened all 42 isolates under four distinct culture conditions, including co-incubation of DNA with bacterial supernatants collected from anaerobic cultures at Ts (FIG. 9H-J, FIG. 37). 18 isolates that caused DNA damage in the primary screening also exhibited strong genotoxicity upon secondary screening (FIG. 1C, FIG. 9H-J). Moreover, supernatants from most of the selected genotoxic isolates also caused significant RIR (%) that were often comparable to co-incubation with live bacteria (FIG. 1C, FIG. 9H-J). Unlike the interstrand cross-links induced by a colibactin-producing E. coli strain (K-12 BW25113 containing the clb locus, designated as clb+ E. coli), these 18 isolates exhibited distinct DNA damage patterns after coincubation with linearized pUC19 DNA. By contrast, a non-colibactin-producing E. coli strain (K-12 BW25113 with an empty bacterial artificial chromosome, designated as clb− E. coli) or medium alone did not induce significant DNA damage (FIG. 1D, FIG. 9K). Notably, most of the newly identified genotoxic isolates induced alkylation or DNase-like DNA damage patterns, and evidence was observed of DNA damage under native gel electrophoresis.

Gut Microbes from Patients with Inflammatory Bowel Disease Produce Small Molecule Metabolites that can Induce DNA Damage

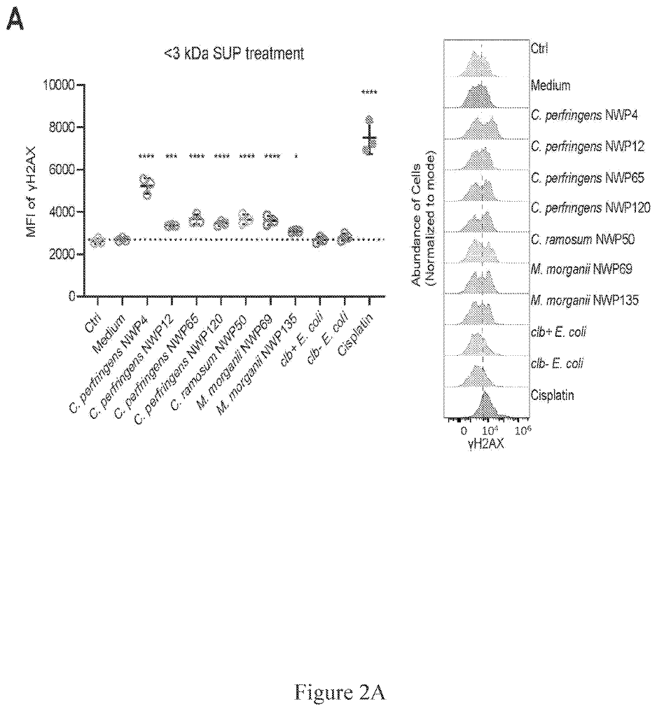

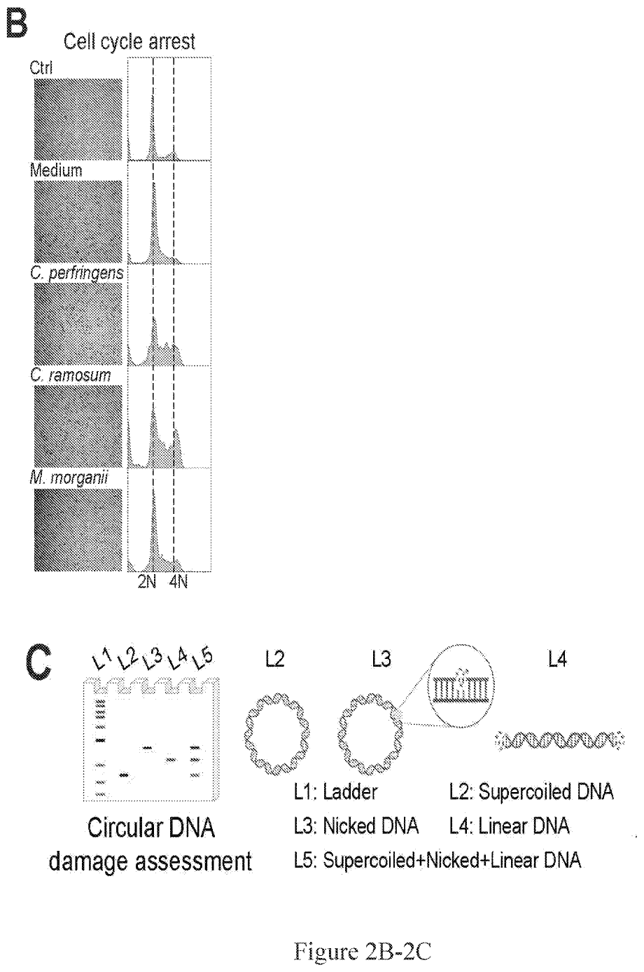

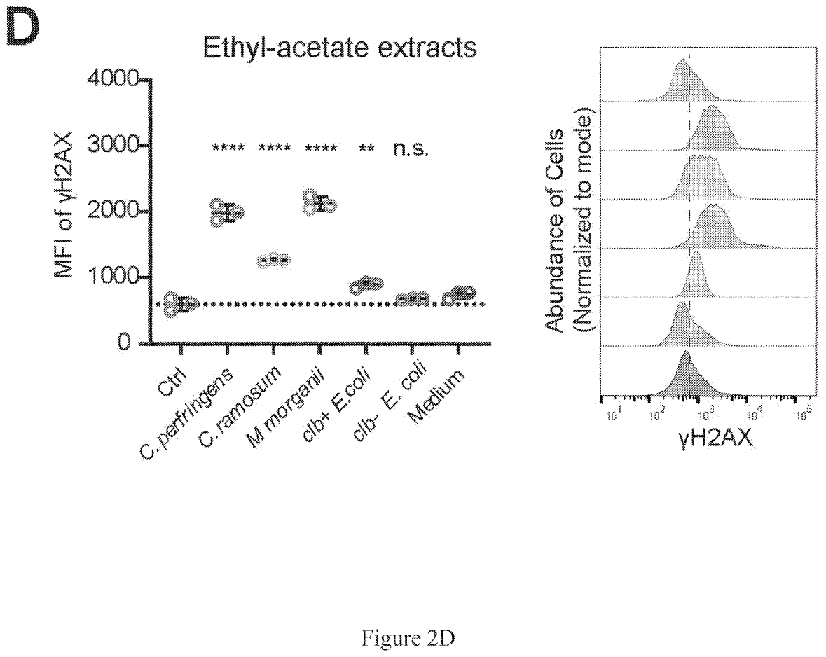

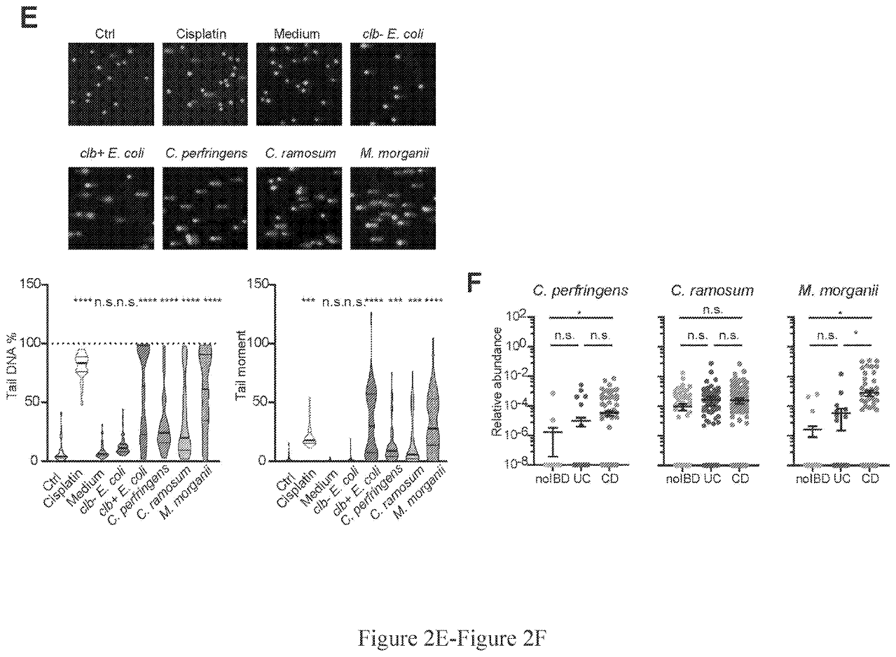

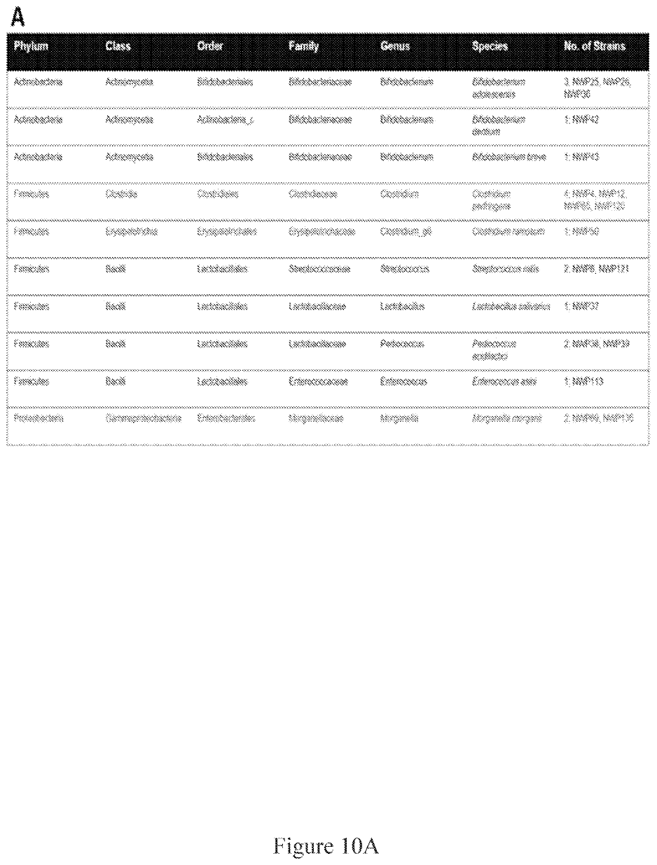

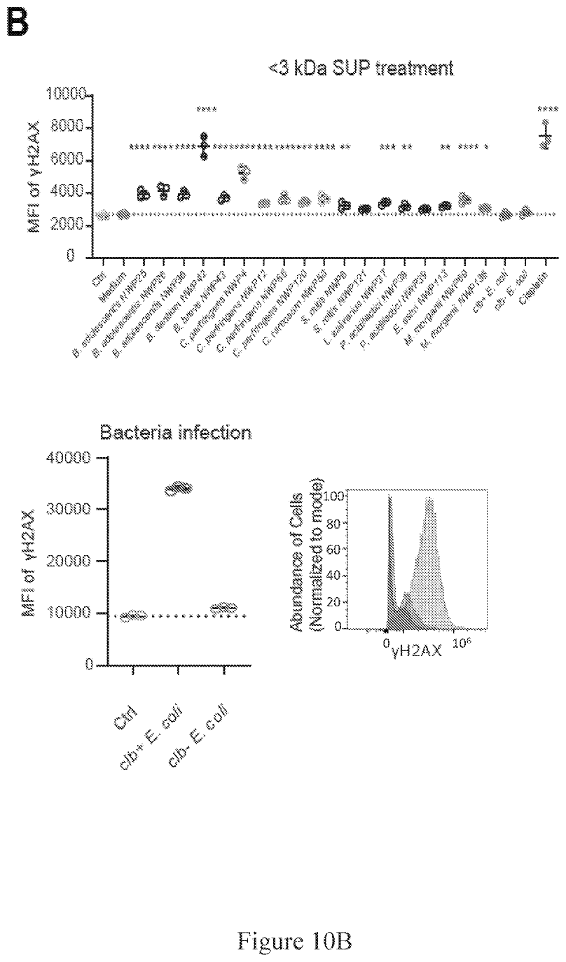

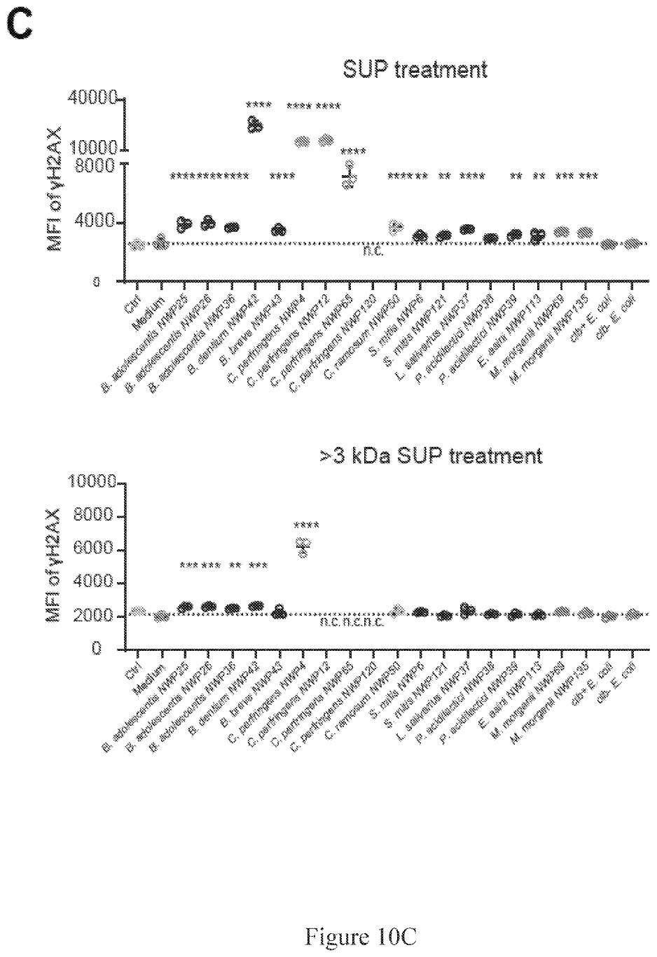

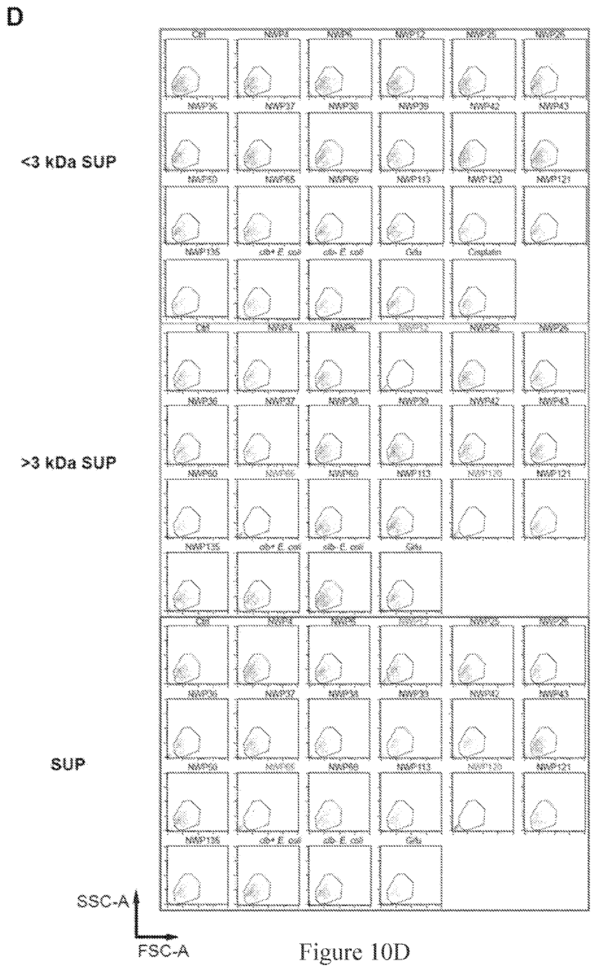

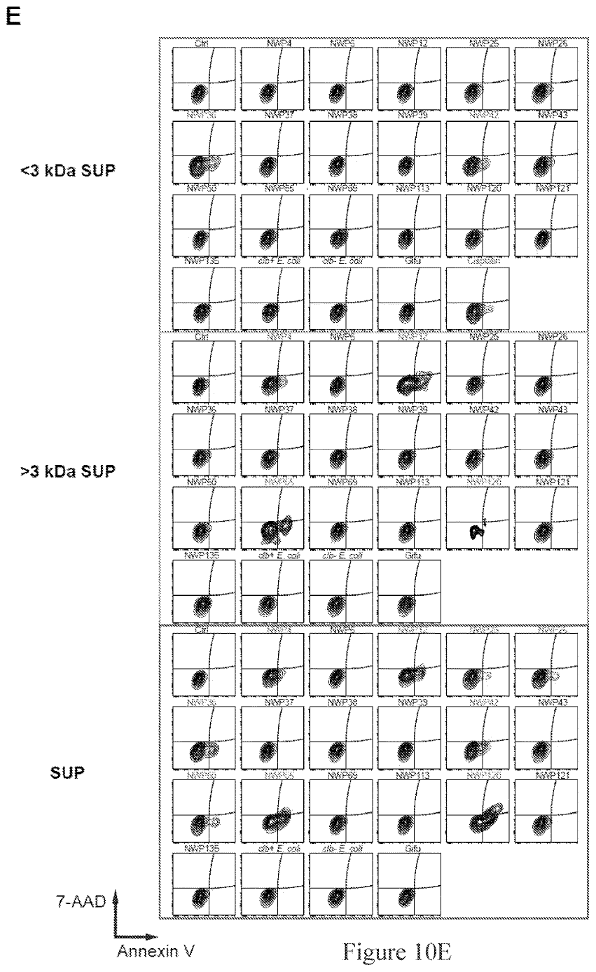

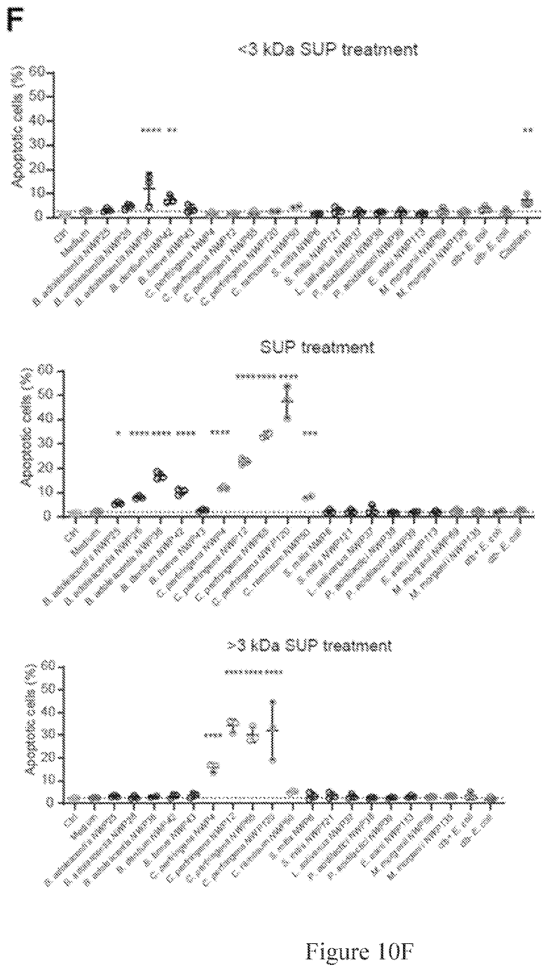

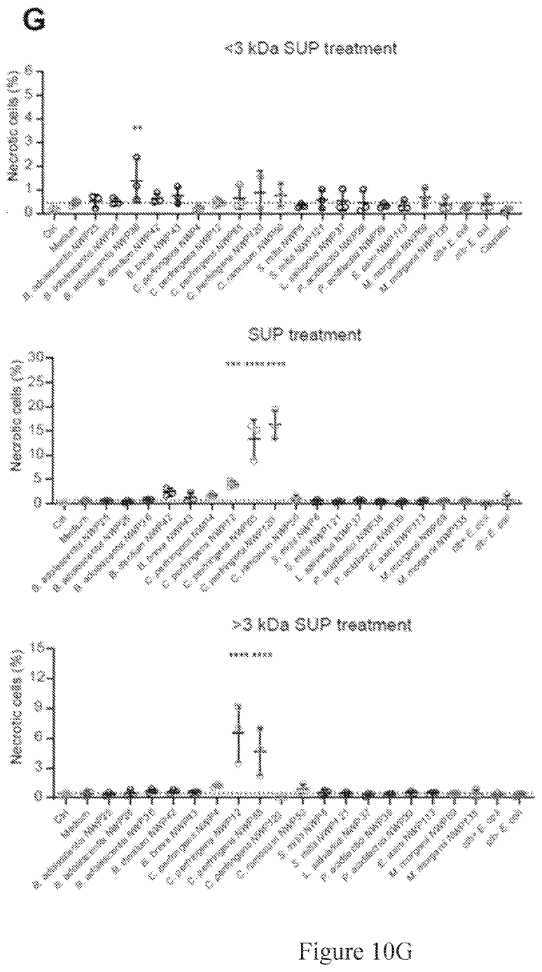

To determine whether the 18 putative genotoxic bacterial isolates were identified via electrophoresis-based screening (FIG. 1, FIG. 10A) produce genotoxic small molecules that cause DNA damage in human cells, supernatants were separated (SUP) into small- (<3 kDa SUP) and large- (>3 kDa SUP) molecular weight fractions and evaluated the genotoxicity of these fractions using HeLa cells. Small molecule metabolites from most selected isolates, including multiple strains of Bifidobacterium adolescentis (3 isolates), Clostridium perfringens (4 isolates), Clostridium ramosum (phylogenetically related to C. perfringens), and Morganella morganii (2 isolates), induced increased γ-H2AX, a marker of DNA double-strand breaks (DSBs) (Kuo and Yang, 2008)) (FIG. 10B). As previously reported, supernatants from clb+ E. coli failed to induce γ-H2AX, which instead required live bacterial infection (Nougayrede et al., 2006, Science 313, 848-851) (FIG. 10B). While both small and large molecules from B. adolescentis and C. perfringens induced increased γ-H2AX, only small molecule metabolites from C. ramosum and M. morganii exhibited genotoxicity (FIG. 10B-C). Small molecule metabolites from B. adolescentis and B. dentium induced increased apoptosis and necrosis, while small molecule metabolites from all other isolates had minimal impacts on cell viability as measured by cell size and granularity (FIG. 10D), Annexin V, or 7-AAD (FIG. 10E-G). Although large molecules from C. perfringens induced significant cell death, likely due to the effects of clostridial toxins (Freedman et al., 2016, Toxins (Basel) 8, 73), small molecule metabolites from C. perfringens caused DNA damage without triggering significant cell death (FIG. 10B-G). Based on these results, C. perfringens, C. ramosum, and M. morganii were selected for further study and confirmed that small-molecule metabolites from these strains induced 7-H2AX in HeLa cells, albeit at a reduced level compared to the well-known DNA-crosslinking chemical cisplatin (FIG. 2A). Small molecule metabolites from C. perfringens, C. ramosum and M. morganii also induced cell cycle arrest in HeLa cells (FIG. 2B), further implicating these taxa as potential genotoxin producers.

To enrich for genotoxic small molecules, ethyl-acetate extraction was performed using supernatants and found that extracts from C. perfringens (NWP4), C. ramosum (NWP50), M. morganii (NWP135), and clb+ E. coli cultures nicked circular pUC19 plasmid DNA, while extracts from clb− E. coli or medium alone had negligible impacts on DNA integrity (FIG. 2C). Similarly, ethyl-acetate extracts from genotoxic species induced γ-H2AX expression in HeLa, HCT116, and MC38 cells (FIG. 2D, FIG. 10H) and caused tailing in an alkaline comet unwinding assay (FIG. 2E).

Recent meta-analyses have identified cross-cohort microbial signatures associated with CRC, including enrichments in Clostridiaceae, Erysipelotrichaceae, and M. morganii (Thomas et al., 2019, Nature Medicine 25, 667-678; Wirbel et al., 2019, Nature Medicine 25, 679-689). Notably, both C. perfringens and M. morganii were also increased in IBD patients (specifically in CD patients) compared to healthy controls in data from the Human Microbiome Project (FIG. 2F), suggesting potential roles in IBD patients who are at an increased risk of CRC diagnosis (Olen et al., 2020, Lancet 395, 123-131; 016n et al., 2020, Lancet Gastroenterol. Hepatol. 5, 475-484). Overall, M. morganii is enriched in fecal samples from both IBD and CRC patients, and the genotoxicity of this species is restricted to its small molecule fractions. Therefore, M. morganii was prioritized for further genotoxin identification and characterization.

M. morganii Produces Genotoxins that are Distinct from Colibactin

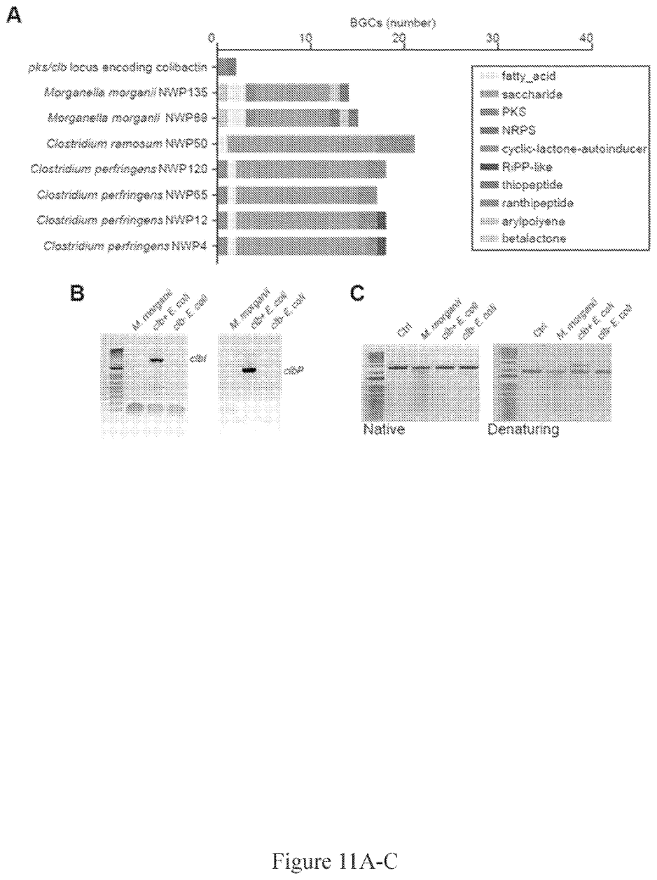

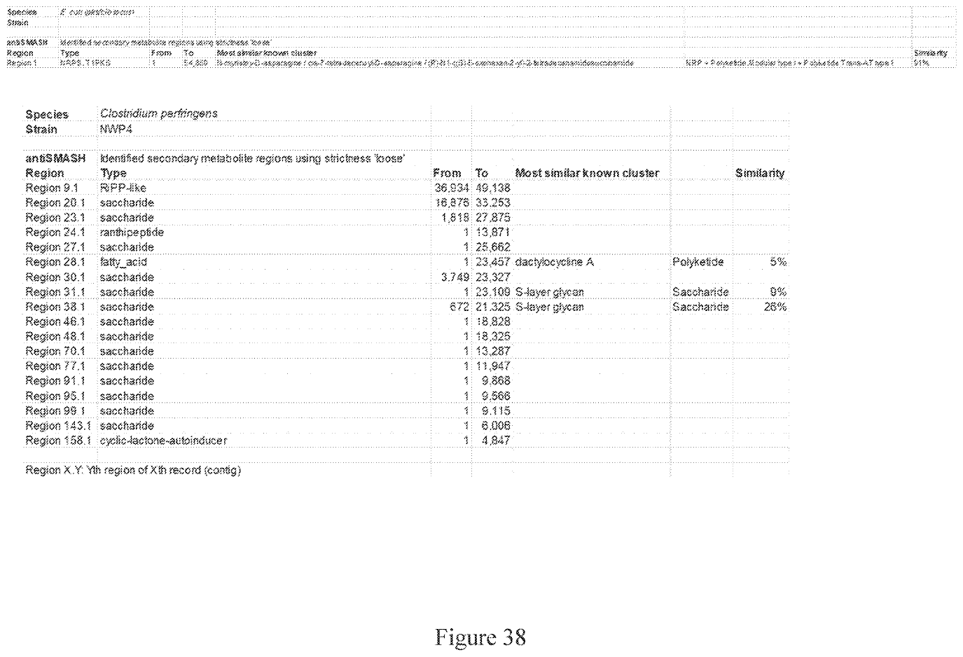

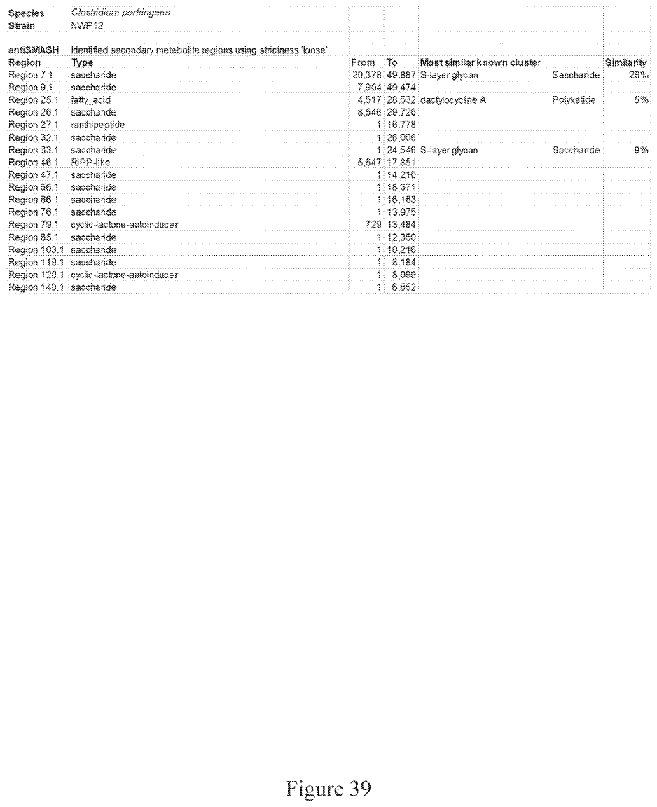

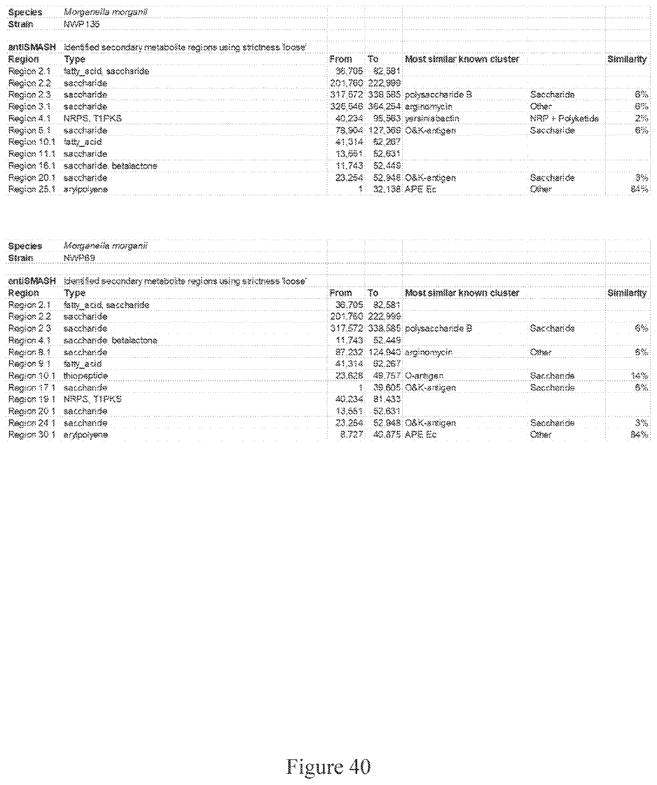

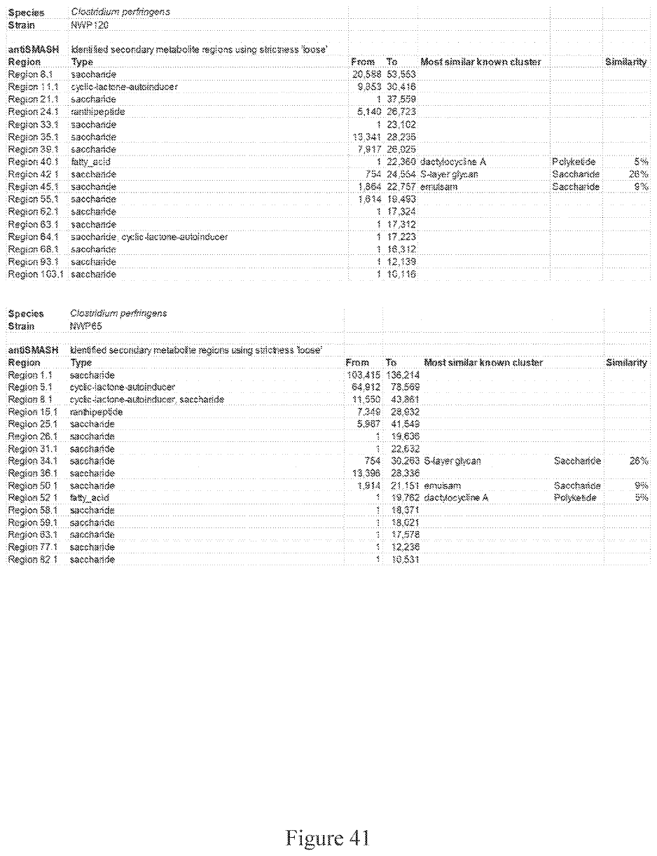

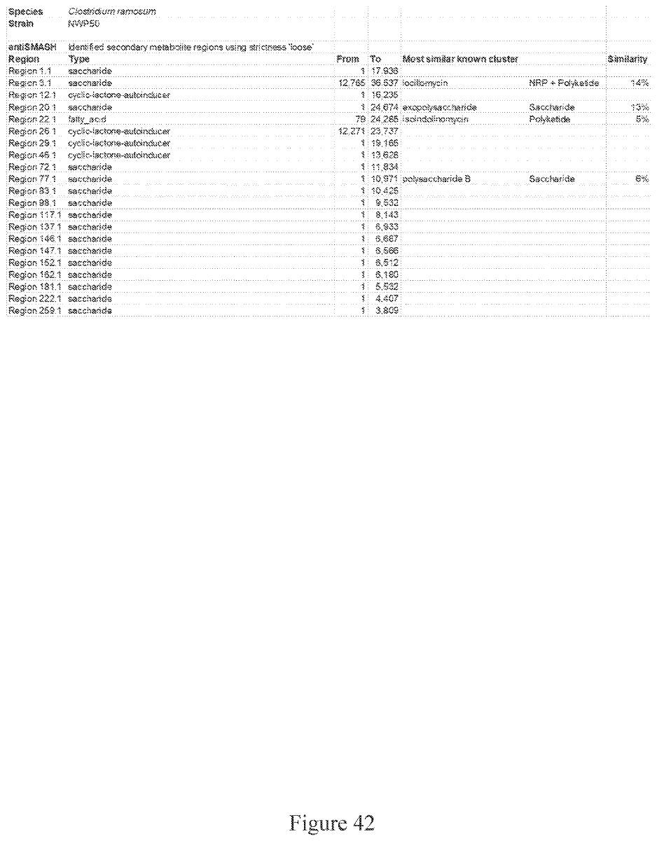

The biosynthetic machinery involved in the production of microbial metabolites, including previously characterized small molecule genotoxins, is often encoded by biosynthetic gene clusters (BGCs) (Shine and Crawford, 2021, Annual review of biochemistry 90, 789-815). For example, colibactin production is encoded by a multimodular PKS-NRPS pathway in E. coli (Nougayrede et al., 2006, Science 313, 848-851; Schneditz et al., 2014, Proc. Natl. Acad. Sci. U.S.A. 111, 13181-13186), and tilimycin and tilivalline are encoded by an NRPS pathway in Klebsiella oxytoca (26). However, BGC analyses of genotoxic C. perfringens, C. ramosum and M. morganii using antiSMASH (Blin et al., 2019, Nucleic Acids Res. 47, W81-W87) failed to detect any known genotoxin-encoding BGCs (FIG. 11A, FIG. 38-42). While M. morganii harbors one NRPS/PKS gene cluster, this BGC is entirely distinct from the c/b genomic island and M. morganii also lacks key genes involved in colibactin synthesis, such as clbI and clbP (FIG. 11B) (Trautman et al., 2017, J. Am. Chem. Soc. 139, 4195-4201; Zha et al., 2017, Nature Chemical Biology 13, 1063-1065). This is consistent with recent analyses of 69 publicly available Morganella genomes, which suggests that clb genes are absent in Morganella genomes (Wami et al., 2021, Microbial Genomics 7, 000577). The genotoxicity caused by M. morganii is also distinct from that caused by colibactin while clb+ E. coli caused DNA crosslinking, DNA exposed to M. morganii displayed a smearing pattern under both native and denaturing conditions (FIG. 11C). Finally, as previously reported, colibactin-induced γ-H2AX required live bacterial infection and supernatants from clb+ E. coli failed to induce dramatic increases in γ-H2AX in cell lines, likely due to documented colibactin instability (Nougayr6de et al., 2006; Shine et al., 2018, ACS Chem. Biol. 13, 3286-3293). By contrast, M. morganii supernatants and small molecule metabolites elicited significant increases in 7-H2AX (FIG. 2, FIG. 10). Together, these data suggest that M. morganii produces previously undescribed genotoxins that are distinct from colibactin and are readily diffusible.

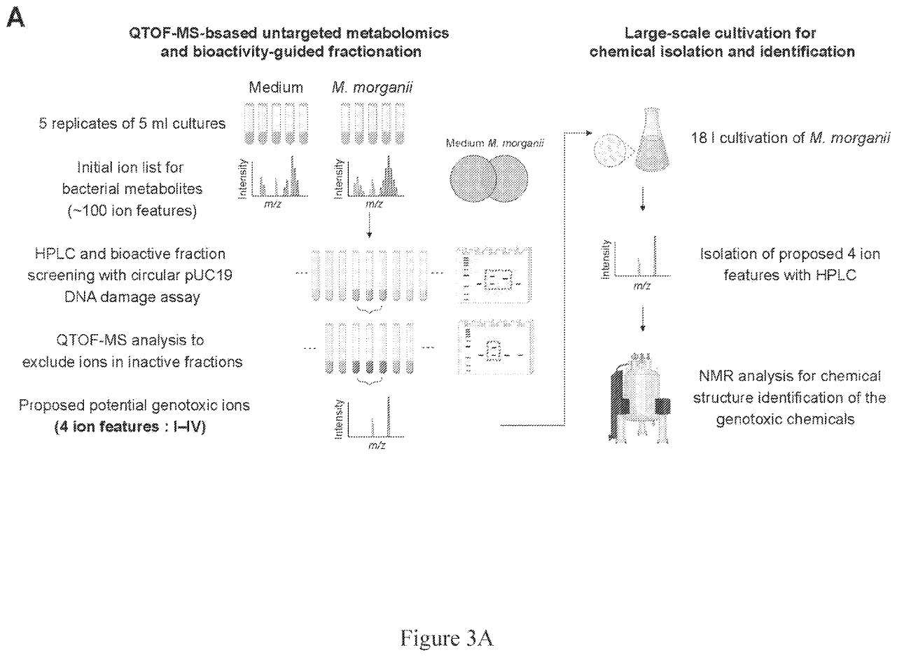

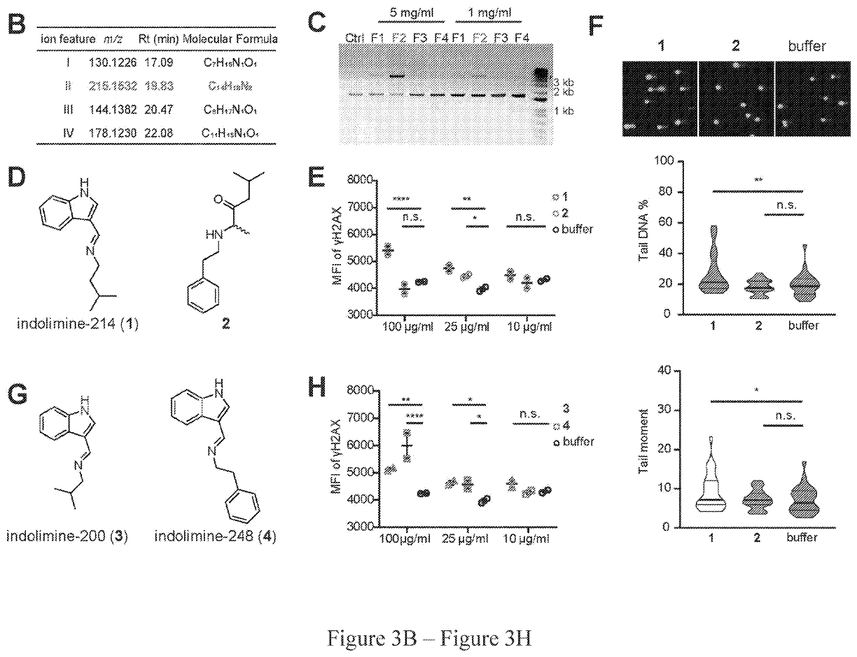

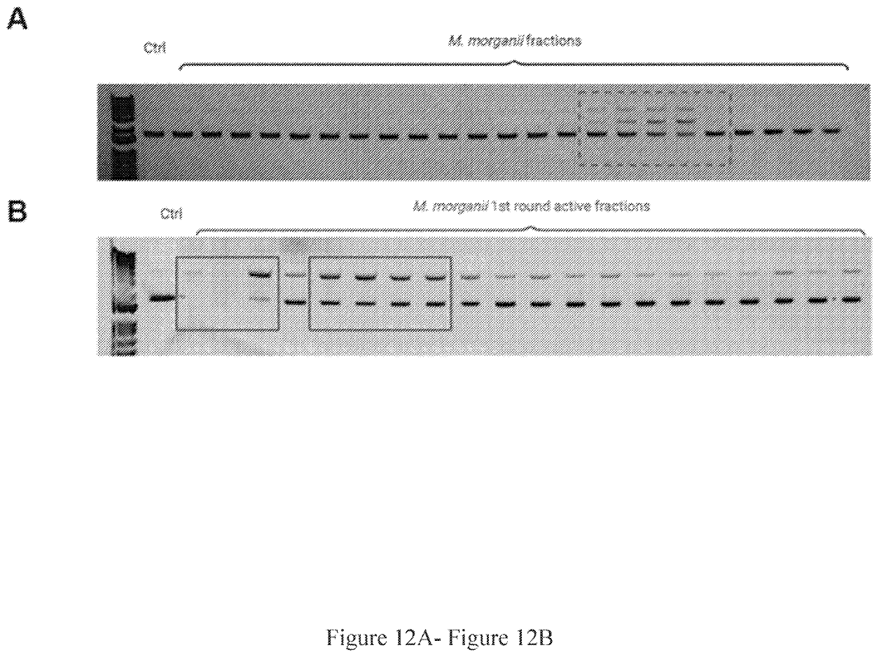

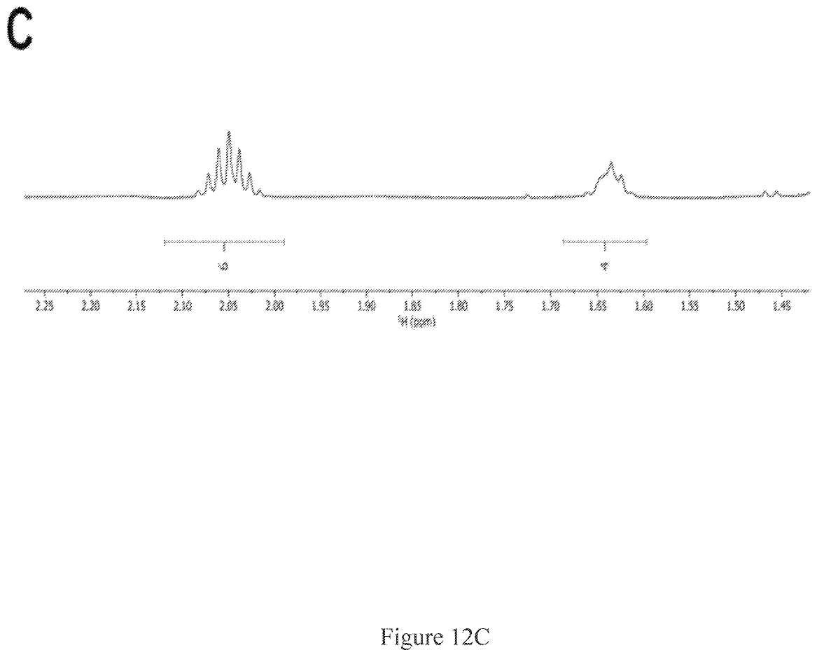

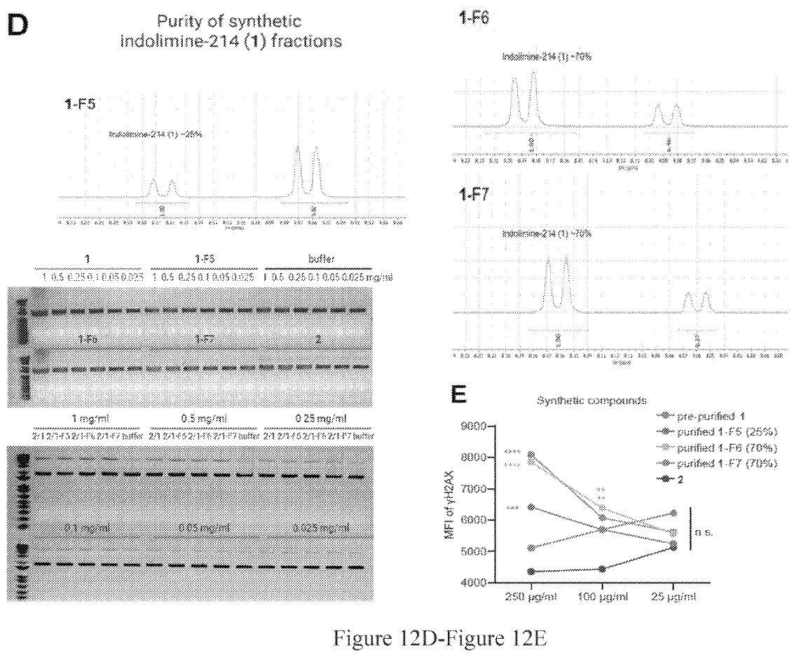

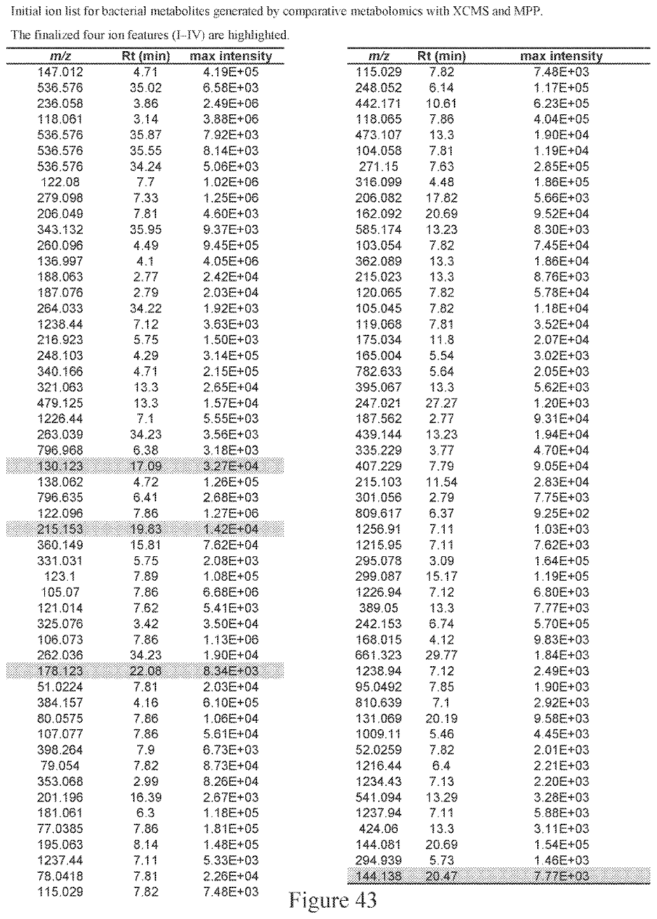

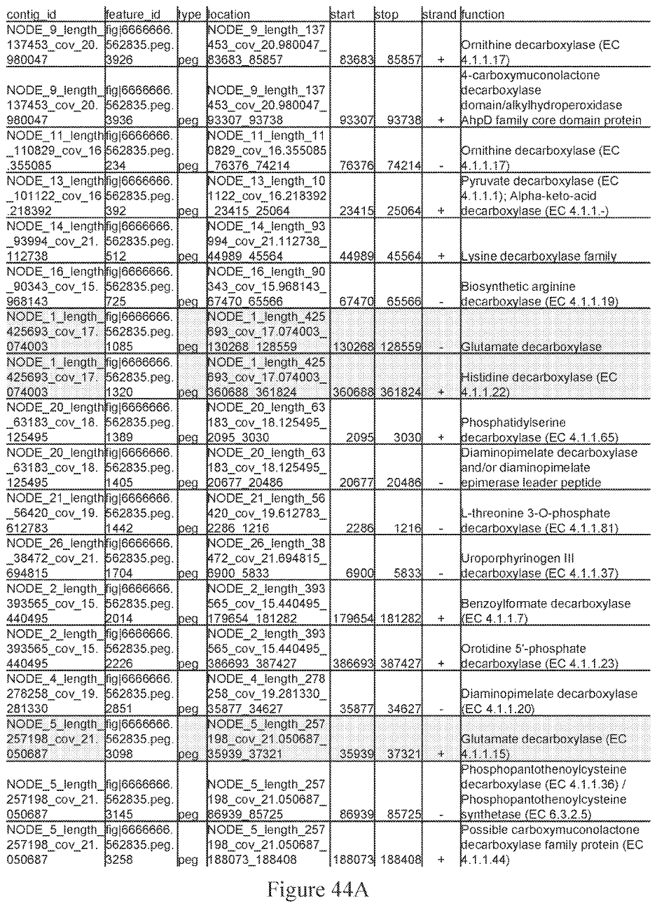

Isolation and identification of a family of genotoxins derived from M. morganii Identifying specific genotoxins produced by M. morganii employed a combination of ultra-performance liquid chromatography quadrupole time-of-flight mass spectrometry (UPLC-QTOF-MS)-based untargeted metabolomics, and bioactivity-guided fractionation using small-scale cultures, followed by large-scale cultivation and isolation for unambiguous structure elucidation and genotoxicity analyses (FIG. 3A). The generated initial candidate ion list of the most abundant M. morganii-derived metabolites relative to Gifu medium control (˜100 ion features, FIG. 38-42) was via comparative metabolomics. The activity-guided fractionation was performed for two rounds using preparative high-performance liquid chromatography (HPLC) and a circular pUC19 plasmid-based genotoxicity assay, then profiled the resulting fractions and subfractions using UPLC-QTOF-MS-based metabolomics (FIG. 12A-B). The potential genotoxins were excluded to identify ions present in inactive fractions from the initial ion list and ultimately identified 4 ion features (I-IV) as potential genotoxic hits (FIG. 3B, FIG. 43). To enable structural elucidation and genotoxic activity assessment for these compounds, large-scale cultivation was performed (18 liters) and ethyl acetate extraction of M. morganii supernatant based on previously observed retention times that imply relatively low polarity of the compounds of interest. The crude extract was subjected to two rounds of HPLC to generate four semi-pure fractions enriched in the four target ion features (F1-F4 enriched in I-IV, respectively). One of these fractions (F2) exhibited dose-dependent genotoxicity in a circular pUC19 plasmid-based genotoxicity assay (FIG. 3C). Based on UPLC-QTOF-MS analyses, F2 was primarily comprised of two metabolites with m/z values of 215.1543 (compound 1, target ion feature II) and 234.1852 (compound 2, a metabolite that was absent from the initial ion list but was co-enriched during extraction and fractionation) at a ratio of 4:6 (FIG. 12C). This fraction was recalcitrant to further purification by preparative HPLC using diverse combinations of stationary and mobile phases. Thus, the chemical structures of the two components were characterized as a mixture using one- and two-dimensional nuclear magnetic resonance (NMR) spectroscopy analyses (FIG. 3D, FIG. 13). Compound 1 consists of a conjugation of indole-3-aldehyde (IAld) and the primary amine isoamylamine, forming a functional imine group. Thus, this previously undescribed metabolite was termed indolimine-214.

To confirm which compound exerted the observed genotoxicity, both indolimine-214 (1) and compound 2 was synthesized. Formation of the reversible imine functional group in compounds such as indolimine-214 (1) varies depending on the chemical environment (e.g., pH, temperature, solvent, and solutes). Therefore, fresh synthetic material was fractionated and assessed the purity of 1 in each fraction using ¹H NMR for genotoxicity analysis (FIG. 12D). Nonetheless, neither synthetic indolimine-214 (1) nor compound 2 alone (up to 1 mg/ml) induced DNA damage in the circular pUC19 plasmid-based genotoxicity assay. However, the mixture of indolimine-214 (1) and compound 2 elicited dose-dependent DNA damage (FIG. 12D), suggesting that the presence of compound 2 may serve as an adjuvant for indolimine-214 (1) (target ion feature 11) in the cell-free assay. In a more biologically relevant context, the genotoxicity of pure synthetic compounds in HeLa cells were assessed to find that indolimine-214 (1) alone, but not compound 2, triggered increased γ-H2AX in a dose-dependent manner (FIG. 3E) and induced tailing in an alkaline comet unwinding assay (FIG. 3F). Furthermore, the genotoxicity of synthetic indolimine-214 (1) correlated directly with its purity (i.e., in relation to its hydrolytic degradation products, FIG. 12E).

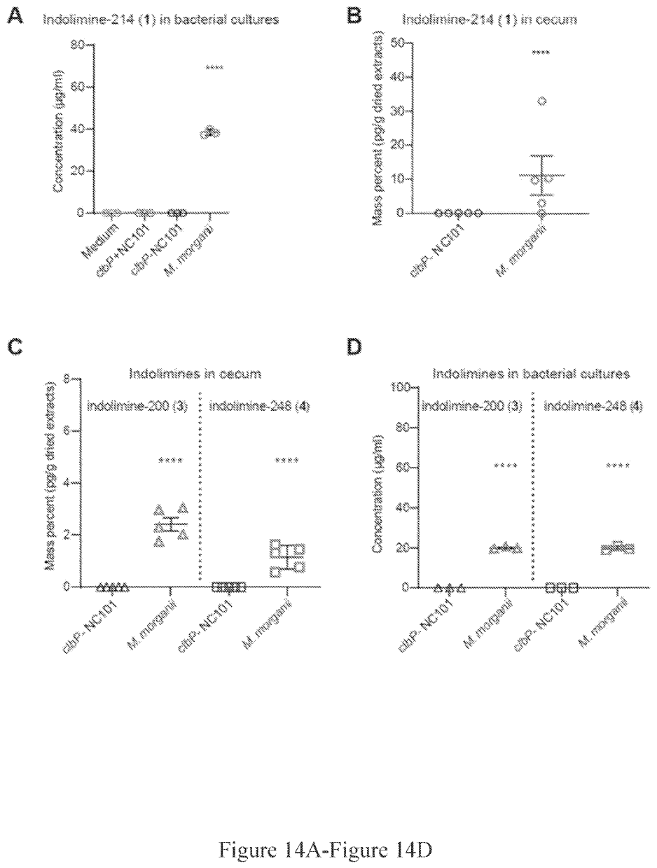

UPLC-QTOF-MS-based quantification found that M. morganii produces a high level of indolimine-214 (1) in vitro (˜40 g/ml, FIG. 14A), which is comparable to the concentrations of the synthetic compound that induced genotoxicity in HeLa cells (FIG. 3E-F). By contrast, indolimine-214 (1) was undetectable in supernatants from wild-type colibactin-producing (clbP+) or isogenic clbP mutant non-colibactin-producing (clbP−) E. coli NC101 strains (Tomkovich et al., 2017, Cancer Res. 77, 2620-2632) (FIG. 14A). This is consistent with the observation that M. morganii—induced DNA damage was distinct from the damage caused by colibactin-producing E. coli (FIG. 11). Cecal contents from mice colonized with M. morganii, but not clbP− E. coli NC101-colonized mice, also contained high levels of indolimine-214 (1) (FIG. 14B). The process of quantifying cecal indolimine-214 (1) observed the presence of two additional new indolimines with similar structures in M. morganii-colonized mice: conjugates of IAld with either isobutylamine (indolimine-200, compound 3, n z 201.1386) or phenethylamine (indolimine-248, compound 4, m/z 249.1386) (FIG. 3G, FIG. 14C, FIG. 15). These additional indolimines were also detected in in vitro M. morganii bacterial cultures, but not clbP− E. coli NC101 cultures, as confirmed by synthetic standards (FIG. 14D). Finally, synthetic indolimine-200 (3) and indolimine-248 (4) also triggered increased 7-H2AX in HeLa cells in a dose-dependent manner (FIG. 3H) Taken together, these data show that M. morganii produces a family of genotoxic indolimines both in vitro and in vivo.

### a Previously Uncharacterized Bacterial Decarboxylase is Necessary for Indolimine Synthesis

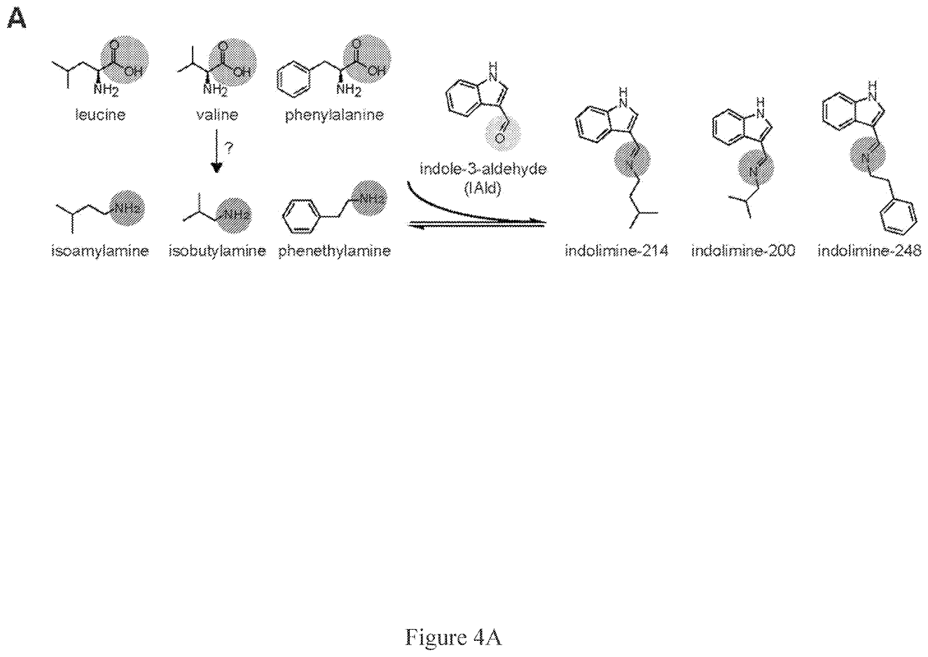

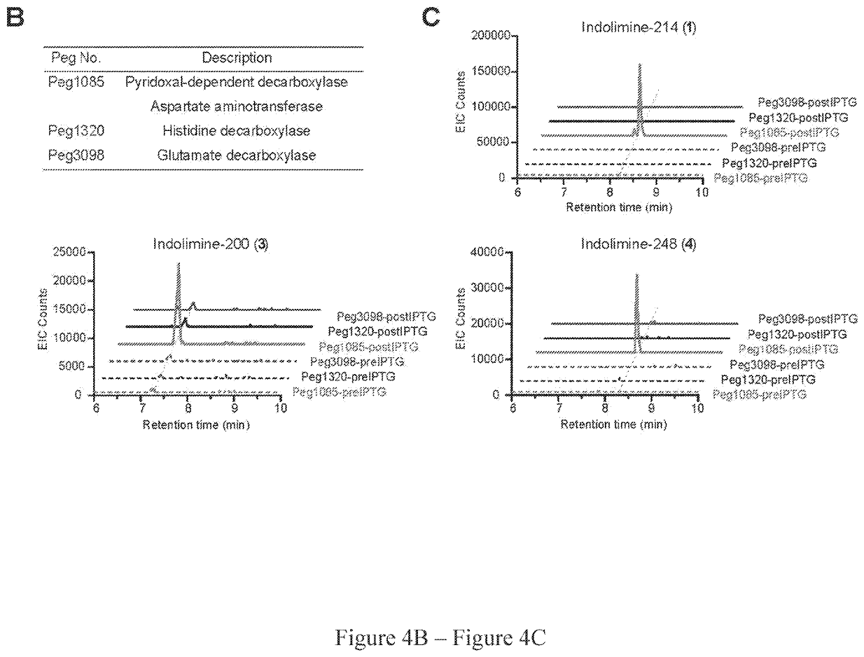

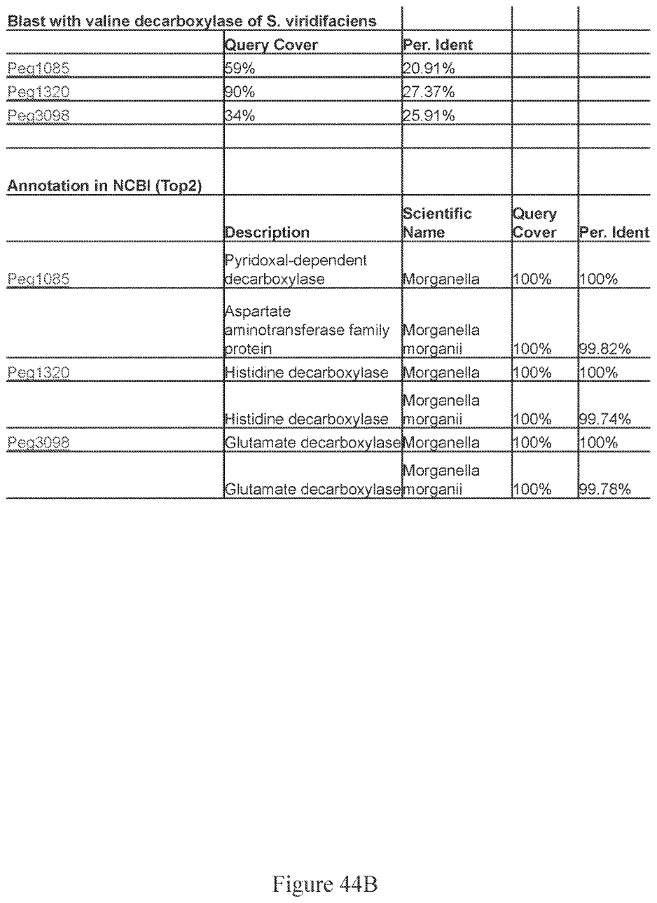

All M. morganii-derived indolimines contain a functional imine group, which is likely derived from the spontaneous condensation of a primary amine (isoamylamine, isobutylamine, and phenethylamine) and the aldehyde of indole-3-aldehyde (IAld) (FIG. 4A). Primary amines are microbial metabolites that can be synthesized from amino acids via a one-step reaction mediated by bacterial decarboxylases (Kim et al., 2021, Nat. Commun. 12, 173). Based on whole-genome sequencing, M. morganii NWP135 encoded 18 predicted decarboxylases, including three decarboxylases (Peg1085, Peg1320, and Peg3098) that were partially homologous to a previously characterized valine decarboxylase from S. viridifaciens (Kim et al., 2021, Nat. Commun. 12, 173) (FIG. 4B, FIG. 43-44). Codon-optimized DNA sequences of these three candidate decarboxylases into E. coli were transformed, induced for protein expression with IPTG, and fed each culture with IAld and the relevant amino acid precursors (leucine, valine, or phenylalanine). Peg1085, annotated as pyridoxal-dependent decarboxylase or aspartate aminotransferase (AAT) superfamily (fold type I) in NCBI (FIG. 4B, FIG. 43-44), expression in E. coli enabled robust production of indolimine-214 (1), indolimine-200 (3), and indolimine-248 (4) based on QTOF-MS identification (FIG. 4C). Therefore, the aat gene encoding AAT_I (Peg1085) enables indolimine synthesis.

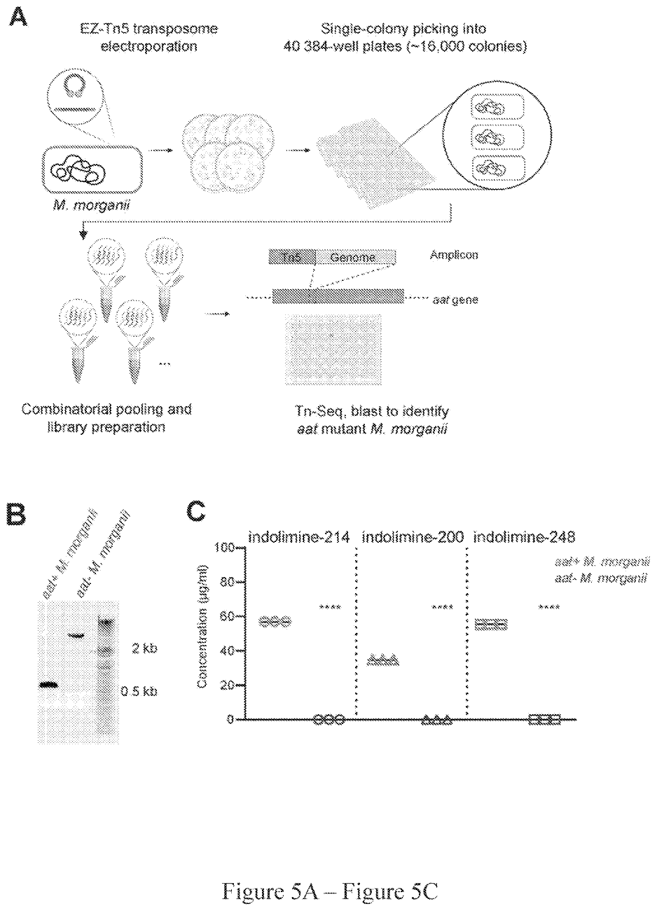

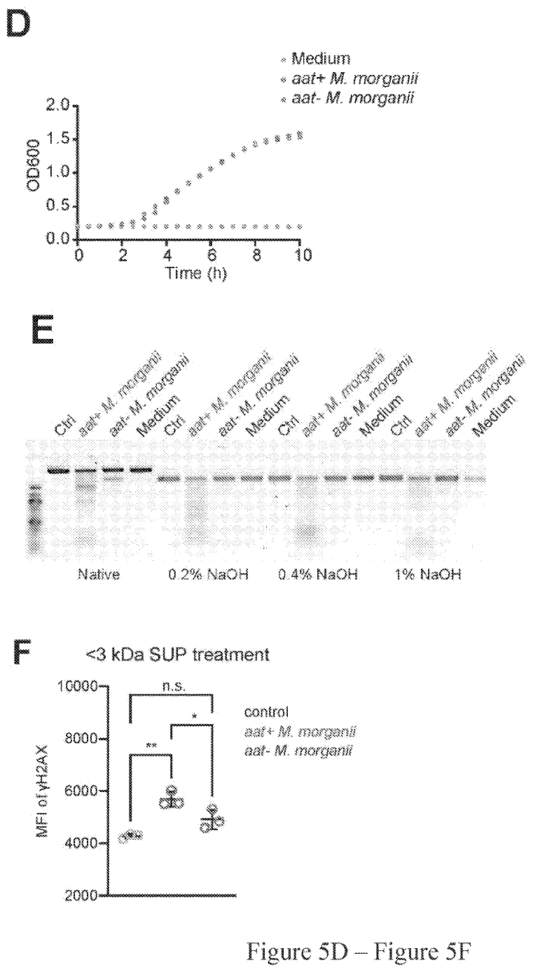

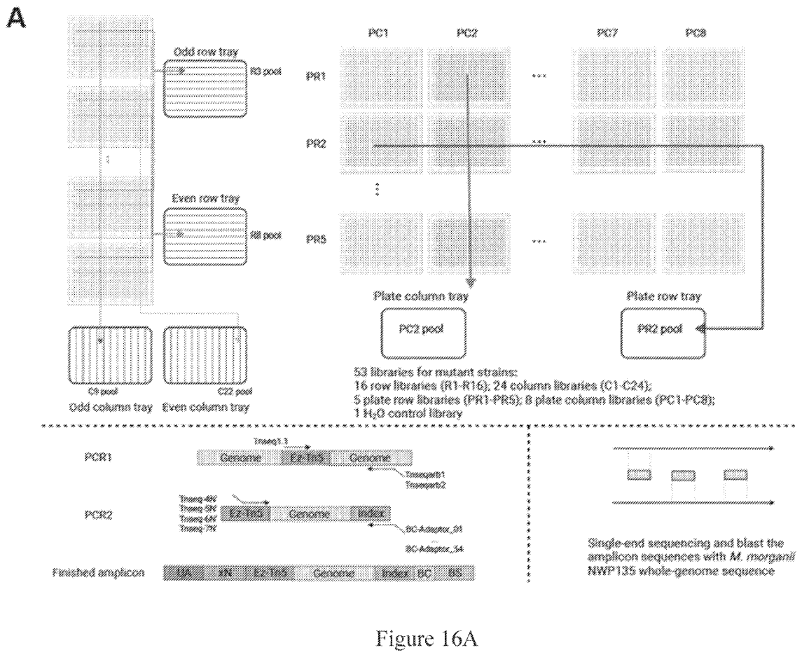

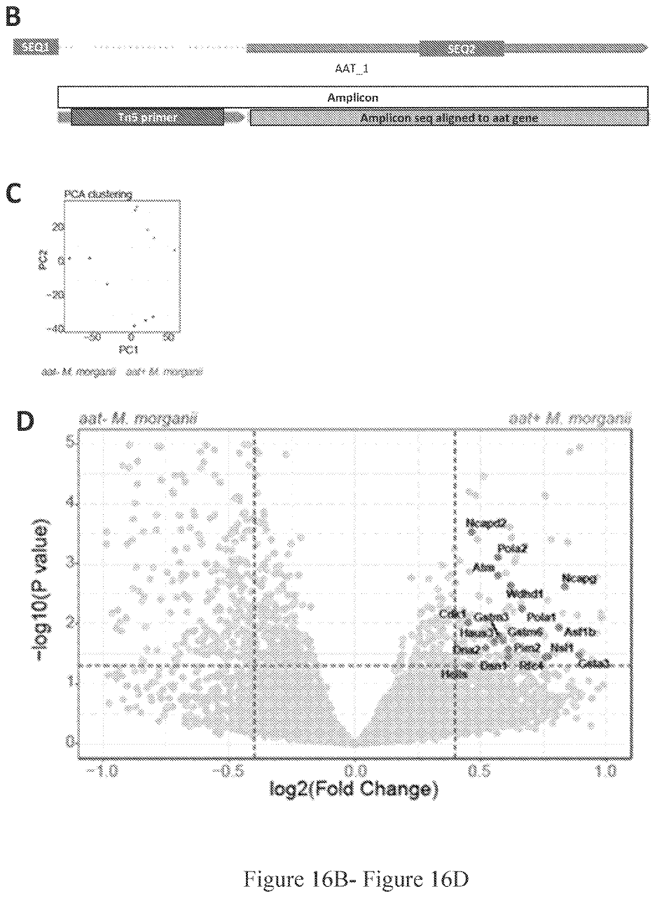

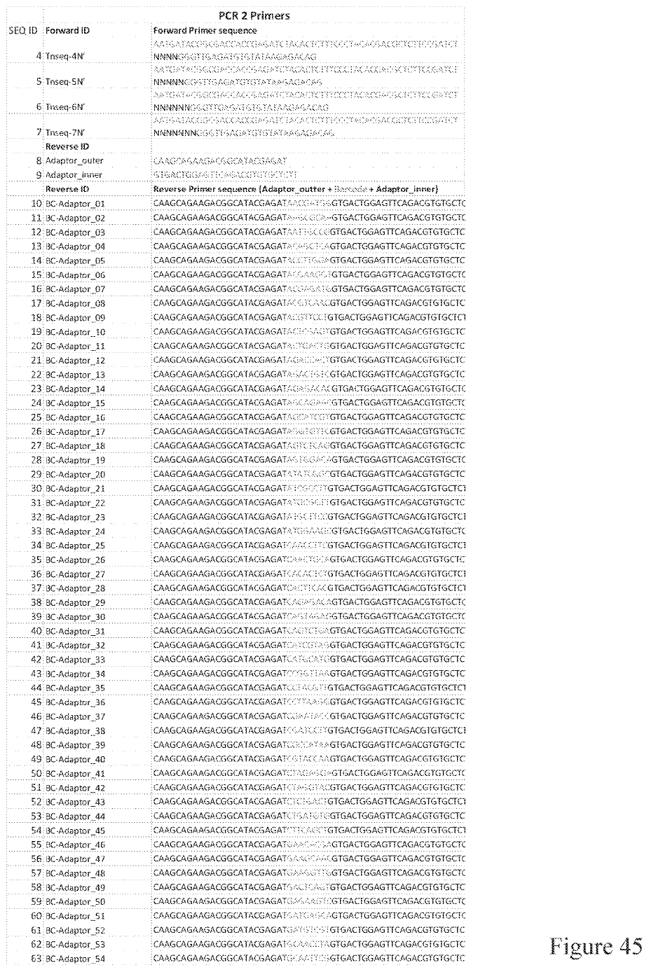

To evaluate if the aat gene is necessary for indolimine synthesis in M. morganii, the random mutagenesis library of M. morganii NWP135 using EZ-Tn5 transposomes and isolated an isogenic aat mutant was constructed (Veeranagouda et al., 2012, FEMS Microbiol Lett. 333, 94-100) (FIG. 5A). Briefly, after optimizing transposome electrotransformation, ˜16,000 were picked and the transposons insertion sites were mapped by combining combinatorial pooling and transposon sequencing (Tn-Seq) using custom primers adapted from Knockout Sudoku (FIG. 16A, FIG. 45-47) (Anzai et al., 2017, Nat. Protoc. 12, 2110-2137; Lariviere et al., 2019, Curr. Biol. 29, 1460-1470). One mutant strain was identified (aat− M. morganii) with a transposon insertion 7 bp after the aat start codon (aat−; FIG. 5B, FIG. 16B). As compared to wild-type (aat|) M. morganii, aat− M. morganii failed to produce indolimines (FIG. 5C) despite exhibiting normal growth dynamics (FIG. 5D). aat− M. morganii also failed to induce DNA damage in a cell-free linearized plasmid DNA electrophoresis assay (FIG. 5E) or cell-based 7-H2AX assay (FIG. 5F) as compared to aat+ M. morganii. Therefore, the aat gene is essential for indolimine synthesis and M. morganii-induced genotoxicity.

TABLE 3
Design of PCR primers adapted from Knockout
Sudoku to identify mutant strains

| | | |
|---|---|---|
| SEQ | | |
| ID | | |
| NO: | Description | Sequence |

PCR1 primers

| | | |
|---|---|---|
| 126 | Tnseq1.1 | ACCTACAACAAAGCTCTCATCAACC |
| | (upstream | |
| | of Tn5) | |
| 127 | Tnseqarb1 | GAGTTCAGACGTGTGCTCTTCCGAT |
| | | CTNNNNNNNNNNGATAT |
| 128 | Tnseqarb2 | GAGTTCAGACGTGTGCTCTTCCGAT |
| | | CTNNNNNNNNNNACGC |

Identification Primers

| | | |
|---|---|---|
| 129 | Tnseq1.2 | GGGTTGAGATGTGTATAAGAGACAG |
| | (sequence | |
| | primer) | |
| 130 | Tnseq2.2 | GTGACTGGAGTTCAGACGTGTGCTCTT |
| 131 | AAT F | TGACATTACCTCAGCGTAATTTAC |
| 132 | AAT R | CAGCATAGACCGTAAACTGC |

Indolimine-Producing M. morganii Induces Increased Gut Permeability and Exacerbates Colon Tumor Burden in gnotobiotic Mice

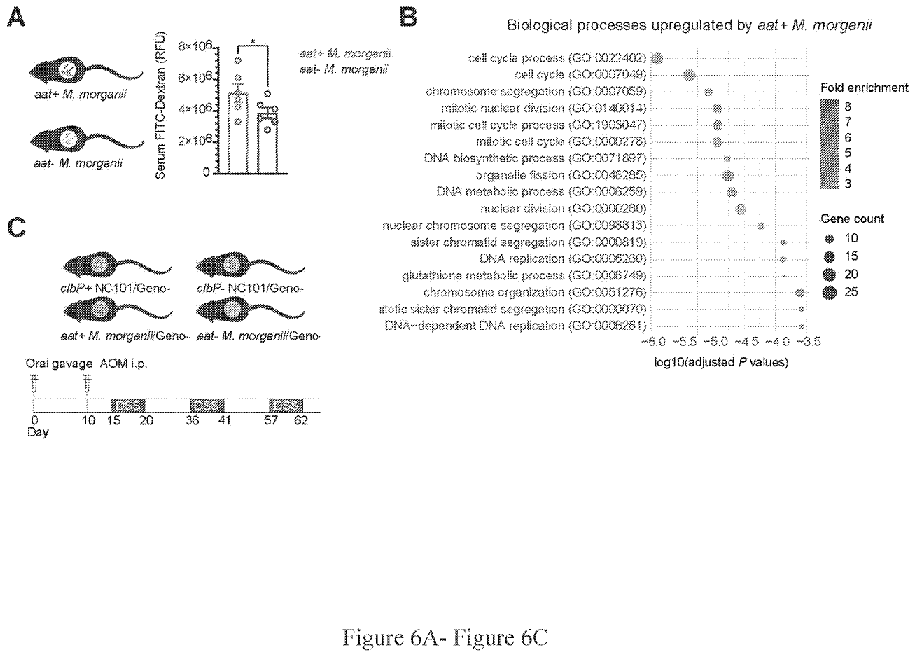

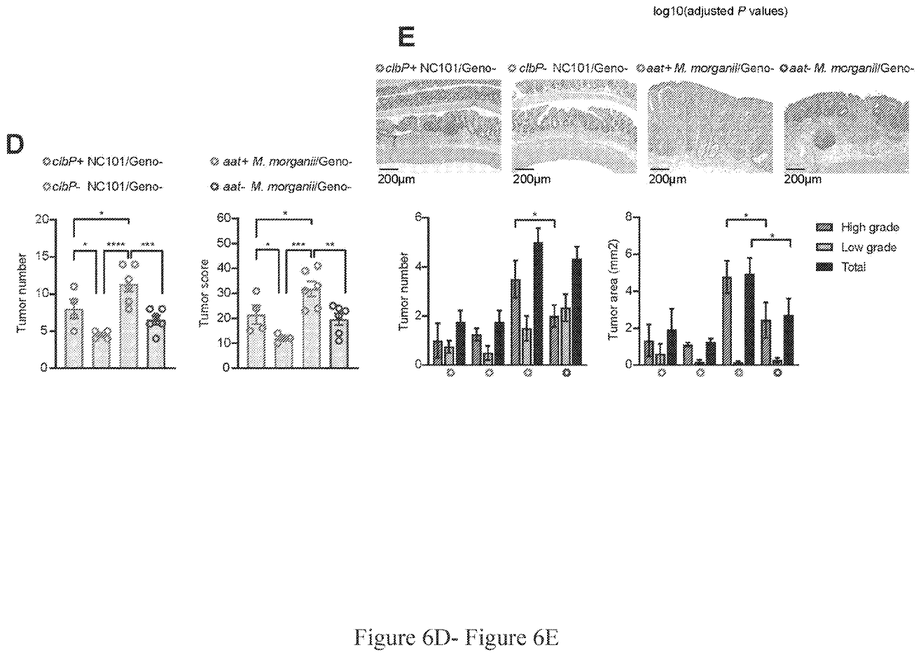

Evaluating the impacts of M. morganii—derived indolimines in vivo, were first compared with the effects of aat+ and aat− M. morganii strains on the intestinal epithelium using monocolonized mice. The mice colonized with aat+ M. morganii and fed IAld and leucine (precursors of indolimine-214) exhibited significantly increased intestinal permeability as compared to aat− M. morganii-colonized mice (FIG. 6A). Furthermore, RNA-seq of colonic intestinal epithelial cells (IECs) and Gene Ontology analyses of differentially expressed genes revealed an increased expression of genes involved in cell cycle regulation, chromosome segregation, and DNA biosynthesis in mice colonized with aat+ M. morganii (FIG. 6B, FIG. 16C-D). Together, these data suggest that the indolimines may cause abnormal DNA replication and IEC proliferation in vivo.

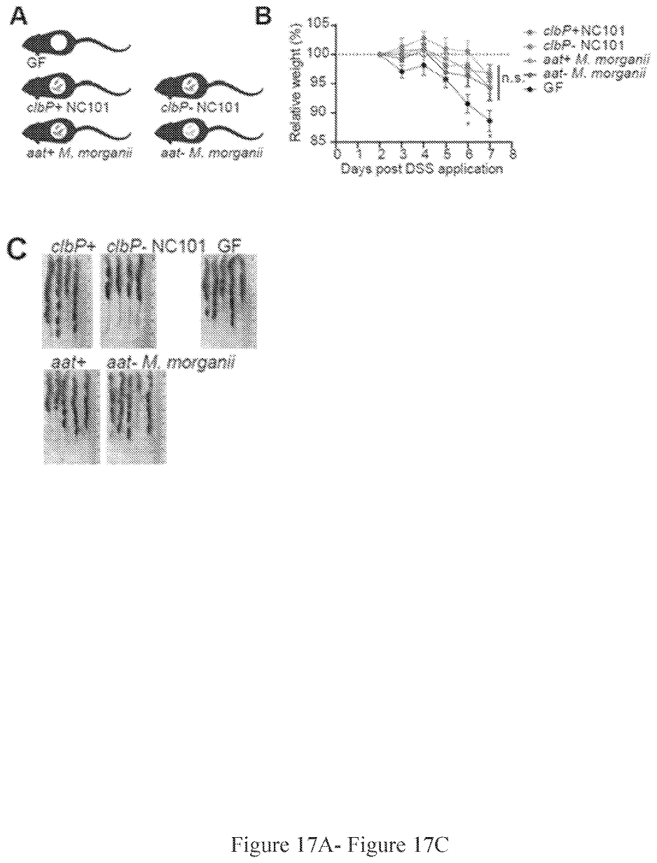

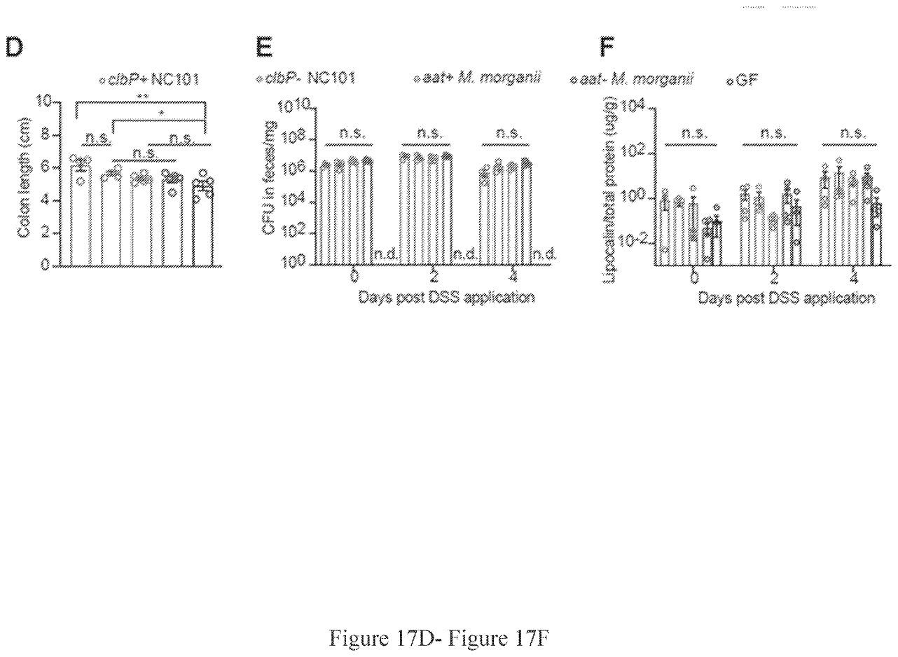

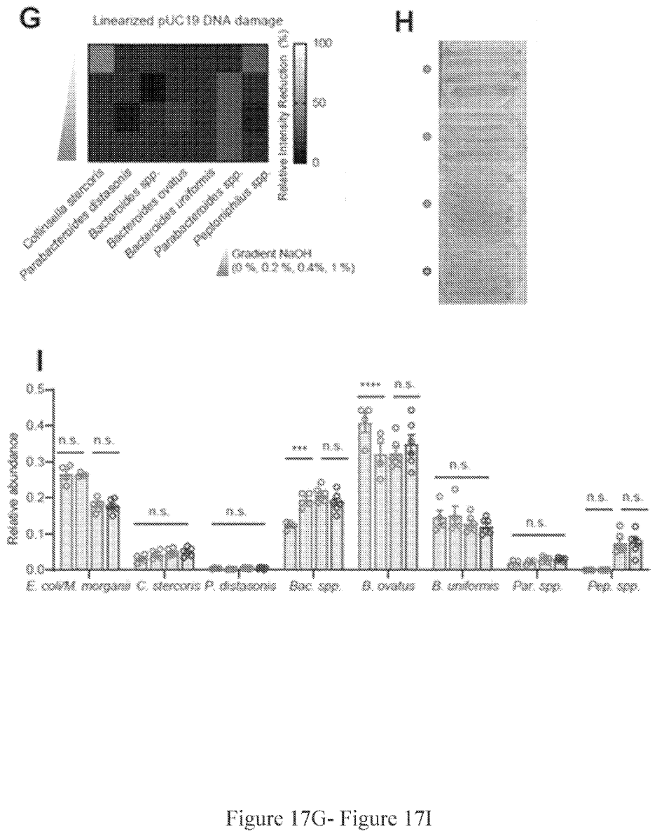

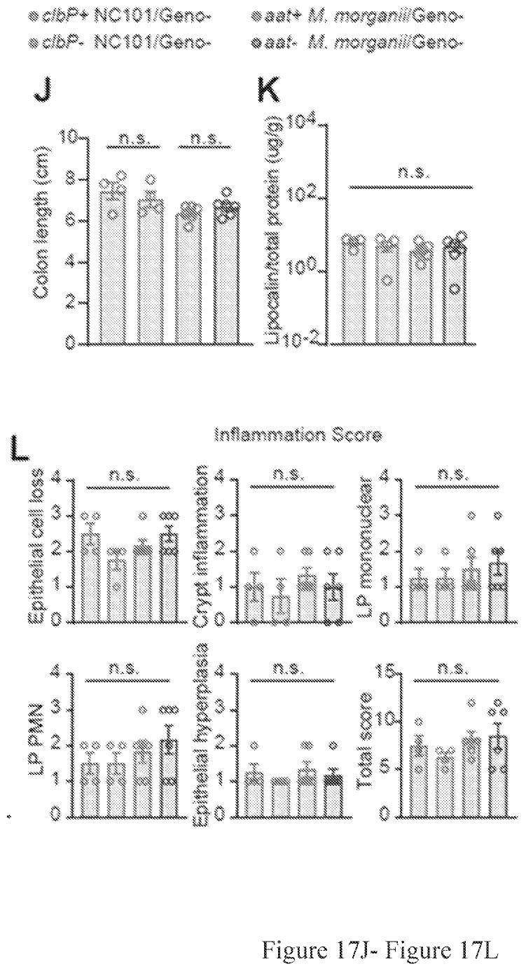

The next experiments evaluated whether indolimine-producing aat+ and non-indolimine-producing aat− M. morganii strains induced differential inflammatory responses, with wild-type colibactin-producing clbP− and non-colibactin-producing clbP− E. coli NC101 strains (Tomkovich et al., 2017, Cancer Res. 77, 2620-2632) as controls (FIG. 17A). All groups of monocolonized mice exhibited similar levels of bacterial colonization and roughly equivalent inflammatory responses after treatment with dextran sulfate sodium (DSS) (FIG. 17B-F), indicating that genotoxin production did not significantly alter bacterial colonization or gross inflammatory phenotypes in a model of acute colitis.

Gnotobiotic mice with aat+ M. morganii or aat− M. morganii were tested for the potential effects of indolimine production on colon tumorigenesis, in the context of a mock community of human gut microbes and then treated with azoxymethane (AOM) and three cycles of DSS (FIG. 6C). Seven non-genotoxic human gut isolates were selected based on the prior in vitro cell-free genotoxicity screening (FIG. 1, FIG. 17G) to construct a mock community (Geno- community) and used wild-type colibactin-producing clbP+ or its isogenic mutant non-colibactin-producing clbP− E. coli NC101 strains (Tomkovich et al., 2017, Cancer Res. 77, 2620-2632) as genotoxin-producing or non-genotoxin-producing positive and negative controls (FIG. 6C). As expected, colonization with clbP+ E. coli NC101 induced increased colorectal tumor burdens as compared to clbP− E. coli NC10¹; similarly, mice colonized with indolimine-producing aat+ M. morganii also exhibited increased tumor numbers and tumor scores (indicating overall tumor burden) as compared to mice colonized with non-indolimine-producing aat− M. morganii (FIG. 6D, FIG. 17H). Moreover, mice colonized with aat+ M. morganii exhibited an increased ratio of adenomatous lesions with high-grade dysplasia (FIG. 6E). Nonetheless, mice colonized with aat+ or aat− M. morganii exhibited similar levels of M. morganii colonization (FIG. 17I) and intestinal inflammation, as measured by colon length (FIG. 17J), levels of fecal lipocalin 2 (FIG. 17K) and histopathology (FIG. 17L). These data suggest that genotoxic indolimine-producing aat+ M. morganii exacerbate colon tumorigenesis in this model, but do not elicit dramatic increases in intestinal inflammation as compared to a non-genotoxic control.

Finally, Cancer Microbiome database (Poore et al., 2020, Nature 579, 567-574) was mined to find that Morganella exhibited increased prevalence or abundance in primary gastrointestinal (GI) tumors, including colon adenocarcinomas (TCGA-COAD), rectum adenocarcinomas (TCGA-READ), and stomach adenocarcinomas (TCGA-STAD), as compared to multiple non-GI tumors (FIG. 18A). Morganella was also significantly enriched in tumors from TCGA-READ and TCGA-STAD as compared to adjacent solid normal tissues (FIG. 18B), consistent with a recent report that Morganella is enriched in cancerous tissues as compared to luminal contents (Chen et al., 2012, PLoS One 7, e39743). Notably, aat was conserved across nearly all M. morganii strains with full genome sequences in the NCBI database (51 of 52 genomes, FIG. 18C), suggesting that indolimine production may be a conserved feature of M. morganii. Overall, the data, in combination with prior reports, suggest that genotoxic indolimines from M. morganii may serve as tumor-inducing factors in humans.

The methods and materials used in this example are now described

### Bacteria Strains and Plasmids

NWP strains were isolated from 11 IBD patients via anaerobic culturomics as previously described and identified via 16S rRNA gene sequencing (V4 region) (20). The E. coli K-12 BW25113 strains with and without the clb island (clb|E. coli or clb− E. coli) were generated by the Crawford laboratory (15). The E. coli NC101 wild-type (clbP+) and isogenic clbP mutant (clbP−) strains were generated by the Jobin laboratory (38). 25 g/ml of chloramphenicol or 50 μg/ml kanamycin were added for bacteria culturing when appropriate.

NWP strains were cultured in Gifu Anaerobic Broth (GAM Broth) at 37° C. under anaerobic conditions in a Coy Laboratory Products Inc. chamber (10% CO₂,4% H₂.86% N₂). For screening based on cell-free electrophoresis, bacterial growth curves for all isolates were measured for 48 h starting at OD₆₀₀=0.01 using a BioTek PowerWave HT Microplate Spectrophotometer.

The 2686 bp plasmid pUC19 was purchased from New England Biolabs (N3041) and linearized with the endonuclease EcoRI-HF (NEB, R3101). Linearized plasmid DNA was purified using the Monarch® PCR and DNA Cleanup Kit (NEB, T1030) and eluted with 10 mM Tris-1 mM EDTA pH 7.5 buffer. pET-28 plasmids were from the Crawford laboratory.

Individual isolates were co-cultured with linearized plasmid DNA under anaerobic conditions to stationary phase (T_{S}); co-cultured under anaerobic conditions to exponential phase (T_{E}), and then under aerobic conditions (atmospheric conditions, usually 20% O₂, for oxygen stress) to stationary phase (T_{S}); co-cultured under anaerobic conditions to exponential phase (T_{E}), or co-cultured linearized plasmid DNA with bacterial supernatants collected from anaerobic cultures at stationary phase (T_{S}).

### Media

Gifu Anaerobic Medium (GAM Broth) was purchased from Himedia Laboratories (M1801); Luria Broth Base (Miller's LB Broth Base)™ was purchased from Invitrogen (12795027); Chopped Meat Medium (CM, AS-811), Brucella Broth (BRU, AS-105), Yeast Casitone Fatty Acids Broth with Carbohydrates (YCFAC, AS-680), and MTGE Anaerobic Enrichment Broth (MTGE, AS-778) were purchased from Anaerobe Systems; BD Difco™ Lactobacilli MRS Broth (MRS, 288130), Reinforced Clostridial Medium (RCM, 218081), Malt Extract Broth (211320), and Bacto™ Brain Heart Infusion (BHI, 237500) were purchased from BD Biosciences; Todd Hewitt Broth (THB, DST47500) was purchased from DOT Scientific, Inc.; M9 minimal medium was prepared with 5× M9 salts (30 g Na₂HPO₄, 15 g KH₂PO₄, 2.5 g NaCl, 5 g NH₄Cl for 11 stock), 0.4% glucose, 2 mM MgSO₄, and 0.1 mM CaCl₂); M9-CA was prepared with M9 minimal medium supplemented with 0.2% Bacto™ Casamino Acids (BD Biosciences, 223050); Standard amino acid complete (SACC) medium was prepared as published (Dodd et al., 2017, Nature 551, 648-652).

### In Vitro Linearized DNA Gel Electrophoresis Assay

All bacterial strains were classified into seven groups with similar growth dynamics based on their individual growth curves. Screening experiments were designed based on approximate T_{E} and Ts within these seven groups. Two-day cultures of each isolate were diluted to OD₆₀₀=0.01 in 250 μl liquid media and co-incubated with 1 g linearized pUC19 plasmid DNA in 96-well deep well plates under indicated conditions. After co-incubation, bacterial cultures were centrifuged to remove bacterial cells and supernatants were collected for linear DNA extraction using the Monarch® PCR and DNA Cleanup Kit (NEB, T1030). Purified linearized DNA samples were eluted in 20 μl DEPC-treated water.

For native gel electrophoresis, 1 μl (˜50 ng) of DNA was mixed with 6× purple gel loading dye without SDS (NEB). For denaturing conditions, 1 μl of DNA was treated with 0.2%, 0.4%, or 1% NaOH denaturing buffers on ice for 30 min, then mixed with 6× purple gel loading dye. Gel electrophoresis was performed using 1% agarose TBE gels for 1-2 h at 90 V. Gels were stained with 5000× SYBR Gold (Thermo Fisher Scientific, S11494) for 2 h at room temperature before UV visualization. DNA band intensity (double-stranded DNA under native condition or single-stranded DNA under denaturing conditions) was quantified with ImageJ and the relative intensity reduction (RIR, %) resulting from DNA damage was calculated by comparing DNA band intensity after co-incubation with live bacterial cultures or bacterial supernatants versus medium only controls

### Ethyl-Acetate Extraction of Bacterial Metabolites

Bacterial cultures were centrifuged to remove bacterial cells and other large particles. The resulting clarified bacterial supernatants were extracted three times using two equivalent volumes of ethyl acetate, and the organic layers were collected and dried via rotary evaporator. The dried samples were placed under vacuum for another 6 h before weighing the dried extracts. Dried crude extracts were reconstituted with dimethyl sulfoxide as a stock solution at 500 mg/ml.

### In Vitro Circular DNA Gel Electrophoresis Assay

Circular pUC19 plasmid DNA (100 ng) was co-incubated with ethyl-acetate extracts or HPLC fractions at 37° C. under aerobic conditions for 6 h or overnight. Native DNA gel electrophoresis was performed using 1% agarose TBE gels containing ethidium bromide for 1-2 h at 90 V.

### Cell Culture

HeLa, MC38 and HCT116 cells were cultured in high glucose Dulbecco's Modified Eagle's Medium (DMEM, Sigma-Aldrich, D6429) supplemented with 10% FBS (Sigma-Aldrich, 12306C) and 1% Penicillin-Streptomycin (Thermo Fisher Scientific, 15140122).

To assess DNA damage caused by bacterial supernatants, anaerobic bacterial cultures were collected and filtered using Amicon Ultra-0.5 Centrifugal Filter Unit, 3KDa (EMD Millipore, UFC500324). HeLa cells were treated with unfractionated SUP (bacterial supernatants), <3 kDa SUP (small-molecule metabolites) or >3 kDa SUP (large-molecules) diluted 40% (v/v) in culture medium supplemented with 10 mM HEPES (Thermo Fisher Scientific, 155630080) for 4-6 h.

To assess DNA damage caused by bacterial infection with colibactin-producing E. coli, E. coli cultures were incubated overnight, collected, washed with PBS and resuspended in culture medium supplemented with 10 mM HEPES. HeLa cells (˜70% confluent) were infected for 4 h at a multiplicity of infection of 100.

To assess DNA damage using chemical extracts of bacterial metabolites, HeLa, MC38 or HCT116 cells were treated with 5 mg/ml ethyl-acetate extracts diluted in culture medium supplemented with 10 mM HEPES for 4-6 h.

To assess DNA damage using pure compounds, HeLa cells were treated with 100 g/ml cisplatin, 100 μg/ml, 25 μg/ml, or 10 μg/ml indolimine-214 (1), compound 2, indolimine-200 (3), indolimine-248 (4) or 0.1% DMSO diluted in culture medium supplemented with 10 mM HEPES for 4-6 h.

After treatment, cells were washed with PBS and harvested using 0.25% Trypsin-EDTA (Sigma-Aldrich, T4049) for assessment via flow cytometry or comet assay.

### Flow Cytometry

For intracellular γ-H2AX staining, harvested post-treated cells were washed with PBS once and fixed with Ebioscience™ IC Fixation Buffer (Thermo Fisher Scientific, 00822249) in the dark for 20 min at room temperature. Intracellular γ-H2AX staining (1:100 AF647 anti-phospho-H2AX; BioLegend, 613408) was conducted in Ebioscience™ Permeabilization Buffer (Thermo Scientific, 00833356) for 1 h after 20 min permeabilization at room temperature. 10,000-20,000 events per sample were collected using a CytoFLEX flow cytometer (Beckman Coulter, Inc.) and MFI (geometric mean fluorescence intensity) was analyzed with FlowJo_v10.7.1.

For cell cycle analysis, harvested post-treated HeLa cells were washed once with PBS and fixed with 90% ice-cold ethanol in the dark for 30 min on ice. After washing, cells were suspended in PBS containing 50 g/ml propidium iodide (Abcam, ab14083) and 100 μg/ml RNaseA. Cells were incubated for 30 min at 37° C. before being collected using a CytoFLEX flow cytometer (Beckman Coulter, Inc.).

For cell death analysis, harvested post-treated cells were analyzed with FITC Annexin V Apoptosis Detection Kit with 7-AAD (BioLegend, 640922) following the manufacture's protocol. Briefly, cells were washed twice with cold BioLegend Cell Staining Buffer, and then resuspend cells in Annexin V Binding Buffer. After adding FITC Annexin V and 7-AAD Viability Staining Solution, cells were incubated for 15 min at room temperature in the dark. Cells were analyzed with CytoFLEX flow cytometer (Beckman Coulter, Inc.) after adding Annexin V Binding Buffer.

### Comet Assay

After treatment with ethyl-acetate extracts or pure synthetic compounds, HeLa cells were washed with PBS and collected via trypsinization. DNA damage was assessed via alkaline single cell gel electrophoresis using the Trevigen Comet Assay kit (Trevigen, Inc.) according to the manufacturer's instructions. Briefly, cells were embedded in Comet LMAgarose and loaded onto CometSlide™. Slides were placed flat at 4° C. in the dark for 10 min and then immersed overnight in Lysis Solution at 4° C. After overnight incubation, slides were immersed in Alkaline Unwinding Solution for 20 min at room temperature and then subjected to electrophoresis under 15 V for 70 min at 4° C. After gently washing slides with water and 70% ethanol, DNA was stained with Invitrogen™ SYBR™ Gold Nucleic Acid Gel Stain (Thermo Fisher Scientific, S11494) for 30 min in the dark. Comet images were acquired using a Leica DMRB fluorescence microscope. Tail DNA % (Tail DNA content as a percentage of Comet DNA content) and Tail moment (Tail length multiplied by Tail DNA %) were analyzed with OpenComet v1.3.1., and 20-50 comets were recorded for each sample.

Chemical Isolation, Identification, Synthesis, and Quantification Ultraviolet/visible (UV/Vis) spectra were obtained on an Agilent 1260 Infinity system equipped with a photo diode array (PDA) detector (Agilent Technologies, Inc.). The nuclear magnetic resonance (NMR) spectroscopy data were generated at 25° C. on an Agilent 600 MHz NMR spectrometer (DD2) equipped with an inverse cold probe (3 mm), using standard NMR pulse libraries. High-performance liquid chromatography mass spectrometry (HPLC-MS) analysis was performed on an Agilent 1260 Infinity system using a Phenomenex Luna C₁₈(2) column (100 Å, 5 μm, 4.6×150 mm, Phenomenex) or a Phenomenex phenyl-hexyl column (100 Å, 5 μm, 4.6×150 mm) using the PDA detector coupled with a single quadrupole electrospray ionization mass spectrometry instrument (ESI-MS, Agilent Technologies, Inc. 6120). Purification of metabolites addressed in the current study was implemented using an Agilent Prepstar HPLC system with a preparative Agilent Polaris C₁₈-A 5 μm column (21×250 mm), a Phenomenex Luna C₁₈(2) column (100 Å, 10 μm, 10×250 mm), or an Agilent phenyl-hexyl column (100 Å, 10 μm, 10×250 mm). High-resolution ESI-MS (HR-ESI-MS) data were recorded on an Agilent iFunnel 6550 quadrupole time-of-flight (QTOF) MS instrument fitted with an electrospray ionization (ESI) source (positive mode) linked to an Agilent 1290 Infinity HPLC system with the aforementioned analytical columns. Experimental electronic circular dichroism spectra were obtained on a Chirascan CD spectrometer (Applied Photophysics, Inc.).

Initial untargeted metabolomics at a small scale was performed to identify M. morganii-derived metabolites against the rich Gifu medium components. 100 μl of each supernatant from M. morganii overnight cultures or Gifu medium alone (5×5 ml) were subjected to UPLC-QTOF-MS without extraction and the resultant raw data were processed using Mass Profiler Professional (Agilent Technologies, Inc.) or XCMS online (Tautenhahn et al., 2012, Analytical chemistry 84, 5035-5039). Metabolomics analyses led to the identification of ˜100 ion features mainly present in M. morganii relative to the rich Gifu medium control (initial ion list, FIG. 43). The bacterial cultivations were then pooled and concentrated, and the residue was fractionated into ˜30 fractions utilizing preparative HPLC (Fr.1 to Fr.30; 5→50% MeCN in water with 0.01% trifluoroacetic acid (TFA) for 30 min, 8 ml/min, 1 min elution collection window). These fractions (100 μl) were analyzed utilizing UPLC-QTOF-MS and concentrated for cell-free electrophoresis assays with circular pUC19 plasmid DNA and for investigation of bioactive entities in the fractions. Fr.20-24 exhibited genotoxic activity which led these active fractions to be combined (FIG. 12A). Ions present in inactive fractions were excluded from the initial ion list, which initially suggested ˜20 ion features for potential genotoxic small molecules in the active fractions. The combined active fractions (Fr.20-24) were again subjected to preparative HPLC for further fractionation (Fr.20-24-1 to Fr.20-24-30; 5→30% MeCN in water with 0.01% TFA for 30 min, 4 ml/min, 1 min elution collection window). The sub-fractions were assessed for their genotoxicity as described above and analyzed with UPLC-QTOF-MS. This second round of HPLC fraction screening revealed that Fr.20-24-1 to Fr.20-24-3 and Fr.20-24-5 to Fr.20-24-8 were active in the assay (FIG. 12B) and the exclusion process of ions in the inactive fraction was applied to ultimately identify 4 potential genotoxic small molecules (I-IV, FIG. 3).

Having identified those potential hits, a large cultivation of M. morganii (18 liters) was performed to characterize their structures and assess genotoxicity. The supernatant was extracted twice with ethyl acetate (20 liters each) and the extracted residue was chromatographed over a preparative HPLC system (Fr.1 to Fr.60; 5-50% MeCN in water with 0.01% trifluoroacetic acid (TFA) for 1 h, 8 ml/min, 1 min elution collection window). Single quadrupole MS analyses showed that Fr.19 to Fr.23 possessed the 4 ion features, leading them to be pooled together (Fr.19-23). The combined fraction was further separated into 60 fractions (Fr.19-23-1 to Fr.19-23-60; 5→30% MeCN in water with 0.01% TFA for 1 h, 4 ml/min, 1 min elution collection window), and the sub-fractions were analyzed. Fr.19-23-13 to Fr.19-23-15 was combined into F1, Fr.19-23-17 to Fr.19-23-20 into F2, Fr.19-23-22 to Fr.19-23-23 into F3, and Fr.19-23-26 to Fr.19-23-30 into F4, based upon each pooled fraction (i.e., F1-F4) being enriched with the targeted ion features I-IV, respectively. These 4 semi-pure fractions were evaluated for their genotoxicity utilizing the genotoxicity assay. Chromatographic analysis of genotoxic F2 demonstrated that II (indolimine-214 (1), one of the targeted ion features) and another metabolite (compound 2) were present at a ratio of 4:6.

The structures of the two compounds were characterized as a mixture utilizing one- and two-dimensional NMR analyses, including ¹H NMR, correlation spectroscopy (COSY), heteronuclear single quantum correlation (HSQC), and heteronuclear multiple bond correlation (HMBC) experiments.

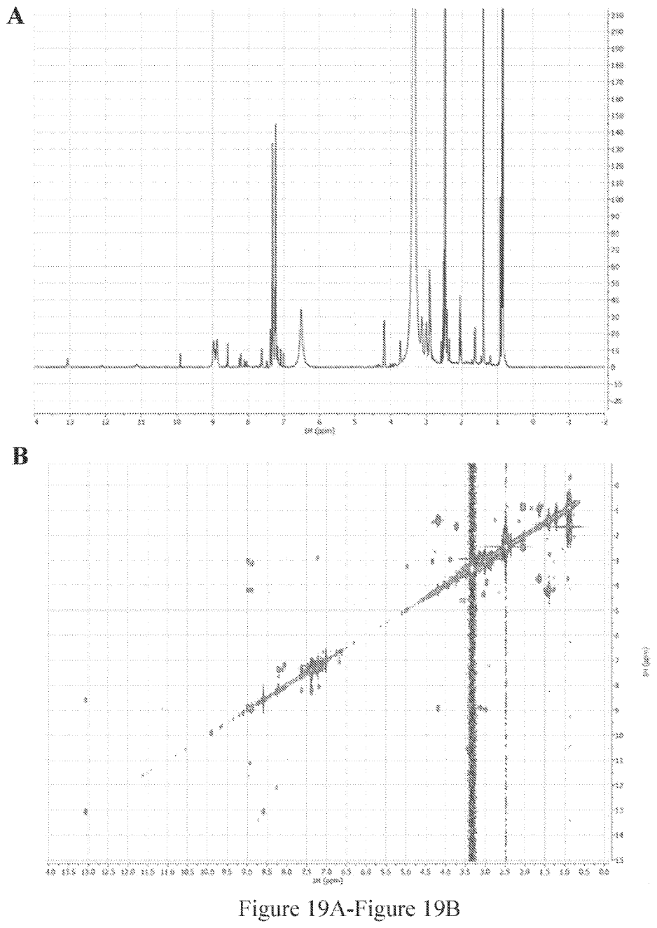

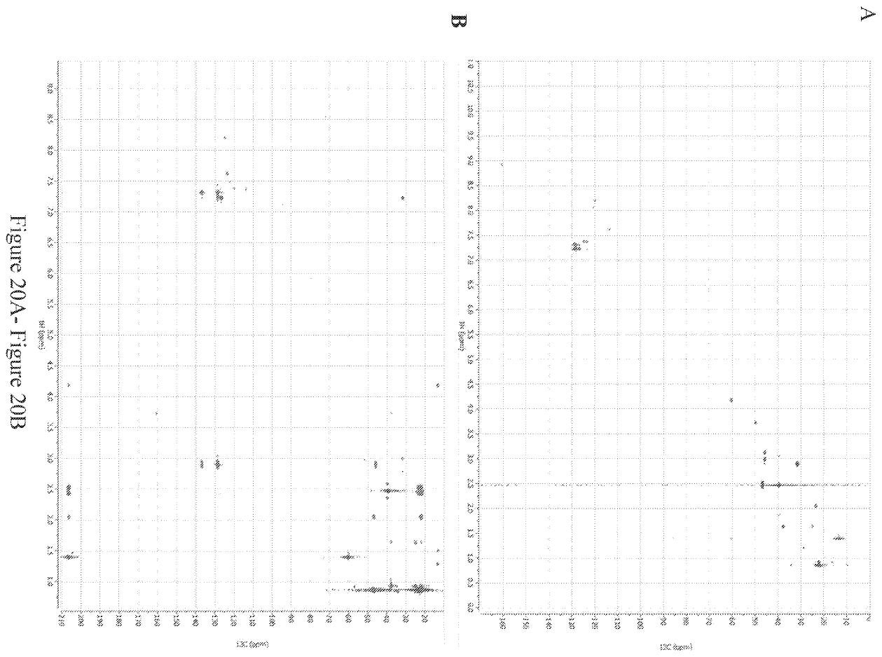

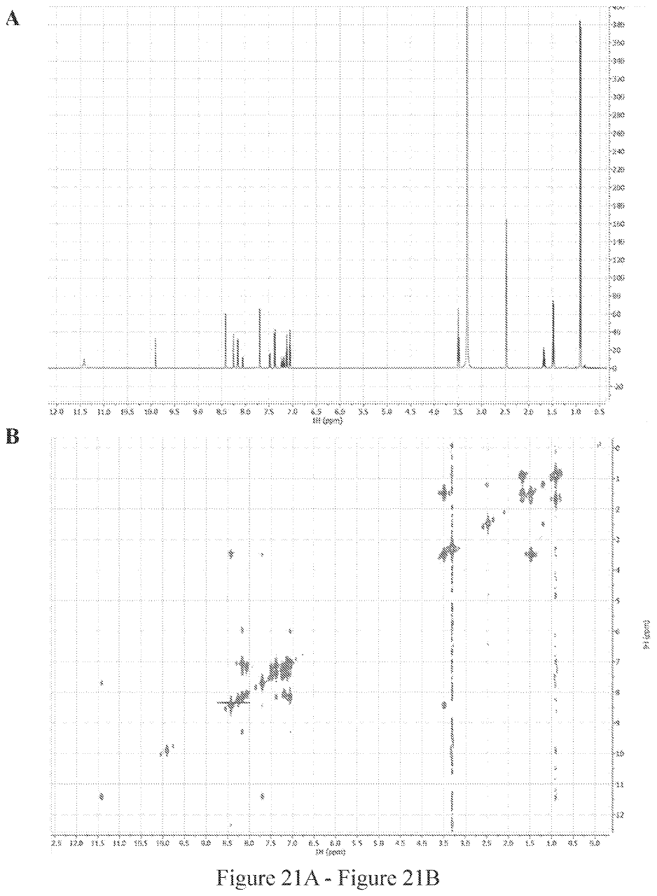

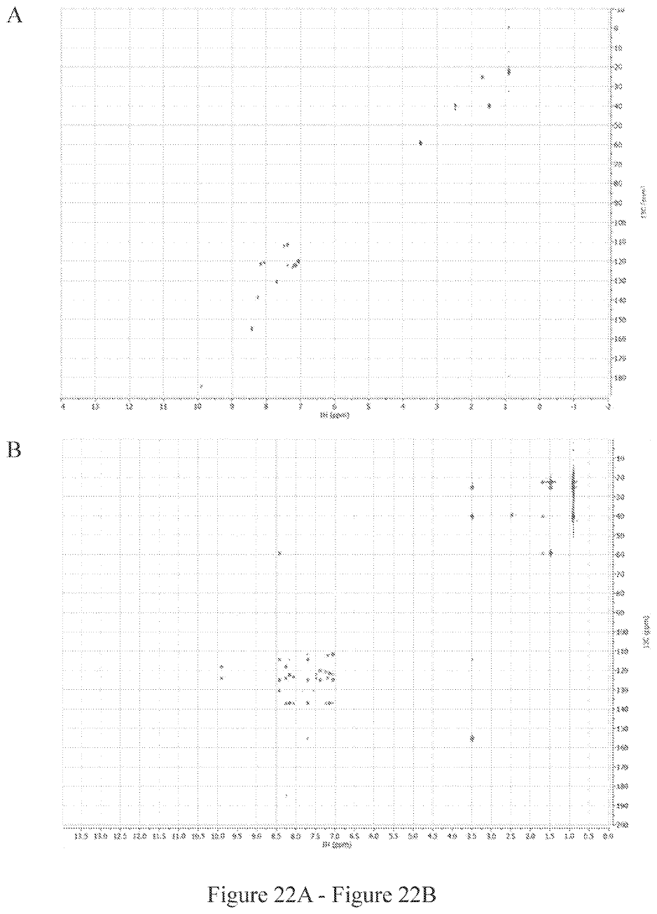

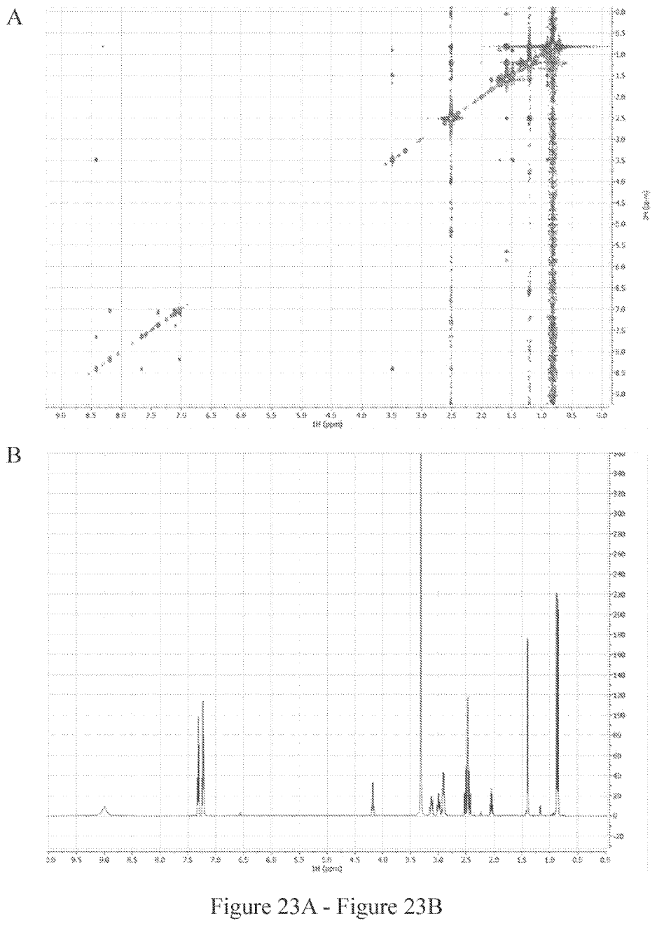

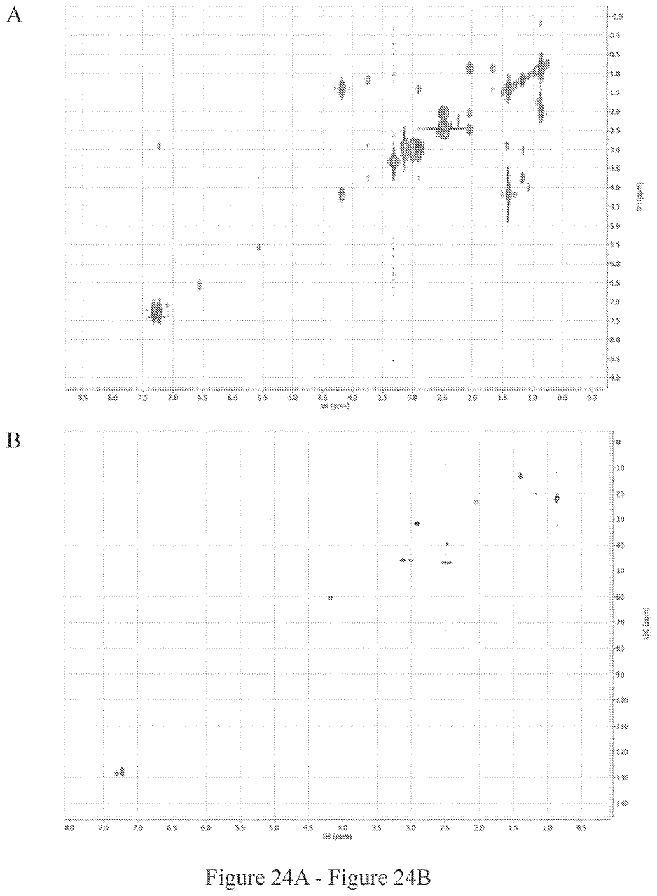

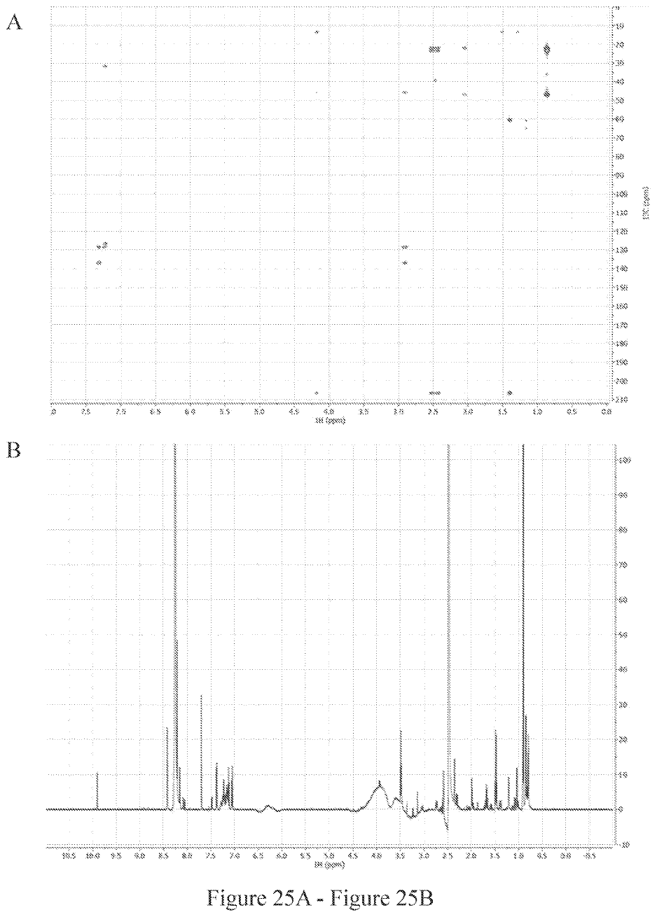

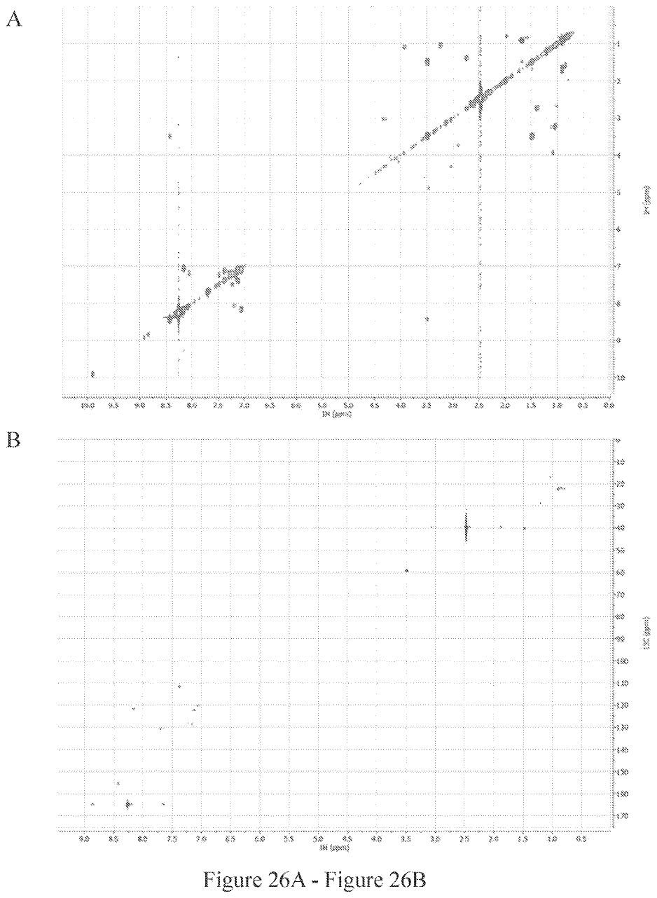

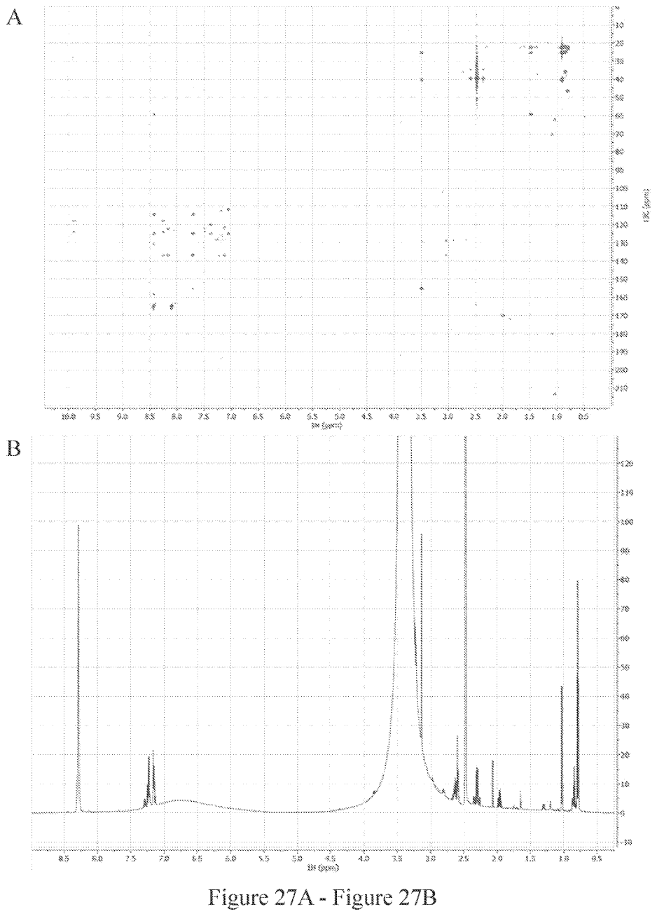

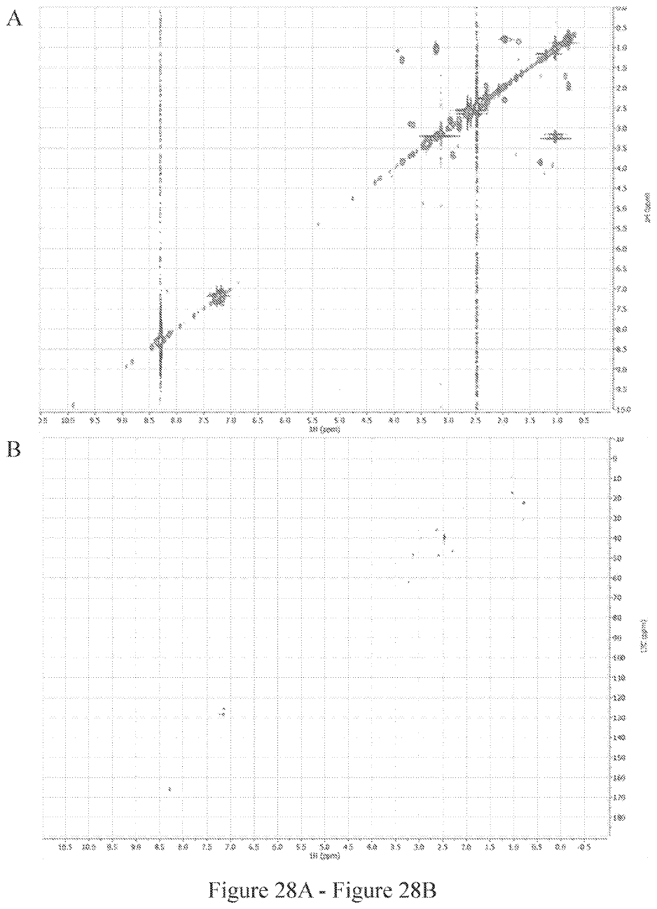

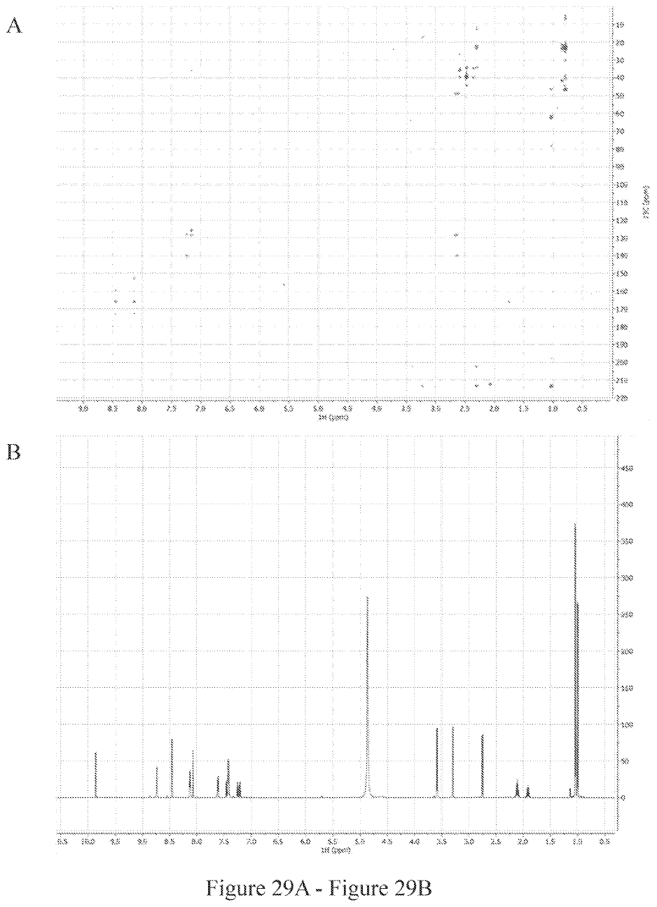

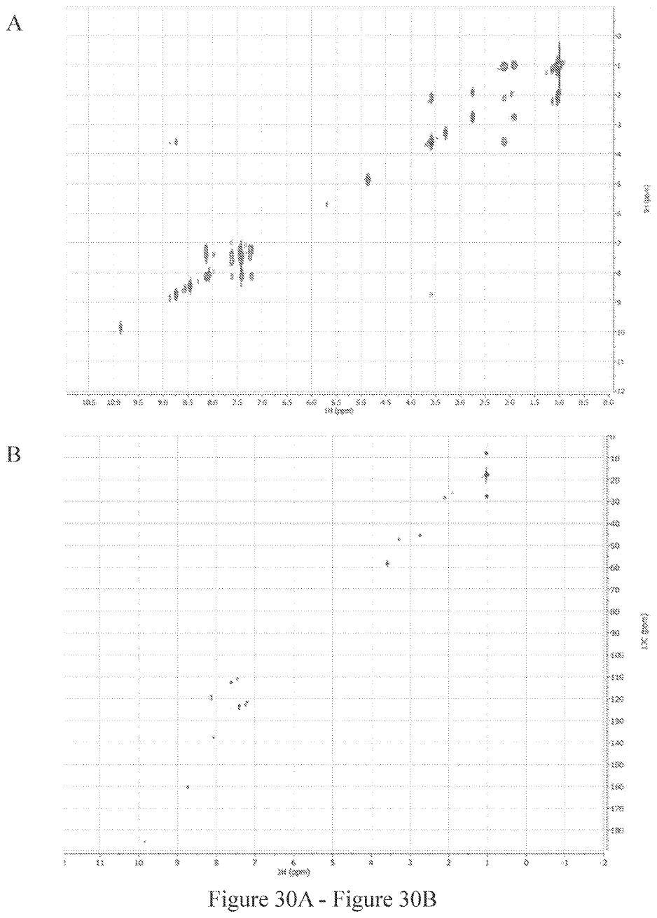

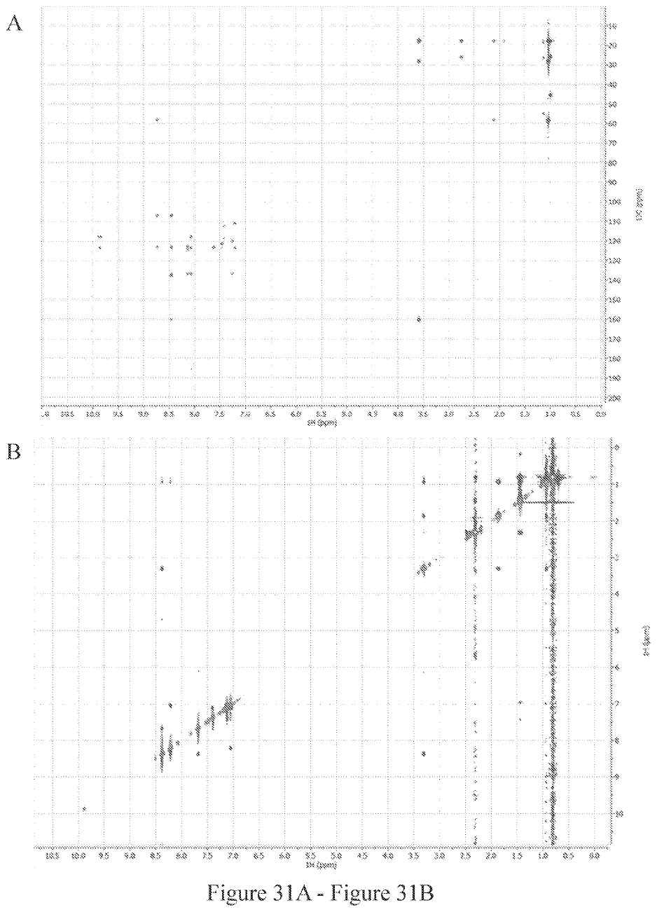

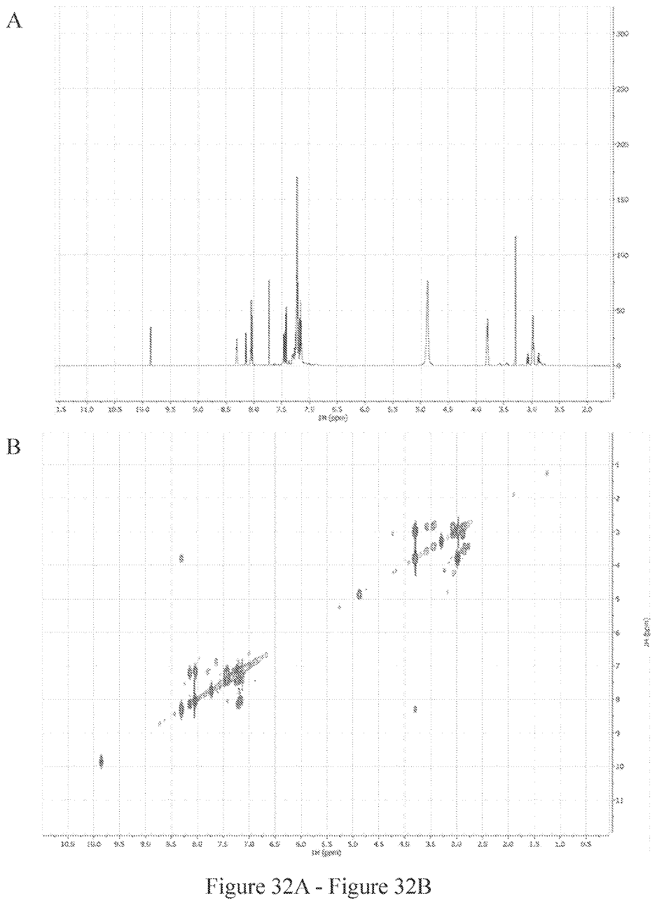

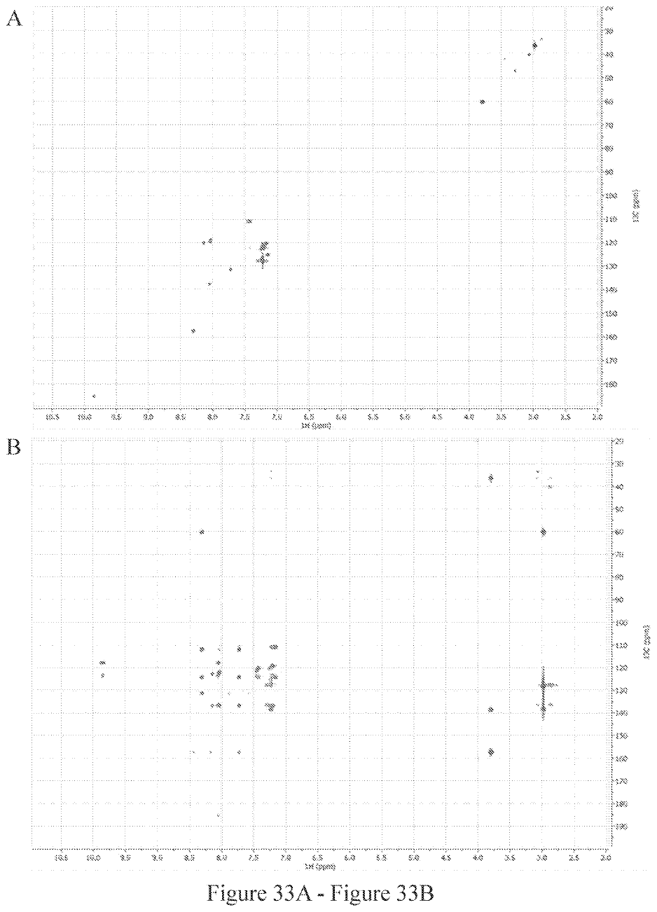

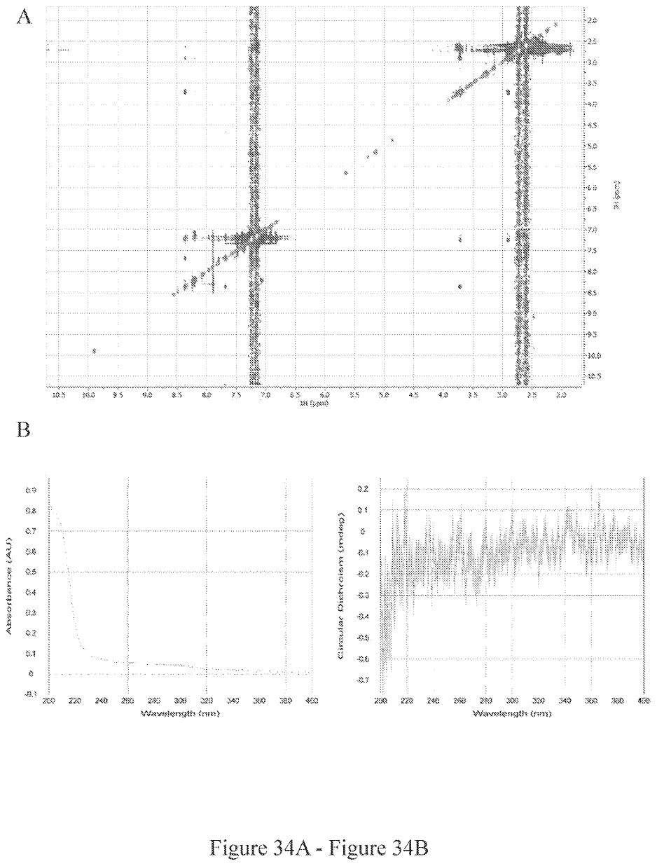

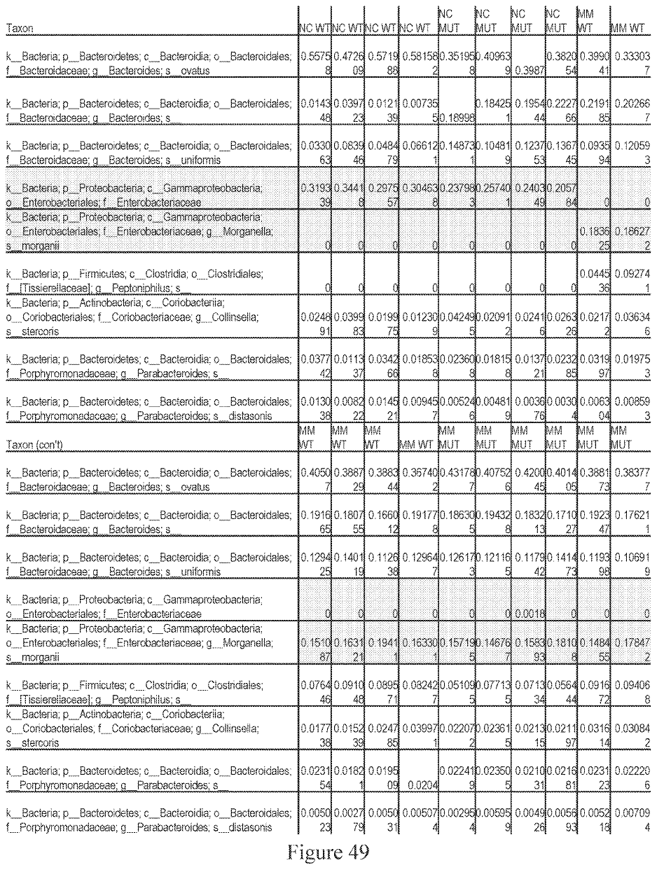

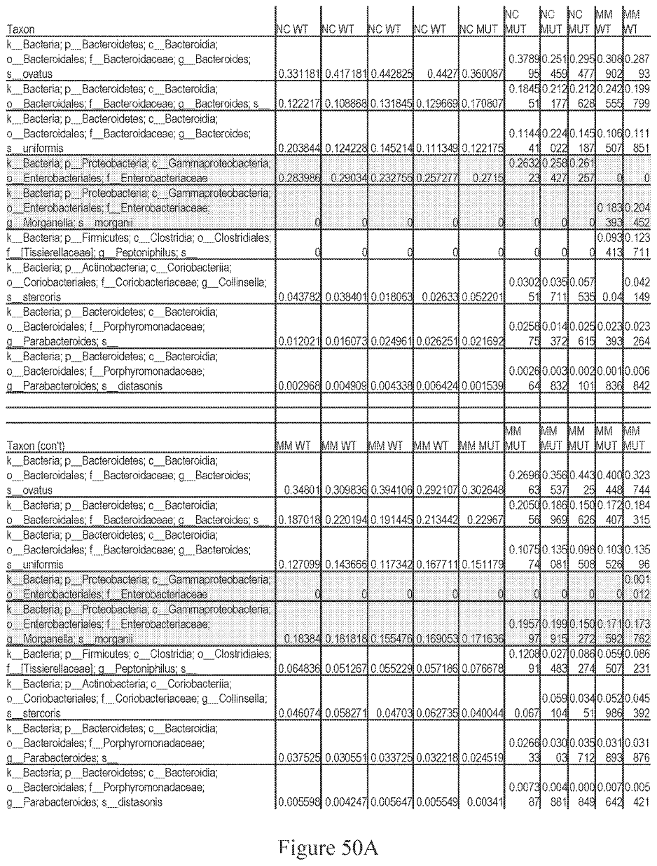

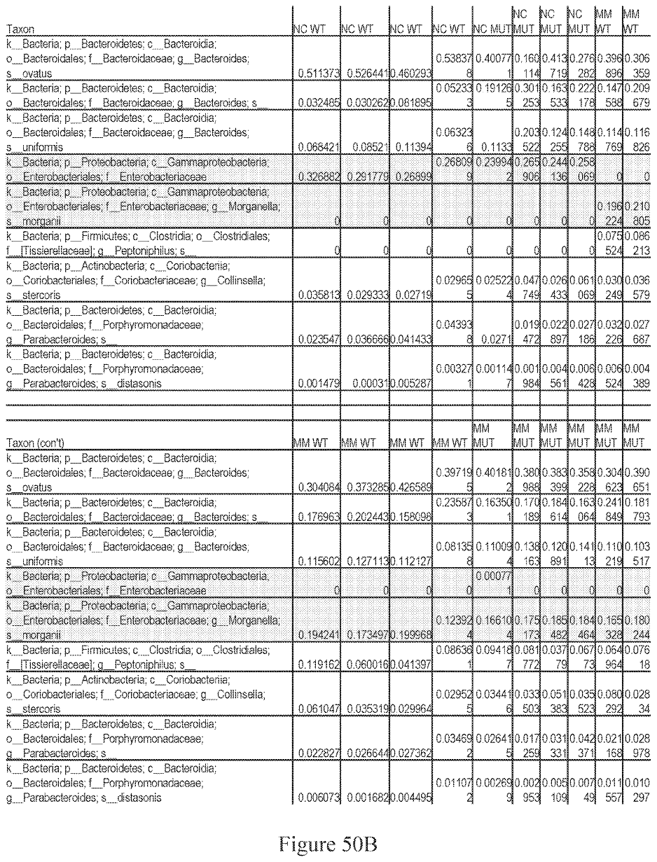

Indolimine-214 (1) was synthesized by the addition of indole-3-aldehyde (1 g) and isoamylamine (1 g) to methanol (10 ml) at room temperature. The reaction was screened by single quadrupole MS analysis and after 3 h, the solvent was evaporated, and the residue was purified utilizing preparative HPLC application. Upon purification, NMR analysis of indolimine-214 (1) showed at least ˜20% degradation into the two starting materials, due to the reversible reaction mechanism in water for formation of the target compound. Compound 2 was synthesized by dissolving phenethylamine (500 mg) and 5-methylhexane-2,3-dione (500 mg) in a mixture of water and methanol (1:1, 5 ml) followed by addition of sodium cyanoborohydride (1 g). The reaction mixture was warmed to 50° C. and incubated overnight. The crude mixture was concentrated and subjected to preparative HPLC to obtain compound 2. The aforementioned semi-fraction F2 was purified using analytical HPLC application: phenyl-hexyl column (Phenomenex, 100 Å, 250×4.6 mm, 5 μm), 18→19% MeCN in water with 0.1% formic acid for 30 min, 0.7 mL/min, 35 injections. Pure compound 2 was then garnered and its electronic circular dichroism was measured to establish that the compound is a racemic mixture. Other indolimines (3 and 4) were detected in bacterial and cecum samples and their structures were validated upon synthesis employing a similar manner for indolimine-214 (1). Indolimine-200 (3) was synthesized with indole-3-aldehyde and isobutylamine. Indolimine-248 (4) was synthesized with indole-3-aldehyde and phenethylamine. All NMR structure raw data are shown in FIG. 19-FIG. 50.

The UPLC-QTOF-MS-based quantification of indolimines was performed by a standard curve using synthetic standards followed by integration of ion counts. For quantification in bacterial production, synthetic standards were added to a Gifu medium and extracted in an identical manner to bacterial supernatants to account for extraction efficiencies in the quantification workflow. To quantify the metabolites in cecum contents, collected materials were dried and resuspended with methanol and water (1:1, 2 ml). The supernatants were concentrated and resuspended with methanol and water (1:1, 100 μl) and analyzed utilizing QTOF-MS. The synthetic standards were prepared and analyzed in the identical mixture of solvents.

### Whole-Genome-Sequencing (WGS) and Biosynthetic Gene Cluster (BGC) Analysis

Raw whole-genome sequence reads for all bacterial genomes have been deposited in the NCBI Sequence Read Archive (SRA) database in FASTQ format (Chen et al., 2019). Genome assemblies were performed as described in the previous study (Chen et al., 2022, Proc. Natl. Acad. Sci. U.S.A. 119, e2121180119). Briefly, all Illumina paired-end reads were filtered and trimmed using Trimmomatic v.0.38 (Bolger et al., 2014, Bioinformatics 30, 2114-2120) with the following parameters: ILLUMINACLIP: NexteraPE-PE.fa:2:30:12:1 true LEADING:3 TRAILING:3 MAXINFO:40:0.994 MINLEN:36. The four output files after trimming included two (forward and reverse) FASTQ files with paired reads and two FASTQ files with unpaired reads. All four files from each strain were assembled into contigs using SPAdes 3.13.0 (Nurk et al., 2013, J. Comput. Biol. 20, 714-737) with the default parameters for paired-end libraries.

AntiSMASH analysis: Assembled contigs were input into the antiSMASH portal (antismash.secondarymetabolites.org/ #!/start) for biosynthetic gene cluster (BGC) exploration with default settings (Detection strictness: loose) (Blin et al., 2019, Nucleic Acids Res. 47, W81-W87). Results are summarized in FIG. 38-42.

WGS annotation: For genome assembly of M.morganii NWP135, contigs were uploaded to the Rapid Annotation using Subsystem Technology (RAST) server (Brettin et al., 2015, Sci. Rep. 5, 8365) using the default RASTtk settings. The annotated genome was downloaded as spreadsheets (FIG. 43-44).

### Human Microbiome Project 2 (HMP2) and TCGA Data Mining

Publicly available merged taxonomic profiles (ibdmdb.org/tunnel/products/HMP2/WGS/1818/taxonomic_profiles.tsv.gz) and metadata (ibdmdb.org/tunnel/products/HMP2/Metadata/hmp2_metadata.csv) of the HMP2 (The Integrative HMP Research Network Consortium, The Integrative Human Microbiome Project, 2019) were downloaded from The Inflammatory Bowel Disease Multi'omics Database (IBDMDB), which was funded by the NIH Human Microbiome Project NIDDK U54DE023798 (ibdmdb.org). Then the relative abundance of C. perfringens, C. ramosum and M. morganii were analyzed to compare the differences between healthy people with IBD patients (UC or CD patients).

TCGA data is available from the website (cancermicrobiome.ucsd.edu/CancerMicrobiome_DataBrowser). Links are also directly available on the website to the data repository (microbio.me/pub/cancer_microbiome_analysis) (43).

### Gene Identification and IPTG-Inducing Indolimine Synthesis

The 3 potential proteins were found after NCBI blast with the AA sequence of valine decarboxylase in Streptomyces viridifaciens (39) and contained significant orthologs (FIG. 44). The codon-optimized DNA sequences (IDT) of Peg1085, Peg1320 and Peg3098 were listed in FIG. 44. Add BamHI and HindIlI restriction enzyme sites on both ends, synthesize gBlocks and construct over-expression system with pET-28 plasmids (primers were listed in FIG. 44). Then the plasmids were separately electrotransformed into E. coli BL21(DE3) (NEB, C2527I).

To induce production of indolimines from transformed E. coli, single colonies were picked and grown overnight at 37° C. in LB with carbenicillin (Carb). Dilute 1:100 in 2 ml TB medium with Carb and grow 3-4 h at 37° C. Prepare 1 ml TB+Carb+1 mM IPTG+1 mM IAld+1 mM amino acids (leucine, valine or phenylalanine) and prewarm to 37° C. before use. After 3-4 h remove 1 ml at 37° C. and collect supernatants as pre-IPTG samples. Add prewarmed 1 ml TB+Carb+1 mM IPTG+1 mM IAld+1 mM amino acids and incubate at room temperature overnight for slow induction. Then collect supernatants as post-IPTG samples and bring pre-IPTG and post-IPTG samples for QTOF-MS based identification of indolimines.

### Random Mutagenesis Library Construction

The random mutagenesis library of M. morganii NWP135 was constructed with EZ-Tn5™<R6Kγori/KAN-2>Tnp Transposome™ Kit (Lucigen, TSM08KR) following the manufacture's protocol. Briefly, M. morganii NWP135 was inoculated into medium from single colony and cultured overnight. Electroporation materials including transposome, electroporation buffer (0.5 M sucrose) and 2 mm Gene Pulser/MicroPulser Cuvettes (Bio-rad, 1652086) were put on ice in advance. Overnight growth culture was diluted into OD600=0.01 and incubated into exponential stage in Gifu medium (3˜4 h). 15 ml bacteria were collected through centrifuging at 6,500 g for 10 min at 4° C., washed 3 times with ice-cold 0.5 M sucrose and resuspend in 100 μl 0.5 M sucrose (×300 concentrated). 1 μl Transposome (˜100 ng transposon DNA) was added into bacteria and incubated on ice for 30 min-1 h. Then the mixture was transferred into 2 mm cuvettes and electroporated at 2500 V, 25 μF, 200Ω. The mixture was immediately transferred into 900 μl pre-warmed SOC medium (NEB, B9020) and recover bacteria for 3 generation (˜2 h). The recovered bacteria were plated on ˜25 LB agar plates with kanamycin and incubated in overnight (do not incubate for longer time to avoid false-positive colonies) at 37° C.

˜150 ml fresh M. morganii cultures were used to make ˜250 agar plates. An automatic colony picker was used (Molecular Devices QPix 420) to pick ˜16, 000 single colonies into 40 384-well plates containing LB (Kan+) medium.

### Tn-Seg Based Mutant Identification

Overnight-growth M. morganii mutant bacterial cultures were mixed with 40% glycerol/LB at 1:1 to make frozen stocks. Fresh bacterial cultures were performed combinatorial pooling with Biomek NXP Automated Liquid Handler (Beckman Coulter) and Tn-seq with customized primers as described by Knockout Sudoku (Anzai et al., 2017, Nat. Protoc. 12, 2110-2137Anzai et al., 2017, Nat. Protoc. 12, 2110-2137). Briefly, 40 384-well plates containing fresh bacterial cultures were aligned into 5 rows and 8 columns. Then ˜16, 000 single colonies were volume-equally pooled into 5 plate row libraries (PR1-PR5), 8 plate column libraries (PC1-PC8), 16 row libraries (R1-R16) and 24 column libraries (C1-C24), 54 libraries in total with H₂O as a blank control library (FIG. 16A, FIG. 45-47). Genomic DNA from pooled libraries were extracted with DNeasy UltraClean Microbial Kit (Qiagen, 12224-250). 200 ng DNA from every library was used to construct amplicon sequencing libraries through a two-step hemi-nested PCR reaction with customized primers (FIG. 45-47). The first reaction amplified a portion of the genome adjacent to the EZ-Tn5 transposon with forward Tnseq1.1 and reverse Tnseqarb1 and Tnseqarb2 primers. The second reaction added Illumina universal adaptor (UA) and xN (random sequences to avoid saturation of any color channel) with 4 universal forward primers, and Illumina index sequence (Index), diverse barcode sequence (unique barcode for every library, BC) and flow-cell-binding sequence (BS) with reverse primers (FIG. 16A, FIG. 45-47). All PCR reactions were performed with OneTaq DNA polymerase (NEB, M0480L) following the protocol of Knockout Sudoku. 5 μg pooled PCR products were purified at 0.5-1 kb size by gel extraction with Monarch DNA Gel Extraction Kit (NEB, T1020S). After DNA quantification with Qubit™ dsDNA HS Assay Kit (Thermo Fisher Scientific, Q32854), the library was diluted into 2-4 nM for denaturation and 10 pM for loading into Miseq Reagent kit v3 (Illumina, MS-102-3001). Sequencing was performed in single-index, 100-bp single-end read mode with Miseq machine (Illumina).

Sequencing results from Miseq were used to identify a M. morganii mutant strain with Tn5 transposon-inserted aat gene. Briefly, the raw sequences were firstly trimmed to discard EZ-Tn5 transposon sequences with cutadapt

$cutadapt -j 10 -g GGGTTGAGATGTGTATAAGAGACAG
(SEQ ID NO: 129) -o *.fastq.gz *.fastq.gz

Then the trimmed sequences were blasted to aat gene (Peg1085) of M. morganii NWP135 with Geneious Prime. One out of ˜16, 000 colonies was found to be blasted with aat gene, indicating that the transposon was inserted into this gene. This strain was in [R3, C9, PR1, PC4], Plate4 C9.

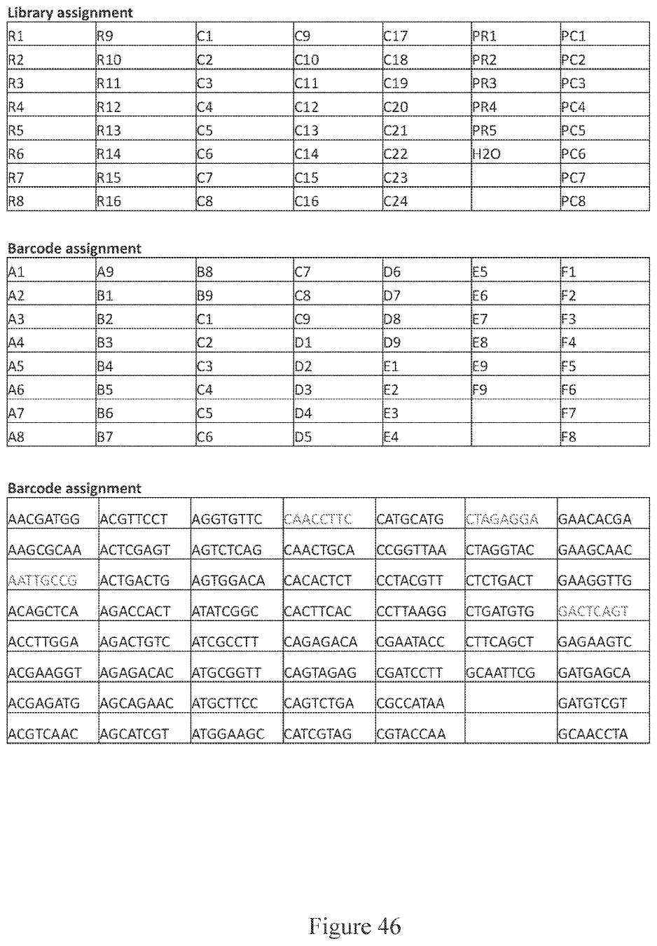

To confirm the transposon insertion in aat gene of the selected mutant strain, bacterial pellets of mutant and wild-type strains were collected, and genomic DNA were extracted. Then part of the aat gene was amplified with primers (FIG. 45-46): transposon-inserted aat gene was expected to be ˜2.5 kb while wide-type aat gene was expected to be ˜500 bp.

### FITC-Dextran Permeability Assay

Mice were colonized with aat+ or aat− M. morganii for 1-2 weeks. Then IAld and leucine were added into drinking water as 2 g/l for 2 weeks to increases the production of indolimine-214 (1). FITC-Dextran (TdB Labs, 60842-46-8) was dissolved in sterile water at a final concentration of 80 mg/ml. Mice were orally garaged with 150 μl FITC-Dextran and collected retroorbital blood after 3-4 h. Plasma samples were collected after centrifuging at 3,000 rpm for 10 min and diluted 1:10 in PBS. 100 μl samples were transferred into black opaque-bottom 96-well plate and relative fluorescence units were read at 530 nm with excitation at 485 nm.

### Bulk RNA-Seq

Mice from the FITC-Dextran assay were sacrificed for collection of colons. Colon tissues were dissected and opened longitudinally, washed with ice-cold PBS, cut into small pieces and digested in 20 ml DPBS+2 mM EDTA at 100 rpm 37° C. for 15 min in the shaker. Then add BSA to stop isolation, vigorously shake the tube by hand for 30 s and filter the cells with 70 m membrane. Colonic epithelial cells were centrifuged and collected, then RNA was extracted with RNeasy Mini Kit (Qiagen, 74106) and DNase on-column digestion following the protocol. RNAseq libraries were prepared following the Illumina Stranded Total RNA Prep Ligation with Ribo-Zero Plus (Illumina, 20040529). Quality of libraries was checked and HiSeq paired-end, 100 bp sequencing was performed by Yale Center for Genome Analysis staff.

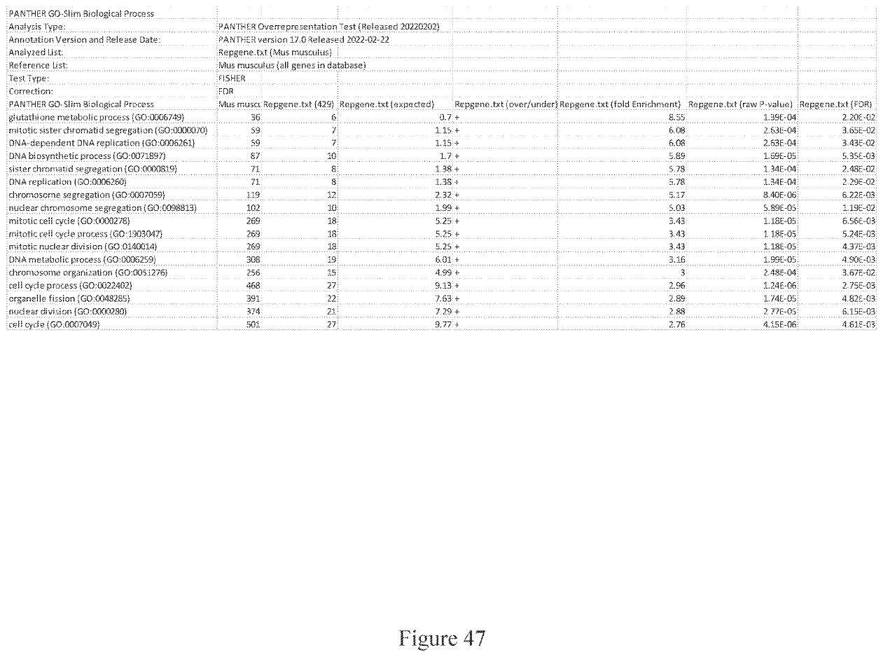

Sequencing data were trimmed, aligned, and gene counts quantified using Partek Flow (v6.0). Upregulated genes in mice colonized with aat+ M. morganii were determined with a threshold of 0.4 on log 2 (fold change) and P<0.05 (FIG. 47). Then the gene list was analyzed for GO enrichment using statistical over-representation test in Panther v14 available at geneontology.org (FIG. 47). GO bubble plot and volcano plot were made with R.

### DSS-Induced Acute Colitis and AOM/DSS Models

Azoxymethane (AOM, Millipore Sigma, A5486) was dissolved in PBS at 2 mg/ml. Dextran sulfate sodium (DSS) was purchased from TdB Labs (batch no. DB001-42).

Age- and sex-matched 5-8-week-old germ-free wild-type C57BL/6 mice were used for all studies. All mice were bred and maintained at the Yale University School of Medicine and all treatments were approved by the Yale Animal Care and Use Committee (IACUC) (IACUC protocol number: 11513, the Yale Animal Welfare Assurance number: D16-00146).

Germ-free mice were colonized via oral gavage with individual bacterial cultures or mixed bacterial communities (˜10⁸ CFU in 100 μl) grown under anaerobic conditions and stored in a sealed glass vial until immediately prior to oral gavage.

For DSS-induced colitis model, after 1-2 weeks of colonization, mice were treated with 2% DSS in the drinking water for 7 days. Fecal samples were collected on day 0, 2, 4 and 7. Mice were weighted every day. On day 7, mice were sacrificed, fecal samples were collected for CFU or fecal lipocalin 2 ELISA; colon tissues were collected, measured for length, and then fixed in Bouin's buffer for 24 h. Fixed tissues were transferred into 70% EtOH and stored for longer time.

For AOM/DSS model, after 10 days of colonization, wild-type mice received intraperitoneal injections of AOM (10 mg/kg). 5 days after the first AOM injection, mice were treated with 2% DSS in the drinking water for 5 days, followed by 16 days of untreated, sterile water. This cycle was repeated twice with 1.5% DSS. Fecal samples were collected at the experimental endpoint (day 78) for 16S rRNA profiling or fecal lipocalin 2 ELISA. Mice were sacrificed on day 78, colon tissues were collected, and tumor number and overall tumor load were recorded. Macroscopically-visible tumors were enumerated for each mouse and tumor load was calculated as a sum of tumor scores (graded by size of all tumors) per mouse. Tumor sizes were graded from 1 to 5 as follows: Grade 1, very small but detectable tumor; Grade 2, tumor covering up to one-eighth of colonic circumference; Grade 3, tumor covering up to one-fourth of the colonic circumference; Grade 4, tumor covering up to half of the colonic circumference; and Grade 5, tumor covering more than half of the colonic circumference.

Colon tissues were swiss-rolled (lumen side out) and fixed in 4% PFA (J. T. Baker®™, S89807) in PBS overnight. Paraffin embedding and H&E staining was performed by Yale Pathology Tissue Services (YPTS). Histopathologic evaluation of colon tumor and inflammatory scoring was performed by a gastrointestinal pathologist (WJH). For inflammatory scoring, previously reported method was used (Dubé et al., 2012, J. Clin. Invest. 122, 2780-2792). Briefly, epithelial cell loss, crypt inflammation, lamina propria (L.P.) mononuclear cells, L.P. polymorphonuclear cells (PMNs), and epithelial hyperplasia were evaluated in 0-3 scale (total 0-15).

### ELISA

Fecal pellets were weighed, dissolved in 1 ml PBS per 100 mg fecal material, and disrupted via bead beating (MP Biomedicals, Lysing Matrix D, 6913) for 10 s in a Biospec BeadBeater. Fecal water samples were collected after spinning at 10,000 g for 5 min.

Fecal lipocalin-2 ELISAs were performed using the Mouse Lipocalin-2/NGAL DuoSet ELISA kit per the manufacturer's instructions (R&D Systems, DY-1857). Briefly, plates were coated for 2 h at 37° C. with 50 μl of Capture Antibody diluted in PBS. After blocking for 1 h at room temperature with 100 μl of 1% BSA/PBS, 50 μl fecal water samples or standards were incubated for 2 h at room temperature. 50 μl of Detection Antibody diluted in 0.1% BSA/PBS was incubated for 1 h at room temperature followed by Streptavidin-HRP diluted 1:200 in 0.1% BSA/PBS for 30 min at room temperature.

Absorbance was measured at 450 nm after development using the Pierce TMB Substrate Kit (Thermo Fisher Scientific, 34021) and 2M H₂SO₄. Total protein levels were measured using the Pierce BCA Protein Assay Kit (Thermo Fisher Scientific, 23225) according to the manufacturer's instructions.

16S rRNA Sequencing

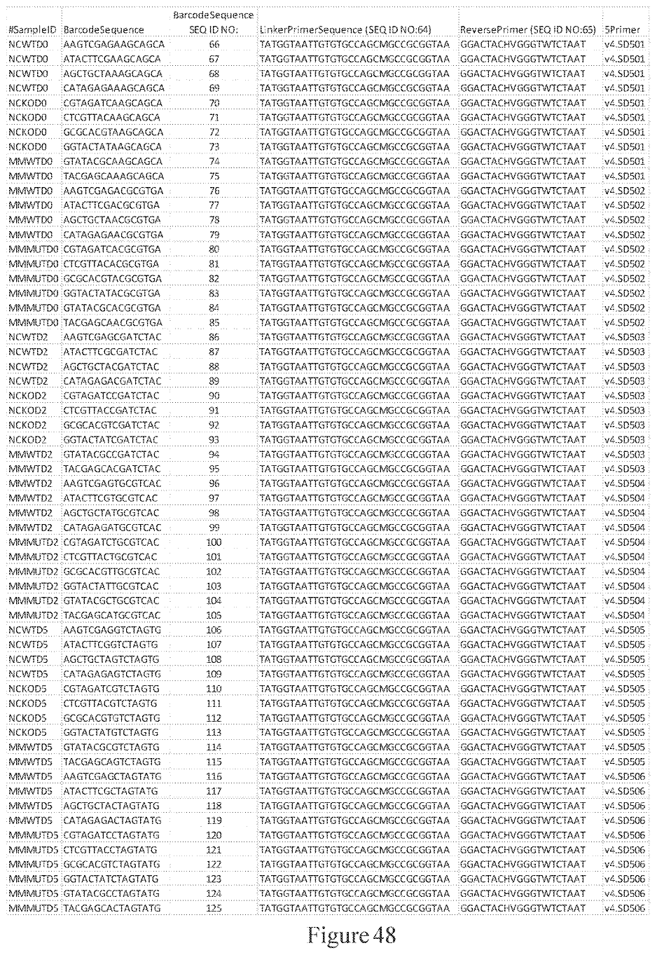

Collected fecal samples were collected and DNA were extracted followed by genomic DNA isolation (Qiagen DNeasy PowerLyzer PowerSoil Kit, 12855). Then 16S rRNA gene V4 region was amplified from each sample by PCR according to a dual index multiplexing strategy as previously described (Palm et al., 2014, Cell 158, 1000-1010). Amplicons were normalized and cleaned (Agencourt AMPure XP purification beads; Beckman Coulter, A63881) before pooling and library quantification (KAPA Biosystems KK4835; Applied Biosystems QuantStudio 6 Flex instrument). Sequencing was performed on an Illumina Miseq in 2×250 PE configuration, using a 500 cycle V2 reagent kit (MS-102-2003). Metadata of 16S rRNA sequencing and analyzed relative abundance results were summarized in FIG. 48-50.

### Statistical Analysis

Statistical analyses were performed with GraphPad Prism 9 (GraphPad Software). Representative data were presented as mean±SEM of three or two independent experiments unless otherwise stated. In the figure legends, n=the number of independent biological replicates (cells or animals) per group, and N=the number of independent experimental replicates. Differences between groups were calculated by Student's unpaired t-test, one- or two-way ANOVA. Significant differences are labeled as n.s., no significance or * P<0.05; ** P<0.01; *** P<0.001; **** P<0.0001.

### Figure Design

Phylogenetic trees were generated and annotated with GraPhlAn. Schematic figures were created using BioRender.

### Sequences

Peg1085; codon-optimized nucleic acid sequence
(SEQ ID NO: 1)
ATGAATCAATCGGAATTGATTACACTGGCCTCCGCGAATGCTCTG
AACAAAGACTTTGAAGTAAAATATCAAAACGTGATTTCCGACTTC
TTTTCCCGTGACCCTGGAAAATGGCCCATTTTCAATCACCCCCAA
ATCCAAGCAATTACACAGTTCCGTCAGACCACAGATGCGGATGTA
CAGCAGATCAATCGCTACCCTCAGGGTAAGGACTTGTTCGCGCAA
CTGGCAGGCGAATCGCACGTCCGTCAGAACGTTATCCGTCCAGGT
GAAGGTCAGGACGATTTACTTGTGTTCGCATCAGCCTTATGCAAA
AATTGGGAGAACCCCTTAGCCGTAGAGAACGTCATTGCTATGCCA
AGCGACCCAGCGGTCTATGGCTCCATGTTGGGTTTATTAGGAAAT
CCGAACATGGTATACTGTGAATATTCTGGCGTAGCAGACAATATG
GAGAAAACCGTGATTCGTAAGGTGGCAAATTTAATTGGATACGAT
GCTGACAAAGCTAGCGGCTTGTTCACCCAGGGGGGAACTATGTGT
AATTTGTATGGATACTTATTTGGTATTCGCAAATCGCTTAAACAA
AGTAAGCATTTAGGCATGTCAGTCGACCAGGATTTTCGTATTATC
AATTCGCAGGGGGGTCACTACAGCAACATGACTAATCTTTCTCTT
CTTGGGGTAGACATCACGAACAAAACTATCCGCATCCGTGTGGCC
AGCGATAACACTATTGATCTTGCGGACCTTGAGCAACAAATTCGT
GCCTGTTACACTGTTCATTGCAAAATTCCTGTCATCTTGCTTACC
GCTGGGACAACCGATACGTTTGGTGTTGACGAAATCAAACAAGTC
TACGATCTGCGCAATCGTCTTTGCGAGGAGTTCGAGATCACGGAG
AAACCGCATATCCATGTAGACGCTGCGGTTGGGTGGCCAATCATT
TTCTTTATCGACTATGATTTCAATACGAATCCCCTTGCTATTAAT
GATGCAACCTTGGCAGGGTTGCGCCATAATGTAGAGAAATTCAAG
CAATTAAAATATGCGGATTCCATTACAATCGACTTCCACAAATGG
GGCTACGTCCCATACACGAGCTCCCTGGTTATGGTGCGCGACGGA
GACGATTTCAAGGCGTTAGAAAATGATCCTGAAAATTTTACATAT
TTTGAACATGCGCTTGAAGGGCAAACACATTTACAATCAACAATT
GAATGTAGCCGCTCGGGCGTAGGGGTGTTCGGAGCGTACGCGGGT
CTTCACTACCTTGGTGTAGAGGGCTATCAGACTATCATTGCCCAC
TGTCTGCAAAACGCGAATTATATGCGTAACCAGCTGTTATCAATG
GGGAACGCCTGCGTCATGGTCCCTGAGAACCAAGGTCCCTCCGTC
GGTTTCCGTTTATACTCACCAAAACTGGTCAACGACCCGCAAGCT
ATGTTTGCTCACGAATTAACGTGTGCTGGGGATAAGACCGCTTAC
GACATGATGGTGCGTAATTCACGTTGGCACCGCGAGTTATTCCTG
AAACGCGGAAAGGCCGGTTTGTTCACTAACTGGGTGGACTCTATC
GCTTGTAGTGCCTATGCTGAGCACAACCGCTTTGCCTATATTCCG
GGAGAAAAGGCAGTCTTCATGAACCCTGTTACCCAACGCCATCAC
ATCGACGCGTTCGCTAAGATGCTGAAGACGATGTCAGCCGAGTAA
Peg1085; nucleic acid sequence
(SEQ ID NO: 2)
gtgaatcagtcagaacttatcacgctcgcctctgcgaatgcgtta
aataaagatttcgaagttaaataccaaaacgtcatcagtgatttc
ttcagccgggatcccggcaaatggccgatttttaatcacccgcaa
attcaggcaattactcagttccgccagaccaccgacgcggatgtg
cagcagatcaaccgctatccgcagggtaaggatctttttgcacaa
ctggccggtgagtcacatgtccgtcagaacgtgatccgtccggga
gaagggcaggatgatttactggtgtttgcgtccgcgctgtgcaaa
aactgggaaaacccgctggccgtggaaaacgttatcgccatgccg
tcagacccggcagtttacggctctatgctgggcttgctgggtaac
ccgaacatggtgtactgcgaatactccggtgtggcggataacatg
gaaaaaaccgttatccgtaaagtggccaatctgattggctatgat
gcggacaaagcatccggtctgttcacccagggcggaaccatgtgc
aatctgtacggctatctcttcgggatccgtaaatcgctgaaacag
tcaaaacatctcgggatgtccgttgatcaggatttccggatcatc
aactcacagggcggtcactactccaatatgactaacctgtccctg
ttaggtgtggatatcacaaataaaacaatccgtatccgggtggcg
tcagataacaccattgacctggcggatctggagcagcaaatccgt
gcctgctataccgtccactgtaagatcccggtgatcctgctgacc
gcgggaacaaccgatacctttggtgtcgatgaaatcaaacaggtt
tatgatctgcgcaaccgcctgtgtgaagagttcgaaattacagaa
aaaccgcatattcacgtggatgcggcggtcggctggccgattatt
ttctttattgattatgattttaacaccaacccgctggcgattaac
gatgccaccctggctggtctgcgtcacaatgttgagaaatttaaa
cagctgaaatatgcagattccatcactatcgacttccacaaatgg
ggttatgtgccgtatacctcgagtctggtgatggtgcgtgacggt
gatgatttcaaagcactggaaaatgacccggaaaacttcacctat
tttgagcatgcgctggaaggacagactcacctgcaatccaccatt
gaatgcagccgcagcggtgtcggtgtgttcggggcttatgccgga
ttgcattatctgggggttgagggttatcagaccatcattgcgcac
tgtctgcaaaatgcgaactacatgcgcaaccaactgctatccatg
ggcaatgcctgtgtgatggtgccggaaaaccaggggccgagtgtc
ggtttccgtctctattcaccgaaactggtgaatgatccgcaggca
atgtttgcacacgaactgacctgtgccggagataaaacggcctat
gacatgatggtgcgcaacagccgctggcatcgtgagctgttcctg
aaacgcggtaaagcaggactgtttaccaactgggtggattctatc
gcctgctcggcctatgcggagcataaccggttcgcgtatattccg
ggggaaaaggcggtctttatgaacccggtgacgcagcgtcatcac
atcgatgcgtttgcaaaaatgcttaaaaccatgagtgcagaataa
Peg1085; amino acid sequence:
(SEQ ID NO: 3)
MNQSELITLASANALNKDFEVKYQNVISDFFSRDPGKWPIFNHPQ
IQAITQFRQTTDADVQQINRYPQGKDLFAQLAGESHVRQNVIRPG
EGQDDLLVFASALCKNWENPLAVENVIAMPSDPAVYGSMLGLLGN
PNMVYCEYSGVADNMEKTVIRKVANLIGYDADKASGLFTQGGTMC
NLYGYLFGIRKSLKQSKHLGMSVDQDFRIINSQGGHYSNMTNLSL
LGVDITNKTIRIRVASDNTIDLADLEQQIRACYTVHCKIPVILLT
AGTTDTFGVDEIKQVYDLRNRLCEEFEITEKPHIHVDAAVGWPII
FFIDYDENTNPLAINDATLAGLRHNVEKFKQLKYADSITIDFHKW
GYVPYTSSLVMVRDGDDFKALENDPENFTYFEHALEGQTHLQSTI
ECSRSGVGVFGAYAGLHYLGVEGYQTIIAHCLQNANYMRNQLLSM
GNACVMVPENQGPSVGFRLYSPKLVNDPQAMFAHELTCAGDKTAY
DMMVRNSRWHRELFLKRGKAGLFTNWVDSIACSAYAEHNRFAYIP
GEKAVFMNPVTQRHHIDAFAKMLKTMSAE

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of identifying a gut microbe genotoxin able to damage nucleic acid, the method comprising the steps of:
a) culturing a population of gut microbes,
b) contacting a test nucleic acid with the cultured population of gut microbes, or the culture supernatant thereof,
c) assessing the test nucleic acid for damage,
d) identifying damage to the test nucleic acid,
e) isolating the gut microbe genotoxin from the gut microbe, or the culture supernatant thereof, when the test nucleic acid exhibits damage,
f) identifying the gut microbe genotoxin.

2. The method of claim 1, wherein the gut microbe genotoxin is an indolimine.

3. The method of claim 2, wherein the indolimine is indolimine-214, indolimine-200, or indolimine-248.

4. A gut microbe genotoxin identified by the method of claim 1.

5. The method of claim 1, further comprising the steps of:
a) identifying the gene responsible for genotoxin synthesis.

6. An indolimine.

7. The indolimine of claim 6, wherein the indolimine is indolimine-214, indolimine-200, or indolimine-248.

8. The indolimine of claim 6, wherein the indolimine synthesis is mediated by an enzyme encoded by a codon-optimized nucleic acid sequence of Peg1085 comprising SEQ ID NO:1.

9. A method of preventing or treating a disease or disorder in a subject in need thereof, comprising administering to the subject an effective amount of an inhibitor of indolimine, an inhibitor of an indolimine synthesis or an inhibitor of an indolimine-producing bacterium.

10. The method of claim 9, wherein the inhibitor of indolimine is at least one selected from the groups consisting of an antibody, an antibody fragment, a single chain antibody (scFv), an anticalin, a nucleic acid, an antisense nucleic acid, an antibiotic, or probiotic.

11. The method of claim 9, wherein the inhibitor of indolimine synthesis comprises an inhibitor of a decarboxylase.

12. The method of claim 11, wherein the decarboxylase is encoded by SEQ ID NO:1.

13. The method of claim 9, wherein the inhibitor of indolimine synthesis is at least one selected from the groups consisting of an antibody, an antibody fragment, a single chain antibody (scFv), an anticalin, a nucleic acid, an antisense nucleic acid, an antibiotic, or probiotic.

14. A method of identifying a subject at increased risk of developing cancer, comprising detecting in a biological sample of the subject, one or more species or strains of bacteria that produce a metabolite associated with DNA damage.

15. The method of claim 14, wherein the metabolite associate with DNA damage is an indolimine.

16. The method of claim 14, wherein the one or more species or strains of bacteria is selected from the group consisting of Bifidobacterium adolescentis, Bifidobacterium dentium, Bifidobacterium breve, Clostridium perfringens, Clostridium ramosum, Streptococcus mitis, Lactobacillus salivarius, Pediococcus acidilactici, Enterococcus asini, and Morganella morganii.

17. A genetically modified bacteria, wherein the genetically modified bacteria is genetically modified so that it is unable to express one or more genes necessary to produce an indolimine.

18. The genetically modified bacteria of claim 17, where the one of more genes necessary to produce an indolimine is a decarboxylase.

19. The genetically modified bacteria of claim 17, wherein the decarboxylase is encoded by SEQ ID NO:1.
